(19) United States
(12) Patent Application Publication
(10) Pub. No.: US 2020/0010842 A1
(43) Pub. Date: Jan. 9, 2020

(54) PLANT WITH INCREASED SILICON UPTAKE

(71) Applicant: UNIVERSITE LAVAL, Quebec (CA)

(72) Inventors: Richard BELANGER, Quebec (CA); Rupesh DESHMUKH, Quebec (CA); Francois BELZILE, Quebec (CA); Caroline LABBE, Levis (CA)

(21) Appl. No.: 15/574,414

(22) PCT Filed: May 19, 2016
(86) PCT No.: PCT/CA2016/050568

§ 371 Date: Nov. 15, 2017

Related U.S. Application Data
(60) Provisional application No. 62/164,031, filed on May 20, 2015.

(51) Int. Cl. C12N 15/82; C12Q 1/6895

(52) U.S. Cl. CPC C12N 15/8243; C12Q 1/6895; C12Q 2600/172; C12Q 2600/156; C12Q 2600/13

(57) ABSTRACT

The invention relates to nucleic acid sequences defining a genomic region conferring high silicon (Si) accumulation as discovered in the soybean (Glycine max) cultivar Hikmok sorip. Plants having this region, named HiSil, introduced in its nucleic acid exhibit increased Si uptake. Furthermore, markers associated with high Si accumulation and 5 methods of identifying high Si accumulating plants using the markers are provided. The method provided by the invention can be used to develop new plants with high Si accumulation capacity, through breeding, genetic modification or any other forms of plant propagation.

### FIELD OF THE INVENTION

The present invention relates to chromosomal intervals, marker loci, and genes that are associated with and/or confer high silicon accumulation in soybean. More specifically, the present invention relates to silicon accumulation and its benefits achieved in plants in which these chromosomal intervals, loci, and genes are introduced (by breeding, grafting or genetic engineering), thus achieving high silicon uptake. The present invention also relates to markers that may be used identify and/or select plants containing these chromosomal intervals, loci, and genes for silicon accumulation and its applications.

### BACKGROUND OF THE INVENTION

Silicon (Si) is one of the most abundant elements on the earth's surface and it comprises 50-70% of soil mass (Epstein, 1994). Si absorption in plants plays an important role in alleviating both biotic and abiotic stress tolerance. Many studies have reported Si as beneficial element and its accumulation has been corroborated with enhanced plant vigor and growth. More particularly, Si fertilization has been found to be effective against powdery mildew diseases in several crop plants including wheat, barley, rose, cucumber, muskmelon, zucchini squash, grape, and dandelion (Bowen et al., 1992; Menzies et al., 1992; Fawe et al., 2001; Belanger et al., 2003; Rodrigues et al., 2003). Si was also found to be beneficial to manage other diseases such as blast (Pyricularia grisea) and brown spot (Bipolaris oryzae) on rice, and soybean rust and Phytophthora stem and root rot on soybean (Rodrigues et al., 2003, Arsenault-Labrecque et al., 2012, Guerin et al, 2014). Si plays similar roles to alleviate abiotic stresses like salinity, heavy metals, drought tolerance and stress of extreme temperature regimes (Tuna et al., 2008, Gu et al., 2011, Chen et al., 2011, XiaoYu et al., 2013). A recent review by Epstein (2009) concluded that the beneficial role of Si is very prominent under stress whereas under normal growth conditions its role is often minimal or even nonexistent. Therefore, Si is not considered a primary essential nutrient, but rather a ‘quasi-essential’ element providing protection under stress.

Si gets absorbed in plants by the root system in the form of silicic acid and is eventually deposited as polymerized Si in its shoots and leaves (Sangster et al., 2001). Si absorption and accumulation in leaf is not uniform across plant species. In general, monocots such as rice, sugarcane and most cereals absorb large quantities of Si (up to 10% dry weight) and derive positive benefits from Si feeding (Ma and Yamaji, 2006). On the other hand, many dicots appear to be impervious to the element and gain minimal benefits from Si supplements (Hodson et al., 2005). This difference in Si accumulation has been attributed to the ability of the roots to take up Si, This would explain why experiments with Si feeding and reported benefits have yielded irregular results depending on whether the plant tested was a high or low accumulator. Therefore application of Si as a fertilizer has limitations related to whether the plant species is capable of uptake, or not.

In monocots like rice, Si influx in roots has been found to be controlled by an aquaporin termed Lsi1 (Ma et al, 2006). Later on, the molecular mechanisms involved in Si uptake were better defined with the finding of another gene, Lsi2, encoding for the efflux transport of Si (Ma et al., 2007). Both genes Lsi1 and Lsi2 were discovered using mutant resources and no natural variant has been reported yet. Si uptake and accumulation mechanisms in plants have been further validated in other monocot species such as sorghum and maize (Mitani et al., 2009). However, as with rice, natural variation appears to be lacking in sorghum and maize.

Si accumulation in dicots is less understood compared to monocots. Efforts have been made to demonstrate that Si uptake capability of dicots can be improved through transgenic approaches. Arabidopsis, a species that does not carry Lsi1 transporters, when transformed with Lsi1 genes from wheat and rice showed a 4-5 fold increase in Si accumulation (Montpetit et at., 2012). A similar approach was attempted in soybean, whereby soybean plants transformed with Lsi1 from wheat or horsetail were tested for improved Si accumulation (Guérin, 2014). However, transformed plants absorbed similar amounts as controls, a result explained by the recently identified genes GmNIP2-1 and GmNIP2-2 facilitating Si influx in soybean (Deshmukh et al., 2013). This leads to the conclusion that soybean already carries a functional Si influx transporter (Lsi1) and introgression of additional transporters (natural or transgenic) will not increase Si uptake. As a matter of fact, DNA sequences and expression of both Lsi1 genes in soybean have been found to be similar across different genotypes, thereby suggesting a lack of natural variation for Si influx transporter genes. Therefore, the possibility to breed novel varieties using these Si influx transporters is improbable.

However, there is evidence that some soybean genotypes absorb more Si than others and can thus better resist stresses such as the ones imposed by diseases under Si fertilization (Arsenault-Labrecque et al., 2012: Guérin et al., 2014). At this point, the mechanisms or genes that could confer such a property are unknown. Accordingly, identification of natural soybean variants for Si uptake capability and the mechanisms/genes responsible for the variation could definitely represent a valuable resource for soybean improvement.

### SUMMARY OF THE INVENTION

Compositions and methods for identifying, selecting and/or producing soybean plants with increased silicon accumulation and/or uptake are provided. As described herein, a marker associated with the HiSil trait may comprise, consist essentially of, or consist of: a single allele or a combination of alleles at one or more genetic loci (e.g. see Tables 15-21).

In a first aspect of the invention, there is provided a plant having introduced into its genome a nucleic acid sequence encoding a HiSil protein wherein introduction into its genome confers increased Si accumulation in the plant as compared to a control plant (i.e. LoSil plant) not comprising the nucleic acid sequence encoding a HiSil protein.

In a further aspect of the invention, there is provided a plant (e.g. elite Glycine max) which comprises in its genome a chromosomal interval comprising a H1 haplotype associated with Si accumulation.

In a further aspect of the invention, there is provided a plant which comprises in its genome a chromosomal interval associated with Si accumulation corresponding to a genomic region or portion thereof from Hikmok sorip chromosome 16 at about 92.6 cM to about 132 cM distance as indicated on a genetic linkage map from Hikmok sorip (PI372415A).

In a further aspect of the invention, there is provided a plant which comprises in its genome a chromosomal interval associated with Si accumulation corresponding to a genomic region or portion thereof from Hikmok sorip chromosome 16 corresponding to physical positions 31.15 M base-pairs to 36.72 M base-pairs. Particularly, the numbering of base pairs corresponds to the Willaims82 genomic map (i.e. Soybean genome assembly from JGI release 8. Based on the original Glyma v1.(January 2012), Herein, “Williams82 map”).

In a further aspect of the invention, there is provided a plant having introduced into its genome a chromosomal interval associated with Si accumulation of a H1 haplotype soybean plant. Particularly, a H1 haplotype derived from Hikmok sorip and wherein the plant is an elite Glycine max plant and in another embodiment wherein the chromosome interval comprises at least one molecular marker as displayed in Tables 15-21.

In a further aspect of the invention, there is provided a plant having introduced into its genome a chromosomal interval associated with Si accumulation corresponding to a genomic region or portion thereof from Hikmok sorip chromosome 16 at about 92.6 cM to about 132 cM distance as indicated on a genetic linkage map from Hikmok sorip (P1372415A). Another embodiment the chromosomal interval comprises at least one molecular marker as displayed in Tables 15-21.

In a further aspect of the invention, there is provided a plant having introduced into its genome a chromosomal interval associated with Si accumulation corresponding to a genomic region or portion thereof from Hikmok sorip chromosome 16 from physical positions 31.15M base-pairs to 36.72 M base-pairs corresponding to the Williams82 map.

In a further aspect, there is provided a plant which comprises in its genome a chromosomal interval associated with Si accumulation corresponding to a genomic region or portion thereof from Hikmok sorip chromosome 16 at about 92.6 cM to about 132 cM distance or from physical positions 33.15M base-pairs to 36.72M base-pairs as indicated on a genetic linkage map from Hikmok sorip (P1372415A).

In a further aspect, there is provided a plant having introduced into its genome a chromosomal interval associated with Si accumulation corresponding to a genomic region or portion thereof from Hikmok sorip chromosome 16 at about 92.6 cM to about 132 cM distance or from physical positions 33.15M base-pairs to 36.72M base-pairs as indicated on a genetic linkage map from Hikmok sorip (PI372415A).

In a further aspect, there is provided a plant wherein said plant comprises a HiSil trait. Further is provided a plant comprising a HiSil trait derived from Hikmok sorip or a progeny thereof.

In a further aspect, there is provided a plant comprising a HiSil allele which confers increased Si uptake, and wherein the HiSil allele comprises at least one single nucleotide polymorphism (SNP) selected from the group consisting of A(33673022), G(33673483), C(33681630), T(33682500), G(33683047), and C(33683049) as indicated on a genetic linkage map from Hikmok sorip (P1372415A).

In accordance with a particular aspect of the invention, there is provided a plant as defined herein, wherein the presence/introduction of the nucleic acid confers increased resistance to at least one pathogen from the group consisting of: nematode, rust, smut, Golovinomyces cichoracearum, Eiysiphe cichoracearum, Blumeria graminis, Podosphaera xanthil, Sphaerotheca fuliginea, Pythium ultimum, Uncinula necator, Mycosphaerella pinodes, Magnaporthe grisea, Bipolans oryzae, Magnaporthe grisea, Rhizoctonia solani, Phytophthora sojae. Schizaphis graminum, Bemisia tabaci, Rhopalosiphum maidis, Deroceras reticuiaturn, Diatraea saccharalis, Schizaphis graminum and Myzus persicae; or a combination thereof.

In accordance with a particular aspect of the invention, there is provided a plant having increased resistance to a stress selected from the group consisting of: diseases (such as powdery mildew, Pythium ultimum, Phytophthora root rot, leaf spot, blast, brown spot, root-knot nematode, soybean cyst nematode, soybean vein necrosis virus, soybean stem canker, soybean sudden death syndrome, leaf and neck blast, rust, frogeye leaf spot, brown stem rot, Fusarium, or sheath blight); insect pests (such as whitefly, aphid, grey field slug, sugarcane borer, green bug, or aphid); abiotic stress (such as drought tolerance, flooding, high level of salinity, heavy metal, aluminum, manganese, cadmium, zinc, UV-B, boron, iron deficiency chlorosis or cold tolerance (i.e. extreme temperatures)).

In a further aspect, there is also provided the plant as defined herein having improved agronomical traits such as seedling vigor, yield potential, phosphate uptake, plant growth, seedling growth, phosphorus uptake, lodging, reproductive growth, or grain quality.

In accordance with a further aspect, there is provided a disease-resistant plant, comprising an introgression from a Hikmok sorip accession P1372415A or progeny thereof, wherein the introgression comprises a Si uptake conferring QTL linked to at least one marker located on the chromosome equivalent to linkage group J (Chromosome 16), and wherein said marker is located within a chromosome interval corresponding to about 95 cM to about 102 cM distance or from physical positions 33104446 bp to 35762786 bp, or a portion thereof, on a genetic linkage map from Hikmok sorip (P1372415A). In another embodiment said introgression is from any one of: PI209332, PI404166, PI437655, PI89772, PI372415A, PI90763, or a progeny thereof.

In accordance with a further aspect, there is provided a plant that can uptake and accumulate Si into its leaf or stem tissue at an increased rate as compared to a LoSil or control plant grown under hydroponic conditions.

In accordance with a further aspect, there is provided a plant comprising a HiSil allele which confers increased Si uptake, and wherein the HiSil allele comprises at least one single nucleotide polymorphism (SNP) selected from the group consisting of: G(33672717), A(33673022), G(33673483), C(33681630), T(33681946), T(33681961), T(33682500), G (33683047), and C (33683049) corresponding to a chromosomal interval from Hikmok sorip chromosome 16 at about 95 cM to about 102 cM distance or from physical positions 33104446 base-pairs to 3576286 base-pairs as indicated on a genetic linkage map from Hikmok sorip (P1372415A).

In a further aspect, there is provided a plant cell, plant seed or plant part derived from the HiSil Glycine max plant. There is also provided a progeny plant derived from the HiSil Glycine max plant.

Particularly, with reference to the plants as defined herein, the plant is a crop plant. More particularly, the crop plant is a soybean or Glycine max plant. Most particularly, the Glycine max plant is an elite Glycine max plant.

In a further aspect, there is provided a method for producing a Glycine max plant having a HiSil trait, the method comprising the steps of: a) providing a first Glycine max plant line, or progeny thereof comprising an H1 haplotype; b) crossing the Glycine max plant provided in step a) with a second Glycine max plant; c) collecting the seeds resulting from the cross in step b); d) regenerating the seeds of c) into plants; e) providing one or more backcross generations by crossing the plants of step d) or selfed offspring thereof with Glycine max breeding material to provide backcross plants; f) selfing plants of step e) and growing the selfed seed into plants; g) evaluating the plants of step f) for high silicon uptake (i.e. HiSil trait); and h) identifying and selecting plants that are high accumulators of Si wherein the identifying is performed by genotyping the plant for a marker that associates with the HiSil trait (e.g. a marker within 20 cM, 10 cM, 5 cM or less from the a chromosomal interval corresponding to about 95 cM to about 102 cM distance or from physical positions 33104446 bp to 35762786 bp, or a portion thereof, on a genetic linkage map from Hikmok sorip (P1372415A).

In accordance with a particular aspect of the invention, there is provided a method for producing a Glycine max plant having the HiSil trait, the method comprising the steps of: a) providing any one of the following Glycine max plant lines, or progeny thereof, selected from the group consisting of PI372415A, P1209332, P1404166, P1437655, P189772, P1372415A or P190763; b) crossing the Glycine max plant provided in step a) with a second Glycine max plant; c) collecting the seeds resulting from the cross in step b); regenerating the seeds of c) into plants; d) providing one or more backcross generations by crossing the plants of step c) or selfed offspring thereof with Glycine max breeding material to provide backcross plants; e) selfing plants of step d) and growing the selfed seed into plants; f) evaluating the plants of step e) for high silicon uptake (i.e. HiSII trait); and g) identifying and selecting plants that are high accumulators of Si wherein the identifying is performed by genotyping the plant for a marker that associates with the HiSil trait (e.g. a marker within 20 cM, 10 cM, 5 cM or less from the a chromosomal interval corresponding to about 95 cM to about 102 cM distance or from physical positions 33104446 bp to 35762786 bp, or a portion thereof, on a genetic linkage map from Hikmok sorip (P1372415A).

In accordance with a particular aspect of the invention, there is provided a method for producing seeds that result in Glycine max plants having a HiSil trait, the method comprising the steps of: a) providing a first Glycine max plant line, or progeny thereof comprising an H1 haplotype; b) crossing the Glycine max plant provided in step a) with a second Glycine max plant; c) collecting the seeds resulting from the cross in step b); d) regenerating the seeds of c) into plants; e) providing one or more backcross generations by crossing the plants of step d) or selfed offspring thereof with Glycine max breeding material to provide backcross plants; f) selfing plants of step e) and growing the selfed seed into plants; and g) selecting and identifying seeds that result in Glycine max plants that are high accumulators of Si wherein the identifying is performed by genotyping the plant for a marker that associates with the HiSil trait (e.g, a marker within 20 cM, 10 cM, 5 cM or less from the a chromosomal interval corresponding to about 95 cM to about 102 cM distance or from physical positions 33104446 bp to 35762786 bp, or a portion thereof, on a genetic linkage map from Hikmok sorip (P1372415A).

In accordance with a further aspect, the invention provides a method for producing seeds that result in Glycine max plants having the HiSil trait, the method comprising the steps of: providing any one of the following Glycine max plant lines, or progeny thereof, selected from the group consisting of PI372415A, PI209332, PI404166, PI437655, PI89772, PI372415A or PI90763; crossing the Glycine max plant provided in step a) with a second Glycine max plant; collecting the seeds resulting from the cross in step b); regenerating the seeds of c) into plants; providing one or more backcross generations by crossing the plants of step d) or selfed offspring thereof with Glycine max breeding material to provide backcross plants; selfing plants of step e) and growing the selfed seed into plants; and selecting and identifying seeds that result in Glycine max plants that are high accumulators of Si wherein the identifying is performed by genotyping the plant for a marker that associates with the HiSil trait (e.g. a marker within 20 cM, 10 cM, 5 cM or less from the a chromosomal interval corresponding to about 95 cM to about 102 cM distance or from physical positions 33104446 bp to 35762786 bp, or a portion thereof, on a genetic linkage map from Hikmok sorip (PI372415A).

In accordance with a particular aspect of the invention, there is provided a method of producing a soybean plant having increased Si uptake, the method comprising the steps of: a) crossing a first Glycine max plant having high Si uptake with a second Glycine max plant having low Si uptake, wherein said first Glycine max plant comprises in its genome a chromosomal interval comprising a H1 haplotype; and b) producing a progeny plant from the plant cross of a), wherein said progeny plant comprises in it genome a chromosomal interval comprising a H1 haplotype; thereby producing a soybean plant having increased Si uptake.

In accordance with a particular aspect of the invention, there is provided a method of controlling any one of the following diseases in a crop: Asian soybean rust, soy cyst nematode, nematode, rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum and Myzus persicae, the method comprising the steps of: a) planting in a field a soybean HiSil plant as described herein; and b) ensuring that said plant is provided with a supply of Si at a concentration of at least about 0.8 mM,

In accordance with a particular aspect of the invention, there is provided a method of reducing abiotic stress damage in a crop wherein the abiotic stress is caused by any one of the following: drought, flooding/excess water, high level of salinity, heavy metal, aluminum, manganese, cadmium, zinc, UV-B, boron, cold temperature, heat, or herbicide, the method comprising the steps of: a) planting in a field a soybean HiSil plant as described herein; and b) ensuring that said plant is provided with a supply of Si at a concentration of at least about 0.8 mM (e.g. hydroponic or field conditions).

In accordance with a particular aspect of the invention, there is provided a method of increasing yield in a crop, the method comprising the steps of: a) planting in a field a soybean HiSil plant as described herein; and b) ensuring that said plant is provided with a supply of Si at a concentration of at least about 0.8 mM.

In accordance with a particular aspect of the invention, there is provided a method of growing a crop, the method comprising the steps of: a) planting in a field a HiSil plant as described herein; and b) applying a compound to the field that comprises silicon: prior to planting, at planting, or after planting.

In accordance with a particular aspect of the invention, there is provided a method of growing a crop, the method comprising planting in a field a HiSil plant as described herein, wherein the soil of the field comprises silicon at the level of at least about 0.8 mM.

In accordance with a particular aspect of the invention, there is provided a method of identifying or selecting a first plant having increased Si uptake, the method comprising the steps of: a) isolating a nucleic acid from a first plant; b) detecting in the nucleic acid the presence of a molecular marker that associates with increased Si uptake and wherein the molecular marker is: associated with a Hi haplotype; or located within 20 cM, 10 cM, 5 cM, 1 cM or 0.5 cM of a chromosomal interval corresponding to a genomic region from Hikmok sorip chromosome 16 at about 92.6 cM to about 132 cM distance; or located from physical positions 33.15M base-pairs to 36.72M base-pairs as indicated on a genetic linkage map from Hikmok sorip (PI372415A); and c) identifying or selecting said soybean plant on the basis of the presence of the molecular marker of b); thereby identifying or selecting a first soybean plant having increased Si uptake.

In accordance with the HiSil plant as defined herein, the plant or first plant is a crop plant. More particularly, the crop plant is a soybean crop.

In accordance with a further aspect, there is provided a method of producing a soybean plant having increased Si uptake, the method comprising the steps of: crossing a first Glycine max plant having low Si uptake with a second Glycine max plant having high

Si uptake, wherein said second Glycine max plant comprises a chromosomal interval associated with Si accumulation corresponding to a genomic region from Hikmok sorip chromosome 16 at about 95 cM to about 102 cM distance or from physical positions 33104446 base-pairs to 3576286 base-pairs as indicated on a genetic linkage map from Hikmok sorip (PI372415A); and producing a progeny plant from the plant cross of a), wherein said progeny plant comprises the chromosomal interval associated with Si accumulation in a) or a portion thereof; thereby producing a soybean plant having increased Si uptake.

According to a further aspect, the invention provides a method of producing a Glycine max plant with high silicon uptake, the method comprising the steps of: a) isolating a nucleic acid from a Glycine max plant; b) genotyping the nucleic acid of a); c) identifying a plant as comprising at least one molecular marker associated with increased Si uptake wherein said molecular marker is located within 20 cM, 10 cM, ScM, 1 cM or 0.5 cM of a chromosomal interval corresponding to a genomic region from Hikmok sorip chromosome 16 at about 95 cM to about 102 cM distance or from physical positions 33104446 base-pairs to 3576286 base-pairs, or portion thereof as indicated on a genetic linkage map from Hikmok sorip (PI372415A); and d) producing a Glycine max progeny plant from the plant of c) identified as having said molecular marker associated with increased Si uptake.

In accordance with a further aspect, there is provided a method of producing a Glycine max plant having increased silicon uptake, said method comprising the steps of: a) introducing into a Glycine max plant's genome a HiSil chromosomal interval comprising nucleic acids comprising base pairs corresponding to positions: 1-2658341 of SEQ ID NO: 1; 565530-578331 of SEQ ID NO: 1; 565530-568778 of SEQ ID NO: 1; 567613-568778 of SEQ ID NO: 1; 575050-578331 of SEQ ID NO:1; or 577172-578331 of SEQ ID NO: 1; b) selecting for a Glycine max plant, plant germplasm or plant seed comprising the chromosomal interval of a) by isolating a nucleic acid from said plant and genotyping the nucleic acid for a molecular marker which associates with the presence of the chromosomal interval as well as the trait of increased Si uptake; and c) producing a Glycine max plant having increased silicon uptake.

According to a further embodiment, there is provided a method of producing a Glycine max plant with high silicon uptake, the method comprising the steps of: a) isolating a nucleic acid from a Glycine max plant; b) genotyping the nucleic acid of a); c) identifying a plant as comprising at least one molecular marker associated with the presence of a Si transporter gene (e.g. any molecular marker described in Tables 15-21) wherein the gene encodes a protein comprising any one of SEQ ID NO: 15 or SEQ ID NO: 17; and d) producing a Glycine max progeny plant from the plant of c) identified as having said molecular marker associated with increased Si uptake.

According to a further embodiment, there is provided a plant, plant part, or plant seed produced by the method as defined herein.

In accordance with a further aspect, the invention provides an agronomically elite Glycine max plant capable of accumulating Si in leaf tissue at a concentration of at least 1% Si concentration when plants are provided with a supply of Si at a concentration of about 0.8mM under hydrophonic conditions, wherein the Glycine max comprises a genomic region introduced into its genome corresponding to any one of SEQ ID NO: 14 or 16.

In accordance with a further aspect, the invention provides a plant of a soybean variety or lineage having high Si uptake, provided that said variety is not Hikmok

In accordance with a further aspect, the invention provides seeds produced by the HiSil plant as defined herein.

In accordance with a further aspect, the invention provides a plant having introduced into its genome a nucleic acid sequence encoding a protein having 60%, 70%, 80%, 90%, 95%, or 99% sequence identity to any one of SEQ ID NO: 15 or SEQ ID NO:

17.

According to a particular aspect, the plant is a soybean or Glycine max plant. More particularly, the Glycine max plant is an elite Glycine max plant, provided that the soybean plant is not Hikmok sorip (PI372415A).

In accordance with a further aspect of the invention, there is provided an isolated polynucleotide encoding a Si transporter selected from the group consisting of SEQ ID NOs: 14 and 16 for use in transforming a plant not comprising a copy of said polynucleotide in its genome for improving Si uptake of the plant.

In accordance with a further aspect of the invention, there is provided a vector comprising the polynucleotide or an expression cassette as defined herein.

In accordance with a further aspect of the invention, there is provided a plant expression cassette comprising the polynucleotide as defined herein (e.g. polynucleotide encoding a protein comprising either SEQ ID NO: 15 or 17).

In accordance with a further aspect, the invention provides a plant expression cassette encoding a Si transporter selected from the group consisting of SEQ ID NOs: 14 and 16.

In accordance with a further aspect of the invention, there is provided a transgenic plant comprising the plant expression cassette as defined herein.

In accordance with a further aspect of the invention, there is provided a transgenic seed comprising the plant expression cassette as defined herein.

According to a further aspect of the invention, there is provided a method of producing a plant having increased silicon uptake, said method comprising the steps of: a) introducing into a plant's genome a nucleic acid encoding a HiSil protein; b) selecting for a plant, plant germplasm or plant seed comprising the nucleic acid of a); and c) producing a plant having increased silicon uptake.

According to a further aspect of the invention, there is provided a method of producing a disease-resistant plant, the method comprising the step of: stably introducing into a plant genome the plant expression cassette as described herein, wherein said introduction of said plant expression cassette confers increased Si uptake in said plant; thereby producing a disease-resistant plant.

According to a further aspect of the invention, there is provided a method of producing a plant with increased yield, the method comprising the steps of: stably introducing into a plant genome the plant expression cassette as described herein, wherein said introduction of said plant expression cassette confers increased Si uptake in said plant; thereby producing a plant with increased yield.

According to a further aspect of the invention, there is provided an agronomically elite soybean seed which is the progeny of a transgenic female ancestor soybean plant having in its genome a recombinant DNA which expresses a Si transporter comprising an amino acid sequence with at last about 80%, 90%, 95%, 99% or 100% sequence identity to the amino acid sequence of any one of SEQ ID NOs: 15 or 17.

According to a further aspect of the invention, there is provided a method for producing a soybean plant with increased Si uptake, the steps comprising: introducing into a plant cell a recombinant DNA molecule comprising a polynucleotide encoding a polypeptide, wherein the nucleotide sequence of the polynucleotide is selected from the group consisting of: a) a nucleotide sequence set forth as SEQ ID NO: 14 or 16; b) a nucleotide sequence encoding a protein having the amino acid sequence of SEQ ID NO: 15 or 17; c) a nucleotide sequence with at least 90%, at least 91%, at least 92%, at least 93%, at least 94%, at least 95%, at least 96%, at least 97%, at least 98%, at least 99% identity to SEQ ID NO: 14, or 16; and d) a nucleotide sequence encoding a protein with at least 90%, at least 91%, at least 92%, at least 93%, at least 94%, at least 95%, at least 96%, at least 97%, at least 98%, at least 99% identity to SEQ ID NO: 15 and 17; and growing a plant from said plant cell.

In accordance with a further aspect of the invention, there is provided a plant, plant part or plant seed produced by the method herein defined.

According to a further aspect of the invention, there is provided a seed for, or a seed from, the plant as defined herein.

According to a further aspect of the invention, there is provided a cell of a seed as defined herein. Particularly, an elite Glycine max plant cell or seed comprising the HiSil trait.

According to a further aspect of the invention, there is provided a cell of a plant as defined herein.

According to a further aspect of the invention, there is provided a kit for producing a silicon high accumulating plant comprising: (a) the seed as defined herein, and (b) at least one constituent for making a silicon soil amendment.

According to a further aspect of the invention, there is provided a method for growing a plant, comprising the steps of: (a) providing a plant as defined herein or a seed as defined herein; (b) growing a plant therefrom; and (c) irrigating said plant with a silicon soil amendment.

In accordance with a further aspect, the invention provides a method of introducing a HiSil trait into a soybean plant, comprising: selecting a soybean plant comprising a nucleic acid sequence in its genome that encodes an a protein having 80% sequence identity to SEQ ID NO: 15 or SEQ ID NO:17, wherein the protein comprises a Threonine at a position relative to position 295 of SEQ ID NO:15, and introducing a modification to the nucleic acid sequence such that the encoded protein comprises an Isoleucine at the position relative to position 295 of SEQ ID NO:15, wherein a site-directed nuclease (SDN) introduces the modification to the nucleic acid sequence.

In accordance with a further aspect, the invention provides a soybean plant produced by one of the method as defined herein.

According to a particular aspect, the soybean plant is an elite Glycine max plant, provided that the soybean plant is not Hikmok sorip (PI372415A). In another embodiment, the soybean plant is an elite Glycine max plant, provided the soybean plant is not any one of: PI209332, PI404166, PI437655, PI89772, PI372415A, PI90763, or a progeny thereof.

In accordance with a further aspect, the invention provides an elite soybean plant comprising a nucleic acid sequence that encodes a protein having at least 80% sequence identity to SEQ ID NO: 15 or SEQ ID NO: 17, wherein the protein comprises an Isoleucine at a position corresponding to position 295 of SEQ ID NO:15.

In accordance with a further aspect, the invention provides a method of growing a soybean crop, the method comprising the steps of: a) planting in a field a soybean plant as described herein and b) applying a compound to the field that comprises silicon: prior to planting, at planting, or after planting.

In accordance with a further aspect, the invention provides a method of growing a soybean crop, the method comprising: a) selecting a location for planting the soybean crop, wherein the location comprises soil, said soil having a silicon concentration at a level of at least 7 ppm, at least 10 ppm, at least 15 ppm, at least 20 ppm, at least 30 ppm, at least 40 ppm or at least 50 ppm and b) planting a soybean plant as described herein.

### DESCRIPTION OF THE FIGURES

FIG. 1. Frequency distribution of silicon (Si) accumulation observed in a set of cultivated germplasm. Intervals on x axis are adjusted to make it comparable to FIG. 2.

FIG. 2. Frequency distribution of silicon (Si) accumulation observed in 141 recombinant inbred lines (RILs).

FIG. 3. Scanning electron microscopy and X-ray microanalysis mapping images showing silicon (Si) accumulation in leaves harvested from Hikmok sorip and Majesta grown with Si supplementation (1.7 mM). Observations are representative analyses of three samples.

FIG. 4. Genome-wide association study performed using a set of 139 cultivated soybean germplasm.

FIG. 5. QTL analysis for silicon (Si) accumulation in soybean leaves among 141 recombinant inbred lines (RILs) derived from crossing Majesta and Hikmok sorip.

FIG. 6. Genetic map position of the HiSil interval derived from crossing Majesta and Hikmok sorip identified on chromosome 16 from 95 cM to 102 cM.

FIG. 7. Genetic map position of the Hisil locus for silicon accumulation in soybean leaves identified on chromosome 16 at 95 cM distance.

FIG. 8. Genome-wide analysis of epistatic interaction for Silicon uptake in soybean leaves from 141 Majesta×Hikmok sorip RILs as verified by EPlstatic QTL mapping performed by ICIMapping.

FIG. 9. Sequences alignment at HiSil-Del (˜286 bp deletion) locus which was used to develop marker linked to HiSil.

FIG. 10. Agrose gel showing segregation pattern of HiSil-Del marker in RIL population derived from Hikmok sorip and Majesta.

FIG. 11. Digested PCR product amplified with HiSil-Mboll in Williams, Hikmok sorip and Majesta showing detectable polymorphism.

FIG. 12. High resolution QTL of the Hisil locus for silicon accumulation in soybean leaves Hikmok X Majesta RILs.

FIG. 13. Genetic map position of the HiSil interval on chromosome 16 from 92.6 cM to 132 cM distance.

FIG. 14. Frequency distribution of average leaf silicon (Si) content observed in F3 (F2:3) lines derived from a cross Hamilton×PI 89772

FIG. 15. QTL comparison between Hikmok×Majesta and Hamilton×PI89772.

FIG. 16. Genetic map showing markers and significance of markers in Hamilton×PI89772.

FIG. 17. Genetic map showing confirmed interval at 5.57 Mb in Majesta×Hikmok sorip and Hamilton×PI89772.

FIG. 18. Silicon uptake in soybean accession carrying different haplotypes defined based on single nucleotide present in coding sequences of Glyma16g30000 and Glyma16g30020.

FIG. 19. Protein homology based model of HiSil (Glyma16g30020) constructed using 1-TASSER server.

FIG. 20. Results of BLASTp search at NCBI server performed to identify HiSil homologs in rice.

FIG. 21. Photographs of split plant stems after being inoculated with BSR. A. Resistant control under water treatment. B. Resistant control under AgSil treatment. C. Susceptible control under AgSil treatment. D. Susceptible control under water treatment.

FIG. 22. Photographs of general symptomology and assay layout from Example 8. A. Susceptible control under water treatment. B. Susceptible control under AgSil treatment,

FIG. 23. Histograms of the trait % BSR within control and treated groups. Please note that both histograms do not include observations of lines “Corsoy 79Nonlnoc A” and “Corsoy 79Nonlnoc B” because they did not get the same inoculation treatment as all other lines in the experiment.

FIG. 24. Bar graphs representing all treated and non-treated groups from Example 8.

FIGS. 25. Photographs of Soybean Cyst Nematode (SCN) trial post inoculation. A. AgSil treatment. B. Water treatment.

FIG. 26. Histograms of the Cyst Counts within A. control and B. treated groups.

FIG. 27. Photograph of Root-knot Nematode (RKN) trial layout.

FIG. 28. Histograms of RKN damage rates within the treated and untreated groups.

FIG. 29. Histograms of RKN damage rates for tested lines only (i.e. no checks included) within the treated and untreated groups,

FIG. 30. Treated group: bar plots of rates means (over 4 reps) versus MATID; MATID's are arranged according to High and Low (Si accumulators) subgroups,

FIG. 31. Untreated group: bar plots of rates means (over 4 reps) versus MATID; MATID's are arranged according to High and Low (Si accumulators) subgroups,

FIG. 32. Boxplots of soybean lines' rates means by High and Low (Si accumulators) subgroups.

FIG. 33. Effect of silicon (Si) amendment on soybean resistance to Phytophthora sojae race-25. (a) Survival rate differences among plants grown without and with Si; (b) Increased survival rate with Si application in LoSil and HiSil RILs; Average gain in (c) dry weight and (d) plant height with Si.

FIG. 34. Effect of silicon (Si) amendment on soybean resistance to cocktail of five Phytophthora sojae races (4, 7, 13, 17 and 25). (a) Roots of P. sojae infected soybean plants grown with and without Si; average gain in (b) shoot dry weight and (d) root dry weight with Si; (c) increased survival rate with Si application in LoSil and HiSil RILs.

FIG. 35. Leaf wilting score of soybean plants grown under hydroponic conditions for three weeks and then imposed water stress by drowning-off water from system. Wilting scale is—1 for no wilting, 2 very slight wilting, 3 wilting, 4 high wilting, 5 dying, and 6 is for dead.

FIG. 36. Photographs of major steps involved in the grafting of soybean plants

FIG. 37. Leaf wilting score of soybean plants grown under hydroponic conditions for three weeks and submitted to water stress. Wilting scale is—0 —no wilting; 1—very slight wilting; 2—slight wilting; 3—wilting; 4—high; 5—dying, and 6—dead. MajestaiH represents Majesta shoots grafted on Hikmok rootstock, and Hikmok/M represents Hikmok root grafted on Majesta rootstock.

FIG. 38. Validation of HiSil in transgenic Arabidopsis. (a) Expression of GUS with root specific promoters CASP2 and NIP5;1. (b) Si accumulation by transgenic Arabidopsis lines for Glyma16g30000 and Glyma16g30020 with alleles representing Williams and Hikmok HiSil

FIG. 39. Average Si accumulation in HiSil and null plants.

FIG. 40. Silicon (Si) efflux transport facilitated by Williams and Hikmok type alleles of Glyma16g30020 gene evaluated in Xenopus oocyte assay.

FIG. 41. Silicon (Si) transport evaluated in Xenopus oocyte assay of different constructs (Hikmok and Williams alleles of Glyma16g:30000 and Glyma16g:30020 without or with point mutations).

FIG. 42. Schematic map of plasmid clone pCR-GmHiSil1aNrul containing GmHiSil gene sequence. The GmHiSil is flanked by two Nrul sites.

FIG. 43. Transformation vector for expressing Cas9 and sgRNAs.

### DESCRIPTION OF INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Abbreviations and Definitions

### Abbreviations

bp: Base-pairs; cM; centimorgan; CMLM: Compressed mixed linear models; GAPIT: Genomic Association and Prediction Integrated Tool; GBS: Genotyping by sequencing; GLM: general linear model; GWAS: genome-wide association study; IGST-GBS: IBIS Genotyping by Sequencing Tool; ICIM: inclusive composite interval mapping; LOD: Logarithm of odds; Mb: million base; PCA: principal component analysis; PVE: phenotypic variance explained; QTL: quantitative trait locus; SNP: single nucleotide polymorphism; RIL: recombinant inbred lines. CAPS: Cleaved Amplified Polymorphic Sequences; CRISPR: Clustered Regularly Interspaced Short Palindromic Repeats; TALENs: Transcription activator-like effector nucleases; BSR: Brown Stem Rot; SCN: Soybean Cyst Nematode; RKN: Root-Knot Nematode.

Definitions

The term “about” as used herein refers to a margin of + or 10% of the number indicated. For sake of precision, the term about when used in conjunction with, for example: 90% means 90%+/−9% i.e. from 81% to 99%. More precisely, the term about refer to + or −5% of the number indicated, where for example: 90% means 90%+/−4.5% i.e. from 86.5% to 94.5%.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the transitional words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “HiSil Chromosomal interval” means a chromosomal interval corresponding to a genomic region from Hikmok sorip chromosome 16 at about 92.6 cM to about 132 cM distance or from physical positions 31.15Mbase-pairs to 36.72Mbase-pairs, particularly at about 95 cM to about 102 cM distance or from physical positions 33104446 base-pairs to 3576286 base-pairs, or portion thereof as indicated on a genetic linkage map from Hikmok sorip (PI372415A).

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “allele” refers to one of two or more different nucleotides or nucleotide sequences that occur at a specific locus (e.g. Table 18 illustrates unfavorable and favorable alleles for the HiSil trait).

A “locus” is a position on a chromosome where a gene or marker or allele is located. In some embodiments, a locus may encompass one or more nucleotides. For example, any marker listed in Tables 15-21 depicts a “locus” that is associated with the HiSil trait. Further, any marker within the HiSil Chromosomal interval can be a locus associated with the HiSil trait.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype. In some embodiments of this invention, the phrase “desired allele”, “target allele” or “allele of interest” refers to an allele(s) that is associated with the HiSiI trait in a soybean plant relative to a control soybean plant not having the target allele or alleles. Thus, for example, a soybean plant comprising one or more desired alleles as indicated in Table 18 or markers closely associated with markers in Tables 15-21 may be utilized in selecting, identifying or producing soybean plants with increased Si accumulation as compared to a control plant not comprising said markers (e.g. HiSil Soybean Plants).

As used herein, the terms “marker” and “genetic marker” are used interchangeably to refer to a nucleotide and/or a nucleotide sequence that has been associated with a phenotype and/or trait. A marker may be, but is not limited to, an allele, a gene, a haplotype, a chromosome interval, a restriction fragment length polymorphism (RFLP), a simple sequence repeat (SSR), a random amplified polymorphic DNA (RAPD), a cleaved amplified polymorphic sequence (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sol, USA 86:2766 (1989)), an inter-simple sequence repeat (ISSR) (Blair et al., Theor Appl. Genet. 98:780 (1999)), an inter-retrotransposon amplified polymorphism (IRAP), a retrotransposon-microsatellite amplified polymorphism (REMAP) (Kalendar et al., Theor Appi. Genet. 98:704 (1999)), an isozyme marker, an RNA cleavage product (such as a Lynx tag) or any combination of the markers described herein. A marker may be present in genomic or expressed nucleic acids (e.g., ESTs). A large number of soybean genetic markers are known in the art, and are published or available from various sources, such as the SoyBase internet resource (www.soybase.org). In some embodiments, a genetic marker of this invention is a SNP allele (e.g. see Table 15-20), a SNP allele located in a chromosome interval corresponding to the HiSil Chromosomal interval) and/or a haplotype (e.g. H1 haplotype) or a combination of SNP alleles from Table 20, each of which are associated with the HiSil Trait.

Markers corresponding to genetic polymorphisms between members of a population can be detected by methods well-established in the art. These include, but are not limited to, nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of randomly amplified polymorphic DNA (RAPD), detection of single nucleotide polymorphisms (SNPs), and/or detection of amplified fragment length polymorphisms (AFLPs). Thus, in some embodiments of this invention, such well known methods can be used to detect the SNP alleles as defined herein.

Accordingly, in some embodiments of this invention, a marker is detected by amplifying a Glycine sp. nucleic acid with two oligonucleotide primers by, for example, an amplification reaction such as the polymerase chain reaction (PCR).

A “marker allele,” also described as an “allele of a marker locus,” can refer to one of a plurality of polymorphic nucleotide sequences found at a marker locus in a population that is polymorphic for the marker locus.

Marker-assisted selection (herein, “MAS”) or interchangeably marker-assisted breeding (herein, “MAB”) is a process by which phenotypes are selected based on marker genotypes. Marker assisted selection includes the use of marker genotypes for identifying plants for inclusion in and/or removal from a breeding program or planting.

As used herein, the terms “marker locus”, “marker loci”, “locus” or “loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the term “molecular marker” may be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Some of the markers described herein can also be referred to as hybridization markers when located on an indel region. This is because the insertion region is, by definition, a polymorphism vis-a-vis a plant without the insertion. Thus, the marker need only indicate whether the indel region is present or absent. Any suitable marker detection technology may be used to identify such a hybridization marker, e.g., technology for SNP detection.

A marker is “associated with” a trait when said trait is linked to the marker and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker. For example, “a marker associated with the HiSil trait” refers to a marker whose presence or absence can be used to predict whether a plant will display increased Si accumulation (e.g. markers within the

HiSil chromosomal interval or those closely associated with said HiSil chromosomal interval, also see Tables 15 to 21),

As used herein, the term “probe” refers to a single-stranded oligonucleotide sequence that will form a hydrogen-bonded duplex with a complementary sequence in a target nucleic acid sequence analyte or its cDNA derivative. Thus, a “marker probe” and “probe” refers to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides may be used for nucleic acid hybridization. Alternatively, in some aspects, a marker probe refers to a probe of any type that is able to distinguish (i.e., genotype) the particular allele that is present at a marker locus. Non-limiting examples of a probe of this invention may be found in the Table 19 and the Sequence Listing (i.e. SEQ ID NOs 278 to 495).

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing, Primers can be prepared by any suitable method, Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No, 4,458,066. Primers can be labeled, if desired, by incorporating detectable moieties by for instance spectroscopic, fluorescence, photochemical, biochemical, immunochemical, or chemical moieties. Non-limiting examples of primers of the invention include Tables 13, 14 and/or 19 and the Sequence Listing (e.g, SEQ ID NOs: 27 to 277).

As used herein, the terms “backcross” and “backcrossing” refer to the process whereby a progeny plant is crossed back to one of its parents one or more times (e.g., 1, 2, 3, 4, 5, 6, 7, 8, 9, 10, or more.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on. In some embodiments, the number of backcrosses can be about 1 to about 10 (e.g., 1, 2, 3, 4, 5, 6, 7, 8, 9, 10). In some embodiments, the number of backcrosses is about 7.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “Introgression”, “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, 5, 6, 7, 8, 9, 10 or more times) to a line having a desired genetic background, selecting for the desired allele, with the result being that the desired allele becomes fixed in the desired genetic background. For example, a marker associated with the HiSil trait may be introgressed from a donor into a recurrent parent that is a LoSil plant. The resulting offspring could then be backcrossed one or more times and selected until the progeny comprises the genetic marker(s) associated with the HiSil trait (e.g. markers as illustrated in Tables 15-21) in the recurrent parent background.

As used herein, the term “linkage” refers to the degree with which one marker locus is associated with another marker locus or some other locus (for example, a BSR or FLS resistance locus). The linkage relationship between a genetic marker and a phenotype may be given as a “probability” or “adjusted probability.” Linkage can be expressed as a desired limit or range. For example, in some embodiments, any marker is linked (genetically and physically) to any other marker when the markers are separated by less than about 50, 40, 30, 25, 20, or 15 map units (or cM). For example, one aspect of the invention are the use of markers associated with the HiSil trait to identify or produce HiSil plants wherein the markers are located within 50, 40, 30, 25, 20, or 15 map units (or cM) from any marker listed in Tables 15-21 or from the HiSil chromosome interval.

A centimorgan (“cM”) or a genetic map unit (m.u.) is a unit of measure of recombination frequency and is defined as the distance between genes for which one product of meiosis in 100 is recombinant. One cM is equal to a 1% chance that a marker at one genetic locus will be separated from a marker at a second locus due to crossing over in a single generation. Thus, a recombinant frequency (RF) of 1% is equivalent to 1 m.u.

As used herein, the phrase “linkage group” refers to all of the genes or genetic traits that are located on the same chromosome. Within the linkage group, those loci that are close enough together can exhibit linkage in genetic crosses. Since the probability of crossover increases with the physical distance between loci on a chromosome, loci for which the locations are far removed from each other within a linkage group might not exhibit any detectable linkage in direct genetic tests. The term “linkage group” is mostly used to refer to genetic loci that exhibit linked behavior in genetic systems where chromosomal assignments have not yet been made. Thus, the term “linkage group” is synonymous with the physical entity of a chromosome, although one of ordinary skill in the art will understand that a linkage group can also be defined as corresponding to a region of (Le., less than the entirety) of a given chromosome.

As used herein, the term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and, by definition, are separated by less than 50 cM on the same chromosome). As used herein, linkage can be between two markers, or alternatively between a marker and a phenotype. A marker locus can be “associated with” (linked to) a trait, e.g., HiSil trait. The degree of linkage of a genetic marker to a phenotypic trait is measured, e.g., as a statistical probability of co-segregation of that marker with the phenotype.

The term “gene” as used herein refers to any DNA sequence comprising several operably linked DNA fragments such as a promoter and a 5′ regulatory region, a coding sequence and an untranslated 3′ region comprising a polyadenylation site.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make up for all the genes in its genome. Genotypes can be indirectly characterized, e.g., using markers and/or directly characterized by, e.g., nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.). In some embodiments, germplasm includes but is not limited to tissue culture.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “H1 haplotype” refers to a marker locus comprising a A at position 33673022; a G at position 33673483; a C at position 33681630; a T at position 33682500; a G at position 33683047 and a C at position 33683049 corresponding to a genomic region from Hikmok sorip chromosome 16 at about 92.6 cM to about 132 cM distance or from physical positions 31.15 Mbase-pairs to 36.72 Mbase-pairs, particularly at about 95 cM to about 102 cM distance or from physical positions 33104446 base-pairs to 3576286 base-pairs as indicated on a genetic linkage map from Hikmok sorip (PI372415A) (also see for example, Table 9).

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes. One embodiment of the invention is a elite soybean plant that is homozygous for the HiSil trait.

The PCR method is well described in handbooks and known to the skilled person. After amplification by FOR, target polynucleotides can be detected by hybridization with a probe polynucleotide, which forms a stable hybrid with the target sequence under stringent to moderately stringent hybridization and wash conditions. If it is expected that the probes are essentially completely complementary (Le., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

Different nucleotide sequences or polypeptide sequences having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein, the phrase “nucleotide sequence homology” refers to the presence of homology between two polynucleotides. Polynucleotides have “homologous” sequences if the sequence of nucleotides in the two sequences is the same when aligned for maximum correspondence. The “percentage of sequence homology” for polynucleotides, such as 50, 55, 60, 65, 70, 75, 80, 85, 90, 95, 96, 97, 98, 99 or 100 percent sequence homology, can be determined by comparing two optimally aligned sequences over a comparison window (e.g., about 20-200 contiguous nucleotides), wherein the portion of the polynucleotide sequence in the comparison window can include additions or deletions (Le., gaps) as compared to a reference sequence for optimal alignment of the two sequences. Optimal alignment of sequences for comparison can be conducted by computerized implementations of known algorithms, or by visual inspection. Readily available sequence comparison and multiple sequence alignment algorithms are, respectively, the Basic Local Alignment Search Tool (BLAST; Altschul et al. (1990) J Mol Biol 215:403-10; Altschul et al. (1997) Nucleic Acids Res 25:3389-3402) and ClustalX (Chenna et al. (2003) Nucleic Acids Res 31:3497-3500) programs, both available on the Internet. Other suitable programs include, but are not limited to, GAP, BestFit, PlotSimilarity, and FASTA, which are part of the Accelrys GCG Package available from Accelrys Software, Inc, of San Diego, Calif., United States of America.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleotide sequences have at least about 50%, 60%, 70%, 75%, 80%, 85%, 90% or 95% sequence identity. In some embodiments, two nucleotide sequences can have at least about 75%, 80%, 85%, 90%, 95%, or 100% sequence identity, and any range or value therein. In representative embodiments, two nucleotide sequences can have at least about 95%, 96%, 97%, 98%, 99% or 100% sequence identity, and any range or value therein.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and

TFASTA available as part of the GCG® Wisconsin Package@ (Accelrys Inc., Burlington, Mass.). The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math,, 2:482-489, 1981, Smith et al., Nucleic Acids Res, 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073(1988)), More particularly, preferred computer programs for determining sequence identity include but are not limited to the Basic Local Alignment Search Tool (BLAST) programs, which are publicly available from National Center

Biotechnology Information (NCB') at the National Library of Medicine, National Institute of Health, Bethesda, Md. 20894; see BLAST Manual, Altschul et al., NCBI, NLM, NIH; (Altschul et al., J. Mol. Biol. 215:403-410 1990)); version 2.0 or higher of BLAST programs allows the introduction of gaps (deletions and insertions) into alignments; for peptide sequence, BLASTX can be used to determine sequence identity: and for polynucleotide sequence, BLASTN can be used to determine sequence identity.

As used herein, the terms “phenotype,” “phenotypic trait” or “trait” refer to one or more traits of an organism. The phenotype can be observable to the naked eye, or by any other means of evaluation known in the art, e.g., microscopy, biochemical analysis, and/or an electromechanical assay. In some cases, a phenotype is directly controlled by a single gene or genetic locus, i.e., a “single gene trait.” In other cases, a phenotype is the result of several genes. For example, the following invention comprises two genes that are causative for the HiSil trait wherein the genes independently or together confer the increased Si accumulation in a soybean plant.

As used herein, the term “polymorphism” refers to a variation in the nucleotide sequence at a locus, where said variation is too common to be due merely to a spontaneous mutation. A polymorphism can be a single nucleotide polymorphism (SNP), or an insertion/deletion polymorphism, also referred to herein as an “indel.” Additionally, the variation can be in a transcriptional profile or a methylation pattern. The polymorphic site or sites of a nucleotide sequence can be determined by comparing the nucleotide sequences at one or more loci in two or more germplasm entries.

As used herein, the term “plant part” includes but is not limited to embryos, pollen, seeds, leaves, flowers (including but not limited to anthers, ovules and the like), fruit, stems or branches, roots, root tips, cells including cells that are intact in plants and/or parts of plants, protoplasts, plant cell tissue cultures, plant calli, plant clumps, and the like. Thus, a plant part includes soybean tissue culture from which soybean plants can be regenerated. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ. One embodiment of the invention is a plant part from a plant having the HiSil trait.

As used herein, the term “population” refers to a genetically heterogeneous collection of plants sharing a common genetic derivation.

As used herein, the terms “progeny,” “progeny plant,” and/or “offspring” refer to a plant generated from a vegetative or sexual reproduction from one or more parent plants. A progeny plant may be obtained by cloning or selfing a single parent plant, or by crossing two parental plants and includes selfings as well as the F1 or F2 or still further generations. An F1 is a first-generation offspring produced from parents at least one of which is used for the first time as donor of a trait, while offspring of second generation (F2) or subsequent generations (F3, F4, and the like) are specimens produced from selfings or crossings of F1s, F2s and the like. An F1 can thus be (and in some embodiments is) a hybrid resulting from a cross between two true breeding parents (the phrase “true-breeding” refers to an individual that is homozygous for one or more traits), while an F2 can be an offspring resulting from self-pollination of the F1 hybrids.

As used herein, the term “reference sequence” refers to a defined nucleotide sequence used as a basis for nucleotide sequence comparison (e.g., Chromosome 16 of Glycine max cultivar Williams 82). The reference sequence for a marker, for example, can be obtained by genotyping a number of lines at the locus or loci of interest, aligning the nucleotide sequences in a sequence alignment program, and then obtaining the consensus sequence of the alignment. Hence, a reference sequence identifies the polymorphisms in alleles at a locus. A reference sequence may not be a copy of an actual nucleic acid sequence from any particular organism; however, it is useful for designing primers and probes for actual polymorphisms in the locus or loci.

Genetic loci correlating with particular phenotypes, such as increased Si accumulation, can be mapped in an organism's genome. By identifying a marker or cluster of markers that co-segregate with a trait of interest, the breeder is able to rapidly select a desired phenotype by selecting for the proper marker (a process called marker-assisted selection, or MAS). Such markers may also be used by breeders to design genotypes in silico and to practice whole genome selection.

As used herein, unless specified otherwise, or referring the a specific SEQ ID NO., all numbering of chromosomes, genes, base pairs, amino acids or other sequences are based on the reference sequence of soybean variety Williams82 as found in publicly available Williams82 reference line (SOYBASE); Soybean genome assembly is from JGI release 8, based on the original Glyma v1 (January 2012).

The term “chimeric gene” as used herein refers to a gene wherein, in nature, the coding sequence is not associated with the promoter or with at least one other regulatory region of the DNA in the gene.

The term “expression cassette” as used herein refers to a transferable region of DNA comprising a chimeric gene which is flanked by one or more restriction or other sites which facilitate precise excision from one DNA locus and insertion into another.

The term “HiSil protein” as used herein means a protein that, when introduced into a plant genome, confers increased Si accumulation/uptake. Particularly, the HiSil protein comprises a protein sequence having at least 60%, 65%, 70%, 75%, 80%, 85%, 90%, 95%, 99% sequence identity with SEQ ID NO: 15, where the polypeptide comprises at least one amino acid corresponding to a proline at position 5, a isoleucine at position 295 or a valine at position 439; and/or SEQ ID NO: 17, where the polypeptide comprises at least one amino acid corresponding to a histidine at position 322 or a glycine at position 431; and its introduction into a plant's genome confers high Si uptake in the plant.

The term “HiSil trait” as used herein means having a nucleotide encoding for a HiSil Protein in its genome. Therefore, a plant comprising that trait will have a dry weight silicon of at least 1% after at least 28 days when grown and supplied with a silicon concentration of at least about 0.5 mM, 0.6 mM, 0.7 mM, 0.75 mM, or 0.8 mM, under hydroponic conditions (temperature about 20° C.-26° C.; humidity about 55%-65%). More particularly, a high Si uptake plant comprises a Si concentration higher than about 1.53% in leaf when the plant is provided with a supply of Si at a concentration of at least about 1.5mM, Most particularly, a high Si uptake plant comprises a Si concentration higher than 1.53%; 1.54%; 1.55%; 1.56%; 1.57%; 1.58%, 1.59%; or 1.6% Si concentration in leaf when the plant is provided with a supply of Si at a concentration of at least about 1.5mM.

A “HiSil Plant” is a plant having the HiSil trait. More specifically, a “HiSil Soybean Plant” is a soybean plant having the HiSil trait. A “HiSil Glycine max Plant” is a Glycine max plant having the HiSil Trait.

A “LoSil Plant” is a plant not having the HiSil trait.

As used herein, a plant having “high Si uptake” means increased silicon accumulation when compared to average silicon accumulation in the same plant.

Particularly, average silicon accumulation is established in a soybean plant of the Williams82 variety when grown under hydroponic conditions (as defined herein).

Therefore, a plant having high Si uptake will have a dry weight silicon of at least about 1% when grown with silicon concentration of at least about 0.4 mM, 0.5 mM, 0.6 mM, 0.7 mM, or 0.8 mM, under hydroponic conditions. For example, increased Si accumulation in high Si uptake plant represents an increase in Si uptake of about 0.1% to about 3.0% when compared to the original low Si uptake plant. For example, an increased accumulation of about 10% to about 300% in total Si concentration in at least one plant part is considered an increased in Si uptake when compared to a low Si uptake plant, when both plants are supplied with Si at a concentration of at least about 0.8 mM.

Particularly, an increased SI accumulation of about 1.1×, 1.2×, 1.3× 1.4×, 1.5×, 1.6×, 1.7×, 1.8×, 1.9×, 2×, 2.5× or 3× when compared to a LoSil plant under the same growing conditions, is considered an increased in Si uptake.

The term “LoSil protein” as used herein means a protein that, when present into a plant genome, confers average Si accumulation. As used herein, a plant having “low Si uptake” means average Si accumulation in non-Si accumulating plants. For example, a LoSil soybean plant has a silicon uptake corresponding about to the level of Williams82.

Particularly, the term “low Si uptake” as used herein means a plant having a dry weight silicon of less than about 1% after about 28 days with silicon concentration of about 0.8 mM, when grown under hydroponic conditions. For example, low/normal/basic/average Si accumulation in plants is around from 0.65% to about 1.5% Si accumulation. More particularly, a plant having low Si uptake comprises a Si concentration lower than about 1.5% Si concentration in leaf when the plant is provided with a supply of Si at a concentration of at least about 1.5 mM. Most particularly, a plant having low Si uptake comprises a Si concentration less than 1.49%; 1.50%; 1.51%; 1.52% or 1.53% Si concentration in leaf when the plant is provided with a supply of Si at a concentration of at least about 1.5 mM.

The term “introduced” as used herein, in connection to a plant, means accomplished by any manner including, but not limited to; introgression, transgenic, Clustered Regularly Interspaced Short Palindromic Repeats modification (CRISPR), Transcription activator-like effector nucleases (TALENs) (Feng et al. 2013, Joung & Sander 2013), meganucleases, or zinc finger nucleases (ZFNs).

The term “plant” as used herein means a living organism of the kind exemplified by cereals, trees, shrubs, herbs, grasses, ferns, and mosses, that usually has a stem, leaves, roots and flowers, and produces seeds and typically grows in a permanent site (such as soil), absorbing water and inorganic substances through its roots, and synthesizing nutrients in its leaves by photosynthesis using the green pigment chlorophyll; or a tissue culture thereof.

The term “crop plant”, means in particular monocotyledons such as cereals (wheat, millet, sorghum, rye, triticale, oats, barley, teff, spelt, buckwheat, fonio and quinoa), rice, maize (corn), and/or sugar cane; or dicotyledon crops such as beet (such as sugar beet or fodder beet); fruits (such as ponies, stone fruits or soft fruits, for example apples, pears, plums, peaches, almonds, cherries, strawberries, raspberries or blackberries); leguminous plants (such as beans, lentils, peas or soybeans); oil plants (such as rape, mustard, poppy, olives, sunflowers, coconut, castor oil plants, cocoa beans or groundnuts); cucumber plants (such as marrows, cucumbers or melons); fibre plants (such as cotton, flax, hemp or jute); citrus fruit (such as oranges, lemons, grapefruit or mandarins); vegetables (such as spinach, lettuce, cabbages, carrots, tomatoes, potatoes, cucurbits or paprika); lauraceae (such as avocados, cinnamon or camphor); tobacco; nuts; coffee; tea; vines; hops; durian; bananas; natural rubber plants; and ornamentals (such as flowers, shrubs, broad-leaved trees or evergreens, for example conifers). This list does not represent any limitation.

Particularly, the crop plant is monocotyledonous plant. More suitably, the crop plant is a cereal, in particular wheat or barley. In particular, the crop plant is a rice plant, more particularly, a sugar cane plant. Still, more particularly, the crop plant is a corn plant.

For example, the crop plant can be a monocot plant or a member of the family Poaceae, such as wheat plant, maize plant, sweet corn plant, rice plant, wild rice plant, barley plant, rye, millet plant, sorghum plant, sugar cane plant, turfgrass plant, bamboo plant, oat plant, brume-grass plant, Miscanthus plant, pampas grass plant, switchgrass (Panicum) plant, and/or teosinte plant; or is a member of the family Alliaceae, such as onion plant, leek plant, or garlic plant.

For example, the crop plant may be a dicot plant or a member of the family Amaranthaceae, such as spinach plant, quinoa plant; a member of the family Anacardiaceae, such as mango plant; a member of the family Asteraceae, such as sunflower plant, endive plant, lettuce plant, artichoke plant; a member of the family Brassicaceae, such as Arabidopsis thaliana plant, rape plant, oilseed rape plant, broccoli plant, Brussels sprouts plant, cabbage plant, canola plant, cauliflower plant, kohlrabi plant, turnip plant, radish plant; a member of the family Bromeliaceae, such as pineapple plant; a member of the family Caricaceae, such as papaya plant; a member of the family Chenopodiaceae, such as beet plant; a member of the family Curcurbitaceae, such as melon plant, cantaloupe plant, squash plant, watermelon plant, honeydew plant, cucumber plant, pumpkin plant; a member of the family Dioscoreaceae, such as yam plant; a member of the family Ericaceae, such as blueberry plant; a member of the family Euphorbiaceae, such as cassava plant; a member of the family Fabaceae, such as alfalfa plant, clover plant, peanut plant; a member of the family Grossulariaceae, such as currant plant; a member of the family Juglandaceae, such as walnut plant; a member of the family Lamiaceae, such as mint plant; a member of the family Lauraceae, such as avocado plant; a member of the family Leguminosae, such as soybean plant, bean plant, pea plant; a member of the family Malvaceae, such as cotton plant; a member of the family Marantaceae, such as arrowroot plant; a member of the family Myrtaceae, such as guava plant, eucalyptus plant; a member of the family Rosaceae, such as peach plant, apple plant, cherry plant, plum plant, pear plant, prune plant, blackberry plant, raspberry plant, strawberry plant; a member of the family Rubiaceae, such as coffee plant; a member of the family Rutaceae, such as citrus plant, orange plant, lemon plant, grapefruit plant, tangerine plant; a member of the family Salicaceae, such as poplar plant, willow plant; a member of the family Solanaceae, such as potato plant, sweet potato plant, tomato plant, Capsicum plant, tobacco plant, tomatillo plant, eggplant plant, Atropa belladona plant, Datura stramonium plant; a member of the family Vitaceae, such as grape plant; a member of the family Umbelliferae, such as carrot plant; or a member of the family Musaceae, such as banana plant; or wherein the plant is a member of the family Pinaceae, such as cedar plant, fir plant, hemlock plant, larch plant, pine plant, or spruce plant.

Particularly, the crop plant is selected from: soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, rice.

Particularly, the crop plants are dicotyledonous plants. In one embodiment, the crop plants are cereals or soybean. In one embodiment, the crop plants are selected from the group consisting of summer barley, winter rye and soybean. More particularly, the crop plant is soybean. More particularly, the soybean is an elite line of soybean.

An “elite line” or “elite strain” is an agronomically superior line that has resulted from many cycles of breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean.

An elite plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPRO144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33; 93M11, 94M30, 95M30, 97B52, P008T22R2; PI6T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); 500-K5, S11-L2, 528-Y2, 543-B1_{;} 553-Al, 576-L9_{;} 578-G6_{;} 50009-M2; S007-Y4; 504-D3; 514-A6; 520-T6; 521-M7; 526-P3; 528-N6; 530-V6; 535-C3; 536-Y6; 539-C4; S47-K5; 548-D9; 552-Y2; 558-Z4; 567-R6; S73-S8; and 578-G6 (Syngenta Seeds, Henderson, Ky., USA); Richer (Northstar Seed Ltd. Alberta, Calif.); 14RD62 (Stine Seed Co. Ia., USA); or Armor 4744 (Armor Seed, LLC, Ar., USA).

The terms “agronomically elite” as used herein, means a genotype that has a culmination of many distinguishable traits such as emergence, vigor, vegetative vigor, disease resistance, seed set, standability, yield and threshability which allows a producer to harvest a product of commercial significance.

The expression “commercially significant yield” means a yield of grain having commercial significance to the grower represented by an actual grain yield of 103% of the check lines AG2703 and DKB23-51 when grown under the same conditions.

In contrast, an “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

The term “hilum” defines the point at which the soybean seed attaches to the pod. Varieties differ in hilum colour and can be yellow (Y), imperfect yellow (IY), grey (GR), buff (BF), brown (BR), black (BL) or imperfect black (IBL). Yellow hilum soybeans are generally the preferred type for the export market. Particularly, Hilum discolouration may occur on the imperfect yellow (IY) varieties. Affected beans may not be acceptable for export markets.

The term “disease-resistant” encompasses resistance to biotic stresses (e.g. diseases or pests), or abiotic stresses (e.g. environmental conditions).

The term “disease-resistant” as used in the present context, means a plant as defined that is resistant to any one of the following diseases selected from the group consisting of: nematode, bacteria or viruses such as: rust, smut, Golovinomyces cichoracearurn, Erysiphe cichoracearum, Blumeria graminis, Podosphaera xanthii, Sphaerotheca fuliginea, Pythium ultimum, Uncinula necator, Mycosphaereila pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulaturn, Diatraea saccharalis, Schizaphis graminurn, Phakopsora pachyrhizi, and Myzus persicae; or a combination thereof. Resistance against particular diseases such as the following are encompassed within the present invention: powdery mildew, pythiu ultimum, root rot, leaf spot, blast, brown spot, leaf and neck blast, sheath blight; schizaphis graminum; brown-stem rot; soybean cyst nematode; or pests such as: whitefly, aphid, gery field slug, sugarcane borer, green bug, or aphid.

Diseases affecting curcubitacea include closteroviruses, particularly, the dosterovirus is Beet Pseudo-Yellows Virus (BPYV) or Cucurbit Yellow Stunting Disorder Virus (CYSDV),

The term “disease-resistant” also encompasses a plant that is more resistant to abiotic stresses such as: drought, flooding/excess water, high level of salinity, heavy metal, aluminum, manganese, cadmium, zinc, sunlight (e.g. UV-B), boron, hot/cold extreme temperatures, herbicides or wind.

The term “hydroponic” refers to conditions wherein plants are grown using mineral nutrient solutions, in water, without soil. Terrestrial plants may be grown with their roots in the mineral solution only, or in an inert medium, such as perlite or gravel. Nitrogen (N), phosphorus (P), and potassium (K), that are essential to all plant growth and trace elements such as: sulphur, iron, manganese, zinc, copper, boron, magnesium, calcium, chlorine, and molybdenum. For example, physical conditions corresponding to hydroponic culture may be: aeroponics, static solution, continuous flow, fogponics, passive sub-irrigation, ebb and flow or flood and drain sub-irrigation, run to waste, deep water culture, top-fed deep water culture, or rotary. Substrates often used for hydroponics include, without being limited thereto: expanded clay aggregate, growstones, peat, rice husks, vermiculite, pumice, sand, gravel, wood fiber, sheep wool, rock wool, brick shards, or polystyrene packing peanuts.

Particularly, hydroponic conditions suitable for growth of soybean plants are described in: “Hydroponic Growth and the Nondestructive Assay for Dinitrogen Fixation” by John Imsande and Edward J. Ralston. Plant Physiol. (1981) 68, 1380-1384. More particularly, the soybean hydroponic culture conditions in greenhouse can comprise nutrient solution compositions based on Imsande and Ralston 1981 as is, or with a few modifications:

SOLUTION A: Preparation of 20 L of 30× solution for macronutrients (2 L/60 L)

| Macronutrients | (g/20 L) 30X | (mg/L) 1X |
|---|---|---|
| K₂HPO₄ | 10.4 | 17.4 |
| KNO₃ | 60.6 | 101 |
| KCl | 87.3 | 221 |
| CaCL₂ | 141 | 235 |
| MgCl₂•6H₂O | 87 | 145 |
| MgSO₄•7H₂O | 150 | 250 |

SOLUTION B: Preparation of 500 ml of 5000× solution for micronutrients (12 ml60 L)

| Micronutrients | (g) |
|---|---|
| H₃BO₃ | 0.7 |
| MnSO₄•H₂O | 0.75 |
| ZnSO₄•7H₂O | 0.5 |
| CuSO₄•5H₂O | 0.5 |
| Na₂MoO₄•2H₂O | 0.375 |
| Co(NO₃)2•6H₂O | 0.125 |

SOLUTION C: Preparation of 1 L of 3000× FeNa EDTA solution (19.8 ml60 L) FeNa EDTA (13.2% Fe)45 g

SOLUTION D: Kasil 6: Preparation of 200 L of 1× silicon solution (76 g/200 L)

| | |
|---|---|
| KASIL 6 | 22.8 g/60 L |
| HCl 5N | pH 6.5 with supplementary fertilization 2 weeks after planting |

SOLUTION E: Preparation of 20 L of 30× solution for N and P (2 L/60 L)

| Salt | (g) |
|---|---|
| NH₄H₂PO₄ | 36 |
| NH₄NO₃ | 120 |

As used herein, the term “promoter” or “promoter sequence” means a region of DNA or DNA sequence that initiates transcription of a particular gene. Promoters are located near the transcription start sites of genes, on the same strand and upstream on the DNA (towards the 5′ region of the sense strand). Promoters can be about 100-1000 base pairs long. It is understood that that genomic sequences spanning 1000 to 5000 base pairs upstream from the native gene start codon can be utilized as a promoter to initiate gene transcription of the respective gene.

As used herein, the “native” as in “native promoter” refers to a promoter that is naturally and/or originally present in a cell and it is typically designated for the expression of a particular gene. In one embodiment, “native promoter” is encoded in the natural original genome of the cell. In one embodiment, no extra ordinary measures have been taken by another organism to insert the promoter artificially into the cell. As used herein, “the native response element (RE)” or the “native promoter (RE)” refers to the RE that is naturally present in the promoter DNA sequence. For example, the human apolipoprotein C3 (ApoC3) gene is expressed from a HNF4 alpha (HNF4A) transcription factor dependent ApoC3 promoter which has two REs for HNF4A. The two REs for HNF4A (H4RE) are the native RE of the ApoC3 promoter. Likewise, the hepatocyte nuclear factor 1 alpha (HNF1A) transcription factor dependent human HNF4A P2 promoter has one RE for HNF1alpha (H1RE). The HIRE in the native RE of the human HNF4A P2 promoter.

A “non-native promoter” would be a promoter not originally present in a cell and that has been inserted artificially into the cell. In one embodiment, a non-native promoter of a gene is one that that is not naturally associated with the gene. For example, the mouse hepatocyte nuclear factor la Dup4xH4RE (Hnf1 α.sup.Dup4×H4RE) promoter was operably linked with a human hepatocyte nuclear factor 1 alpha (HNF1 alpha) cDNA. The Hnf1 a.sup.Dup4×H4RE is a non-native promoter.

### Detailed Description of Particular Embodiments

### Novel Chromosomal Interval of Glycine max

In accordance with a particular embodiment of the invention, there is provided a novel genomic region found responsible for the increased Si uptake in soybean which was found on chromosome 16 spanning from 92.6 cM to 132 cM, more particularly from 94.9 cM to 101.6 cM distance on Hikmok sorip genetic linkage map.

More particularly, the chromosomal interval comprises any one of, or a portion of: nucleotide base pair corresponding to positions: 1-2658341 of SEQ ID NO: 1; 567613-569933 of SEQ ID NO: 1; 564321-567612 of SEQ ID NO: 1; 577172-579696 of SEQ ID NO: 1; or 573723-577171 of SEQ ID NO: 1. Most particularly, the chromosome interval comprises at least one single nucleotide polymorphism (SNP) selected from the group consisting of: A(33673022), G(33673483), C(33681630), T(33682500), G(33683047), and C(33683049) of Glyma16g:30000 or Gly a 6g:30020 genes wherein presence of the SNP is associated with Si accumulation.

In accordance with a particular embodiment of the invention, the chromosomal interval comprises SEQ ID NO: 14 or 16. Particularly, the chromosomal interval comprises SEQ ID NO.14 or 16 or a portion thereof providing increased silicon uptake in a plant. Particularly, this chromosomal interval is derived from Hikmok sorip soybean variety.

According to a particular embodiment, the invention provides a chromosomal interval or genomic region that comprises a nucleic acid of SEQ ID NO: 16 or a nucleic acid encoding a polypeptide with an amino acid sequence comprising SEQ ID NO 17, where the polypeptide comprises at least one amino acid corresponding to a histidine at position 322 or a glycine at position 431.

According to a particular embodiment, the invention provides a chromosomal interval or genomic region comprises the nucleic acid is SEQ ID NO: 14, or a nucleic acid encoding a polypeptide with an amino acid sequence comprising SEQ ID NO 15, where the polypeptide comprises at least one amino acid corresponding to a proline at position 5, a isoleucine at position 295 or a valine at position 439.

Particularly, the chromosomal interval is derived from a black hilum soybean variety. More particularly, the nucleic acid is derived from a black hilum soybean variety having high Si uptake, particularly the Hikmok sorip variety.

### Plants

In accordance with a particular aspect, the present invention provides a HiSil plant wherein the plant comprises in its genome a chromosomal interval comprising the H1 haplotype. In particular, the resulting plant is a high Si accumulator as compared to a control plant not comprising the nucleic acid corresponding to the Hi haplotype.

In accordance with an alternative aspect, the present invention provides a HiSil plant which comprises in its genome a chromosomal interval associated with Si accumulation corresponding to a genomic region or portion thereof from Hikmok sorip chromosome 16 at about 92.6 cM to about 132 cM distance as indicated on a genetic linkage map from Hikmok sorip (PI372415A). Particularly, wherein the plant is an elite soybean (Glycine max) plant.

According to an alternative embodiment, there is provided a HiSil plant which comprises in its genome a chromosomal interval associated with Si accumulation corresponding to a genomic region or portion thereof from Hikmok sorip chromosome 16 corresponding to physical positions 31.15M base-pairs to 36.72 M base-pairs of Williams82 reference genome.

Therefore, a further aspect of the invention provides a plant having high Si uptake, the plant having introduced into its genome a nucleic acid sequence encoding a HiSil protein as defined by SEQ ID: 15 or 17.

Particularly, the plant comprises a genomic region introduced into its genome comprising any one of SEQ ID NO: 14, 16 or 18. Particularly, wherein the plant is an elite soybean (Glycine max) plant.

According to a particular embodiment, the invention provides a plant having a chromosomal interval or genomic region that comprises a nucleic acid of SEQ ID NO: 16 or a nucleic acid encoding a polypeptide with an amino acid sequence comprising SEQ ID NO 17, where the polypeptide comprises at least one amino acid corresponding to a histidine at position 322 or a glycine at position 431.

According to a particular embodiment, the invention provides a plant having a chromosomal interval or genomic region comprises the nucleic acid is SEQ ID NO: 14, or a nucleic acid encoding a polypeptide with an amino acid sequence comprising SEQ ID NO 15, where the polypeptide comprises at least one amino acid corresponding to a proline at position 5, a isoleucine at position 295 or a valine at position 439.

Particularly, the plant comprises a molecular marker associated with increased Si uptake capable of being amplified and identified with the primer sequences as defined herein. More particularly, the plant comprises a marker capable being amplified and identified with the following sequences: SEQ ID NO. 12, 13 and 278-495. In another instance, the plant is capable of producing an amplicon when amplified with the following sequences: SEQ ID NO, 12, 13 and 278-495.

In particular embodiment, the plant is a Glycine max (i.e. soybean) plant. Particularly, the Glycine max plant is an elite Glycine max plant. More particularly, the elite Glycine max plant comprises a HiSil trait.

In accordance with a particular embodiment, the present invention provides an elite HiSil Glycine max plant that comprises in its genome a H1 haplotype chromosomal interval. In one aspect the H1 haplotype is derived from Hikmok sorip or a progeny thereof.

According to an alternative embodiment, there is provided an elite HiSil Glycine max plant wherein the elite HiSil Glycine max plant comprises in its genome a chromosomal interval associated with Si accumulation corresponding to a genomic region or portion thereof from Hikmok sorip chromosome 16 at about 92.6 cM to about 132 cM distance as indicated on a genetic linkage map from Hikmok sorip (PI372415A).

In accordance with a particular embodiment, the invention provides an elite HiSil Glycine max plant wherein the elite HiSil Glycine max plant comprises in its genome a chromosomal interval associated with Si accumulation corresponding to a genomic region or portion thereof from Hikmok sorip chromosome 16 corresponding to physical positions 31.15M base-pairs to 36.72 M base-pairs of Williams82 reference genome.

In particular embodiment, when the plant is an elite Glycine max plant, it is a commercially elite Glycine max variety having a commercially significant yield. More particularly, the plant is an agronomically elite Glycine max.

In accordance with a particular embodiment, the chromosomal interval of the plant is derived from any one of the plant lines selected from the group consisting of: PI372415A, PI209332, PI404166, PI437655, PI89772, PI372415A or PI90763.

In accordance with a particular embodiment, the plant has improved agronomical traits such as seedling vigor, yield potential, phosphate uptake, plant growth, seedling growth, phosphorus uptake, lodging, reproductive growth, or grain quality.

A particular aspect of the invention provides a plant having introduced into its genome a nucleic acid sequence encoding a HiSil protein wherein introduction into the genome confers increased Si accumulation in the plant as compared to a control plant not comprising the nucleic acid sequence encoding a HiSil protein.

Most particularly, plants having the H1 haplotype introduced therein are hereby encompassed within the present invention, particularly those comprising the H1 haplotypes for the coding sequences of Glyma16g30000 and Glyma16g30020HiSil gene. Particularly, the H1 haplotype is defined by an nucleic acid allelic profile selected from the group consisting of: G (33672717), A(33673022), G(33673483), C(33681630), T(33681946), T(33681961), T(33682500), G(33683047), and C(33683049). Alternatively, the molecular marker associated with high Si uptake is located within HiSil region genes, and can be defined by a nucleic acid selected from the group consisting of: A(33673022), G(33673483), C(33681630), T(33682500), G(33683047), and C(33683049) of genes Glyma16g:30000 or Glyma16g:30020.

Particularly, the H1 haplotype is defined by an amino acid profile selected from the group consisting of: having at least 80% sequence identity to SEQ ID NO: 17 where the polypeptide further comprises at least one amino acid corresponding to a histidine at position 322 or a glycine at position 431. Particularly, the H1 haplotype is defined by an amino acid profile selected from the group consisting of: having at least 80% sequence identity to SEQ ID NO: 15, wherein the protein comprises a proline at position 5, an isoleucine at position 295 or a valine at position 439.

In one embodiment of the invention, it is envisioned that gene homologs within the soybean genome may be modified or introduced through a HiSil plant source (e.g. Hikmok sorip) to create plants having increased Si uptake and/or accumulation. For example coding sequences Glyma09G24930; Glyma09G24943 and Glyma09G24956 (collectively, “Soy Chr9 HiSil homologs”) may be modified to comprise a H1 haplotype and/or comprise a allelic modification corresponding to a G (33672717), A(33673022), G(33673483), 0(33681630), 1(33681946), T(33681961), T(33682500), G(33683047), or a C(33683049), In another instance, not to be limited by theory, any one of the “Soy Chr9 HiSil homologs may be expressed transgenically to create HiSil plants. Alternatively, a elite soybean plant comprising a chromosome interval comprising any on the the “Soy Chr9 HiSil homologs” derived from a HiSil Source (e.g. Hikmok sorip) wherein said introduction of the chromosome interval confers increased Si uptake and/or accumulation , is contemplated. A elite soybean plant comprising in its genome, a chromosome interval comprising any one of Glyma09G24930; Glyma09G24943 or Glyma09G24956 wherein said interval confers increased Si uptake and/or accumulation as compared to a control plant. Further contemplated are methods of identifying or selecting a HiSil plant by detecting in a plant genome a marker associated with the presence of any one of the genes selected from the group consisting of Glyma09G24930; Glyma09G24943 and Glyma09G24956 wherein the presence of said gene is associated with increased Si uptake and/or accumulation.

According to a particular embodiment, the invention provides a plant having introduced into its genome a nucleic acid sequence encoding a protein having 60%, 70%, 80%, 90%, 95%, or 99% sequence identity to any one of SEQ ID NO. 15 or SEQ ID NO. 17. More particularly, the protein comprises, or consists of: SEQ ID NO. 15 or SEQ ID NO, 17.

Particularly, the protein is a functional Si transporter that facilitates Si uptake into the plant. More particularly, the protein confers Si accumulation in any one of the plant leaves, plant stem or plant parts. Most particularly, the protein is active in the plant's roots.

More particularly, the nucleic acid sequence comprises any one of SEQ ID NOs: 14 and 16. Alternatively, the nucleic acid is derived from a Glycine sp. plant having high silicon uptake. Still, particularly, the nucleic acid is derived from a black hilum soybean variety (e.g. Hikmok sorip) having high Si uptake.

Alternatively, at least two nucleic acid sequences are introduced into the plant's genome, where the two nucleic acid sequences encode proteins comprising a polypeptide sequence comprising SEQ ID NO: 15 and SEQ ID NO: 17.

Still, particularly the invention provides an elite HiSil Glycine max plant comprising a HiSil allele which confers increased Si uptake, and wherein the HiSil allele comprises at least one single nucleotide polymorphism (SNP) selected from the group consisting of A(33673022), G(33673483), C(33681630), T(33682500), G(33683047), and C(33683049) as indicated on a genetic linkage map from Hikmok sorip (PI372415A).

### Progeny, Plant Parts, Seeds and Cells

A particular embodiment of the invention provides a plant comprising, or having introduced into its genome, a nucleic acid sequence encoding a HiSil protein wherein introduction into the genome confers increased Si accumulation in the plant as compared to a control plant not comprising the nucleic acid sequence encoding a HiSil protein.

In a particular embodiment, there is provided a progeny plant produced from, or derived from, the plant as defined herein. More particularly, there is provided a plant cell, plant seed or plant part derived from the plant as defined herein.

Particularly, in accordance with all aspects of the invention, the term “plant” means that it comprises any plant part (such as roots, leaves, stock, etc.), seed, or a tissue culture thereof. More particularly, it comprises cells of a plant, seeds from the plant, cells of a seed, or a tissue culture thereof.

In accordance with a further aspect of the invention there is provided a seed for producing the plant as defined herein. Alternatively, the plant comes from the plant itself.

According to a particular embodiment, the plant is a monocot or dicot.

### Crops/Soybean

Particularly, the plants are dicotyledonous plants, such as a crop plant. In one embodiment, the crop plant is a cereal or soybean. In one embodiment, the crop plants are selected from the group consisting of summer barley, winter rye and soybean. More particularly, the crop plant is soybean. More particularly, the soybean is an elite line of soybean, most particularly, an agronomically elite Glycine max,

Particularly, in accordance with an embodiment of the invention, there is provided an elite soybean plant comprising a nucleic acid sequence that encodes a protein having at least 80% sequence identity to SEQ ID NO: 15 or SEQ ID NO: 17, wherein the protein comprises an Isoleucine at a position corresponding to position 295 of SEQ ID NO:15,

Particularly, in accordance with an embodiment of the invention, the plant is a soybean plant and is not Hikmok sorip (Pl372415A), More particularly, the plant is of a soybean variety or lineage having high Si uptake, provided that the variety is not Hikmok sorip.

In accordance with a particular embodiment, the invention provides a method of increasing yield in a soybean crop, the method comprising the steps of: planting in a field a soybean plant as described herein; and ensuring that the plant is provided with a supply of Si at a concentration of at least about 0.8 mM.

According to a particular embodiment, the invention provides a method of growing a soybean crop, the method comprising the steps of: planting in a field a soybean plant as described herein; and applying a compound to the field that comprises silicon: prior to planting, at planting, or after planting.

In accordance with a particular embodiment, the invention provides a method of growing a soybean crop, the method comprising planting in a field a soybean plant as described herein, wherein the soil of the field comprises silicon at the level of at least about 0.8 mM.

### Soybean Parent Variety

In accordance with particular aspects of the invention, the soybean variety having low Si uptake (i.e. “low” meaning “normal” or “average” in this instance) is selected from any soybean variety not containing a molecular marker associated with the HiSil trait (e.g. any marker from Tables 15-20)

In accordance with particular aspects of the invention, the soybean variety having high Si uptake has higher Si uptake such as found in the Hikmok sorip or any other line containing the marker conferring high Si uptake. More particularly, lines, varieties or alleles carrying the H1 haplotype can be used as rootstock for grafting. In an embodiment of the invention, a plant having grafted onto it a plant part comprising the HiSil trait (e.g. the H1 haplotype or any molecular marker from Tables 15-20).

### Hilum Color Varieties

Particularly, the exotic soybean variety having high Si uptake is derived from a black hilum soybean variety, the Hikmok sorip variety.The hilum is the point at which the soybean seed attaches to the pod. Varieties differ inhilum colour and can be yellow (Y), imperfect yellow (IY), grey (GR), buff (BF), brown (BR), black (BL) or imperfect black (IBL). Hilum discolouration may occur on the imperfect yellow (IY) varieties. Particularly, Yellow hilum soybeans are generally the preferred type for the export market.

### Other Plants

In a particular aspect, the plant is selected from the group consisting of soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, rice. Si concentrations found in plants

In accordance with a particular embodiment of the invention, there is provided a plant capable of accumulating Si in leaf tissue at a concentration of at least 1% Si concentration when plants are provided with a supply of Si at a concentration of at least about 0.4 mM to about 0.8mM under hydroponic conditions. According to a particular embodiment, the plant has a leaf Si concentration of at least around one point two (1.2×), one and a half (1.5×), double (2×), or triple (3×) the concentration of a control plant not comprising the genomic region. Still, particularly, the plant has increased Si accumulation in any one of its plant leaves, plant stem or plant parts as compared to a LoSil plant. More particularly, the plant has at least 1.1×, 1.2×, 1.5×, 2×, 3' or higher Si accumulation compared to a LoSil plant.

According to a particular embodiment, the plant comprises a silicon concentration of at least 1% Si concentration in its leaves when it is provided with a supply of Si at a concentration of about 0.8 mM under hydroponic conditions. More particularly, the plant has a leaf Si concentration of at least about double (2×) as compared to a control (LoSil) plant.

Particularly, in accordance with the different aspects of the invention, plants, particularly soybean plants, having a high Si uptake are defined as having above 1%, 1.1%; 1.2%; 1.3%; 1.4%; 1.5% or 1.6% Si concentration in the leaves when the plants are provided with a sufficient supply of Si. Particularly, a sufficient supply of Si is defined at a concentration of at least about 0.8 mM Si in the potting soil or feeding solution. More particularly, high Si uptake may be defined as a plant having between 1.1% and 3% Si concentration in the leaves; most particularly: between 1.5% and 2.75% Si concentration in the leaves.

### Disease Resistance

In accordance with a particular aspect of the invention, there si also provided a plant having increased resistance to a stress, particularly: a biotic stress or an abiotic stress.

In a further aspect of the invention, the plant having high Si uptake is more resistant to a wide variety of diseases, pests and stresses. Benefits of silicon (Si) uptake to crop culture are widely accepted and a reported concept in the agricultural community. There are over a thousand scientific publications describing the beneficial role of Si for plant health, more specifically for biotic and abiotic stress tolerance (Tables 1-4). Si-derived benefits have arguably been most commonly associated with disease resistance.

More particularly, the stress is: a) a disease selected from: such as powdery mildew, Pythium ultimum, Phytophthora root rot, leaf spot, blast, brown spot, root-knot nematode, soybean cyst nematode, soybean vein necrosis virus, soybean stem canker, soybean sudden death syndrome, leaf and neck blast, rust, frogeye leaf spot, brown stem rot, Fusarium, or sheath blight); b) an insect pest such as whitefly, aphid, grey field slug, sugarcane borer, green bug, or aphid); or c) an abiotic stress such as drought tolerance, flooding, high level of salinity, heavy metal, aluminum, manganese, cadmium, zinc, UV-B, boron, iron deficiency chlorosis or cold tolerance (i.e. extreme temperatures).

Particularly, the following diseases are found in soybean crops: Asian soybean rust, soy cyst nematode, nematode, rust, smut, Golovinomyces cichoracearum, Erysiphe dchoracearum, Blumeria graminis, Podosphaera xanthii, Sphaerotheca fuliginea, Pythium ultirnurn, Uncinula necator, Mycosphaerella pinodes, Magnaporthe grisea, Bipolaris oryzae, Magnaporthe grisea, Rhizoctonia solani, Phytophthora sojae. Schizaphis graminum, Bemisia tabaci, Rhopalosiphum maidis, Deroceras reticulatum, Diatraea saccharalis, Schizaphis graminum and Myzus persicae.

In a particular embodiment of the invention, there is provided a method for increasing resistace to a disease in a plant, comprising the steps of: planting in a field a plant as described herein; and ensuring that the plant is provided with a supply of Si at a concentration of at least about 0.8 mM.

In a particular embodiment of the invention, there is provided a method of reducing abiotic stress damage in a crop wherein the abiotic stress is caused by any one of the following: drought, flooding/excess water, high level of salinity, heavy metal, aluminum, manganese, cadmium, zinc, UV-B, boron, cold temperature, heat, or herbicide, the method comprising the steps of: planting in a field a plant as described herein; and ensuring that the plant is provided with a supply of Si at a concentration of at least about 0.8 mM.

Resistance against diseases such as the following are encompassed within the present invention: powdery mildew, pythiu ultimum, root rot, leaf spot, blast, brown spot, leaf and neck blast, sheath blight; schizaphis graminum; brown-stem rot; soybean cyst nematode; and root-knot nematode. As well, resistance against pests such as the following are encompassed within the present invention: whitefly, aphid, gery field slug, sugarcane borer, green bug, or aphid.

Resistance against biotic and abiotic stresses such as the following are also encompassed within the present invention: salt (salinity), drought, aluminum, manganese, cadmium, zinc, UV-B, boron or cold (i.e. extreme temperatures).

In most cases, the beneficial role of Si will be more manifest in plant species accumulating higher amounts of Si, such as members of the grass family. In the case of rice for instance, Si amendments were found to enhance resistance against diseases such as blast, brown spot, and sheath blight (Table 1). The prophylactic effects of Si against insect pests have also been observed in several studies (Table 2). Sugarcane is another high Si accumulator and for which many positive effects have been observed under Si fertilization (Table 2). Similarly, enhancement of resistance against different insect pests has been reported in maize, rice, wheat, and cucumber, particularly, a closterovirus that may be Beet Pseudo-Yellows Virus (BPYV) or Cucurbit Yellow Stunting Disorder Virus (CYSDV).

Abiotic stress tolerance is a major constrain in crop yield production including soybean. Drought imposed by a water limiting environment, flooding, high level of salinity and heavy metal stress are the major concerns of abiotic stress. Si application has shown a great level of yield improvement against these stresses in different plant species (Table 3).

In addition to improving biotic and abiotic stress resistance, Si application has been reported to improve several agronomical traits. Increase in seedling vigor, yield potential and phosphate uptake has been observed with Si application in rice (Table 4).

Agronomical traits improved by high Si uptake are also encompassed within the present invention may be selected from, amongst others: plant growth, yield, seedling growth, phosphorus uptake, lodging, reproductive growth, or grain quality.

TABLE 1
Details of experimental evidence provided in the reports demonstrating the beneficial effects of silicon amendment on the
disease resistance in different plant species

| Table 1. | | | |
|---|---|---|---|
| Crop | Disease resistance | Reference | Title of the article |
| Arabidopsis | Powdery mildew | Vivancos et al. 2015 | Silicon-mediated resistance of Arabidopsis |
| (transgenic) | (Golovinomyces cichoracearum) | | against powdery mildew involves mechanisms |
| | | | other than the salicylic acid (SA)-dependent |
| | | | defence pathway |
| Arabidopsis | Powdery mildew (Erysiphe | Ghanmi et al. 2004 | Powdery mildew of Arabidopsis thaliana: a |
| | cichoracearum) | | pathosystem for exploring the role of silicon in |
| | | | plant-microbe interactions |
| Barley (Hordeum vulgare) | Powdery mildew (Blumeria graminis) | Wiese et al. 2005 | Osmotic stress and silicon act additively in |
| | | | enhancing pathogen resistance in barley against |
| | | | barley powdery mildew |
| Barley (Hordeum vulgare) | Powdery mildew (Blumeria graminis) | Riciout et al. 2006 | Multiple avirulence paralogues in cereal powdery |
| | | | mildew fungi may contribute to parasite fitness |
| | | | and defeat of plant resistance |
| Cucumber (Cucumis | Powdery mildew (Podosphaera | Liang et al. 2005 | Effects of foliar-and root-applied silicon on the |
| sativus) | xanthii) | | enhancement of induced resistance to powdery |
| | | | mildew in Cucumis sativus |
| Cucumber (Cucumis | Powdery mildew (Sphaerotheca | Wei et al. 2004 | Effects of silicon supply and Sphaerotheca |
| sativus) | fuliginea) | | fuliginea inoculation on resistance of cucumber |
| | | | seedlings against powdery mildew |
| Cucumber (Cucumis | Powdery mildew (Sphaerotheca | Menzies et al. 1991 | The influence of silicon on cytological |
| sativus) | fuliginea) | | interactions between Sphaerotheca fuliginea |
| | | | and Cucumis sativus |
| Cucumber (Cucumis | Pythium ultimum | Chérif et al. 1992 | Silicon induced resistance in cucumber plants |
| sativus) | | | against Pythium ultimum |
| Cucumber (Cucumis | Root rot (Pythium ultimum) | Chérif et al. 1994 | Defense responses induced by soluble silicon in |
| sativus) | | | cucumber roots infected by Pythium spp |
| Grape (Vitis vinifera) | Powdery mildew (Uncinula necator) | Bowen et al. 1992 | Soluble silicon sprays inhibit powdery mildew |
| | | | development on grape leaves |
| Oat (Avena sativa) | Powdery mildew (Blumeria graminis) | Carver et al. 1998 | Silicon deprivation enhances localized |
| | | | autofluorescent responses and phenylalanine |
| | | | ammonia-lyase activity in oat attacked by |
| | | | Blumeria graminis |
| Oat (Avena sativa) | Powdery mildew (Blumeria graminis) | Carver et al. 1998 | Phenylalanine ammonia-lyase inhibition, |
| | | | autofluorescence, and localized accumulation of |
| | | | silicon, calcium and manganese in oat epidermis |
| | | | attacked by the powdery mildew fungus |
| Peas (Pisum sativum) | Leaf spot (Mycosphaerella pinodes) | Dann et al. 2002 | Peas grown in media with elevated plant- |
| | | | available silicon levels have higher activities of |
| | | | chitinase and β-1, 3-glucanase, are less |
| | | | susceptible to a fungal leaf spot pathogen and |
| | | | accumulate more foliar silicon |
| Rice (Oryza sativa) | Blast (Magnaporthe grisea) | Kim et al. 2002 | Silicon-induced cell wall fortification of rice |
| | | | leaves: a possible cellular mechanism of |
| | | | enhanced host resistance to blast |
| Rice (Oryza sativa) | Blast (Magnaporthe grisea) | Rodrigues et al. 2004 | Silicon enhances the accumulation of diterpenoid |
| | | | phytoalexins in rice: a potential mechanism for |
| | | | blast resistance |
| Rice (Oryza sativa) | Blast (Magnaporthe grisea) | Rodrigues et al. 2003 | Ultrastructural and cytochemical aspects of |
| | | | silicon-mediated rice blast resistance |
| Rice (Oryza sativa) | Blast (Magnaporthe grisea) | Cai et al. 2008 | Physiological and cytological mechanisms of |
| | | | silicon-induced resistance in rice against blast |
| | | | disease |
| Rice (Oryza sativa) | Blast (Magnaporthe grisea) | Seebold et al. 2001 | The influence of silicon on components of |
| | | | resistance to blast in susceptible, partially |
| | | | resistant, and resistant cultivars of rice |
| Rice (Oryza sativa) | Blast (Magnaporthe grisea) | Seebold et al. 2000 | Effect of silicon rate and host resistance on blast, |
| | | | scald, and yield of upland rice |
| Rice (Oryza sativa) | Blast (Magnaporthe grisea) | Osuna-Canizalez et al. | Nitrogen form and silicon nutrition effects on |
| | | 1991 | resistance to blast disease of rice |
| Rice (Oryza sativa) | Brown spot (Bipolaris oryzae) | Dallagnoi et al. 2009 | Defective active silicon uptake affects some |
| | | | components of rice resistance to brown spot |
| Rice (Oryza sativa) | Brown spot (Bipolaris oryzae) | Zanão Junior et al. 2009 | Rice resistance to brown spot mediated by |
| | | | silicon and its interaction with manganese |
| Rice (Oryza sativa) | Leaf and neck blast (Magnaporthe | Seebold Jr et al. 2004 | Effects of silicon and fungicides on the control of |
| | grisea) | | leaf and neck blast in upland rice |
| Rice (Oryza sativa) | Several | Winslow et al. 1992 | Silicon, disease resistance, and yield of rice |
| | | | genotypes under upland cultural conditions |
| Rice (Oryza sativa) | Several | Ranganathan et al. 2006 | Effects of silicon sources on its deposition, |
| | | | chlorophyll content, and disease and pest |
| | | | resistance in rice |
| Rice (Oryza sativa) | Sheath blight (Rhizoctonia solani) | Peters et al. 2001 | Effect of silicon and host resistance on sheath |
| | | | blight development in rice |
| Rice (Oryza sativa) | Sheath blight (Rhizoctonia solani) | Rodrigues et al. 2003 | Influence of silicon on sheath blight of rice in |
| | | | Brazil |
| Soybean (Glycine max) | Root rot (Phytophthora sojae) | Guérin et al. 2014 | A Zoospore Inoculation Method with |
| | | | Phytophthora sojae to Assess the Prophylactic |
| | | | Role of Silicon on Soybean Cultivars |
| Wheat (Triticum aestivum) | Schizaphis graminum | Gomes et al. 2005 | Resistance induction in wheat plants by silicon |
| | | | and aphids |
| Wheat (Triticum aestivum) | Powdery mildew (Blumeria graminis) | Bélanger et al. 2003 | Cytological evidence of an active role of silicon in |
| | | | wheat resistance to powdery mildew |
| Wheat (Triticum aestivum) | Powdery mildew (Blumeria graminis) | Rémus-Borel et al. 2005 | Silicon induces antifungal compounds in |
| | | | powdery mildew-infected wheat |
| Wheat (Triticum aestivum) | Powdery mildew (Blumeria graminis) | Guével et al. 2007 | Effect of root and foliar applications of soluble |
| | | | silicon on powdery mildew control and growth of |
| | | | wheat plants |
| Wheat (Triticum aestivum) | Several | Rodgers-Gray et al. 2004 | Effects of straw and silicon soil amendments on |
| | | | some foliar and stem-base diseases in |
| | | | pot-grown winter wheat |

TABLE 2
Details of experimental evidence provided in the reports demonstrating the beneficial effects of silicon amendment on the insect
resistance in different plant species

| Table 2. | | | |
|---|---|---|---|
| Crop | Insect resistance | Reference | Title of the article |
| Cucumber (Cucumis | Whitefly (Bemisia tabaci) | Correa et al. 2005 | Silicon and acibenzolar-S-methyl as resistance |
| sativus) | | | inducers in cucumber, against the whitefly Bemisia |
| | | | tabaci biotype B |
| Maize (Zea mays) | Aphid (Rhopalosiphum maidis) | Moraes et al. 2005 | Feeding non-preference of the corn leaf aphid |
| | | | Rhopalosiphum maidis to corn plants |
| Rice (Oryza sativa) | Grey field slug (Deroceras | Wadham et al. 1981 | The silicon content of Oryza sativa L |
| | reticulatum) | | |
| Sugarcane (Saccharum | Sugarcane borer (Diatraea | Anderson et al. 2001 | Effect of silicon on expression of resistance to |
| officinarum) | saccharalis) | | sugarcane borer |
| Sugarcane (Saccharum | Sugarcane borer (Diatraea | Keeping et al. 2002 | Effect of four sources of silicon on resistance of |
| officinarum) | saccharalis) | | sugarcane varieties to Eldana saccharina |
| Sugarcane (Saccharum | Sugarcane borer (Diatraea | Kvedaras et al. 2007 | Silicon impedes stalk penetration by the borer |
| officinarum) | saccharalis) | | Eldana saccharina in sugarcane |
| Sugarcane (Saccharum | Sugarcane borer (Diatraea | Kvedaras et al. 2007 | Larval performance of the pyralid borer Eldana |
| officinarum) | saccharalis) | | saccharina Walker and stalk damage in |
| | | | sugarcane: Influence of plant silicon, cultivar and |
| | | | feeding site |
| Sugarcane (Saccharum | Sugarcane borer (Diatraea | Kvedaras et al. 2005 | Effects of silicon on the African stalk borer, Eldana |
| officinarum) | saccharails) | | saccharina in sugarcane |
| Sugarcane (Saccharum | Sugarcane borer (Diatraea | Keeping et al. 2009 | Epidermal silicon in sugarcane: Cultivar |
| officinarum) | saccharails) | | differences and role in resistance to sugarcane |
| | | | borer |
| Wheat (Triticum aestivum) | Green bug (Schizaphis | Goussain et al. 2005 | Effect of silicon applied to wheat plants on the |
| | graminum) | | biology and probing behaviour of the greenbug |
| | | | Schizaphis graminum |
| Wheat (Triticum aestivum) | Green bug (Schizaphis | Moraes et al. 2004 | Silicon influence on the tritrophic interaction: |
| | graminum) | | wheat plants, the greenbug Schizaphis graminum, |
| | | | and its natural enemies, Chrysoperla externa and |
| | | | Aphidius colemani Viereck |
| Zinnia (Zinnia elegans) | Aphid (Myzus persicae) | Ranger et al. 2009 | Influence of silicon on resistance of Zinnia |
| | | | elegans to Myzus persicae |

TABLE 3
Details of experimental evidence provided in the reports demonstrating the beneficial effects of silicon amendment on the abiotic
stress tolerance in different plant species

| Table 3. | | | |
|---|---|---|---|
| Crop | Abiotic stress | Reference | Title of the article |
| Alfalfa (Medicago sativa) | Salt | Wang et al. 2007 | Effects of NaCl and silicon on ion distribution in the |
| | | | roots, shoots and leaves of two alfalfa cultivars with |
| | | | different salt t |
| Augustinegrass (Stenotaphrum | Drought | Trenholm et al. 2004 | Influence of silicon on drought and shade tolerance of |
| secundatum) | | | St. Augustinegrass |
| Barley (Hordeum vulgare) | Aluminum | Hammond et al. 1995 | Aluminium/silicon interactions in barley |
| Barley (Hordeum vulgare) | Drought | Walker et al. 1991 | Silicon accumulation and 13C composition as indices of |
| | | | water-use efficiency in barley cultivars |
| Barley (Hordeum vulgare) | Manganese | Horiguchi et al. 1987 | Mechanism of manganese toxicity and tolerance of |
| | | | plants VI. effect of silicon on alleviation of manganese |
| | | | toxicity of barley |
| Barley (Hordeum vulgare) | Salt | Liang et al. 1996 | Effects of silicon on salinity tolerance of two barley |
| | | | cultivars |
| Barley (Hordeum vulgare) | Salt | Liang et al. 1999 | Effects of silicon on enzyme activity and sodium, |
| | | | potassium and calcium concentration in barley under |
| | | | salt stress |
| Barley (Hordeum vulgare) | Salt | Liang et al. 2003 | Exogenous silicon (Si) increases antioxidant enzyme |
| | | | activity and reduces lipid peroxidation in roots of salt- |
| | | | stressed barley |
| Barley (Hordeum vulgare) | Salt | Yongchao et al. 1998 | Effect of silicon on leaf ultrastructure, chlorophyll content |
| | | | and photosynthetic activity of barley under salt stress |
| Bayahonda blanca (Prosopis | Salt | Bradbury et al. 1990 | The effect of silicon on the growth of Prosopis juliflora |
| juliflora) | | | growing in saline soil |
| Brassica | Cadmium | Song et al. 2009 | Silicon-enhanced resistance to cadmium toxicity in |
| | | | Brassica chinensis |
| Comon Bean (Phaseolus vulgaris) | Manganese | Horst et al. 1978 | Effect of silicon on manganese tolerance of bean plants |
| Cotton | Aluminum | Li et al. 1989 | Response of cotton cultivars to aluminum in solutions |
| (Gossypium Spp.) | | | with varying silicon concentrations |
| Cowpea (Vigna unguiculata) | Manganese | Iwasaki et al. 2002 | Leaf apoplastic silicon enhances manganese tolerance |
| | | | of cowpea |
| Cowpea (Vigna unguiculata) | Manganese | Iwasaki et al. 2002 | Effects of silicon supply on apoplastic manganese |
| | | | concentrations in leaves and their relation to manganese |
| | | | tolerance in cowpea |
| Cucumber (Cucumis sativus) | Cadmium | Feng et al. 2010 | Silicon supplementation ameliorated the inhibition of |
| | | | photosynthesis and nitrate metabolism by cadmium (Cd) |
| | | | toxicity in Cucum |
| Cucumber (Cucumis sativus) | Drought | Ma et al. 2004 | Effects of silicon application on drought resistance of |
| | | | Cucumber (Cucumis sativus) plants |
| Cucumber (Cucumis sativus) | Manganese | Rogalla et al. 2002 | Role of leaf apoplast in silicon-mediated manganese |
| | | | tolerance of Cucumis sativus L |
| Cucumber (Cucumis sativus) | Salt | Zhu et al. 2004 | Silicon alleviates salt stress and increases antioxidant |
| | | | enzymes activity in leaves of salt-stressed cucumber |
| Maize (Zea mays) | Aluminium | Kidd et al. 2001 | The role of root exudates in aluminium resistance and |
| | | | silicon-induced amelioration of aluminium toxicity in |
| | | | three varieties of |
| Maize (Zea mays) | Aluminum | Wang et al. 2004 | Apoplastic binding of aluminum is involved in silicon- |
| | | | induced amelioration of aluminum toxicity in maize |
| Maize (Zea mays) | Aluminum | Corrales et al. 1997 | Influence of silicon pretreatment on aluminium toxicity in |
| | | | maize roots |
| Maize (Zea mays) | Aluminum | Barcelo et al. 1993 | Silicon amelioration of aluminium toxicity in teosinte |
| Maize (Zea mays) | Cadmium, Zink | da Cunha et al. 2009 | Silicon effects on metal tolerance and structural changes |
| | | | in maize |
| Maize (Zea mays) | Drought | Kaya et al. 2006 | Effect of silicon on plant growth and mineral nutrition of |
| | | | maize grown under water-stress conditions |
| Maize (Zea mays) | Drought | Gao et al. 2005 | Silicon improves water use efficiency in maize plants |
| Maize (Zea mays) | Drought | Li et al. 2007 | Effects of silicon on photosynthesis and antioxidative |
| | | | enzymes of maize under drought stress |
| Maize (Zea mays) | Manganese | Doncheva et al. 2009 | Silicon amelioration of manganese toxicity in Mn- |
| | | | sensitive and Mn-tolerant maize varieties |
| Peanut (Arachis hypogaea) | Cadmium | Shi et al. 2010 | Silicon alleviates cadmium toxicity in peanut plants in |
| | | | relation to cadmium distribution and stimulation of |
| | | | antioxidative enzy |
| Pumpkin (Cucurbita maxima) | Manganese | Iwasaki et al. 1999 | Effect of silicon on alleviation of manganese toxicity in |
| | | | pumpkin |
| Rice (Oryza sativa) | Aluminum | Gu et al. 1998 | Effects of silicon supply on amelioration of aluminum |
| | | | injury and chemical forms of aluminum in rice plants |
| Rice (Oryza sativa) | Cadmium | Wang et al. 2000 | Silicon induced cadmium tolerance of rice seedlings |
| Rice (Oryza sativa) | Cadmium | Zhang et al. 2008 | Long-term effects of exogenous silicon on cadmium |
| | | | translocation and toxicity in rice |
| Rice (Oryza sativa) | Cadmium | Nwugo et al. 2008 | Silicon-induced cadmium resistance in rice |
| Rice (Oryza sativa) | Drought | Chen et al. 2011 | Silicon alleviates drought stress of rice plants by |
| | | | improving plant water status, photosynthesis and |
| | | | mineral nutrient absorpti |
| Rice (Oryza sativa) | Manganese | Horiguchi et al. 1988 | Mechanism of manganese toxicity and tolerance of |
| | | | plants: IV. Effects of silicon on alleviation of manganese |
| | | | toxicity of rice |
| Rice (Oryza sativa) | Salt | Yeo et al. 1999 | Silicon reduces sodium uptake in rice |
| Rice (Oryza sativa) | Uv-b | Li et al. 2004 | Effects of silicon on rice leaves resistance to ultraviolet-B |
| Sorghum | Aluminum, | Galvez et al. 1987 | Silicon interactions with manganese and aluminum |
| | manganese | | toxicity in sorghum |
| Sorghum | Drought | Hattori et al. 2005 | Application of silicon enhanced drought tolerance in |
| | | | Sorghum bicolor |
| Sorghum | Manganese | Galvez et al. 1989 | Effects of silicon on mineral composition of sorghum |
| | | | grown with excess manganese 1 |
| Soybean (Glycine max) | Drought | Shen et al. 2010 | Silicon effects on photosynthesis and antioxidant |
| | | | parameters of soybean seedlings under drought and |
| | | | ultraviolet-B radiation |
| Sugarcane (Saccharum | Aluminum | Fox et al. 1967 | Soil and plant silicon and silicate response by sugarcane |
| officinarum) | | | |
| Wheat (Triticum aestivum) | Boron | Gunes et al. 2007 | Silicon increases boron tolerance and reduces oxidative |
| | | | damage of wheat grown in soil with excess boron |
| Wheat (Triticum aestivum) | Cold | Liang et al. 2008 | Role of silicon in enhancing resistance to freezing stress |
| | | | in two contrasting winter wheat cultivars |
| Wheat (Triticum aestivum) | Drought | Pei et al. 2010 | Silicon improves the tolerance to water-deficit stress |
| | | | induced by polyethylene glycol in wheat |
| Wheat (Triticum aestivum) | Drought | Gong et al. 2005 | Silicon alleviates oxidative damage of wheat plants in |
| | | | pots under drought |
| Wheat (Triticum aestivum) | Drought | Gong et al. 2003 | Effects of silicon on growth of wheat under drought |
| Wheat (Triticum aestivum) | Salt | Ahmad et al. 1992 | Role of silicon in salt tolerance of wheat |
| Wheat (Triticum aestivum) | Salt | Tuna et al. 2008 | Silicon improves salinity tolerance in wheat plants |
| Wheat (Triticum aestivum) | Salt | Tahir et al. 2006 | Beneficial effects of silicon in wheat |
| Wheat (Triticum aestivum) | Salt | Saqib et al. 2008 | Silicon-mediated improvement in the salt resistance of |
| | | | wheat results from increased sodium exclusion and |
| | | | resistance to oxidati |
| Zucchini (Cucurbita pepo) | Salt | Savvas et al. 2009 | Silicon supply in soilless cultivations of zucchini |
| | | | alleviates stress induced by salinity and powdery mildew |
| | | | infections |

TABLE 4
Details of experimental evidence provided in the reports demonstrating the beneficial effects of silicon amendment on
agronomical performance in different plant species

| Table 4. | Agronomical | | |
|---|---|---|---|
| Crop | parameter | Reference | Title of the article |
| Alfalfa (Medicago sativa) | Plant growth | Guo et al. 2006 | Effect of silicon on the morphology of shoots and roots of alfalfa |
| Augustinegrass (Stenotaphrum | Plant growth | Brecht et al. 2004 | Influence of silicon and chlorothalonil on the suppression of gray |
| secundatum) | | | leaf spot and increase plant growth in St. Augustinegrass |
| Banana (Musa × paradisiaca) | Plant growth | Henriet et al. 2006 | Effects, distribution and uptake of silicon in banana |
| Barley (Hordeum vulgare) | Yield | Williams et al. 1957 | The effect of silicon on yield and manganese-54 uptake and |
| | | | distribution in the leaves of barley plants grown in culture solutions |
| Cereals | Seedling growth | Hossain et al. 2002 | Growth promotion and an increase in cell wall extensibility by |
| | | | silicon in rice and some other Poaceae seedlings |
| Cucumber (Cucumis sativus) | Plant growth | Miyake et al. 1983 | Effect of silicon on the growth of solution-cultured cucumber plant |
| Cucumber (Cucumis sativus) | Phosphorus uptake | Marschner et al. 1990 | Growth enhancement by silicon in cucumber plants depends on |
| | | | imbalance in phosphorus and zinc supply |
| Cucumber (Cucumis sativus) | Plant growth | Miyake et al. 1983 | Effect of silicon on the growth of cucumber plant in soil culture |
| Pine (Pinus taeda) | Seedling growth | Emadian et al. 1989 | Growth Enhancement of Loblolly Pine Seedlings by Silicon |
| Rice (Oryza sativa) | Lodging | Idris et al. 1975 | The effect of silicon on lodging of rice in presence of added |
| | | | nitrogen |
| Rice (Oryza sativa) | Phosphorus uptake | Ma et al. 1990 | Effect of silicon on the growth and phosphorus uptake of rice |
| Rice (Oryza sativa) | Plant growth | Ma et al. 1989 | Effect of silicon on the growth of rice plant at different growth |
| | | | stages |
| Rice (Oryza sativa) | Reproductive | Inanaga et al. 2002 | Effect of silicon application on reproductive growth of rice plant |
| | growth | | |
| Rice (Oryza sativa) | Seedling growth | Sistani et al. 1997 | Effect of rice hull ash silicon on rice seedling growth |
| Rice (Oryza sativa) | Yield | Deren et al. 1994 | Silicon concentration, disease response, and yield components of |
| | | | rice genotypes grown on flooded organic histosols |
| Rice (Oryza sativa) | Yield, growth, grain | Korndörfer et al. 1999 | Influence of silicon on grain discoloration and upland rice grown |
| | quality | | on four savanna soils of Brazil |
| Sugarcane (Saccharum officinarum) | Plant growth | Matichenkov et al. | Silicon as a beneficial element for sugarcane |
| | | 2002 | |
| Sunflower (Helianthus annuus) | Plant growth | Kamenidou et al. 2008 | Silicon supplements affect horticultural traits of greenhouse- |
| | | | produced ornamental sunflowers |

### Method of Identifying

In accordance with a further embodiment of the present invention, there is provided a method for identifying a high Si accumulating soybean variety or lineage comprising the step of: a) obtaining a part of a soybean plant; and b) analyzing the part to detect a marker for soybean high Si uptake, the marker comprising nucleic acid comprising at least one single nucleotide polymorphism (SNP) at a position on chromosome 16 from 33104446 bp to 35762786 bp; wherein when the marker is detected, the variety or lineage is identified as a high Si accumulator (for example, any marker selected from Tables 15-20 or markers in close proximity to).

Alternatively, in a particular embodiment, the invention provides a method of identifying or selecting a first soybean plant having increased Si uptake, the method comprising the steps of: a) isolating a nucleic acid from a first soybean plant; b) detecting in the nucleic acid the presence of a molecular marker that associates with increased Si uptake and wherein the molecular marker is: associated with a H1 haplotype; or located within 20 cM, 10 cM, ScM, 1 cM or 0.5 cM of a chromosomal interval corresponding to a genomic region from Hikmok sorip chromosome 16 at about 92.6 cM to about 132 cM distance; or located from physical positions 33.15M base-pairs to 36.72M base-pairs as indicated on a genetic linkage map from Hikmok sorip (PI372415A); and c) identifying or selecting the soybean plant on the basis of the presence of the molecular marker of b); thereby identifying or selecting a first soybean plant having increased Si uptake.

Particularly, this method is used in a commercial soybean plant breeding program More particularly, this the detecting step in this method comprises detecting at least one allelic form of a polymorphic simple sequence repeat (SSR) or a single nucleotide polymorphism (SNP). Most particularly, the detecting comprises amplifying the marker locus or a portion of the marker locus and detecting the resulting amplified marker amplicon (for e.g. a amplicon generated by a primer pair selected from SEQ ID NO. 12, 13 and 278-495).

In accordance with a particular embodiment of the method for identifying or selecting further comprises the step where the chromosome interval associated with increased Si uptake is introgressed into a second soybean plant or germplasm to produce an introgressed soybean plant or germplasm having increased Si uptake wherein the introgressed soybean plant further comprises at least one of: a) a SNP marker selected from the group consisting of: A(33673022), G(33673483), C(33681630), T(33682500), G(33683047), and C(33683049) on genes Glyma30000 or 30020; b) a marker corresponding to a genomic region from Hikmok sorip chromosome 16 at about 92.6 cM to about 132 cM distance or c) from physical positions 33.15M base-pairs to 36.72M base-pairs, or portion thereof as indicated on a genetic linkage map from Hikmok sorip (PI372415A).

Still, according to this method, the second soybean plant or germplasm displays low Si uptake as compared to the first soybean plant or germplasm, wherein the introgressed soybean plant or germplasm displays increased Si uptake as compared to the second plant or germplasm. Particularly, the second soybean plant or germplasm comprises an elite soybean strain or an exotic soybean strain,

In accordance with a particular aspect, the method of identifying may also comprise electronically transmitting or electronically storing data representing the detected allele or molecular marker in a computer readable medium. Still, particularly, the molecular marker or allele is determined using TASSEL, GeneFlow, or MapManager-QTX software.

Particularly, at least one parental line of the plant may be selected or identified by a molecular marker associated with a nucleic acid as defined herein.

### Markers

In particular, the present invention provides at least one marker indicative of high Si uptake for soybean or other plants, particularly located from 33.15 Mb pairs to 36.72 Mb pairs of the Williams82 reference genome. This marker is useful for developing and identifying a soybean plant that has, or has been modified to achieve, high Si uptake.

Still, particularly, the plant originates from a parental line that was selected or identified by a molecular marker located within 20 cM, 10 cM, 5 cM, 1 cM or 0.5 cM of the chromosomal interval, wherein the molecular marker is associated with Si accumulation in the plant, more particularly, high Si accumulation.

According to a particular embodiment, the marker corresponds to: a genomic region from Hikmok sorip chromosome 16 at about 92.6 cM to about 132 cM distance; or a genomic region from physical positions 33.15M base-pairs to 36.72M base-pairs, or portion thereof as indicated on a genetic linkage map from Hikmok sorip (PI372415A). Alternatively the marker corresponds to a SNP selected from the group consisting of: A(33673022), G(33673483), C(33681630), T(33682500), G(33683047), and C(33683049) of genes glyma16g:30000 or glyma16g:30020.

Alternatively, the molecular marker is located within 20 cM, 10 cM, 5 cM, 1 cM or 0.5 cM of a single nucleotide polymorphism (SNP) marker associated with increased Si accumulation selected from the group consisting of: G(33672717), A(33673022), G(33673483), C(33681630), T(33681946), T(33681961), T(33682500), G(33683047), C(33683049) and any marker indicated in Tables 15-18 as indicated on a genetic linkage map from Hikmok sorip (PI372415A).

More particularly, this marker is a nucleic acid that may include a single nucleotide polymorphism selected from the group consisting of: SNP605 (33104446 bp), SNP606 (33527064 bp), SNP607 (33595090 bp), SNP608 (33802005 bp), SNP609 (35218844 bp) and SNP610 (35762786 bp) as found in chromosome 16 of Hikmok sorip.

In particular, the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite.

The genomic region on chromosome 16 corresponding to the markers found is as defined by SEQ ID NO.1. Table 5 lists the high silicon accumulator region from chromosome 16 of Hikmok sorip soybean plant and the corresponding putative gene start and end codons as defined by SEQ ID NO.1.

TABLE 5
List of potential genes present at Hisil region on chromosome
16 from 33104446 bp to 35762786 bp from Hikmok sorip

| Gene Name | Transcript Name | Gene Start (bp) | Gene End (bp) |
|---|---|---|---|
| Glyma16g29287 | Glyma16g29287.1 | 33117480 | 33118554 |
| Glyma16g29300 | Glyma16g29300.2 | 33121274 | 33125742 |
| Glyma16g29300 | Glyma16g29300.3 | 33121274 | 33125742 |
| Glyma16g29315 | Glyma16g29315.1 | 33140575 | 33145668 |
| Glyma16g29315 | Glyma16g29315.2 | 33140601 | 33145666 |
| Glyma16g29330 | Glyma16g29330.1 | 33163898 | 33165670 |
| Glyma16g29340 | Glyma16g29340.1 | 33176754 | 33178509 |
| Glyma16g29370 | Glyma16g29370.1 | 33191101 | 33193066 |
| Glyma16g29380 | Glyma16g29380.1 | 33209634 | 33211251 |
| Glyma16g29400 | Glyma16g29400.1 | 33218464 | 33219888 |
| Glyma16g29411 | Glyma16g29411.1 | 33224507 | 33241114 |
| Glyma16g29420 | Glyma16g29420.1 | 33235180 | 33237175 |
| Glyma16g29430 | Glyma16g29430.1 | 33242250 | 33244325 |
| Glyma16g29440 | Glyma16g29440.3 | 33245292 | 33252887 |
| Glyma16g29440 | Glyma16g29440.4 | 33245292 | 33252887 |
| Glyma16g29440 | Glyma16g29440.2 | 33245292 | 33252887 |
| Glyma16g29450 | Glyma16g29450.3 | 33263382 | 33267806 |
| Glyma16g29450 | Glyma16g29450.1 | 33263382 | 33267786 |
| Glyma16g29450 | Glyma16g29450.4 | 33263382 | 33267786 |
| Glyma16g29463 | Glyma16g29463.1 | 33270817 | 33271810 |
| Glyma16g29476 | Glyma16g29476.1 | 33275084 | 33279607 |
| Glyma16g29490 | Glyma16g29490.2 | 33293375 | 33297776 |
| Glyma16g29490 | Glyma16g29490.3 | 33293375 | 33297776 |
| Glyma16g29501 | Glyma16g29501.1 | 33312431 | 33313552 |
| Glyma16g29510 | Glyma16g29510.1 | 33317104 | 33319767 |
| Glyma16g29520 | Glyma16g29520.2 | 33321294 | 33325497 |
| Glyma16g29541 | Glyma16g29541.1 | 33336584 | 33343085 |
| Glyma16g29561 | Glyma16g29561.1 | 33345959 | 33347937 |
| Glyma16g29580 | Glyma16g29580.1 | 33354370 | 33360885 |
| Glyma16g29590 | Glyma16g29590.4 | 33362742 | 33365896 |
| Glyma16g29590 | Glyma16g29590.3 | 33362742 | 33365896 |
| Glyma16g29600 | Glyma16g29600.2 | 33366648 | 33373909 |
| Glyma16g29600 | Glyma16g29600.3 | 33366648 | 33373909 |
| Glyma16g29611 | Glyma16g29611.1 | 33375473 | 33380054 |
| Glyma16g29620 | Glyma16g29620.1 | 33382294 | 33383795 |
| Glyma16g29630 | Glyma16g29630.1 | 33385941 | 33388630 |
| Glyma16g29640 | Glyma16g29640.1 | 33391337 | 33392933 |
| Glyma16g29650 | Glyma16g29650.2 | 33404884 | 33406256 |
| Glyma16g29650 | Glyma16g29650.1 | 33404884 | 33406256 |
| Glyma16g29661 | Glyma16g29661.1 | 33409758 | 33410957 |
| Glyma16g29670 | Glyma16g29670.1 | 33413333 | 33414359 |
| Glyma16g29680 | Glyma16g29680.2 | 33416009 | 33417784 |
| Glyma16g29690 | Glyma16g29690.1 | 33423741 | 33425662 |
| Glyma16g29690 | Glyma16g29690.2 | 33423741 | 33425662 |
| Glyma16g29701 | Glyma16g29701.1 | 33428773 | 33429954 |
| Glyma16g29710 | Glyma16g29710.1 | 33432555 | 33433447 |
| Glyma16g29720 | Glyma16g29720.1 | 33439338 | 33441275 |
| Glyma16g29740 | Glyma16g29740.1 | 33444567 | 33451843 |
| Glyma16g29750 | Glyma16g29750.1 | 33452984 | 33456955 |
| Glyma16g29760 | Glyma16g29760.1 | 33457391 | 33463325 |
| Glyma16g29760 | Glyma16g29760.2 | 33457391 | 33463325 |
| Glyma16g29780 | Glyma16g29780.1 | 33465753 | 33469045 |
| Glyma16g29790 | Glyma16g29790.1 | 33472525 | 33475361 |
| Glyma16g29810 | Glyma16g29810.2 | 33488916 | 33490567 |
| Glyma16g29841 | Glyma16g29841.1 | 33495788 | 33498544 |
| Glyma16g29841 | Glyma16g29841.2 | 33495788 | 33498544 |
| Glyma16g29841 | Glyma16g29841.3 | 33495836 | 33498541 |
| Glyma16g29841 | Glyma16g29841.4 | 33495940 | 33498153 |
| Glyma16g29830 | Glyma16g29830.1 | 33497194 | 33497346 |
| Glyma16g29850 | Glyma16g29850.2 | 33500401 | 33502384 |
| Glyma16g29860 | Glyma16g29860.1 | 33504174 | 33508434 |
| Glyma16g29860 | Glyma16g29860.2 | 33504174 | 33508434 |
| Glyma16g29870 | Glyma16g29870.2 | 33513548 | 33516668 |
| Glyma16g29880 | Glyma16g29880.2 | 33521922 | 33522569 |
| Glyma16g29890 | Glyma16g29890.1 | 33525365 | 33530003 |
| Glyma16g29900 | Glyma16g29900.1 | 33539909 | 33542679 |
| Glyma16g29910 | Glyma16g29910.2 | 33567442 | 33572345 |
| Glyma16g29910 | Glyma16g29910.3 | 33567460 | 33572332 |
| Glyma16g29910 | Glyma16g29910.1 | 33567460 | 33572332 |
| Glyma16g29920 | Glyma16g29920.2 | 33580523 | 33584738 |
| Glyma16g29920 | Glyma16g29920.1 | 33580799 | 33584738 |
| Glyma16g29930 | Glyma16g29930.2 | 33589335 | 33590105 |
| Glyma16g29950 | Glyma16g29950.1 | 33596241 | 33597276 |
| Glyma16g29960 | Glyma16g29960.1 | 33608683 | 33612574 |
| Glyma16g29980 | Glyma16g29980.2 | 33632785 | 33637232 |
| Glyma16g29990 | Glyma16g29990.2 | 33650887 | 33653599 |
| Glyma16g30000 | Glyma16g30000.1 | 33667117 | 33674724 |
| Glyma16g30000 | Glyma16g30000.2 | 33670072 | 33674724 |
| Glyma16g30020 | Glyma16g30020.2 | 33680052 | 33684676 |
| Glyma16g30030 | Glyma16g30030.1 | 33692439 | 33700420 |
| Glyma16g30041 | Glyma16g30041.1 | 33705120 | 33711897 |
| Glyma16g30050 | Glyma16g30050.3 | 33719023 | 33724462 |
| Glyma16g30060 | Glyma16g30060.1 | 33727942 | 33736003 |
| Glyma16g30070 | Glyma16g30070.2 | 33738529 | 33744838 |
| Glyma16g30081 | Glyma16g30081.3 | 33748982 | 33756820 |
| Glyma16g30081 | Glyma16g30081.8 | 33748982 | 33756820 |
| Glyma16g30081 | Glyma16g30081.2 | 33748982 | 33756820 |
| Glyma16g30081 | Glyma16g30081.7 | 33748982 | 33756820 |
| Glyma16g30081 | Glyma16g30081.4 | 33748982 | 33756820 |
| Glyma16g30081 | Glyma16g30081.11 | 33748982 | 33756820 |
| Glyma16g30081 | Glyma16g30081.12 | 33748982 | 33756820 |
| Glyma16g30081 | Glyma16g30081.5 | 33748982 | 33756820 |
| Glyma16g30081 | Glyma16g30081.9 | 33748982 | 33756820 |
| Glyma16g30081 | Glyma16g30081.10 | 33748982 | 33756820 |
| Glyma16g30081 | Glyma16g30081.6 | 33748982 | 33756820 |
| Glyma16g30081 | Glyma16g30081.1 | 33748982 | 33756820 |
| Glyma16g30090 | Glyma16g30090.1 | 33757760 | 33758933 |
| Glyma16g30100 | Glyma16g30100.2 | 33761031 | 33770049 |
| Glyma16g30110 | Glyma16g30110.1 | 33767168 | 33767729 |
| Glyma16g30120 | Glyma16g30120.3 | 33776196 | 33781762 |
| Glyma16g30120 | Glyma16g30120.4 | 33776196 | 33780563 |
| Glyma16g30120 | Glyma16g30120.1 | 33776196 | 33781762 |
| Glyma16g30130 | Glyma16g30130.3 | 33787390 | 33791448 |
| Glyma16g30130 | Glyma16g30130.2 | 33787390 | 33791448 |
| Glyma16g30130 | Glyma16g30130.1 | 33787390 | 33790230 |
| Glyma16g30140 | Glyma16g30140.1 | 33792950 | 33798397 |
| Glyma16g30160 | Glyma16g30160.2 | 33800079 | 33806673 |
| Glyma16g30160 | Glyma16g30160.5 | 33800079 | 33806706 |
| Glyma16g30160 | Glyma16g30160.6 | 33800079 | 33806673 |
| Glyma16g30160 | Glyma16g30160.8 | 33800079 | 33806706 |
| Glyma16g30160 | Glyma16g30160.4 | 33800079 | 33806706 |
| Glyma16g30160 | Glyma16g30160.3 | 33800079 | 33806673 |
| Glyma16g30160 | Glyma16g30160.7 | 33800079 | 33806706 |
| Glyma16g30160 | Glyma16g30160.1 | 33800079 | 33806673 |
| Glyma16g30171 | Glyma16g30171.1 | 33810198 | 33825843 |
| Glyma16g30180 | Glyma16g30180.1 | 33826554 | 33831319 |
| Glyma16g30190 | Glyma16g30190.2 | 33833508 | 33853555 |
| Glyma16g30190 | Glyma16g30190.1 | 33833508 | 33853573 |
| Glyma16g30200 | Glyma16g30200.2 | 33866910 | 33870690 |
| Glyma16g30226 | Glyma16g30226.1 | 33896142 | 33899032 |
| Glyma16g30253 | Glyma16g30253.1 | 33916604 | 33918157 |
| Glyma16g30280 | Glyma16g30280.2 | 33946050 | 33949661 |
| Glyma16g30300 | Glyma16g30300.2 | 33962631 | 33965663 |
| Glyma16g30313 | Glyma16g30313.1 | 33970078 | 33977963 |
| Glyma16g30326 | Glyma16g30326.1 | 33971597 | 33975074 |
| Glyma16g30340 | Glyma16g30340.2 | 33981493 | 33984969 |
| Glyma16g30350 | Glyma16g30350.2 | 34010075 | 34013595 |
| Glyma16g30363 | Glyma16g30363.1 | 34027748 | 34030346 |
| Glyma16g30376 | Glyma16g30376.1 | 34039150 | 34040734 |
| Glyma16g30390 | Glyma16g30390.2 | 34047074 | 34050258 |
| Glyma16g30410 | Glyma16g30410.2 | 34061204 | 34063904 |
| Glyma16g30420 | Glyma16g30420.2 | 34065972 | 34067637 |
| Glyma16g30430 | Glyma16g30430.2 | 34068410 | 34069518 |
| Glyma16g30440 | Glyma16g30440.2 | 34074623 | 34078309 |
| Glyma16g30470 | Glyma16g30470.2 | 34085996 | 34093659 |
| Glyma16g30480 | Glyma16g30480.1 | 34098513 | 34101191 |
| Glyma16g30510 | Glyma16g30510.2 | 34109171 | 34112170 |
| Glyma16g30521 | Glyma16g30521.1 | 34119569 | 34122367 |
| Glyma16g30531 | Glyma16g30531.1 | 34126143 | 34128364 |
| Glyma16g30540 | Glyma16g30540.2 | 34131280 | 34134472 |
| Glyma16g30550 | Glyma16g30550.1 | 34141781 | 34142371 |
| Glyma16g30561 | Glyma16g30561.1 | 34144846 | 34147584 |
| Glyma16g30570 | Glyma16g30570.2 | 34152774 | 34157131 |
| Glyma16g30590 | Glyma16g30590.2 | 34164509 | 34167564 |
| Glyma16g30600 | Glyma16g30600.2 | 34174100 | 34176898 |
| Glyma16g30616 | Glyma16g30616.1 | 34180572 | 34181194 |
| Glyma16g30616 | Glyma16g30616.2 | 34180572 | 34183280 |
| Glyma16g30633 | Glyma16g30633.1 | 34187958 | 34190245 |
| Glyma16g30650 | Glyma16g30650.2 | 34203165 | 34206147 |
| Glyma16g30665 | Glyma16g30665.1 | 34215892 | 34218959 |
| Glyma16g30681 | Glyma16g30681.1 | 34225159 | 34226075 |
| Glyma16g30695 | Glyma16g30695.1 | 34227416 | 34230758 |
| Glyma16g30711 | Glyma16g30711.1 | 34237913 | 34239715 |
| Glyma16g30725 | Glyma16g30725.1 | 34245189 | 34245914 |
| Glyma16g30741 | Glyma16g30741.1 | 34250763 | 34253567 |
| Glyma16g30755 | Glyma16g30755.1 | 34260057 | 34262063 |
| Glyma16g30771 | Glyma16g30771.1 | 34270990 | 34273566 |
| Glyma16g30785 | Glyma16g30785.1 | 34277297 | 34289608 |
| Glyma16g30801 | Glyma16g30801.1 | 34299880 | 34303752 |
| Glyma16g30815 | Glyma16g30815.1 | 34304254 | 34321039 |
| Glyma16g30830 | Glyma16g30830.2 | 34327790 | 34330278 |
| Glyma16g30845 | Glyma16g30845.1 | 34343103 | 34345507 |
| Glyma16g30860 | Glyma16g30860.2 | 34350267 | 34353513 |
| Glyma16g30875 | Glyma16g30875.1 | 34360118 | 34363708 |
| Glyma16g30890 | Glyma16g30890.1 | 34367958 | 34370189 |
| Glyma16g30901 | Glyma16g30901.1 | 34379874 | 34380816 |
| Glyma16g30911 | Glyma16g30911.1 | 34382570 | 34385836 |
| Glyma16g30921 | Glyma16g30921.1 | 34392915 | 34395142 |
| Glyma16g30931 | Glyma16g30931.1 | 34413874 | 34423850 |
| Glyma16g30941 | Glyma16g30941.1 | 34420445 | 34440658 |
| Glyma16g30950 | Glyma16g30950.2 | 34443330 | 34446271 |
| Glyma16g30961 | Glyma16g30961.1 | 34453769 | 34458986 |
| Glyma16g30972 | Glyma16g30972.1 | 34456414 | 34463881 |
| Glyma16g30984 | Glyma16g30984.1 | 34464676 | 34469018 |
| Glyma16g30996 | Glyma16g30996.1 | 34466336 | 34466572 |
| Glyma16g31008 | Glyma16g31008.1 | 34474322 | 34475303 |
| Glyma16g31020 | Glyma16g31020.2 | 34483428 | 34491541 |
| Glyma16g31030 | Glyma16g31030.2 | 34494095 | 34496893 |
| Glyma16g31040 | Glyma16g31040.2 | 34500758 | 34501087 |
| Glyma16g31060 | Glyma16g31060.2 | 34512137 | 34515667 |
| Glyma16g31081 | Glyma16g31081.1 | 34526372 | 34529015 |
| Glyma16g31101 | Glyma16g31101.1 | 34533563 | 34534851 |
| Glyma16g31120 | Glyma16g31120.2 | 34550423 | 34557696 |
| Glyma16g31130 | Glyma16g31130.1 | 34561725 | 34562879 |
| Glyma16g31140 | Glyma16g31140.2 | 34586468 | 34589865 |
| Glyma16g31180 | Glyma16g31180.2 | 34618840 | 34621584 |
| Glyma16g31210 | Glyma16g31210.2 | 34645941 | 34648739 |
| Glyma16g31220 | Glyma16g31220.2 | 34651183 | 34652734 |
| Glyma16g31220 | Glyma16g31220.3 | 34651183 | 34652734 |
| Glyma16g31231 | Glyma16g31231.4 | 34653118 | 34667155 |
| Glyma16g31231 | Glyma16g31231.3 | 34653118 | 34667155 |
| Glyma16g31241 | Glyma16g31241.1 | 34654850 | 34655666 |
| Glyma16g31231 | Glyma16g31231.2 | 34660035 | 34667155 |
| Glyma16g31231 | Glyma16g31231.1 | 34660035 | 34667155 |
| Glyma16g31250 | Glyma16g31250.1 | 34669813 | 34673540 |
| Glyma16g31260 | Glyma16g31260.1 | 34677365 | 34679392 |
| Glyma16g31270 | Glyma16g31270.3 | 34682085 | 34683529 |
| Glyma16g31270 | Glyma16g31270.1 | 34682085 | 34683529 |
| Glyma16g31270 | Glyma16g31270.2 | 34682544 | 34683529 |
| Glyma16g31280 | Glyma16g31280.1 | 34699439 | 34702318 |
| Glyma16g31280 | Glyma16g31280.2 | 34699439 | 34702318 |
| Glyma16g31290 | Glyma16g31290.1 | 34702979 | 34706487 |
| Glyma16g31310 | Glyma16g31310.2 | 34718954 | 34724418 |
| Glyma16g31310 | Glyma16g31310.3 | 34718954 | 34724418 |
| Glyma16g31320 | Glyma16g31320.1 | 34726089 | 34733867 |
| Glyma16g31331 | Glyma16g31331.1 | 34735450 | 34739788 |
| Glyma16g31341 | Glyma16g31341.1 | 34744896 | 34749567 |
| Glyma16g31350 | Glyma16g31350.2 | 34767912 | 34769477 |
| Glyma16g31360 | Glyma16g31360.2 | 34788417 | 34791395 |
| Glyma16g31370 | Glyma16g31370.2 | 34797428 | 34801525 |
| Glyma16g31385 | Glyma16g31385.1 | 34803261 | 34803842 |
| Glyma16g31401 | Glyma16g31401.1 | 34804278 | 34806566 |
| Glyma16g31415 | Glyma16g31415.1 | 34812089 | 34814921 |
| Glyma16g31431 | Glyma16g31431.1 | 34819229 | 34820385 |
| Glyma16g31445 | Glyma16g31445.1 | 34826067 | 34830437 |
| Glyma16g31461 | Glyma16g31461.1 | 34834622 | 34846900 |
| Glyma16g31475 | Glyma16g31475.1 | 34855375 | 34856025 |
| Glyma16g31490 | Glyma16g31490.1 | 34876162 | 34879472 |
| Glyma16g31510 | Glyma16g31510.2 | 34896690 | 34899605 |
| Glyma16g31540 | Glyma16g31540.2 | 34904513 | 34905951 |
| Glyma16g31551 | Glyma16g31551.1 | 34908930 | 34910778 |
| Glyma16g31560 | Glyma16g31560.2 | 34917788 | 34920680 |
| Glyma16g31571 | Glyma16g31571.1 | 34923276 | 34923578 |
| Glyma16g31580 | Glyma16g31580.2 | 34925971 | 34927766 |
| Glyma16g31591 | Glyma16g31591.1 | 34933244 | 34934152 |
| Glyma16g31600 | Glyma16g31600.2 | 34938490 | 34941771 |
| Glyma16g31611 | Glyma16g31611.1 | 34943360 | 34950329 |
| Glyma16g31620 | Glyma16g31620.2 | 34950787 | 34955126 |
| Glyma16g31630 | Glyma16g31630.2 | 34958517 | 34961556 |
| Glyma16g31647 | Glyma16g31647.1 | 34974721 | 34977976 |
| Glyma16g31664 | Glyma16g31664.1 | 34984443 | 34988916 |
| Glyma16g31682 | Glyma16g31682.1 | 34984790 | 34986122 |
| Glyma16g31700 | Glyma16g31700.2 | 34995984 | 34999108 |
| Glyma16g31712 | Glyma16g31712.1 | 35003517 | 35006525 |
| Glyma16g31724 | Glyma16g31724.1 | 35017391 | 35030565 |
| Glyma16g31736 | Glyma16g31736.1 | 35044545 | 35045905 |
| Glyma16g31748 | Glyma16g31748.1 | 35047856 | 35049836 |
| Glyma16g31760 | Glyma16g31760.2 | 35056954 | 35061145 |
| Glyma16g31780 | Glyma16g31780.2 | 35065425 | 35065778 |
| Glyma16g31790 | Glyma16g31790.2 | 35068379 | 35071542 |
| Glyma16g31800 | Glyma16g31800.2 | 35078773 | 35082273 |
| Glyma16g31820 | Glyma16g31820.2 | 35095991 | 35103464 |
| Glyma16g31840 | Glyma16g31840.2 | 35108885 | 35110857 |
| Glyma16g31851 | Glyma16g31851.1 | 35120596 | 35126759 |
| Glyma16g31862 | Glyma16g31862.2 | 35127702 | 35135066 |
| Glyma16g31862 | Glyma16g31862.6 | 35127702 | 35135025 |
| Glyma16g31862 | Glyma16g31862.1 | 35127702 | 35136429 |
| Glyma16g31862 | Glyma16g31862.5 | 35127702 | 35135025 |
| Glyma16g31862 | Glyma16g31862.4 | 35127702 | 35136429 |
| Glyma16g31862 | Glyma16g31862.3 | 35127702 | 35135066 |
| Glyma16g31873 | Glyma16g31873.1 | 35135542 | 35139375 |
| Glyma16g31884 | Glyma16g31884.1 | 35137172 | 35137823 |
| Glyma16g31896 | Glyma16g31896.1 | 35145762 | 35146782 |
| Glyma16g31908 | Glyma16g31908.2 | 35161155 | 35166736 |
| Glyma16g31908 | Glyma16g31908.1 | 35161247 | 35166736 |
| Glyma16g31908 | Glyma16g31908.3 | 35161247 | 35166736 |
| Glyma16g31920 | Glyma16g31920.1 | 35168787 | 35173247 |
| Glyma16g31930 | Glyma16g31930.2 | 35174993 | 35176036 |
| Glyma16g31936 | Glyma16g31936.1 | 35180966 | 35181906 |
| Glyma16g31942 | Glyma16g31942.1 | 35181912 | 35186830 |
| Glyma16g31949 | Glyma16g31949.1 | 35182912 | 35184891 |
| Glyma16g31956 | Glyma16g31956.1 | 35187593 | 35188254 |
| Glyma16g31963 | Glyma16g31963.1 | 35188890 | 35190443 |
| Glyma16g31970 | Glyma16g31970.1 | 35193211 | 35194159 |
| Glyma16g31980 | Glyma16g31980.5 | 35195308 | 35201419 |
| Glyma16g31980 | Glyma16g31980.4 | 35195308 | 35201419 |
| Glyma16g31980 | Glyma16g31980.6 | 35195322 | 35201419 |
| Glyma16g31980 | Glyma16g31980.7 | 35195322 | 35201419 |
| Glyma16g31980 | Glyma16g31980.8 | 35197420 | 35201419 |
| Glyma16g31980 | Glyma16g31980.11 | 35197420 | 35201419 |
| Glyma16g31980 | Glyma16g31980.9 | 35197420 | 35201419 |
| Glyma16g31980 | Glyma16g31980.10 | 35197420 | 35201419 |
| Glyma16g31990 | Glyma16g31990.1 | 35202968 | 35208716 |
| Glyma16g31990 | Glyma16g31990.3 | 35202988 | 35208716 |
| Glyma16g31990 | Glyma16g31990.2 | 35203113 | 35208716 |
| Glyma16g32000 | Glyma16g32000.1 | 35212289 | 35215006 |
| Glyma16g32010 | Glyma16g32010.1 | 35215939 | 35218572 |
| Glyma16g32022 | Glyma16g32022.1 | 35225028 | 35236073 |
| Glyma16g32034 | Glyma16g32034.1 | 35227845 | 35230577 |
| Glyma16g32046 | Glyma16g32046.2 | 35243719 | 35246547 |
| Glyma16g32046 | Glyma16g32046.1 | 35243719 | 35246547 |
| Glyma16g32058 | Glyma16g32058.1 | 35245500 | 35245851 |
| Glyma16g32070 | Glyma16g32070.1 | 35256457 | 35258288 |
| Glyma16g32080 | Glyma16g32080.4 | 35274147 | 35275762 |
| Glyma16g32080 | Glyma16g32080.3 | 35274147 | 35275762 |
| Glyma16g32090 | Glyma16g32090.1 | 35278581 | 35286898 |
| Glyma16g32121 | Glyma16g32121.2 | 35305441 | 35308719 |
| Glyma16g32121 | Glyma16g32121.1 | 35305441 | 35308719 |
| Glyma16g32110 | Glyma16g32110.1 | 35305462 | 35306107 |
| Glyma16g32121 | Glyma16g32121.4 | 35305462 | 35308701 |
| Glyma16g32121 | Glyma16g32121.3 | 35305462 | 35308701 |
| Glyma16g32130 | Glyma16g32130.5 | 35310409 | 35317298 |
| Glyma16g32130 | Glyma16g32130.3 | 35310409 | 35316403 |
| Glyma16g32130 | Glyma16g32130.4 | 35310409 | 35316886 |
| Glyma16g32130 | Glyma16g32130.2 | 35310409 | 35316403 |
| Glyma16g32150 | Glyma16g32150.1 | 35328496 | 35331807 |
| Glyma16g32161 | Glyma16g32161.1 | 35337880 | 35343544 |
| Glyma16g32170 | Glyma16g32170.1 | 35347974 | 35348867 |
| Glyma16g32180 | Glyma16g32180.2 | 35351288 | 35358069 |
| Glyma16g32196 | Glyma16g32196.1 | 35366791 | 35369264 |
| Glyma16g32196 | Glyma16g32196.2 | 35366791 | 35369264 |
| Glyma16g32190 | Glyma16g32190.1 | 35366950 | 35368897 |
| Glyma16g32196 | Glyma16g32196.3 | 35367707 | 35369264 |
| Glyma16g32203 | Glyma16g32203.1 | 35375597 | 35378168 |
| Glyma16g32210 | Glyma16g32210.1 | 35379222 | 35380970 |
| Glyma16g32220 | Glyma16g32220.1 | 35381367 | 35384474 |
| Glyma16g32230 | Glyma16g32230.1 | 35394552 | 35398580 |
| Glyma16g32236 | Glyma16g32236.2 | 35400692 | 35405759 |
| Glyma16g32236 | Glyma16g32236.1 | 35400692 | 35405759 |
| Glyma16g32243 | Glyma16g32243.1 | 35406930 | 35407139 |
| Glyma16g32250 | Glyma16g32250.1 | 35408840 | 35416022 |
| Glyma16g32260 | Glyma16g32260.2 | 35417721 | 35425030 |
| Glyma16g32260 | Glyma16g32260.1 | 35417721 | 35425030 |
| Glyma16g32270 | Glyma16g32270.2 | 35425055 | 35430939 |
| Glyma16g32270 | Glyma16g32270.3 | 35425055 | 35430939 |
| Glyma16g32270 | Glyma16g32270.1 | 35425177 | 35430891 |
| Glyma16g32280 | Glyma16g32280.2 | 35440162 | 35442849 |
| Glyma16g32280 | Glyma16g32280.1 | 35440162 | 35442849 |
| Glyma16g32290 | Glyma16g32290.1 | 35454865 | 35457541 |
| Glyma16g32290 | Glyma16g32290.2 | 35454865 | 35457541 |
| Glyma16g32300 | Glyma16g32300.2 | 35488415 | 35490103 |
| Glyma16g32311 | Glyma16g32311.1 | 35503073 | 35516852 |
| Glyma16g32321 | Glyma16g32321.2 | 35526837 | 35530790 |
| Glyma16g32321 | Glyma16g32321.1 | 35526837 | 35530790 |
| Glyma16g32330 | Glyma16g32330.1 | 35538228 | 35539639 |
| Glyma16g32340 | Glyma16g32340.2 | 35541179 | 35546270 |
| Glyma16g32360 | Glyma16g32360.1 | 35552398 | 35557322 |
| Glyma16g32360 | Glyma16g32360.3 | 35552401 | 35557156 |
| Glyma16g32370 | Glyma16g32370.1 | 35558691 | 35564779 |
| Glyma16g32380 | Glyma16g32380.1 | 35567986 | 35569243 |
| Glyma16g32390 | Glyma16g32390.1 | 35568177 | 35571909 |
| Glyma16g32400 | Glyma16g32400.1 | 35579749 | 35582107 |
| Glyma16g32410 | Glyma16g32410.1 | 35581736 | 35582053 |
| Glyma16g32420 | Glyma16g32420.2 | 35584421 | 35587867 |
| Glyma16g32430 | Glyma16g32430.1 | 35590370 | 35592994 |
| Glyma16g32440 | Glyma16g32440.1 | 35598614 | 35599743 |
| Glyma16g32450 | Glyma16g32450.2 | 35604237 | 35604446 |
| Glyma16g32470 | Glyma16g32470.2 | 35617324 | 35619664 |
| Glyma16g32480 | Glyma16g32480.1 | 35624252 | 35630761 |
| Glyma16g32480 | Glyma16g32480.2 | 35624252 | 35630761 |
| Glyma16g32490 | Glyma16g32490.1 | 35634515 | 35637657 |
| Glyma16g32500 | Glyma16g32500.2 | 35647927 | 35652132 |
| Glyma16g32510 | Glyma16g32510.4 | 35660223 | 35664653 |
| Glyma16g32510 | Glyma16g32510.8 | 35660223 | 35664653 |
| Glyma16g32510 | Glyma16g32510.7 | 35660223 | 35664653 |
| Glyma16g32510 | Glyma16g32510.6 | 35660223 | 35664653 |
| Glyma16g32510 | Glyma16g32510.5 | 35661377 | 35664653 |
| Glyma16g32530 | Glyma16g32530.1 | 35669990 | 35674391 |
| Glyma16g32540 | Glyma16g32540.1 | 35676577 | 35684221 |
| Glyma16g32540 | Glyma16g32540.2 | 35676581 | 35684221 |
| Glyma16g32550 | Glyma16g32550.2 | 35716377 | 35717742 |
| Glyma16g32560 | Glyma16g32560.1 | 35727559 | 35729561 |
| Glyma16g32571 | Glyma16g32571.1 | 35733139 | 35734235 |
| Glyma16g32580 | Glyma16g32580.2 | 35747649 | 35752613 |

Note:
The physical position of markers on chromosome 16 (in Mb or bp) is based on publicly available Williams82 reference line (SOYBASE); Soybean genome assembly from JGI release 8. Based on the original Glyma v1 (January 2012).

In one embodiment of the invention a HiSil plant may be produced, selected or identified through the introduction or detection of a gene listed in Table 5. Particularly, any of genes Glyma16g29990, Glyma16g30000, Glyma16g30020. In another embodiment about 2 kilobases, 1 kilobase or 0.5 kilobase pairs upstream from the genes listed in Table 5 may be utilized as a promoter to facilitate gene expression in a cell. Particularly, 2, 1 or 0.5 kilobases upstream of the 5′ starting codon of any one of Glyma16g29990, Glyma16g30000, Glyma16g30020 may be used as a root-preferred promoter region. In this aspect any promoter sequence as described or any expression cassette comprising said promoter region and any plant comprising the resulting expression cassette.

A set of five markers in the HiSil region was developed for the discriminant detection of HiSil gene in a segregating population. A first marker called HiSil-Del was designed based on a large deletion (˜286 bp, Gm16:33,712,274 to 33,712,559) present in the cultivar Hikmok sorip when compared to the Wlliams82 reference genome. The HiSil-Del is tightly linked to HiSil since it is separated by a distance of only 28 Kb. Because of the large size difference in PCR amplicons, the marker HiSil-Del can be used to screen the presence of HiSil even using agarose gel electrophoresis.

In accordance with a further aspect to the invention, four gene markers specific to the HiSil gene (including three deletions and one insertion) were developed. Particularly, these markers can be defined by SEQ ID NO. 2, 3, 4, 5, 6, 7, 8, 9, 10 and 11.

In addition, four other gene-specific markers, including three deletions and one insertion were developed. These markers are helpful to follow the HiSil gene in segregating progenies and can be used to identify the gene in any new sources of germplasm. Particularly, these markers can be defined as HiSil-dell; HiSil-de12; HiSil-de13b, HiSil-insl and HiSil-Del and are capable to be amplified and identified with the following primer sequences: SEQ ID NO. 2, 3, 4, 5, 6, 7, 8, 9, 10 and 11.

In accordance with a further aspect of the invention, there is provided Cleaved Amplified Polymorphic Sequences (CAPS) markers linked to the HiSil gene. These markers are specifically cleaved by a restriction enzyme to yield distinct fragments in the HiSil gene. Particularly, these markers can be defined as HiSil-Mboll_F or HiSil-Mboll_R, and are capable to amplified and identified with the following sequences: SEQ ID NO. 12 and 13.

### Nucleic Acids and Proteins Sequences

In accordance with the different aspect of the invention, the genomic region comprising the HiSil gene corresponds to the region defined by SEQ ID NO.1, or can be defined as 14 or 16 or a portion thereof.

TABLE 6
List of Williams & Hikmok sequences

| SEQ | | |
|---|---|---|
| ID. No. | Variety | Definition |
| 1 | Williams | Glyma16g: HiSil region 33104446 . . . 35762786 |
| 14 | Hikmok | Glyma16g: 30020; CDS 577172 . . . 579696 |
| 15 | Hikmok | Glyma16g: 30020 protein |
| 16 | Hikmok | Glyma16g30000 CDS 567613 . . . 569933 |
| 17 | Hikmok | Glyma16g: 300000 protein |
| 18 | Williams82 | Glyma16g: 30000: partial promoter |
| 19 | Williams82 | Glyma16g: 30000: putative promoter |
| 20 | Williams82 | Glyma16g: 30020: putative promoter |
| 21 | Williams 82 | Glyma16g: 30000 CDS |
| 22 | Williams 82 | Glyma16g: 30000 protein |
| 23 | Williams 82 | Glyma16g: 30020; CDS |
| 24 | Williams82 | Glyma16g: 30020 protein |
| 25 | Hikmok | Glyma16g: 30000: 564321 . . . 567612 putative |
| | | promoter |
| 26 | Hikmok | Glyma16g: 30020: 573723 . . . 577171 putative |
| | | promoter |

Still, in accordance with a particular embodiment of the method for identifying, the amplifying comprises: a) admixing an amplification primer or amplification primer pair with a nucleic acid isolated from the first soybean plant or germplasm, wherein the primer or primer pair is complementary or partially complementary to at least a portion of the marker locus, and is capable of initiating DNA polymerization by a DNA polymerase using the soybean nucleic acid as a template; and, b) extending the primer or primer pair in a DNA polymerization reaction comprising a DNA polymerase and a template nucleic acid to generate at least one amplicon. Particuarly, the nucleic acid is selected from DNA or RNA.

According to a particular embodiment of the method ofr identifying, the amplifying step comprises employing a polymerase chain reaction (PCR) or ligase chain reaction (LCR) using a nucleic acid isolated from the first soybean plant or germplasm as a template in the PCR or LCR.

TABLE 7
List of primer sequences for gene markers

| SEQ ID. | | |
|---|---|---|
| No. | Primer_ID | Primer Sequence |
| 2 | HiSil-del1_F | GAATTTTAAGTCAACAGACATGCAC |
| 3 | HiSil-del1_R | TTTCACGGTAAAAATTATCACCAAC |
| 4 | HiSil-del2_F | GCAGGGAGGCAACAAATTAACAAAC |
| 5 | HiSil-del2_R | TGTTTCACAATCTTTCTTCTCACACAC |
| 6 | HiSil-del3b_F | GGAGGATCGCGACCATCATACTTTC |
| 7 | HiSil-del3b_R | TTCCACACCCTCACACATGATTGTA |
| 8 | HiSil-ins1_F | TGTCGCGTTAAATTCGTATGTTTG |
| 9 | HiSil-ins1_R | TCAAATTAAAGGCATGAGGATTTTGG |
| 10 | HiSil-Del_F | CCCACATCATTTTGACTTAACACTAG |
| 11 | HiSil-Del_R | TCTTCTTAGTTCTTAGATTCTCGCAC |

In accordance with a further aspect of the invention, there is provided CAPS (Cleaved Amplified Polymorphic Sequences) markers linked to the HiSil gene. These markers are specifically cleaved by a restriction enzyme to yield distinct fragments in the Hisil gene of Hikmok sorip variety compared to the fragments in the wild-type gene of the Williams82 variety. Particularly, these markers can be found wih the use of the primers selected from: SEQ ID NO. 12-13 (Table 8), and 27-277 (Table 19) and probes selected from: SEQ ID NOs. 278-495 (Table 19).

TABLE 8
List of primer sequences for CAPS markers.

| SEQ ID | | |
|---|---|---|
| No. | Primer_ID | Sequence |
| 12 | HiSil-Mboll_F | CCTTTTATGTCTCTTCCGTTTGAAAAGC |
| 13 | HiSil-Mboll_R | AAGTATGATGGTCGCGATCCTCCTCC |

Alleles & haplotypes

Allele mining was performed in 328 diverse soybean accessions belonging to different soybean maturity groups. Several haplotype groups were identified based on allelic variation in the coding sequences of Glyma16g:30000 and Glyma16g:30020.

In accordance with a further aspect of the invention, there is provided an H1 allele in the coding sequences of Glyma16g:30000 and Glyma16g:30020. Plants that carried the haplotype H1 were found to accumulate high levels of Si, thus confirming the association of haplotype H1 with high Si uptake capacity in soybean. Particularly, the H1 haplotype can be defined by at least one of a nucleic acid selected from the group consisting of: G (33672717), A (33673022), G (33673483), C (33681630), T (33681946), T (33681961), T (33682500), G (33683047), and C (33683049).

Five accessions were found to carry haplotype (H1) similar to Hikmok sorip. Plants from the entire set of accessions carrying haplotype H1 similar to Hikmok sorip were found to accumulate high levels of Si, thus confirming the association of haplotype H1 with high Si uptake capacity in soybean. The H1 and other haplotypes were defined by the single nucleotide variations present at positions 33672717, 33673022, 33673483, 33681630, 33681946, 33681961, 33682500, 33683047, and/or 33683049 of the HiSil gene (SEQ ID NO: 14 or 16). The nucleotides present at these positions are provided in Table 9. These haplotypes can be characterized by sequencing of the region, primers designed for the variation and several other techniques to detect variation, as is well known in the art.

TABLE 9
Nucleotides representative of haplotype H1 (i.e. Hikmok sorip)
and amino acid changes.

| | Glyma16g30000 | | | Glyma16g30020 | | | | | |
|---|---|---|---|---|---|---|---|---|---|
| | SEQ ID NO. 16 | | | SEQ ID NO. 14 | | | | | |
| Haplo-group | 33672717 | 33673022 | 33673483 | 33681630 | 33681946 | 33681961 | 33682500 | 33683047 | 33683049 |
| H5 | T | T | A | T | A | C | C | C | T |
| (Williams 82) | | | | | | | | | |
| H1 | G | A | G | C | T | T | T | G | C |
| (Hikmok sorip) | | | | | | | | | |

| | SEQ ID NO. 17 | | | SEQ ID NO. 15 | | | | |
|---|---|---|---|---|---|---|---|---|
| Amino Acid | — | L322H | E431G | L5P | — | — | T295I | L439V |
| Change in H1 | | | | | | | | |

TABLE 10
Allelic variation for the three candidate genes identified in Hisil QTL
governing Si accumulation in soybean

| SEQ ID. | | Synony- | Non- | |
|---|---|---|---|---|
| No. | | mous | Synonymous | Amino acid |
| (DNA, AA) | Gene ID | SNP | SNP | changes |
| | Glyma16g29990 | 2 | — | No difference |
| 16, 17 | Glyma16g30000 | 1 | 2 | L322H, E431G |
| 14, 15 | Glyma16g30020 | 3 | 3 | L5P, T295I, |
| | | | | L439V |

The HiSil protein sequence (SEQ ID NO. 15 or 17) has 57% homology with the low Si transporter 2 (Lsi2, efflux Si transporter) identified in rice (rice being a monocot). When looking at HiSil homologs in dicots (like soya), one can see around 70% homology. Therefore, the present invention encompasses plants comprising a HiSil protein sequence having greater than 60% homology to SEQ ID NO: 15 or 17 in monocots and greater than 70% homology to SEQ ID NO: 15 or 17 in dicots.

Alternatively, according to a particular embodiment of the invention, the plant comprises a H1 haplotype, provided that it is not Hikmok scrip.

### Methods for Developing HiSil Soybean Varieties

Therefore, in accordance with a further embodiment, the present invention provides a method for developing a soybean variety with high silicon uptake, the method comprising the step of: a) crossing a first variety of soybean having low Si uptake with a second variety of soybean comprises a marker, wherein the marker comprises a nucleic acid comprising at least one single nucleotide polymorphism (SNP) at a position on chromosome 16 from 33104446 bp to 35762786 bp; and b) selecting a progeny comprising the marker; wherein the progeny comprising the marker has high Si uptake.

Therefore, according to a further embodiment, the present invention provides a method for developing a soybean plant having high silicon uptake, the method comprising the step of: a) grafting a first variety of soybean having low Si uptake with a second variety of soybean having high Si uptake inasmuch as it comprises a nucleic acid sequence originating from a region on chromosome 16, from 33104446 bp to 35762786 bp.

Still, in accordance with an alternative embodiment, the present invention provides a method for genetically modifying a line of soybean having low Si uptake for the purpose of creating a line with high silicon uptake, the method comprising the step of introducing in the plant a nucleic acid originating from a region on chromosome 16 from 33104446 bp to 35762786 bp of i-iikmok scrip soybean variety (e.g. any gene selected from Table 5, particularly Glyma16g29990, Glyma16g30000, Glyma16g30020.

### Methods for Producing a Si High Accumulation Plant

In accordance with a further alternative embodiment, the invention provides a method for producing a Glycine max plant having a HiSil trait, the method comprising the steps of: a) providing a first Glycine max plant line, or progeny thereof comprising an H1 haplotype; b) crossing the Glycine max plant provided in step a) with a second Glycine max plant; c) collecting the seeds resulting from the cross in step b); d) regenerating the seeds of c) into plants; e) providing one or more backcross generations by crossing the plants of step d) or selfed offspring thereof with Glycine max breeding material to provide backcross plants; f) selfing plants of step e) and growing the selfed seed into plants; g) evaluating the plants of step f) for high silicon uptake (i.e. HiSII trait); and h) identifying and selecting plants that are high accumulators of Si.

Alternatively, the present invention provides a method for producing seeds that result in Glycine max plants having a HiSil trait, the method comprising the steps of: a) providing a first Glycine max plant line, or progeny thereof comprising an H1 haplotype; b) crossing the Glycine max plant provided in step a) with a second Glycine max plant; c) collecting the seeds resulting from the cross in step b); d) regenerating the seeds of c) into plants; e) providing one or more backcross generations by crossing the plants of step d) or selfed offspring thereof with Glycine max breeding material to provide backcross plants; f) selfing plants of step e) and growing the selfed seed into plants; and g) selecting and identifying seeds that result in Glycine max plants that are high accumulators of Si. Particularly, the H1 haplotype Glycine max plant is selected from any one of: PI372415A, PI209332, PI404166, PI437655, PI89772, PI90763 or a progeny thereof.

According to a further alternative embodiment, the invention provides a method of producing a soybean plant having increased Si uptake, the method comprising the steps of: a) crossing a first Glycine max plant having high Si uptake with a second Glycine max plant having low Si uptake, wherein the first Glycine max plant comprises in its genome a chromosomal interval comprising a H1 haplotype; and b) producing a progeny plant from the plant cross of a), wherein the progeny plant comprises in its genome a chromosomal interval comprising a H1 haplotype; thereby producing a soybean plant having increased Si uptake. Particularly, the first Glycine max plant comprises a chromosomal interval associated with Si accumulation corresponding to a genomic region from Hikmok sorip chromosome 16 as defined herein. Particuarly, the first Glycine max plant is any one of: PI372415A, PI209332, PI404166, PI437655, PI89772, PI90763 or a progeny thereof.

According to a particular embodiment, the first Glycine max plant comprises a Si concentration of at least about 1% Si concentration in leaf when the soybean variety is provided with a supply of Si at a concentration of about 0.8mM under hydroponic conditions. Particulary or alternatively, the second Glycine max plant having low Si uptake comprises a Si concentration less than 1% Si concentration in leaf when the plant is provided with a supply of Si at a concentration of about 0.8mM under hydroponic conditions.

In accordance with a further alternative embodiment, this method comprises further steps including: isolating a nucleic acid from the progeny plant of b); genotyping the nucleic acid for the presence of a molecular marker associated with Si accumulation in the plant, as defined herein.

In accordance with an alternative embodiment, the invention further provides a method of producing a Glycine max plant with high silicon uptake, the method comprising the steps of: a) isolating a nucleic acid from a Glycine max plant; b) genotyping the nucleic acid of a); c) identifying a plant as comprising at least one molecular marker associated with increased Si uptake as defined herein; and d) producing a Glycine max progeny plant from the plant of c) identified as having the molecular marker associated with increased Si uptake.

A method of producing a Glycine max plant having increased silicon uptake, the method comprising the steps of: a) introducing into a Glycine max plant's genome a chromosomal interval as defined herein; b) selecting for a Glycine max plant, plant germplasm or plant seed comprising the chromosomal interval of a) by isolating a nucleic acid from the plant and genotyping the nucleic acid for a molecular marker which associates with the presence of the chromosomal interval as well as the trait of increased Si uptake; and c) producing a Glycine max plant having increased silicon uptake. Particuarly, the plant or seed produced is an elite soybean variety.

In accordance with a particular embodiment, there is provided a method of producing a plant having increased silicon uptake, the method comprising the steps of: a) introducing into a plant's genome a nucleic acid encoding a HiSil protein; b) selecting for a plant, plant germplasm or plant seed comprising the nucleic acid of a); and c) producing a plant having increased silicon uptake. Particularly, the nucleic acid sequence encodes a protein sequence having 60%, 65%, 70%, 75%, 80%, 85%, 90%, 95%, 99, or 100% sequence identity to any one of SEQ ID NOs: 15 or 17. More particularly, the nucleic acid comprisies a sequence having 60%, 65%, 70%, 75%, 80%, 85%, 90%, 95%, 99, or 100% sequence identity to any one of SEQ ID NOs: 14 or 16.

According to a further embodiment, provided is a method of producing a disease-resistant plant, the method comprising the step of: stably introducing into a plant genome the plant expression cassette as described herein, wherein the introduction of the plant expression cassette confers increased Si uptake in the plant; thereby producing a disease-resistant plant.

In accordance with a particular embodiment, there is provided a method of producing a plant with increased yield, the method comprising the steps of: stably introducing into a plant genome the plant expression cassette as described herein, wherein the introduction of the plant expression cassette confers increased Si uptake in the plant; thereby producing a plant with increased yield.

In accordance with a particular embodiment, there is provided a method for producing a soybean plant with increased Si uptake, the steps comprising: a) introducing into a plant cell a recombinant DNA molecule comprising a polynucleotide encoding a polypeptide, wherein the nucleotide sequence of the polynucleotide is selected from the group consisting of: i) a nucleotide sequence set forth as SEQ ID NO: 14 or 16; ii) a nucleotide sequence encoding a protein having the amino acid sequence of SEQ ID NO: 15 or 17; iii) a nucleotide sequence with at least 90%, at least 91%, at least 92%, at least 93%, at least 94%, at least 95%, at least 96%, at least 97%, at least 98%, at least 99% identity to SEQ ID NO: 14, or 16; and iv) a nucleotide sequence encoding a protein with at least 90%, at least 91%, at least 92%, at least 93%, at least 94%, at least 95%, at least 96%, at least 97%, at least 98%, at least 99% identity to SEQ ID NO: 15 and 17; and b) growing a plant from the plant cell.

### Introduction in Plants

In accordance with a further embodiment of the invention, there is provided a method of introducing a HiSil trait into a plant (such as a soybean plant), comprising: a) selecting a soybean plant comprising the HiSil gene as defined herein, or a nucleic acid sequence in its genome that encodes a protein having at least 80% sequence identity to SEQ ID NO: 17 or SEQ ID NO:15, wherein the protein comprises a Threonine at a position corresponding to position 295 of SEQ ID NO:15, and b) introducing a modification to the nucleic acid sequence such that the encoded protein comprises an Isoleucine at the position corresponding to position 295 of SEQ ID NO:15.

In accordance with a further embodiment of the invention, there is provided a method for producing a plant (such as a soybean plant) with increased Si uptake, the steps comprising: a) introducing into a plant cell a recombinant DNA molecule comprising a polynucleotide encoding a polypeptide, wherein the nucleotide sequence of the polynucleotide is selected from the group consisting of: i) a nucleotide sequence set forth as SEQ ID NO: 14 or 16; ii) a nucleotide sequence encoding a protein having the amino acid sequence of SEQ ID NO: 15 or 17; iii) a nucleotide sequence with at least 90%, at least 91%, at least 92%, at least 93%, at least 94%, at least 95%, at least 96%, at least 97%, at least 98%, at least 99% identity to SEQ ID NO: 14, or 16; and iv) a nucleotide sequence encoding a protein with at least 90%, at least 91%, at least 92%, at least 93%, at least 94%, at least 95%, at least 96%, at least 97%, at least 98%, at least 99% identity to SEQ ID NO: 15 and 17; and b) growing a plant from the plant cell.

Particularly, the HiSil nucleic acid sequence used in the present invention may comprise a nucleic acid sequence having 70%, 75%, 80%, 85%, 90%, 95%, 99% sequence identity with SEQ ID NO: 14 or 16 wherein introduction into the genome of a plant confers increased Si accumulation in the plant. More particularly, the HiSil protein used in the present invention may comprise a amino acid sequence having 70%, 75%, 80%, 85%, 90%, 95%, 99% sequence identity with SEQ ID NO: 15 and/or 17 wherein expression of the gene in a plant confers increased Si accumulation in the plant.

The HiSil gene may be introduced into any plant genome either by traditional breeding or transgenic technologies that are well known in the art. As well, introduction may be accomplished by any manner known in the art, including: introgression, transgenic, or site-directed nucleases (SDN). Particularly, the modification to the nucleic acid sequence is introduced by way of site-directed nuclease (SDN). More particularly, the SDN is selected from: meganuclease, zinc finger, Transcription activator-like effector nucleases system (TALEN) or Clustered Regularly Interspaced Short Palindromic Repeats system (CRISPR) system.

### Genome Editing

SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through nonhomologous end-joining (NHEJ) or homologous recombination (HR), resulting in targeted mutations (‘edits’). Particularly SDN may comprises techniques such as: Meganucleases, Zinc finger nucleases (ZFNs), Transcription Activator-Like Effector-based Nucleases (TALEN) (Feng et al. 2013, Joung & Sander 2013), and the Clustered Regularly Interspaced Short Palindromic Repeats (CRISPR-Cas) system.

In according with this particular method, the nucleic acid may be introduced into the plant genome by either CRISPR, TALEN, meganucleases or through specific modification of genomic nucleic acids. Most particularly, introduction of the nucleic acid is accomplished by heterologous or transgenic gene expression.

### Transgenic

According to a particular embodiment, there is further provided a method of producing a plant having increased silicon uptake, the method comprising the steps of: introducing into a plant's genome a nucleic acid encoding a HiSil protein; selecting for a plant, plant germplasm or plant seed comprising the nucleic acid of a); and producing a plant having increased silicon uptake.

Alternatively, the invention also provided a method of producing a disease resistant plant, the method comprising the step of: stably introducing into a plant genome the plant expression cassette as described herein, wherein the introduction of the plant expression cassette confers increased Si uptake in the plant; thereby producing a disease resistant plant.

Alternatively, also provided is a method of producing a plant with increased yield, the method comprising the step of: stably introducing into a plant genome the plant expression cassette as described herein, wherein the introduction of the plant expression cassette confers increased Si uptake in the plant; thereby producing a plant with increased yield. Accordingly, there is also provided a transgenic plant or a transgenic seed comprising the plant expression cassette as defined herein

Still in accordance with this particular embodiment, the invention therefore provides an agronomically elite soybean seed which is the progeny of a transgenic female ancestor soybean plant having in its genome a recombinant DNA which expresses a Si transporter comprising an amino acid sequence as defined herein, particularly an amino acid sequence with at last about 80%, 90%, 95%, 99% or 100% sequence identity to the amino acid sequence of any one of SEQ ID NOs: 15 or 17. More particularly, the protein is active in root tissue. Most particularly, the protein confers Si accumulation in any one of the plant leaves, plant stem or plant parts.

### Expression Cassettes

According to a particular embodiment, the nucleic acid of the present invention is introduced into the plant's genome by a plant expression cassette.

In accordance with a further aspect of the invention, there is provided an expression cassette for introduction and expression in the plant, the expression cassette comprising the nucleic acid encoding for the HiSil gene operably linked to a plant promoter sequence. Particularly, the invention provides a plant expression cassette comprising the isolated polynucleotide encoding a Si transporter as defined herein, particularly a polynucleotide selected from the group consisting of SEQ ID NOs: 14 and 16, or a polynucleotide encoding a protein having 60%, 70%, 80%, 90%, 95%, or 99% sequence identity to any one of SEQ ID NO: 15 or SEQ ID NO: 17. More particularly, the expression cassette encodes a polypeptide selected from the group consisting of SEQ ID NOs: 15 or 17.

According to a particular embodiment, the expression cassette comprises a nucleic acid that encodes a polypeptide with an amino acid sequence comprising SEQ ID NO 17, where the polypeptide further comprises at least one amino acid corresponding to a histidine at position 322 or a glycine at position 431. Particularly, the plant expression cassette's DNA has at least one allelic modification to the polynucleotide native template encoding a polypeptide comprising SEQ ID NO: 17 wherein the polynucleotide allelic modification results in any one of the amino acid changes selected from the group consisting of: a histidine at position 322 or a glycine at position 431.

According to an alternative embodiment, the expression cassette comprises a nucleic acid that encodes a polypeptide with an amino acid sequence comprising SEQ ID NO 15 and further wherein the polypeptide comprises at least one amino acid corresponding to a proline at position 5, an isoleucine at position 295 or a valine at position 439. Particularly, the plant expression cassette's DNA has at least one allelic modification to the polynucleotide native template encoding a polypeptide comprising SEQ ID NO: 15 wherein the polynucleotide allelic modification results in any one of the amino acid changes selected from the group consisting of: a proline at position 5, an isoleucine at position 295 or a valine at position 439.

More particularly, the expression cassette is introduced into the plant genome by genome editing such as, for example: meganucleases, zinc finger nucleases (ZFNs), transcription activator-like effector nucleases (TALENs), and the Cas9-guideRNA system (adapted from the CRISPR prokarotic immune system), or through specific modification of genomic nucleic

In accordance with an alternative embodiment, the plant expression comprises the polynucleotide as defined herein, operably linked to a native or non-native promoter. Particularly, the plant expression cassette comprises the polynucleotide as defined herein, that is operably-linked to a root-specific or root-preferred promoter, particularly, a promoter as defined herein.

In accordance with an alternative embodiment, the invention provides a vector comprising the plant expression cassette as defined herein.

### Promoters

A promoter is a region of DNA or DNA sequence that initiates transcription of a particular gene. Promoters are located near the transcription start sites of genes, on the same strand and upstream on the DNA (towards the 5′ region of the sense strand). Promoters can be about 100-1000 base pairs long. In the present invention, native or non-native promoter can initiate transcription of the HiSil gene in plants.

The native promoter refers to a promoter that is naturally and/or originally present in a cell and it is typically designated for the expression of a particular gene, such as one that is encoded in the natural original genome of the cell. Therefore, in addition to the nucleic acid, an operably-linked root-specific or root-preferred promoter is introduced into the plant genome, particularly an operably linked HiSil promoter sequence is introduced into the plant genome.

Particularly, the HiSil promoter sequence comprises a nucleic acid sequence defined by SEQ ID NO: 18, 19 or 20. More particularly, the promoter comprises a nucleic acid having 70%, 75%, 80%, 85%, 90%, 95%, 99% sequence identity with SEQ ID NO: 18, 19 or 20. In particular, the promoter sequence comprises a nucleic acid sequence comprising a nucleic acid having 60%, 65%, 70%, 75%, 80%, 85%, 90%, 95%, 99% sequence identity with SEQ ID NO: 18, 19 or 20,

A non-native promoter can be a promoter not originally present in a cell and that has been inserted artificially into the cell such as a promoter of a gene that is not naturally associated with the gene. Particularly, the promoter sequence is a root-specific or a root-preferred promoter. More particularly, the root-specific or root-preferred promoter is selected from the group consisting of: RCc3, PHT1, MtPT1, MtPT2, Pyk10, Beta-tubulin, LRX1, BTG-26, LeAMT1, LeNRT1-1, KDC1, TobRb7, OsRAB5a, ALFS, NRT2, RB7, RD2 and Gns1 glucanase root promoter. Other examples of root-specific promoters include, but are not limited to, the RB7 and RD2 promoters described in U.S. Pat. Nos. 5,459,252 and 5,837,876 respectively.

Still, the promoter can be selected from: RolD promoter, RolD-2 promoter, glycine rich protein promoter, GRP promoter, ADH promoter, maize ADH1 promoter, PHT promoter, Phtl gene family promoter, metal uptake protein promoter, maize metallothionein protein promoter, 35S CaMV domain A promoter, pDJ3S promoter, SIREO promoter, pMe1 promoter, Sad1 promoter, Sad2 promoter, TobRB7 promoter, RCc3 promoter, FaRB7 promoter, SPmads promoter, IDS2 promoter, pyk10 promoter, Lbc3 leghemoglobin promoter, PEPC promoter, Gnsl glucanase root promoter, 35S2 promoter, G14 promoter, G15 promoter, and GRP promoter.

### Introgression or Breeding

In accordance with a particular embodiment, the method of the present invention is carried out where introduction of the nucleic acid is accomplished by plant introgression, plant breeding or marker assisted breeding (MAB).

### Method for Growing a Si High Accumulation Plant

According to a particular embodiment, the present invention further provides a method for growing a plant, comprising the steps of: a) providing the plant as defined herein, or the seed as defined herein; b) growing a plant therefrom; and c) irrigating the plant with a silicon soil amendment.

In particular, the silicon soil amendment can be selected from the group consisting of: mine slag, wollastonite, steel mills slag, crushed rock, calcium silicate, magnesium silicate, amorphous diatomaceous earth (DE), calcium magnesium silicate, phosphorous furnace byproduct, calcium silicate, potassium silicate, silicic acid, organic silicone, sodium silicate. More particularly, the silicon soil amendment can be selected from: Ca₂SiO₄, CaSiO₂, SiO₂, CaSiO₃, MgSiO₃, or K₂SiO₃, (Si(OH)₄, H₄SiO₄, and R₂SiO, wherein R is an organic group such as methyl, ethyl, or phenyl.

According to a particular embodiment, the present invention provides a method of growing a crop (such as a soybean crop), the method comprising the steps of: a) planting in a field the soybean plant as described herein; and b) pplying a compound to the field that comprises silicon: i) prior to planting, ii) at planting, or iii) after planting.

According to a particular embodiment, there is provided a method of growing a soybean crop, the method comprising: a) selecting a location for planting the soybean crop, wherein the location comprises soil, the soil having a silicon concentration at a level of at least 7 ppm, at least 10 ppm, at least 15 ppm, at least 20 ppm, at least 30 ppm, at least 40 ppm or at least 50 ppm and b) planting and growing the soybean plant as described herein.

### Si Soil Amendment and Si Constituent or Source

According to a particular embodiment, the Si amendment may comprise a silicon concentration at a level of: at least 0.4 mM, at least about 0.5 mM, at least about 0.6 mM, at least about 0.7 mM, or at least about 0.8 mM.

Particularly, the Si constituent of the soil amendment comes a source selected from the group comes from: mine slag, wollastonite, steel mills slag, crushed rock, calcium silicate, magnesium silicate, amorphous diatomaceous earth (DE), calcium magnesium silicate, phosphorous furnace byproduct, calcium silicate, potassium silicate, silicic acid, organic silicone, sodium silicate. More particularly, the Si source is selected from: Ca₂SiO₄, CaSiO₂, SiO₂, CaSiO₃, MgSiO₃, or K₂SiO₃, (Si(OH)₄, H₄SiO₄, and R₂SiO, wherein R is an organic group such as methyl, ethyl, or phenyl.

### Kit for Combined Sale

In accordance with a further aspect of the invention there is provided a kit for he combined sale of a seed of the plant as defined herein, and at least one constituent for making a Si soil amendment. In accordance with a particular aspect, the kit further comprises instructions on how to dilute the silicon constituent in a liquid such as water, for making the silicon soil amendment; and, optionally instructions for irrigating the plants.

### List of Specific Embodiments

In accordance with a further aspect of the invention, the following specific embodiments are provided:
1. An elite HiSil Glycine max plant wherein said elite HiSil Glycine max plant comprises in its genome a chromosomal interval comprising a H1 haplotype.
2. An elite HiSil Glycine max plant wherein said elite HiSil Glycine max plant comprises in its genome a chromosomal interval associated with Si accumulation corresponding to a genomic region or portion thereof from Hikmok sorip chromosome 16 at about 92.6 cM to about 132 cM distance as indicated on a genetic linkage map from Hikmok sorip (PI372415A).
3. An elite HiSil Glycine max plant wherein said elite HiSil Glycine max plant comprises in its genome a chromosomal interval associated with Si accumulation corresponding to a genomic region or portion thereof from Hikmok sonp chromosome 16 corresponding to physical positions 31.15M base-pairs to 36.72 M base-pairs of Williams82 reference genome.
4. The plant of any one of paragraphs 1-3, wherein the elite Glycine max is a commercially elite Glycine max variety having a commercially significant yield.
5. The plant of any one of paragraphs 1-4, wherein the chromosomal interval comprises any one of, or a portion of nucleotide base pairs corresponding to positions: 1-2658341 of SEQ ID NO: 1; 567613-569933 of SEQ ID NO: 1; 564321-567612 of SEQ ID NO: 1; 577172-579696 of SEQ ID NO: 1; or 573723-577171 of SEQ ID NO: 1.
6. The plant of any one of paragraphs 1-5, wherein said plant has increased Si accumulation in any one of the plant leaves, plant stem or plant parts as compared to a LoSil plant.
7, The plant of paragraph 6, wherein said plant has at least 1.2×, 1.5×, 2×, 3× or higher Si accumulation compared to a LoSil plant.

18. The plant of any one of paragraphs 1-7, wherein at least one parental line of said plant was selected or identified by a molecular marker located within 20 cM, 10 cM, ScM, 1 cM or 0.5 cM of said chromosomal interval, wherein said molecular marker is associated with Si accumulation in said plant.
9. The plant of paragraph 8, wherein the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite.
10. The plant of any one of paragraphs 8-9, wherein the molecular marker is located within 20 cM, 10 cM, ScM, 1 cM or 0.5 cM of a single nucleotide polymorphism (SNP) marker associated with increased Si accumulation selected from the group consisting of: G(33672717), A(33673022), G(33673483), C(33681630), T(33681946), T(33681961), T(33682500), G(33683047), and C(33683049) as indicated on a genetic linkage map from Hikmok sonp (PI372415A).
11. The plant of any one of paragraphs 1-10, wherein said plant comprises a Si concentration of at least about 1% Si concentration in leaf when said plant is provided with a supply of Si at a concentration of about 0.8 mM, under hydroponic conditions.
12. The plant of any one of paragraphs 1-11, wherein the chromosomal interval is derived from any one of the plant lines selected from the group consisting of: PI372415A, PI209332, PI404166, PI437655, PI89772, PI372415A or PI90763.
13. A progeny plant derived from the plant of any one of paragraphs 1-12.
14. A plant cell, plant seed or plant part derived from the plant of any one of paragraphs 1-13.
15. The plant of any one of paragraphs 1-14, wherein said plant has increased resistance to a stress selected from the group consisting of: diseases (such as powdery mildew, Pythium ultimum, Phytophthora root rot, leaf spot, blast, brown spot, root-knot nematode, soybean cyst nematode, soybean vein necrosis virus, soybean stem canker, soybean sudden death syndrome, leaf and neck blast, rust, frogeye leaf spot, brown stem rot, Fusarium, or sheath blight); insect pests (such as whitefly, aphid, grey field slug, sugarcane borer, green bug, or aphid); abiotic stress (such as drought tolerance, flooding, high level of salinity, heavy metal, aluminum, manganese, cadmium, zinc, UV-B, boron, iron deficiency chlorosis or cold tolerance (i.e. extreme temperatures)).
16. The plant of any one of paragraphs 1-13 or 15, wherein said plant has improved agronomical traits such as seedling vigor, yield potential, phosphate uptake, plant growth, seedling growth, phosphorus uptake, lodging, reproductive growth, or grain quality.
17, An elite Glycine max plant wherein said plant comprises a HiSil trait.
18. An elite HiSil Glycine max plant comprising a HiSil allele which confers increased Si uptake, and wherein the HiSil allele comprises at least one single nucleotide polymorphism (SNP) selected from the group consisting of A(33673022), G(33673483), C(33681630), T(33682500), G(33683047), and C(33683049) as indicated on a genetic linkage map from Hikmok sorip (PI372415A).
19. The plant of paragraph 18, wherein the chromosome interval comprises any one of, or portion of nucleotide base pairs corresponding to positions: 1-2658341 of SEQ ID NO: 1; 567613-569933 of SEQ ID NO: 1; 564321-567612 of SEQ ID NO: 1; 577172-579696 of SEQ ID NO: 1; or 573723-577171 of SEQ ID NO: 1.
20. A method for producing a Glycine max plant having a HiSil trait, the method comprising the steps of:
   a) providing a first Glycine max plant line, or progeny thereof comprising an H1 haplotype;
   b) crossing the Glycine max plant provided in step a) with a second Glycine max plant;
   c) collecting the seeds resulting from the cross in step b);
   d) regenerating the seeds of c) into plants;
   e) providing one or more backcross generations by crossing the plants of step d) or selfed offspring thereof with Glycine max breeding material to provide backcross plants;
   f) selfing plants of step e) and growing the selfed seed into plants;
   g) evaluating the plants of step f) for high silicon uptake (i.e. HiSil trait); and
   h) identifying and selecting plants that are high accumulators of Si.
21. A method for producing seeds that result in Glycine max plants having a HiSil trait, the method comprising the steps of:
   a) providing a first Glycine max plant line, or progeny thereof comprising an H1 haplotype;
   b) crossing the Glycine max plant provided in step a) with a second Glycine max plant;
   c) collecting the seeds resulting from the cross in step b);
   d) regenerating the seeds of c) into plants;
   e) providing one or more backcross generations by crossing the plants of step d) or selfed offspring thereof with Glycine max breeding material to provide backcross plants;
   f) selfing plants of step e) and growing the selfed seed into plants; and
   g) selecting and identifying seeds that result in Glycine max plants that are high accumulators of Si.
22. The method of paragraph 20 or 21, wherein the H1 haplotype Glycine max plant is selected from any one of: PI372415A, PI209332, PI404166, PI437655, PI89772, PI90763 or a progeny thereof.
23. A method of producing a soybean plant having increased Si uptake, the method comprising the steps of:
   a) crossing a first Glycine max plant having high Si uptake with a second Glycine max plant having low Si uptake, wherein said first Glycine max plant comprises in its genome a chromosomal interval comprising a H1 haplotype; and
   b) producing a progeny plant from the plant cross of a), wherein said progeny plant comprises in its genome a chromosomal interval comprising a H1 haplotype;
thereby producing a soybean plant having increased Si uptake.
24. The method of paragraph 23, wherein the first Glycine max plant comprises a chromosomal interval associated with Si accumulation corresponding to a genomic region from Hikmok sorip chromosome 16 at about 92.6 cM to about 132 cM distance or from physical positions 33.15M base-pairs to 36.72M base-pairs as indicated on a genetic linkage map from Hikmok sorip (PI372415A).
25. The method of any one of paragraphs 20-24, wherein the first Glycine max plant is any one of: PI372415A, PI209332, PI404166, PI437655, PI89772, PI90763 or a progeny thereof.
26. The method of any one of paragraphs 24, wherein the chromosomal interval comprises any one of, or portion of nucleotide base pairs corresponding to positions: 1-2658341 of SEQ ID NO: 1; 567613-569933 of SEQ ID NO: 1; 564321-567612 of SEQ ID NO: 1; 577172-579696 of SEQ ID NO: 1; or 573723-577171 of SEQ ID NO: 1.
27. The method of any one of paragraphs 20-26, wherein the first Glycine max plant comprises a Si concentration of at least about 1% Si concentration in leaf when said soybean variety is provided with a supply of Si at a concentration of about 0.8 mM under hydroponic conditions.
28. The method of paragraphs any one of 20-27, wherein the second Glycine max plant having low Si uptake comprises a Si concentration less than 1% Si concentration in leaf when said plant is provided with a supply of Si at a concentration of about 0.8 mM under hydroponic conditions.
29. The method of any one of paragraphs 20-28, comprising further steps including isolation of a nucleic acid from the progeny plant of b); genotyping said nucleic acid for the presence of a molecular marker located within 20 cM, 10 cM, 5 cM, 1 cM or 0.5 cM of the chromosomal interval corresponding to a genomic region from Hikmok sorip chromosome 16 at about 92.6 cM to about 132 cM distance or from physical positions 33.15M base-pairs to 36.72M base-pairs or a portion thereof as indicated on a genetic linkage map from Hikmok sorip (PI372415A), further wherein said molecular marker is associated with Si accumulation in said plant.
30. The method of paragraph 29, wherein the molecular marker is located within 20 cM, 10 cM, 5 cM, 1 cM or 0.5 cM of a single nucleotide polymorphism (SNP) marker associated with increased Si accumulation selected from the group consisting of: A(33673022), G(33673483), C(33681630), T(33682500), G(33683047), and C(33683049) corresponding to a chromosomal interval from Hikmok sorip chromosome 16 at about 92.6 cM to about 132 cM distance or from physical positions 33.15 Mb base-pairs to 36.72 Mb base-pairs as indicated on a genetic linkage map from Hikmok sorip (PI372415A)
31. A method of producing a Glycine max plant with high silicon uptake, the method comprising the steps of:
   a) isolating a nucleic acid from a Giycine max plant;
   b) genotyping the nucleic acid of a)
   c) identifying a plant as comprising at least one molecular marker associated with increased Si uptake wherein said molecular marker is located within 20 cM, 10 cM, 5 cM, 1 cM or 0.5 cM of a chromosomal interval corresponding to a genomic region from Hikmok sorip chromosome 16 at about 92.6 cM to about 132 cM distance; or from physical positions 33.15Mb base-pairs to 36.72Mb base-pairs, or portion thereof as indicated on a genetic linkage map from Hikmok sorip (PI372415A); and
   d) producing a Glycine max progeny plant from the plant of c) identified as having said molecular marker associated with increased Si uptake.
32. A method of producing a Glycine max plant having increased silicon uptake, said method comprising the steps of:
   a) introducing into a Glycine max plant's genome a chromosomal interval comprising a nucleic add comprising nucleotide base pairs corresponding to positions: 1-2658341 of SEQ ID NO: 1; 567613-569933 of SEQ ID NO: 1; 564321-567612 of SEQ ID NO: 1; 577172-579696 of SEQ ID NO: 1; or 573723-577171 of SEQ ID NO: 1; b) selecting for a Glycine max plant, plant germplasm or plant seed comprising the chromosomal interval of a) by isolating a nucleic acid from said plant and genotyping the nucleic add for a molecular marker which associates with the presence of the chromosomal interval as well as the trait of increased Si uptake; and c) producing a Glycine max plant having increased silicon uptake.
33. The method of paragraph 31 or32, wherein the molecular marker is located within 20 cM, 10cm, 5 cM, 1 cM, 0.5 cM or within said chromosomal interval or said marker is located within 20 cM, 10 cM, 5 cM, 1 cM or 0.5 cM of a SNP selected from the group consisting of: A(33673022), G(33673483), C(33681630), T(33682500), G(33683047), and C(33683049) corresponding to a genomic region from Hikmok sorip chromosome 16 at about 92.6 cM to about 132 cM distance or from physical positions 33.15 Mb base-pairs to 36.72 Mb base-pairs, or portion thereof as indicated on a genetic linkage map from Hikmok sorip (PI372415A).
34. The method of paragraph 30-33, wherein the plant or seed produced comprises at least one SNP from the group consisting of: A(33673022), G(33673483), C(33681630), T(33682500), G(33683047), and C(33683049) corresponding to a genomic region from Hikmok sorip chromosome 16 at about 92.6 cM to about 132 cM distance or from physical positions 33.15Mbase-pairs to 36.72M base-pairs, or portion thereof as indicated on a genetic linkage map from Hikmok sorip (PI372415A).
35. The method of paragraphs 20-34, wherein the plant or seed produced is an elite soybean variety.
36. A plant, plant part, or plant seed produced by the method of paragraphs 20-35.
37. A method of producing a Glycine max plant with high silicon uptake, the method comprising the steps of:
   a) isolating a nucleic acid from a Glycine max plant;
   b) genotyping the nucleic acid of a)
   c) identifying a plant as comprising at least one molecular marker associated with the presence of a Si transporter gene wherein the gene encodes a protein comprising any one of SEQ ID NO: 15 or SEQ ID NO: 17; and
   d) producing a Glycine max progeny plant from the plant of c) identified as having said molecular marker associated with increased Si uptake.
38. A method of controlling any one of the following diseases in a soybean crop: Asian soybean rust, soy cyst nematode, nematode, rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolans oryzae, Magnaporthe grisea, Rhizoctonia solani, Phytophthora sone, Schizaphis graminum, Bemisia tabaci, Rhopalosiphum maidis, Deroceras reticulatum, Diatraea saccharalis, Schizaphis graminum and Myzus persicae, the method comprising the steps of:
   a) planting in a field an soybean plant as described in any one of paragraphs 1-13, 15-19; or 36; and
   b) ensuring that said plant is provided with a supply of Si at a concentration of at least about 0.8 mM.
39. A method of reducing abiotic stress damage in a soybean crop wherein the abiotic stress is caused by any one of the following: drought, flooding/excess water, high level of salinity, heavy metal, aluminum, manganese, cadmium, zinc, UV-B, boron, cold temperature, heat, or herbicide, the method comprising the steps of:
   a) planting in a field a soybean plant as described in any one of paragraphs 1-13; 15-19; or 36; and
   b) ensuring that said plant is provided with a supply of Si at a concentration of at least about 0.8mM.
40. A method of increasing yield in a soybean crop, the method comprising the steps of:
   a) planting in a field a soybean plant as described in any one of paragraphs 1-13; 15-19; or 36; and
   b) ensuring that said plant is provided with a supply of Si at a concentration of at least about 0.8 mM.
41. A method of growing a soybean crop, the method comprising the steps of:
   a) planting in a field a soybean plant as described in any one of paragraphs 1-13; 15-19; or 36; and
   b) applying a compound to the field that comprises silicon:
      i. prior to planting,
      ii. at planting, or
      iii. after planting.
42. A method of growing a soybean crop, the method comprising planting in a field a soybean plant as described in any one of paragraphs 1-13; 15-19; or 36, wherein the soil of the field comprises silicon at the level of at least about 0.8 mM.
43. A method of identifying or selecting a first soybean plant having increased Si uptake, the method comprising the steps of:
   a) isolating a nucleic acid from a first soybean plant;
   b) detecting in the nucleic acid the presence of a molecular marker that associates with increased Si uptake and wherein the molecular marker is: associated with a H1 haplotype; or located within 20 cM, 10 cM, ScM, 1 cM or 0.5 cM of a chromosomal interval corresponding to a genomic region from Hikmok sorip chromosome 16 at about 92.6 cM to about 132 cM distance; or located from physical positions 33.15M base-pairs to 36.72M base-pairs as indicated on a genetic linkage map from Hikmok sorip (PI372415A); and
   c) identifying or selecting said soybean plant on the basis of the presence of the molecular marker of b);
   thereby identifying or selecting a first soybean plant having increased Si uptake.
44. The method of paragraph 43, wherein the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite.
45. The method of paragraph 43 or 44, wherein the chromosomal interval comprises any one of, or a portion of a nucleic acid comprising nucleotide base pairs corresponding to positions: 1-2658341 of SEQ ID NO: 1; 567613-569933 of SEQ ID NO: 1; 564321-567612 of SEQ ID NO: 1; 577172-579696 of SEQ ID NO: 1; or 573723-577171 of SEQ ID NO: 1.
46. The method of any one of paragraphs 43-45, wherein the plant identified or selected comprises at least one marker corresponding to:
   a) a genomic region from Hikmok sorip chromosome 16 at about 92.6 cM to about 132 cM distance; or a genomic region from physical positions 33.15M base-pairs to 36.72M base-pairs, or portion thereof as indicated on a genetic linkage map from Hikmok sorip (Pl372415A); or a SNP selected from the group consisting of: A(33673022), G(33673483), C(33681630), T(33682500), G(33683047), and C(33683049) of genes Glyma16g:30000 or Glyrna16g:30020.
47. The method of paragraphs 43-46, wherein the chromosomal interval comprises a nucleic acid encoding a polypeptide with an amino acid sequence comprising SEQ ID NO. 15 and further wherein the polypeptide comprises at least one amino acid corresponding to a proline at position 5, an isoleucine at position 295 or a valine at position 439.
48. The method of paragraphs 43-47, wherein the chromosomal interval comprises a nucleic acid encoding a polypeptide with an amino acid sequence comprising SEQ ID NO. 17 further, wherein the polypeptide comprises at least one amino acid corresponding to a histidine at position 322 or a glycine at position 431.
49. The method of paragraphs 43-48, wherein the method is used in a commercial soybean plant breeding program.
50. The method of paragraphs 43-49, wherein the detecting comprises detecting at least one allelic form of a polymorphic simple sequence repeat (SSR) or a single nucleotide polymorphism (SNP).
51. The method of paragraphs 43-50, wherein the detecting comprises amplifying the marker locus or a portion of the marker locus and detecting the resulting amplified marker amplicon.
52. The method of paragraph 51, wherein the amplifying comprises: a) admixing an amplification primer or amplification primer pair with a nucleic acid isolated from the first soybean plant or germplasm, wherein the primer or primer pair is complementary or partially complementary to at least a portion of the marker locus, and is capable of initiating DNA polymerization by a DNA polymerase using the soybean nucleic acid as a template; and, b) extending the primer or primer pair in a DNA polymerization reaction comprising a DNA polymerase and a template nucleic acid to generate at least one amplicon.
53. The method of paragraph 52, wherein the nucleic acid is selected from DNA or RNA.
54. The method of any one of paragraphs 51-53, wherein the amplifying comprises employing a polymerase chain reaction (PCR) or ligase chain reaction (LCR) using a nucleic acid isolated from the first soybean plant or germplasm as a template in the PCR or LCR.
55. The method of any one of paragraphs 43-54, further comprising the step, wherein the chromosome interval associated with increased Si uptake is introgressed into a second soybean plant or germplasm to produce an introgressed soybean plant or germplasm having increased Si uptake wherein the introgressed soybean plant further comprises at least one of:
   a) a SNP marker selected from the group consisting of: A(33673022), G(33673483), C(33681630), T(33682500), G(33683047), and C(33683049) on genes Glyma30000 or 30020;
   b) a marker corresponding to a genomic region from Hikmok sorip chromosome 16 at about 92.6 cM to about 132 cM distance or
   c) from physical positions 33.15M base-pairs to 36.72M base-pairs, or portion thereof as indicated on a genetic linkage map from Hikmok sorip (PI1372415A).
56. The method of paragraph 55, wherein the second soybean plant or germplasm displays low Si uptake as compared to the first soybean plant or germplasm, wherein the introgressed soybean plant or germplasm displays increased Si uptake as compared to the second plant or germplasm.
57. The method of any one of any one of paragraphs 55-56, wherein the second soybean plant or germplasm comprises an elite soybean strain or an exotic soybean strain.
58. The method of any one of any one of paragraphs 43-57, comprising electronically transmitting or electronically storing data representing the detected allele or molecular marker in a computer readable medium.
59. The method of any one of paragraphs 43-58, wherein the molecular marker or allele is determined using TASSEL, GeneFlow, or MapManager-QTX software.
60. The method of any one of paragraphs 43-59, wherein said chromosome interval comprises at least one single nucleotide polymorphism (SNP) selected from the group consisting of: A(33673022), G(33673483), C(33681630), T(33682500), G(33683047), and C(33683049) of Glyma16g:30000 or Glyma16g:30020 genes wherein presence of said SNP is associated with Si accumulation.
61. The plant of paragraphs 1-13; 15-19; or 36, wherein said chromosomal interval comprises SEQ ID NO.14 or 16 or a portion thereof providing increased silicon uptake in a Glycine max plant.
62. The plant of paragraphs 1-13; 15-19; or 36 or 61, wherein said plant comprises a molecular marker associated with increases Si uptake capable of being amplified and identified with the following primer sequences: SEQ ID NO. 2, 3, 4, 5, 6, 7, 8, 9, 10, 11 and 27-277.
63. The plant of any one of paragraphs 1-13; 15-19; or 36 or 61-62, wherein said plant comprises a marker capable being amplified and identified with the following sequences: SEQ ID NO. 12, 13 and 278-495.
64. The plant of any one of paragraphs 61-63, wherein said molecular marker is located within HiSil region genes, as defined by an nucleic acid selected from the group consisting of: A(33673022), G(33673483), C(33681630), T(33682500), G(33683047), and C(33683049) of genes Glyma30000 or 30020.
65. An agronomically elite Glycine max plant capable of accumulating Si in leaf tissue at a concentration of at least 1% Si concentration when plants are provided with a supply of Si at a concentration of about 0.8 mM under hydroponic conditions, wherein the Glycine max comprises a genomic region introduced into its genome comprising any one of SEQ ID NO: 14, 16 or 18.
66. The plant of paragraph 65, wherein said plant has a leaf Si concentration of at least around one point two (1.2×), one and a half (1.5×), double (2×), or triple (3×) the concentration of a control plant not comprising said genomic region.
67. The plant of any one of paragraphs 1-13; 15-19; or 36 or 61-66, wherein, said chromosomal interval or genomic region comprises a nucleic acid encoding a polypeptide with an amino acid sequence comprising SEQ ID NO 15 and further wherein the polypeptide comprises at least one amino acid corresponding to a proline at position 5, a isoleucine at position 295 or a valine at position 439.
68. The plant of any one of paragraphs 1-13; 15-19; or 36 or 61-67, wherein, said chromosomal interval or genomic region comprises a nucleic acid encoding a polypeptide with an amino acid sequence comprising SEQ ID NO 17 further, wherein the polypeptide comprises at least one amino acid corresponding to a histidine at position 322 or a glycine at position 431.
69. The plant of paragraph 68, wherein the nucleic acid is SEQ ID NO: 16.
70. The plant of paragraph 67, wherein the nucleic acid is SEQ ID NO: 14.
71. A plant of a soybean variety or lineage having high Si uptake, provided that said variety is not Hikmok sorip.
72. The plant of paragraph 71, wherein the soybean variety or lineage comprises in its genome a chromosomal interval comprising SEQ ID NO: 14 or 16 wherein said chromosomal interval is derived from Hikmok sorip.
73. Seeds produced by the plant of paragraphs 61-72.
74. The plant of paragraphs 1-13; 15-19; or 36 or 61-72, wherein said plant additionally has in it genome a transgene that confers any one of the traits selected from the group consisting of: herbicide resistance or insect resistance.
75. A plant having introduced into its genome a nucleic acid sequence encoding a protein having 60%, 70%, 80%, 90%, 95%, or 99% sequence identity to any one of SEQ ID NO: 15 or SEQ ID NO: 17.
76. The plant of paragraph 75, wherein the plant is a monocot or dicot.
77. The plant of any one of paragraphs 75-76, wherein the plant is selected from the group consisting of soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, and rice.
78. The plant of any one of paragraphs 75-77, wherein the protein is a functional Si transporter that facilitates Si uptake into the plant.
79. The plant of any one of paragraphs 75-78, wherein the nucleic acid sequence comprises any one of SEQ ID NOs: 14 or 16.
80. The plant of any one of paragraphs 75-79, wherein the nucleic acid encodes a protein comprising or consisting of SEQ ID NO: 15 or SEQ ID NO: 17.
81. The plant of any one of paragraphs 75-80, wherein the nucleic acid is derived from a Glycine sp. plant having high silicon uptake.
82. The plant of any one of paragraphs 75-81, wherein the nucleic acid is derived from a black hilum soybean variety (e.g. Hikmok sorip) having high Si uptake.
83. The plant of any one of paragraphs75-82, wherein at least two nucleic acid sequences are introduced into its genome, wherein the two nucleic acid sequences encode proteins comprising a polypeptide sequence comprising SEQ ID NO: 15 and SEQ ID NO: 17.
84. The plant of any one of paragraphs 75-83, wherein the protein is active in said plant's roots.
85. The plant of any one of paragraphs 75-84, wherein the protein confers Si accumulation in any one of the plant leaves, plant stem or plant parts.
86. The plant of any one of paragraphs 75-85, wherein introduction of said nucleic acid is accomplished by heterologous or transgenic gene expression.
87. The plant of any one of paragraphs 75-86, wherein the nucleic acid introduced into said plant's genome is introduced by a plant expression cassette.
88. The plant of paragraph 87, wherein the plant expression cassette comprises a promoter operably linked to said nucleic acid wherein said promoter facilitates expression of the nucleic acid in said plant's root tissue.
89. The plant of paragraph 88, wherein the promoter sequence comprises a nucleic acid sequence comprising a nucleic acid having 60%, 65%, 70%, 75%, 80%, 85%, 90%, 95%, 99% sequence identity with SEQ ID NO: 18, 19 or 20.
90. The plant of any one of paragraphs 88-89, wherein the promoter is a root specific promoter or a root preferred promoter.
91. The plant of paragraph 90, wherein the root specific or root preferred promoter is selected from the group consisting of RCc3, PHT1, MtPT1, MtPT2, Pyk10, Beta-tubulin, LRX1, BTG-26, LeAMT1, LeNRT1-1, KDC1, TobRb7, OsRAB5a, ALF5, and NRT2.
92. The plant of any one of paragraphs 75-86, wherein the nucleic acid has been introduced into the plant genome by either CRISPR, TALEN, meganucleases or through modification of genomic nucleic acids,
93. The plant of any one of paragraphs 75-92, wherein the nucleic acid encodes a polypeptide with an amino acid sequence comprising SEQ ID NO 15 and further wherein the polypeptide comprises at least one amino acid corresponding to a proline at position 5, a isoleucine at position 295 or a valine at position 439.
94. The plant of any one of paragraphs 75-93, wherein the nucleic acid encodes a polypeptide with an amino acid sequence comprising SEQ ID NO 17 further, wherein the polypeptide comprises at least one amino acid corresponding to a histidine at position 322 or a glycine at position 431.
95. The plant of any one of paragraphs 75-94, wherein the plant is a high Si accumulator as compared to a control plant not comprising said nucleic acid.
96. The plant of any one of paragraphs 75-86, wherein introduction of said nucleic acid is accomplished by plant introgression or plant breeding.
97. The plant of paragraph 96, wherein at least one parental line of said plant was selected or identified by a molecular marker associated with said nucleic acid.
98. The plant of any one of paragraphs 75-97, wherein the introduction of the nucleic acid confers any one of increased biotic resistance or tolerance, increased abiotic resistance or tolerance, increased yield, increased biomass, quality or a combination thereof.
99. The plant of any one of paragraphs 75-98, wherein the introduction of the nucleic acid confers increased resistance to at least one pathogen from the group consisting of: nematode, rust, smut, Golovinomyces cichoracearum, Erysiphe cichoracearum, Blumefia graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopaiosiphum maidis, Deroceras reticulatum, Diatraea saccharalis, Schizaphis graminum and Myzus persicae; or a combination thereof.
100. The plant of any one of paragraphs 75-99, having increased resistance to a stress selected from the group consisting of: diseases (such as powdery mildew, Pythium ultimum, root rot, leaf spot, blast, brown spot, leaf and neck blast, or sheath blight); insect pests (such as whitefly, aphid, grey field slug, sugarcane borer, green bug, or aphid); abiotic stress (such as drought, flooding, high level of salinity, heavy metal, aluminum, manganese, cadmium, zinc, UV-B, boron or cold tolerance (i.e. extreme temperatures)).
101. The plant of any one of paragraphs 75-100, having improved agronomical traits such as seedling vigor, yield potential and phosphate uptake, plant growth, seedling growth, phosphorus uptake, lodging, reproductive growth, or grain quality.
102. The plant of any one of paragraphs 75-101, wherein the plant is a crop plant.
103. The plant of any one of paragraphs 75-102, wherein said plant is a soybean plant and is not Hikmok sorip (PI372415A).
104. The plant of any one of paragraphs 75-103, wherein the plant is an elite soybean plant.
105. The plant of any one of paragraphs 75-104, wherein said plant comprises a silicon concentration of at least 1% Si concentration in leaf when plants are provided with a supply of Si at a concentration of about 0.8 mM under hydroponic conditions.
106. The plant of any one of paragraphs 75-105, wherein said plant has a leaf Si concentration of at least about double (2×) as compared to a control plant.
107. A plant expression cassette comprising an isolated polynucleotide encoding a Si transporter selected from the group consisting of SEQ ID NOs: 14 and 16.
108. The expression cassette of paragraph 107, wherein said polynucleotide encodes a polypeptide selected from the group consisting of SEQ ID NOs: 15 or 17.
109. The plant expression cassette of any one of paragraphs 107-108, wherein the polynucleotide is operably linked to a non-native promoter.
110. The plant expression cassette of anyone of paragraphs 107-109, wherein the DNA has at least one allelic modification to said polynucleotide native template encoding a polypeptide comprising SEQ ID NO: 15 wherein the polynucleotide allelic modification results in any one of the amino acid changes selected from the group consisting of: a proline at position 5, a isoleucine at position 295 or a valine at position 439.
111. The plant expression cassette of paragraphs 107-110, wherein the DNA has at least one allelic modification to said polynucleotide native template encoding a polypeptide comprising SEQ ID NO: 17 wherein the polynucleotide allelic modification results in any one of the amino acid changes selected from the group consisting of: a histidine at position 322 or a glycine at position 431.
112. The plant expression cassette of any one of paragraphs 110-111, wherein the allelic modification is achieved through CRISPR, TALEN, Meganucleases, or genome editing technologies.
113. A vector comprising the plant expression cassette of any one of paragraphs 107-112.

1114. A plant expression cassette comprising the polynucleotide of any one of paragraphs 107-112.
115. The plant expression cassette of any one of paragraphs 107-112, wherein said polynucleotide is operably-linked to a root-specific or root-preferred promoter.
116. The plant expression cassette of paragraph 115, wherein said promoter comprises SEQ ID NO: 18, 19 or 20.
117. A transgenic plant comprising the plant expression cassette of paragraphs 114-116.
118. A transgenic seed comprising the plant expression cassette of paragraphs 114-116.
119. The transgenic plant of paragraph 117, wherein the plant is selected from the group consisting of soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, and rice.
120. The transgenic seed of paragraph 119, wherein said seed is from a transgenic plant selected from the group consisting of soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, and rice.
121. A method of producing a plant having increased silicon uptake, said method comprising the steps of:
   a) introducing into a plant's genome a nucleic acid encoding a HiSil protein;
   b) selecting for a plant, plant germplasm or plant seed comprising the nucleic acid of a); and
   c) producing a plant having increased silicon uptake.
122. The method of paragraph 121, wherein the nucleic acid sequence encodes a protein sequence having 60%, 65%, 70%, 75%, 80%, 85%, 90%, 95%, 99, or 100% sequence identity to any one of SEQ ID NOs: 15 or 17.
123. The method of any one of paragraph 121-122, wherein the plant is a dicot or monocot.
124. The method of any one of paragraphs 121-123, wherein the plant is a high Si accumulator as compared to a control plant not comprising said nucleic acid.
125. The method of any one of paragraphs 121-124, wherein the plant is soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, or rice.
126. The method of any one of paragraphs 121-125, wherein the plant has introduced into its genome a nucleic acid sequence comprising a nucleotide sequence having 60%, 65%, 70%, 75%, 80%, 85%, 90%, 95%, 99, or 100% sequence identity to any one of SEQ ID NOs: 14 or 16.
127. The method of any one of paragraphs 121-126, wherein the nucleic acid sequence encodes a protein that facilitates Si uptake.
128. The method of paragraph 127, wherein the nucleic acid sequence encodes a HiSil protein.
129. The method of any one of paragraphs 121-128, wherein the protein is active in root tissue.
130. The method of any one of paragraphs 121-129, wherein the protein confers Si accumulation in any one of the plant leaves, plant stem or plant parts.
131. The method of any one of paragraphs 121-130, wherein, in addition to the nucleic acid, an operably-linked root-specific or root-preferred promoter has been introduced into said plant genome.
132. The method of any one of paragraphs 121-131, wherein, in addition to said nucleic acid, an operably linked HiSil promoter sequence has been introduced into said plant genome.
133. The method of paragraph 132, wherein the promoter sequence comprises a nucleic acid sequence comprising a nucleic acid having 60%, 65%, 70%, 75%, 80%, 85%, 90%, 95%, 99% sequence identity with SEQ ID NO: 18, 19 or 20.
134. The method of paragraph 131, wherein the root specific or root preferred promoter is selected from the group consisting of: RCc3, PHT1, MtPT1, MtPT2, Pyk10, Beta-tubulin, LRX1, BTG-26, LeAMT1, LeNRT1-1, KDC1, TobRb7, OsRAB5a, ALF5, and NRT2.
135. The method of any one of paragraphs 121-130, wherein the nucleic acid has been introduced into the plant genome by either CRISPR, TALEN, meganucleases or through specific modification of genomic nucleic acids.
136. The method any one of paragraphs 121-130, wherein introduction of said nucleic acid is accomplished by heterologous or transgenic gene expression.
137. The method of any one of paragraphs 121-130, wherein introduction of said nucleic acid is accomplished by plant introgression, plant breeding or marker assisted breeding (MAB).
138. A method of producing a disease resistant plant, the method comprising the step of:
   a) stably introducing into a plant genome the plant expression cassette as described in any one of paragraphs 108-112 and 114-116, wherein said introduction of said plant expression cassette confers increased Si uptake in said plant; thereby producing a disease resistant plant.
139. A method of producing a plant with increased yield, the method comprising the step of:
   a) stably introducing into a plant genome the plant expression cassette as described in any one of paragraphs 114-116, wherein said introduction of said plant expression cassette confers increased Si uptake in said plant; thereby producing a plant with increased yield
140. The method of any one of paragraphs 138 and 139, wherein the plant s soybean, tomato, melon, maize, sugarcane, canola, broccoli, cabbage, cauliflower, pepper, oilseed rape, sugarbeet, celery, squash, spinach, cucumber, watermelon, zucchini, common bean, wheat, barley, sweet corn, sunflower, or rice.
141. An agronomically elite soybean seed which is the progeny of a transgenic female ancestor soybean plant having in its genome a recombinant DNA which expresses a Si transporter comprising an amino acid sequence with at last about 80%, 90%, 95%, 99% or 100% sequence identity to the amino acid sequence of any one of SEQ ID NOs: 15 or 17.
142. A method for producing a soybean plant with increased Si uptake, the steps comprising:
   a) introducing into a plant cell a recombinant DNA molecule comprising a polynucleotide encoding a polypeptide, wherein the nucleotide sequence of the polynucleotide is selected from the group consisting of:
      i) a nucleotide sequence set forth as SEQ ID NO: 14 or 16;
      ii) a nucleotide sequence encoding a protein having the amino acid sequence of SEQ ID NO: 15 or 17;
      iii) a nucleotide sequence with at least 90%, at least 91%, at least 92%, at least 93%, at least 94%, at least 95%, at least 96%, at least 97%, at least 98%, at least 99% identity to SEQ ID NO: 1 14, or 16; and iv) a nucleotide sequence encoding a protein with at least 90%, at least 91%, at least 92%, at least 93%, at least 94%, at least 95%, at least 96%, at least 97%, at least 98%, at least 99% identity to SEQ ID NO: 15 and 17;
   and
   b) growing a plant from said plant cell.
143. The method of paragraph 142, further comprising selecting a plant with an enhanced trait selected from: increased yield, increased nitrogen use efficiency, increased disease resistance, increased abiotic stress tolerance, increased insect resistance, and increased water use efficiency or drought tolerance as compared to a control plant.
144. A seed for the plant as defined in any one of paragraphs 1-19; 36; 74-106; 119-120 and 141.
145. A seed from the plant as defined in any one of paragraphs 1-19; 36; 61-72; 74-106; 119-120 and 141.
146. A kit for producing a silicon high accumulating plant comprising:
   a) the seed of paragraph 144 or 145, and
   b) at least one constituent for making a silicon soil amendment.
147. The kit of paragraph 146, wherein said constituent is selected from the group consisting of: mine slag, wollastonite, steel mills slag, crushed rock, calcium silicate, magnesium silicate, amorphous diatomaceous earth (DE), calcium magnesium silicate, phosphorous furnace byproduct, calcium silicate, potassium silicate, silicic acid, organic silicone, sodium silicate.
148. The kit of paragraph 147, wherein said constituent is selected from: Ca₂SiO₄, CaSiO₂, SiO₂, CaSiO₃, MgSiO₃, or K₂SiO₃, (Si(OH)₄, H₄SiO₄, and R₂SiO, wherein R is an organic group such as methyl, ethyl, or phenyl.
149. The kit of any one of paragraphs 146-148, further comprising instructions on how to dilute said silicon constituent in water for applications in soil.
150. A cell of a seed as defined in paragraph 144 or 145.
151. A cell of a plant as defined in any one of paragraphs 1-19; 36; 61-72; 74-106; 119-120 and 141.
152. A method for growing a plant, comprising the steps of:
   a) providing a plant according to any one of paragraph 1-19; 36; 61-72; 74-106; 119-120 and 141 or a seed as defined in paragraph 144 or 145;
   b) growing a plant therefrom; and
   c) irrigating said plant with a silicon soil amendment.
153. The method of paragraph 152, wherein said silicon soil amendment is selected from the group consisting of: mine slag, wollastonite, steel mills slag, crushed rock, calcium silicate, magnesium silicate, amorphous diatomaceous earth (DE), calcium magnesium silicate, phosphorous furnace byproduct, calcium silicate, potassium silicate, silicic acid, organic silicone, sodium silicate.
154. The method of paragraph 153, wherein said silicon soil amendment is selected from: Ca₂SiO₄, SiO₂, CaSiO₃, MgSiO₃, or K₂SiO₃, (Si(OH)₄, H₄SiO₄, and R₂SiO, wherein R is an organic group such as methyl, ethyl, or phenyl.
155. A method of introducing a HiSil trait into a soybean plant, comprising:
   a) selecting a soybean plant comprising a nucleic acid sequence in its genome that encodes a protein having at least 80% sequence identity to SEQ ID NO: 15 or SEQ ID NO:17, wherein the protein comprises a Threonine at a position corresponding to position 295 of SEQ ID NO:15, and
   b) introducing a modification to the nucleic acid sequence such that the encoded protein comprises an Isoleucine at the position corresponding to position 295 of SEQ ID NO:15,
   wherein a site-directed nuclease (SDN) introduces the modification to the nucleic acid sequence.
156. The method of paragraph 155, wherein the SDN is selected from: meganuclease, zinc finger, Transcription activator-like effector nucleases system (TALEN) or Clustered Regularly Interspaced Short Palindromic Repeats system (CRISPR) system.
157. A soybean plant produced by the method of paragraph 155.
158. An elite soybean plant comprising a nucleic acid sequence that encodes a protein having at least 80% sequence identity to SEQ ID NO: 15 or SEQ ID NO: 17, wherein the protein comprises an Isoleucine at a position corresponding to position 295 of SEQ ID NO:15.
159. A method of growing a soybean crop, the method comprising the steps of:
   a) planting in a field a soybean plant as described in any one of paragraphs 152 to 154 and
   b) applying a compound to the field that comprises silicon:
      i. prior to planting,
      ii. at planting, or
      iii. after planting.
160. A method of growing a soybean crop, the method comprising:
   a) selecting a location for planting the soybean crop, wherein the location comprises soil, said soil having a silicon concentration at a level of at least 7 ppm, at least 10 ppm, at least 15 ppm, at least 20 ppm, at least 30 ppm, at least 40 ppm or at least 50 ppm and
   b) planting and growing a soybean plant as described in any one of paragraphs 152-154.
161. The plant of any one of paragraphs 72-106, wherein the plant comprises a H1 haplotype.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Discovery of the HiSil Region in Hikmok sorip Soybean

Materials and methods

### Plant Material

A set of 139 soybean cultivars representing early maturity groups was evaluated for Si accumulation, Subsequently, a cross was made between the known high absorbing line Hikmok sorip and a typical absorbing line (Majesta) and we developed 141 recombinant inbred lines (RIL) that were also evaluated. Soybean plants, three per line, were grown in a greenhouse under controlled conditions. Surface sterilization of seed was performed using 2% sodium hypochloride treatment for 5 min followed by three subsequent washes with distilled water. Plants were grown in potting soil with or without 1.7 mM Si prepared from potassium silicate (Kasil #6, 23.6% SiO₂, National Silicates).

### Quantification of Silicon in Soybean Leaf Samples

The first trifoliate leaf of each plant was collected for Si concentration analysis three weeks after the first Si amendment. Dried leaves were ground to a powder in a bead homogenizer (Omni Bead Ruptor, Omni International). Measurements were made with a portable X-ray fluorescence spectrometer (Niton XL3t900 GOLDD XRF analyser; Thermo Scientific) at the University of York, UK, according to the methods of Reidinger et al., (2012). The Si rate assay was carried out with non-inoculated plants.

### X-ray Microanalysis and Scanning Electron Microscopy

Si distribution in leaves of different soybean genotypes was analyzed by using scanning electron microscopy coupled with an energy dispersive X-ray (DXR) micro-analyzer. A single fully expanded healthy leaf without any symptoms of disease or physical damage was harvested from each plant species grown with or without Si. Small sections (approx. 10×10 mm) were cut from the central region of the leaf, avoiding midribs. The cut pieces of leaves were lyophilized and coated with gold and paladium to provide conductivity. Coated samples were examined using a CAMECA SX-100 Universal EPMA microscope (Cameca instruments Inc., Trumbull, USA). Voltage of 15 kV and a current of 20 nA were used for processing to get the elemental concentration profiles across the leaf sample.

### Genotyping-by-Sequencing of Soybean Cultivars

SNP genotyping previously performed using a GBS approach was used (Sonah et al. 2014). The ApeK1 restriction enzyme was used for library preparation following Elshire's protocol (Elshire et al,, 2011) with minor modifications described in Sonah et al. (2013). Single-end sequencing of multiplex GBS libraries was performed using the Illumina HiSeq2000 at the Genome Québec Innovation Center, McGill University (Montreal, QC, Canada). Illumine sequence read processing, mapping, SNP calling and genotyping were performed using the IGST-GBS pipeline (Sonah et al., 2013). Vcftools and several in-house scripts were used to obtain good quality SNPs. Imputation of missing data was performed with fastPHASE 1.3 (Scheet & Stephens, 2006). Functional and structural annotation of SNPs was performed using SnpEff (version 3.3H) and the soybean genome annotation provided in the Phytozome database (Goodstein et al., 2012, Cingolani et al., 2012).

### Genome Wide Association Study (GWAS)

GWAS was performed using software tools like TASSEL 3.0 and the Genomic Association and Prediction Integrated Tool (GAPIT) (Bradbury et al., 2007; Lipka et al,, 2012). A general linear model (GLM) was used with or without the covariate P from principal component analysis (PCA) and the covariate Q obtained from STRUCTURE. A kinship matrix was calculated either using the VanRaden method (K) or the EMMA method (K*) to determine relatedness among individuals (Kang et al., 2008; Loiselle et al., 1995), Compressed mixed linear models (CMLM) incorporating a kinship matrix (K or K*) along with P or Q were tested. The negative log(1/n) was used to establish a significance threshold.

### QTL Mapping

Genotypic data were obtained using GBS for the 141 RILs derived from the Majesta×Hikmok sorip cross and used for QTL mapping. QTL mapping was performed using the QTL IciMapping software (version 3.3, released July 2013, www.isbreeding.net).

### Grafting Experiments

Grafts were made among four cultivars Jack, Majesta, Williams 82 and Hikmok sorip. To promote branching, a shoot meristem was plucked at the V1 stage. Of two arising branches, one branch was grafted very close to the branching point. Leaf samples were taken from both branches to compare Si accumulation. Plants of the same genotype were grafted with each other and used as controls.

### Results

### Evaluation of Silicon (Si) Uptake in Soybean Germplasm

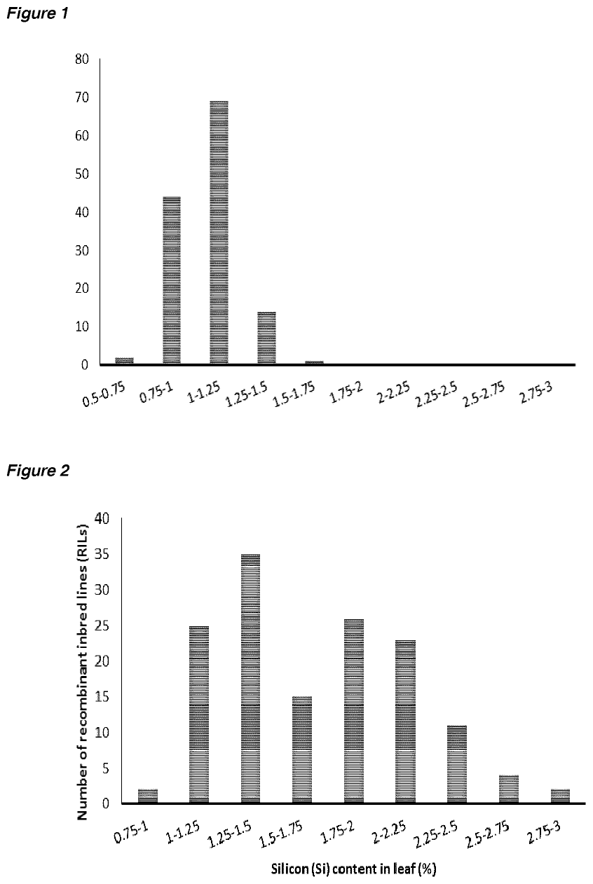

The cultivated soybean germplasm set was evaluated under greenhouse conditions to measure Si uptake ability. Values ranged between 0.65% and 1.53% with an average of ca. 1.0% and a standard deviation of 0.15 (FIG. 1). The frequency distribution indicated a limited variability for this trait.

Evaluation of Silicon (Si) Iptake in Majesta×Himok sorip RILs

Since Hikmok sorip appeared to be a line with exceptional ability to absorb Si based on our own observations, it was crossed with Majesta, a cultivated line showing average Si accumulation, to create 141 RILs in an attempt to map the genetic loci that could govern Si accumulation. X-ray microscopy of leaf tissues corroborated the higher accumulation of Si in Hikmok sorip compared to Majesta (FIG. 3),

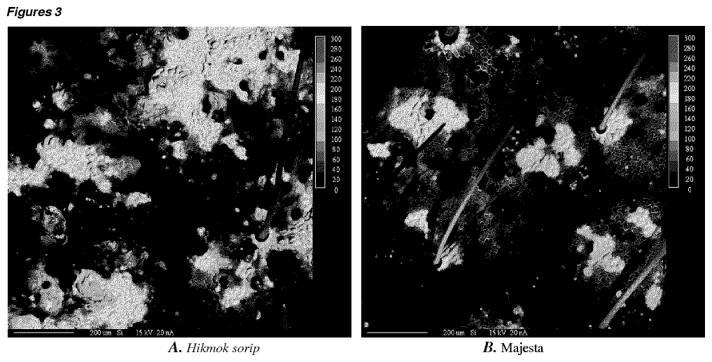

The Si accumulation in leaf tissues of all 141 RILs derived from crosses between Majesta and Hikmok sorip showed a range of nearly 2.0% between the lowest and highest values. The average value was 1.69% with a standard deviation of 0.45. Unlike the data with the Canadian germplasm lines, frequency distribution showed a bimodal distribution pattern suggesting the involvement of specific genes in the Si uptake regulation (FIG. 2).

### Genome-Wide Association Study (GWAS) for Si Accumulation in Soybean

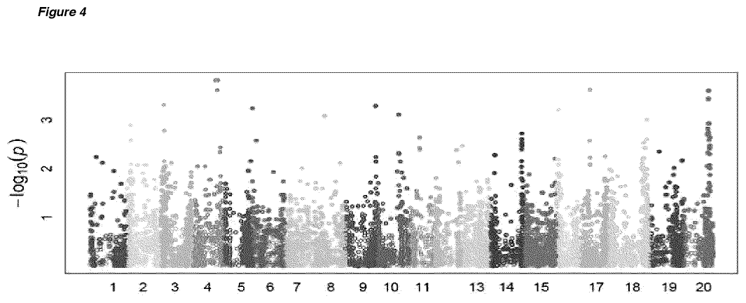

GWAS was initially performed using a set of 139 cultivated lines. Based on this analysis, none of the markers showed a significant association with Si accumulation in soybean leaves (FIG. 4). Subsequently, the 95 PI (Plant introduction) lines were combined with the Canadian lines for an additional GWAS. Once again, none of the markers showed a significant association with Si accumulation in spite of the seemingly wider range of phenotypes in the PI lines.

Identification of a Quantitative Trait Locus (QTL) for Si Accumulation in Hikmok sorip

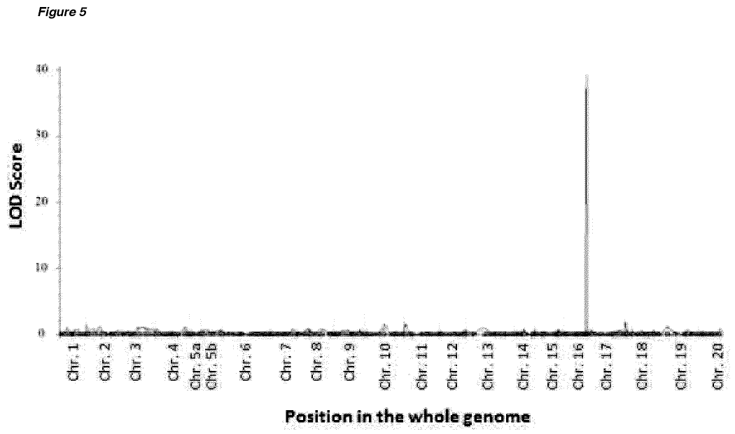

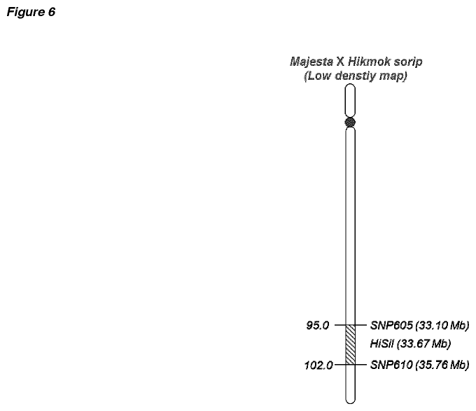

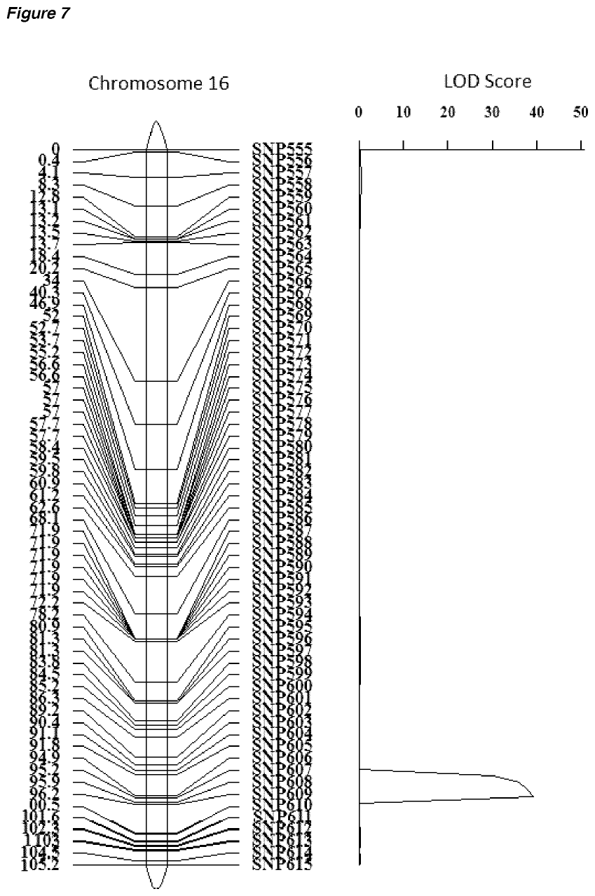

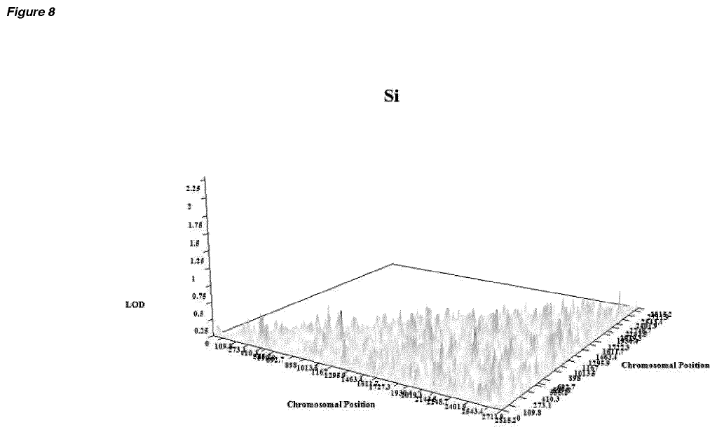

A linkage map of 768 SNP markers was used for QTL mapping of Si accumulation using the 141 RIIs from Majesta×Hikmok sorip. A single large effect QTL (named thereafter Hisil locus) was observed on chromosome 16 with a LOD score of 39.33 (FIG. 5), This QTL alone explained over 66% of the phenotypic variation (Table 11). This Hisil locus was found to be located at ca. 95 cM on the genetic map of chromosome 16 (FIGS. 6 & 7) No significant epistatic interactions were detected using EPlstatic QTL mapping as performed by ICIMapping (FIG. 8),

TABLE 11
Details of quantitative trait loci (QTL) identified for silicon accumulation
in soybean leaf using different software tool

| | Mapping | | | Left | Right | | | Add. |
|---|---|---|---|---|---|---|---|---|
| Software | method | Chr. | Position | Marker | Marker | LOD | PVE (%) | effect |
| ICIMapping | ICIM | 16 | 95.2 | SNP606 | SNP607 | 39.33 | 66.62 | −0.37 |
| | IM | 16 | 95.2 | SNP606 | SNP607 | 38.29 | 70.91 | −0.38 |
| | | 16 | 97.0 | SNP609 | SNP610 | 35.79 | 70.54 | −0.38 |

ICIM—Inclusive composite interval mapping;
IM—Interval mapping;
Chr.—Chromosome;
PVE—phenotypic variance explained;
Add. effect—Additive effect.

### Grafting Experiments

To further characterize the Si uptake trait, different cultivars were grafted onto a Hikmok sorip rootstock and vice versa and evaluated for Si absorption. Results showed that Si accumulation in a given graft was determined by the rootstock and not the aerial portion of the plant. In addition, grafts with Hikmok sorip as rootstock absorbed as much Si as Hikmok sorip hence confirming the unique trait of Hikmok sorip to absorb higher quantities of Si (Table 12).

TABLE 12
Silicon (Si) uptake observed in leaves of different soybean
cultivar grafted on Hikmok sorip rootstock and vice versa

| | | Average | Standard |
|---|---|---|---|
| Scion | Rootstock | Si (%) | Deviation |
| Majesta | Hikmok sorip | 2.81 | 0.29 |
| Jack | Hikmok sorip | 2.85 | 0.54 |
| Williams 82 | Hikmok sorip | 2.85 | 0.06 |
| Hikmok sorip | Majesta | 1.32 | 0.23 |
| Hikmok sorip | Jack | 1.36 | 0.10 |
| Hikmok sorip | Williams 82 | 1.39 | 0.42 |
| Majesta | Majesta | 1.23 | 0.12 |
| Jack | Jack | 1.31 | 0.18 |
| Williams 82 | Williams 82 | 1.16 | 0.34 |
| Hikmok sorip | Hikmok sorip | 2.93 | 0.26 |

### Discussion

In this work, we discovered a specific genomic region, thereafter named Kish', in a specific soybean cultivar known as Hikmok sorip that confers the ability to accumulate higher quantities of silicon (Si). Si is known to provide plants with many benefits, mostly in the prevention of biotic and abiotic stresses, when it is sufficiently available or amended in a growth substrate.

The protective role of silicon against stresses will be greatly influenced by the ability of the plant species under treatment to absorb the element. For this reason, some plant species will not respond to a Si treatment and results will often be interpreted as a failure by Si to confer protection, rather than a biological limitation. As a general rule, all monocots are Si accumulators. For dicots, the picture is not as clear as most dicots are unable to accumulate Si, For instance, the model plant Arabidopsis will only accumulate limited amounts. Notable exceptions among dicots are the Cucurbitaceae that are well known to benefit from Si feeding. Other exceptions include some species within the legumes such as pigeon pea and soybean (Hodson et al., 2005).

At the intraspecific level, limited variation in Si absorption ability has been reported or observed. For that purpose, monocots and more specifically rice have been studied and variations between the tested cultivars never exceeded 30%. It was therefore quite unexpected to observe variation as high as 200% between Hikmok sorip and other soybean cultivars tested (Arsenault-Labrecque et al., 2012: Guérin et al., 2014).

To determine how common high silicon uptake is within soybean germplasm, we tested 139 cultivated soybean varieties. Our results showed that there was very little variation among the germplasm tested, most of the lines averaging around 1% Si. Expectedly, the GENAS analysis failed to identify associated SNP markers given the limited variation. These observations suggest that soybean germplasm is limited in its variation for Si absorption, a characteristic that appears to be shared by most if not all species in the plant kingdom.

Within our collection of RILs based on Majesta X Hikmok sorip, we observed a much wider variation of Si accumulation compared to the original set of 139 cultivated lines. As a matter of fact, two distinct peaks emerged suggesting that very few loci controlled this trait. This was confirmed by our QTL analysis that revealed that almost all the phenotypic variation could be explained by a single locus on chromosome 16. Our results further indicated the absence of epistatic interaction for this trait.

From a breeding point of view, this discovery brings a new and unique opportunity to create soybean lines with improved Si uptake and thus a greater resistance to biotic and abiotic stresses. Considering that Si-associated benefits are wide reaching, soybean lines carrying this trait could display multiple and durable resistance to the numerous constraints affecting soybean production.

### Example 2

### Markers Development

### Materials and Methods for Marker Development

Whole genome re-sequencing data of Hikmok sorip aligned with Williams 82 was used to predict Hisil-Del a large deletion of about 286 bp, Flanking primers to target Hisil-Del was designed using Primer3 software tool (bioinfo.utee/primer3-0.4.0/). Similarly, primers for the other deletions and insertion were designed using Primer3 software tool. PCR amplification of these primers was performed using DNA from Hikmok sorip, Williams, and recombinant inbred lines (RILs) were developed from the cross between Hikmok sorip and Magesta. PCR amplicons were resolved by agarose gel electrophoresis.

### Results

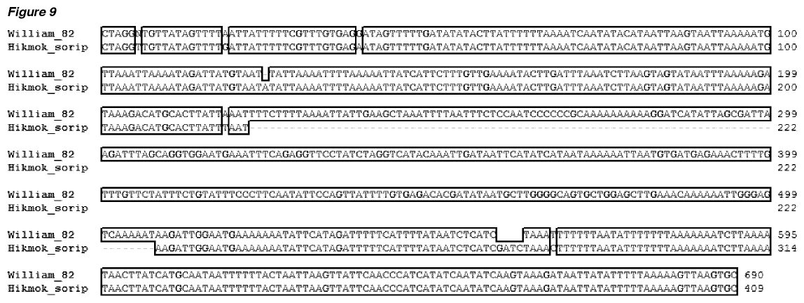

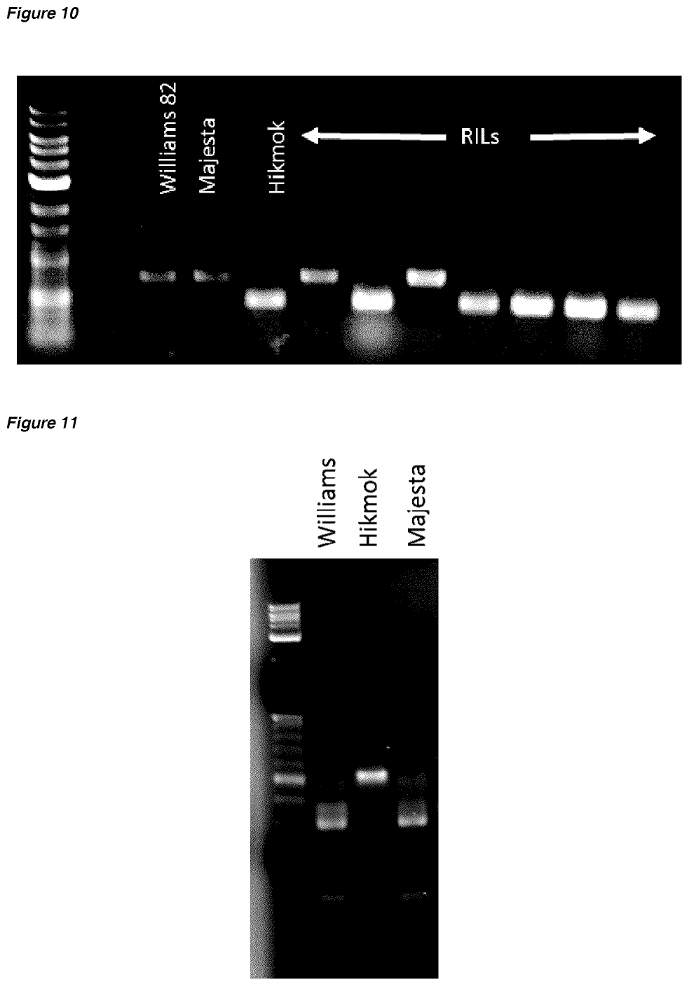

A set of five markers in the HiSil region was developed for the discriminant detection of HiSil gene in a segregating population. The marker HiSil-Del was designed based on a large deletion (-286 bp, Gm16:33,712,274 to 33,712,559) present in the cul ivar Hikmok sorip when compared to Williams 82 reference genome (G. max V1.1, FIG. 9) The HiSil-Del is tightly linked to HiSil since it is separated by a distance of only 28 Kb. Because of the large size difference in PCR amplicons, the marker HiSil-Del can be used to screen the presence of HiSil even using agarose gel electrophoresis (FIG. 10).

In addition, four gene-specific markers, including three deletions and one insertion in Hikmok sorip compared to Williams 82 reference genome, were developed (Table 13). These markers are helpful to follow the HiSil gene in segregating progenies and can be used to identify the gene in any new sources of germplasm.

TABLE 13
Details of markers linked to HiSil gene

| | | | Product | |
|---|---|---|---|---|
| | | | Size (bp) | |
| SEQ ID. | | | William | Hikmok |
| No. | Primer_ID | Primer Sequence | 82 | Sorip |
| 2 | HiSil-del1_F | GAATTTTAAGTCAACAGACATGCAC | 227 | 192 |
| 3 | HiSil-del1_R | TTTCACGGTAAAAATTATCACCAAC | | |
| 4 | HiSil-del2_F | GCAGGGAGGCAACAAATTAACAAAC | 328 | 0 |
| 5 | HiSil-del2_R | TGTTTCACAATCTTTCTTCTCACACAC | | |
| 6 | HiSil-del3b_F | GGAGGATCGCGACCATCATACTTTC | 398 | 278 |
| 7 | HiSil-del3b_R | TTCCACACCCTCACACATGATTGTA | | |
| 8 | HiSil-ins1_F | TGTCGCGTTAAATTCGTATGTTTG | 159 | 181 |
| 9 | HiSil-ins1_R | TCAAATTAAAGGCATGAGGATTTTGG | | |
| 10 | HiSil-Del_F | CCCACATCATTTTGACTTAACACTAG | 734 | 448 |
| 11 | HiSil-Del_R | TCTTCTTAGTTCTTAGATTCTCGCAC | | |

We have also designed a Cleaved Amplified Polymorphic Sequences (CAPS) marker linked to the HiSil gene. Conveniently, the Mboll restriction enzyme cleaves the PCR product into two fragments in the Hisil of Hikmok sorip variety and three fragments in the wild-type gene of the Williams variety (Table 14, FIG. 11),

TABLE 14
Details of Cleaved Amplified Polymorphic Sequences
(CAPS) markers linked to HiSil gene

| | | | Products after | |
|---|---|---|---|---|
| | | | cleavage | |
| SEQ ID | | | Williams | Hikmok |
| No. | Primer ID | Sequence | 82 | Sorip |
| 12 | HiSil- | CCTTTTATGTCTCTTCCGTTTGAAAAGC | 3 (73 bp, | 2 (73 bp, |
| | Mboll_F | | 169 bp, | 464 bp) |
| 13 | HiSil- | AAGTATGATGGTCGCGATCCTCCTCC | 295 bp) | |
| | Mboll_R | | | |

### Example 3

### Confirmation of QTL with High Density Genetic Map of Majesta×Hikmok sorip

Based on the QTL idenitifed in the linkage group J between the flanking markers of SNP605 and SNP610, the targeted region was further saturated with 94 new SNP markers. Genetic mapping was done by JoinMap (version 3.0) using regression mapping with the Kosambi's mapping function. A high density genetic map of 132 cM was constructed for the linkage group J.

All the 155 marker data (61 earlier mapped and 94 newly genotyped markers) of linkage group J was analyzed to find out the significance of association with the leaf Si content phenotype.

QTL mapping was performored in in-house workflow, where interval mapping, multiple interval mapping and composite interval mapping algorithms are integrated. The LR test statistics significance threshold of 13.8145 (LOD=2.0) was used to declare QTL.

The QTL mapping using the high density genetic map also detected a single major QTL in the same interval in the linkage group J which was detected in low density map.

### New HiSII Interval

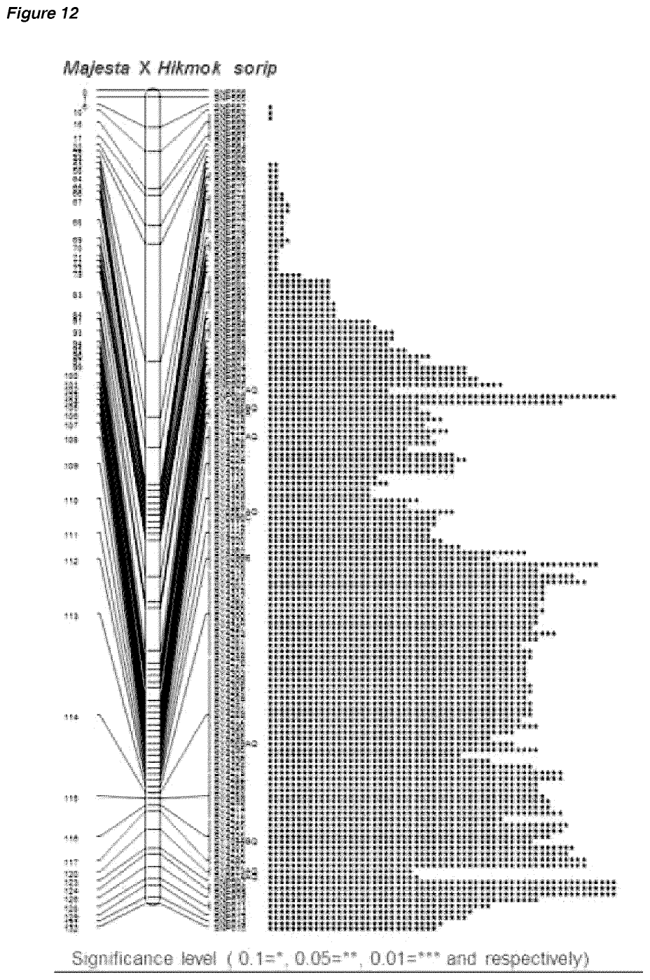

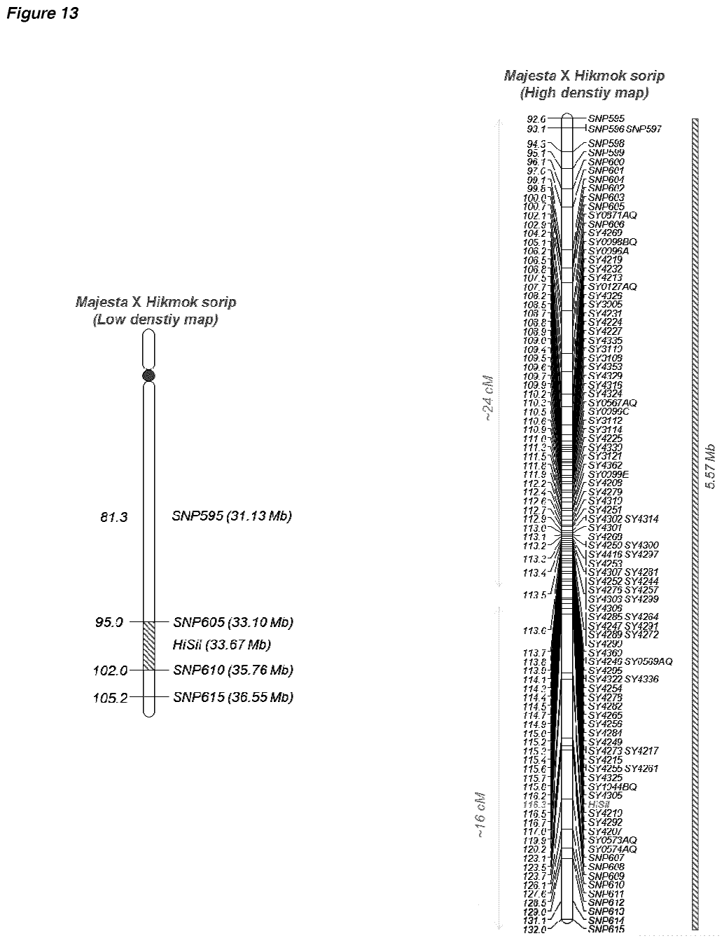

Marker analysis indicates that within the Hikmok×Majesta population, a chromosomal interval spanning from about SNP595 (31.13Mb) to SNP615 (36.55 Mb) (FIGS. 12 and 13) is highly associated with the HiSil trait.

A total of 155 markers were identified within this chromosomal interval to have a P value of less than or equal to 0.05 indicating that markers within this interval may be used to produce and/or select for lines having the HiSil Trait.

### Example 4

### QTL Mapping Performed in an Alternatve Mapping Population of Hamilton×PI 89772

As a PI 89772 has the same haplotype of Hikmok in the HiSil gene region, an alternate F2:3 mapping population of Hamilton×PI 89772 was used to confirm the HiSil QTL identified in Majesta×Hikmok

### Methods

### Phenotyping of Hamilton×PI 89772 Mapping Population

A mapping population derived from a cross Hamilton×PI 89772 was used for the QTL mapping. A total of 100 F3 (F2:3) lines were evaluated for Si uptake in the greenhouse at University Laval. Soybean plants, five per line, were grown in a greenhouse under controlled conditions. Plants were grown in potting soil with adequate supply of Si (1.7 mM) prepared from potassium silicate (Kasil #6, 23.6% SiO2, National Silicates), The first trifoliate leaf of each plant (5×100) was collected, dried and crushed to a fine powder. Leaf Si content was estimated by using a Niton XL3t Ultra Analyzer XRF according to the method described by Reidinger et al. (2012).

Genotyping, map construction and QTL mapping with Hamilton×PI89772 mapping population

Progeny of mapping population Hamilton x PI 89772 F2:3 were genotyped by 2990 genome wide markers. After removing the monomorphic markers, 1149 markers were used for genetic mapping. Genetic mapping was done by JoinMap (version 3.0) using regression mapping with the Kosambi's mapping function. A high density genetic map of 178 cM was constructed.The marker order between genetic and physical mapping is highly conserved.

QTL mapping was done in in-house workflow, where interval mapping, multiple interval mapping and composite interval mapping algorithms are integrated. The QTL are indentifed with a LR test statistics significance threshold of 13.8145 (LOD=2.0).

### Results

### Segregation of Leaf Silicon Content in Hamilton×PI 89772 Mapping Population

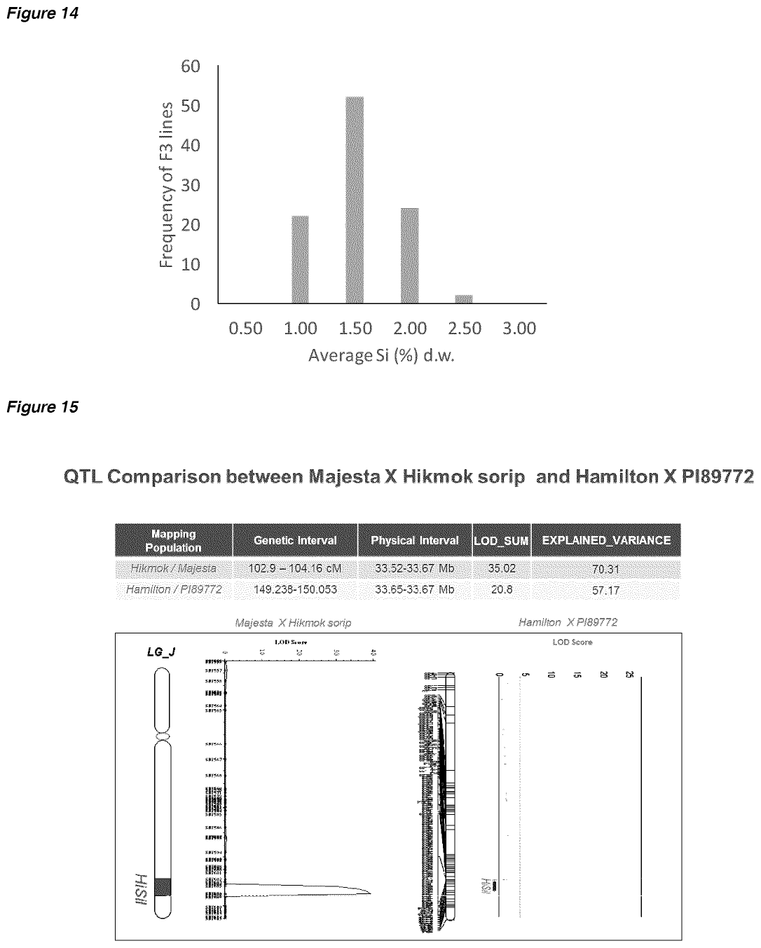

The F3 lines grown for three weeks with Si supplementation showed an average of 1.30% Si with a maximum of 2.03% and a minimum 0.71% Si. A typical 1:2:1 segregation was observed suggesting a single locus regulation of Si absorption (FIG. 14).

### Genetic Map and QTL for Leaf Silicon Content

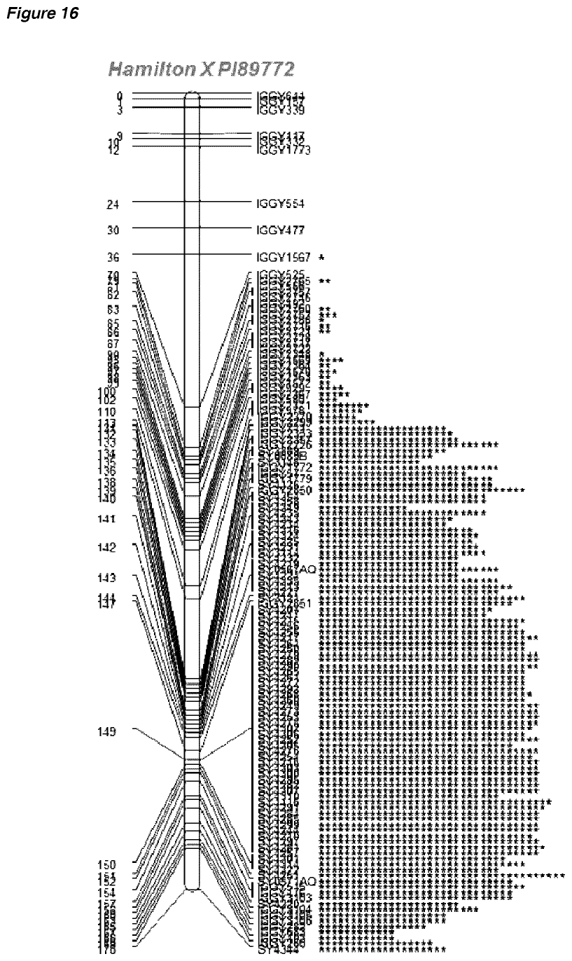

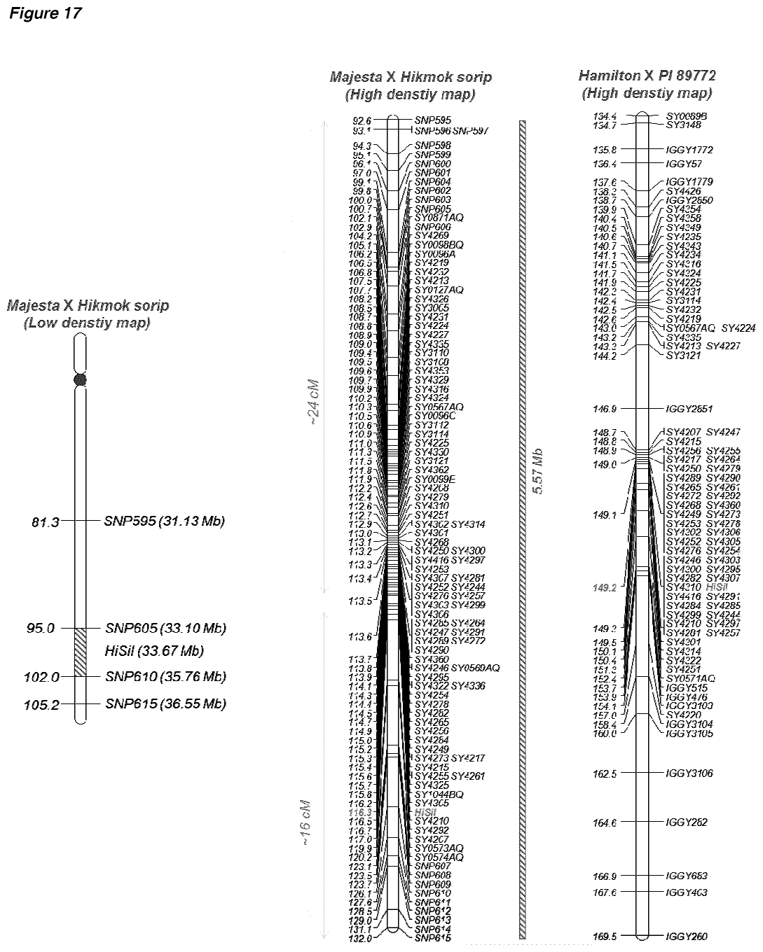

Based on the two mapping populations high-density genetic linkage map and the individual marker association with the leaf Si content pheonotype the defined interval for HiSil gene region is between the markers SY0089B to IGGY260. This interval in genetic map of Majesta X Hikmok sorip is between 92.6 cM to 132 cM, and corresponds to the physical map position of 31.15 Mb to 36.72 Mb (5.57 Mb fragment)) in chromosome 16 (FIGS. 13, 15, 16 & 17). The markers within this interval in both mapping populations have highly significant p-values for silicon uptake.

There are 135 markers developed in this interval, some of which are described in Table 15 below. More markers and favorable HiSil allel calls, targeted sequence, primer sequences and SNPs are presented in Tables 16-20.

TABLE 15
Markers p-values for each population

| | Physical | Hikmok × Majesta | Hamilton × PI89772 |
|---|---|---|---|
| Marker | Position | p-value | p-value |
| IGGY2746 | 12198849 | | 0.47973 |
| IGGY2752 | 12975991 | | 0.00622 |
| SNP574 | 14100150 | 0.00346 | |
| IGGY2757 | 14550460 | | 0.55375 |
| SNP575 | 14557684 | 0.00674 | |
| IGGY492 | 15662425 | | 0.13595 |
| IGGY566 | 16552349 | | 0.51714 |
| SNP576 | 17115818 | 0.00573 | |
| IGGY2760 | 17335057 | | 0.01356 |
| IGGY2765 | 18941830 | | 0.01866 |
| SNP577 | 19047353 | 0.00787 | |
| SNP578 | 19361529 | 0.00591 | |

TABLE 16
Markers for Majesta X Hikmok sorip

| Table 16. | | | | | | | Candidate | HiSil |
|---|---|---|---|---|---|---|---|---|
| Marker | Physical | | Sum of | Mean | | Significance level (0.1 = *, 0.05 = **, | Gene | interval |
| Name | Position | DF | Squares | Square | Prob > F | 0.01 = *** and respectively) | region | region |
| SNP555 | 68523 | 1 | 0.151353 | 0.15135 | 0.328348454 | | | |
| SNP556 | 185022 | 1 | 0.080349 | 0.08035 | 0.534313513 | | | |
| SNP557 | 1327055 | 1 | 0.555785 | 0.55579 | 0.065154167 | * | | |
| SNP558 | 2403360 | 1 | 0.494472 | 0.49447 | 0.078001122 | * | | |
| SNP559 | 2746738 | 1 | 0.335189 | 0.33519 | 0.141672748 | | | |
| SNP560 | 2767293 | 1 | 0.281512 | 0.28151 | 0.175995487 | | | |
| SNP561 | 2912151 | 1 | 0.32543 | 0.32543 | 0.147209825 | | | |
| SNP562 | 3038182 | 1 | 0.507504 | 0.5075 | 0.075809502 | * | | |
| SNP563 | 3047968 | 1 | 0.366235 | 0.36624 | 0.125732429 | | | |
| SNP564 | 3946949 | 1 | 0.086747 | 0.08675 | 0.506399746 | | | |
| SNP565 | 4256100 | 1 | 0.134143 | 0.13414 | 0.36258166 | | | |
| SNP566 | 4937234 | 1 | 0.003755 | 0.00375 | 2.966347664 | | | |
| SNP567 | 5703382 | 1 | 0.666382 | 0.66638 | 0.04558601 | ** | | |
| SNP568 | 6196089 | 1 | 0.817139 | 0.81714 | 0.028321054 | ** | | |
| SNP569 | 7138469 | 1 | 0.642251 | 0.64225 | 0.048512289 | ** | | |
| SNP570 | 7391200 | 1 | 0.85974 | 0.85974 | 0.024582701 | ** | | |
| SNP571 | 8023117 | 1 | 1.139265 | 1.13927 | 0.010683357 | ** | | |
| SNP572 | 8713046 | 1 | 1.204528 | 1.20453 | 0.00882985 | *** | | |
| SNP573 | 11563819 | 1 | 1.491718 | 1.49172 | 0.00385849 | **** | | |
| SNP574 | 14100150 | 1 | 1.529828 | 1.52983 | 0.003460473 | **** | | |
| SNP575 | 14557684 | 1 | 1.297166 | 1.29717 | 0.006748991 | *** | | |
| SNP576 | 17115818 | 1 | 1.353506 | 1.35351 | 0.005736309 | *** | | |
| SNP577 | 19047353 | 1 | 1.243958 | 1.24396 | 0.007873597 | *** | | |
| SNP578 | 19361529 | 1 | 1.342919 | 1.34292 | 0.005913919 | *** | | |
| SNP579 | 21610613 | 1 | 1.342919 | 1.34292 | 0.005913919 | *** | | |
| SNP580 | 21614498 | 1 | 1.466068 | 1.46607 | 0.004152226 | **** | | |
| SNP581 | 24007766 | 1 | 1.305901 | 1.3059 | 0.006580507 | *** | | |
| SNP582 | 24196632 | 1 | 1.088614 | 1.08861 | 0.012397597 | ** | | |
| SNP583 | 25768284 | 1 | 0.769365 | 0.76937 | 0.032458681 | ** | | |
| SNP584 | 26760058 | 1 | 0.652906 | 0.65291 | 0.046875849 | ** | | |
| SNP585 | 28073122 | 1 | 1.154882 | 1.15488 | 0.01020619 | ** | | |
| SNP586 | 29895735 | 1 | 2.514687 | 2.51469 | 0.000213473 | ****** | | |
| SNP587 | 30374483 | 1 | 4.462075 | 4.46208 | 7.83333E−07 | *********** | | |
| SNP588 | 30374503 | 1 | 4.462075 | 4.46208 | 7.83333E−07 | *********** | | |
| SNP589 | 30395866 | 1 | 4.996338 | 4.99634 | 1.59109E−07 | ************ | | |
| SNP590 | 30402864 | 1 | 4.462075 | 4.46208 | 7.83333E−07 | *********** | | |
| SNP591 | 30402865 | 1 | 4.668914 | 4.66891 | 4.24076E−07 | ************ | | |
| SNP592 | 30442126 | 1 | 4.462075 | 4.46208 | 7.83333E−07 | *********** | | |
| SNP593 | 30505518 | 1 | 4.739114 | 4.73911 | 3.44012E−07 | ************ | | |
| SNP594 | 30805487 | 1 | 7.256661 | 7.25666 | 1.29248E−10 | ****************** | | |
| SNP595 | 31136175 | 1 | 7.457938 | 7.45794 | 6.63242E−11 | ******************* | | HiSil |
| SNP596 | 31178190 | 1 | 8.378529 | 8.37853 | 2.89427E−12 | ********************** | | inteval |
| SNP597 | 31178205 | 1 | 8.378529 | 8.37853 | 2.89427E−12 | ********************** | | Region |
| SNP598 | 31472093 | 1 | 8.18326 | 8.18326 | 5.68859E−12 | ********************* | | |
| SNP599 | 31565242 | 1 | 9.030765 | 9.03076 | 2.88883E−13 | ************************ | | |
| SNP600 | 31840074 | 1 | 10.47006 | 10.47006 | 1.34716E−15 | **************************** | | |
| SY4353 | 31848568 | 2 | 8.055602 | 4.0278 | 1.61744E−10 | ****************** | | |
| SY3108 | 31860682 | 2 | 9.065398 | 4.5327 | 5.09474E−12 | ********************* | | |
| SY3110 | 31863327 | 2 | 8.373514 | 4.18676 | 5.07125E−11 | ******************* | | |
| SY0871AQ | 31869001 | 2 | 8.858589 | 4.42929 | 7.23307E−12 | ********************* | | |
| SY4329 | 31898811 | 2 | 7.854068 | 3.92703 | 3.15757E−10 | ****************** | | |
| SY3005 | 31996339 | 2 | 9.81321 | 4.9066 | 3.48357E−13 | ************************ | | |
| SNP601 | 32026703 | 1 | 9.915038 | 9.91504 | 1.12118E−14 | ************************** | | |
| SY4316 | 32039454 | 2 | 10.35878 | 5.17939 | 4.58243E−14 | ************************** | | |
| SNP602 | 32076322 | 1 | 11.96807 | 11.96807 | 3.13004E−18 | ********************************** | | |
| SY4324 | 32083583 | 2 | 10.10366 | 5.05183 | 1.24227E−13 | ************************ | | |
| SY3112 | 32084966 | 2 | 11.35494 | 5.67747 | 9.53431E−16 | ***************************** | | |
| SY0096C | 32100624 | 2 | 11.40449 | 5.70224 | 9.76298E−16 | ***************************** | | |
| SY0096A | 32101062 | 2 | 10.95473 | 5.47736 | 2.62075E−15 | **************************** | | |
| SY4225 | 32283031 | 2 | 11.10175 | 5.55088 | 2.12492E−15 | **************************** | | |
| SNP603 | 32329390 | 1 | 12.43003 | 12.43003 | 4.30751E−19 | ************************************ | | |
| SY4219 | 32343705 | 2 | 11.51485 | 5.75742 | 2.20571E−16 | ****************************** | | |
| SY3114 | 32474449 | 2 | 11.24478 | 5.62239 | 1.47978E−15 | **************************** | | |
| SY4231 | 32494752 | 2 | 12.29487 | 6.14743 | 3.11925E−17 | ******************************** | | |
| SY4326 | 32507776 | 2 | 11.42993 | 5.71496 | 7.05647E−16 | ***************************** | | |
| SY4232 | 32533983 | 2 | 10.80177 | 5.40088 | 8.42102E−15 | *************************** | | |
| SNP604 | 32547296 | 1 | 12.15319 | 12.15319 | 1.42361E−18 | ********************************** | | |
| SY4224 | 32843154 | 2 | 12.42758 | 6.21379 | 3.4057E−18 | ********************************** | | |
| SY0567AQ | 32881385 | 2 | 12.12912 | 6.06456 | 3.94449E−17 | ******************************** | | |
| SY0098BQ | 32881404 | 2 | 10.35748 | 5.17874 | 4.60527E−14 | ************************** | | |
| SY0127AQ | 32890833 | 2 | 9.815027 | 4.80751 | 3.2444E−15 | **************************** | | |
| SY4335 | 32906255 | 2 | 12.31108 | 6.15554 | 1.84737E−17 | ******************************** | | |
| SY4213 | 32946342 | 2 | 11.95973 | 5.97986 | 8.08869E−17 | ******************************* | | |
| SY4227 | 33021575 | 2 | 12.25133 | 6.12566 | 2.38034E−17 | ******************************** | | |
| SNP605 | 33104446 | 1 | 13.4811 | 13.4811 | 3.7692E−21 | **************************************** | | |
| SY4426 | 33104446 | 2 | 11.55566 | 5.77783 | 4.24728E−18 | ****************************** | | |
| SY4330 | 33204904 | 2 | 11.78663 | 5.89332 | 1.6544E−16 | ****************************** | | |
| SY3121 | 33263666 | 2 | 12.56897 | 6.28448 | 6.11656E−18 | ********************************* | | |
| SY4336 | 33463159 | 2 | 13.94979 | 8.97489 | 1.90832E−20 | ************************************** | | |
| SY0099E | 33474867 | 2 | 13.97061 | 6.9853 | 1.30428E−20 | ************************************** | | |
| SNP606 | 33527064 | 1 | 19.5685 | 19.5685 | 3.67287E−37 | ***************************************** | | |
| | | | | | | ****************** | | |
| SY4435 | 33540839 | 2 | 15.92787 | 7.96394 | 7.04519E−25 | ****************************************** | | |
| | | | | | | ***** | | |
| SY4325 | 33562531 | 2 | 16.12569 | 8.06285 | 1.5349E−25 | ******************************************** | | |
| | | | | | | **** | | |
| SNP607 | 33595090 | 1 | 19.40175 | 19.40175 | 1.39109E−36 | ********************************************** | | |
| | | | | | | ************* | | |
| SY4421 | 33595090 | 2 | 14.77934 | 7.38967 | 2.01678E−22 | ****************************************** | | |
| SY4439 | 33611752 | 2 | 15.89571 | 7.94785 | 1.09623E−24 | ********************************************** | | |
| SY4432 | 33636446 | 2 | 16.14538 | 8.07269 | 4.07334E−26 | ********************************************** | | |
| | | | | | | **** | | |
| SY4217 | 33654456 | 2 | 16.26075 | 8.13037 | 4.5184E−26 | ********************************************* | | |
| | | | | | | ***** | | |
| SY4310 | 33655743 | 2 | 16.92317 | 8.46158 | 1.46227E−27 | ******************************************** | | |
| | | | | | | ******** | | |
| SY4250 | 33655875 | 2 | 15.8712 | 7.9356 | 6.34085E−25 | ********************************************* | | |
| | | | | | | ** | | |
| SY4290 | 33655946 | 2 | 15.51561 | 7.75781 | 4.37898E−24 | ********************************************** | | |
| SY4297 | 33657467 | 2 | 15.33372 | 7.66686 | 6.69048E−24 | ********************************************* | | |
| SY4278 | 33658314 | 2 | 16.48768 | 8.24384 | 1.60274E−26 | ******************************************** | | |
| | | | | | | ****** | | |
| SY4284 | 33660305 | 2 | 15.888 | 7.944 | 5.77951E−25 | ******************************************** | | |
| | | | | | | *** | | |
| SY4261 | 33661778 | 2 | 16.6182 | 8.3091 | 1.73392E−26 | ******************************************* | | |
| | | | | | | ******* | | |
| SY4302 | 33662550 | 2 | 15.80226 | 7.90113 | 9.26426E−25 | ***************************************** | | |
| | | | | | | ****** | | |
| SY4252 | 33667338 | 2 | 15.37192 | 7.68596 | 9.41153E−24 | ********************************************* | | |
| SY4307 | 33667499 | 2 | 15.75358 | 7.87679 | 1.59139E−24 | ********************************************** | | |
| SY4255 | 33667587 | 2 | 16.60542 | 8.30271 | 7.72718E−27 | ******************************************* | | |
| | | | | | | ******* | | |
| SY4253 | 33667829 | 2 | 16.24036 | 8.12018 | 6.17405E−26 | ******************************************* | | |
| | | | | | | ***** | | |
| SY4247 | 33667974 | 2 | 15.49338 | 7.74669 | 6.2044E−24 | ******************************************** | HiSil | |
| SY4300 | 33668038 | 2 | 15.72529 | 7.86284 | 1.4109E−24 | ********************************************* | Candidate | |
| SY4305 | 33668118 | 2 | 16.84613 | 8.42306 | 2.32598E−27 | ********************************************* | Gene | |
| | | | | | | ****** | Region | |
| SY4257 | 33668227 | 2 | 15.37199 | 7.686 | 1.81392E−23 | ******************************************* | | |
| SY4289 | 33668347 | 2 | 15.51937 | 7.75969 | 8.90505E−24 | ******************************************** | | |
| SY4285 | 33668427 | 2 | 15.45654 | 7.72827 | 6.0043E−24 | ******************************************** | | |
| SY4276 | 33668501 | 2 | 14.45145 | 7.22572 | 1.65539E−23 | ******************************************* | | |
| SY4279 | 33668652 | 2 | 15.71191 | 7.85596 | 1.3399E−24 | ********************************************* | | |
| SY4246 | 33668680 | 2 | 13.70768 | 6.85384 | 1.52018E−20 | ************************************** | | |
| SY4306 | 33669577 | 2 | 15.45654 | 7.72827 | 6.0043E−24 | ********************************************* | | |
| SY4292 | 33669600 | 2 | 17.15298 | 8.57649 | 3.59046E−28 | ********************************************* | | |
| | | | | | | ********* | | |
| SY4314 | 33669639 | 2 | 15.97883 | 7.98941 | 5.84916E−25 | ******************************************* | | |
| | | | | | | **** | | |
| SY4299 | 33670119 | 2 | 15.4045 | 7.70225 | 1.64609E−23 | ******************************************** | | |
| SY4251 | 33670154 | 2 | 17.13755 | 8.56877 | 4.2901E−28 | ******************************************** | | |
| | | | | | | ********** | | |
| SY4301 | 33670204 | 2 | 15.86657 | 7.93329 | 6.50489E−25 | ****************************************** | | |
| | | | | | | ***** | | |
| SY4291 | 33670373 | 2 | 15.58439 | 7.78219 | 5.47082E−24 | ********************************************* | | |
| SY4207 | 33673022 | 2 | 17.3205 | 8.66025 | 1.26548E−28 | ********************************************* | | |
| | | | | | | ********* | | |
| SY4265 | 33673244 | 2 | 15.57149 | 7.78575 | 1.37076E−24 | ********************************************** | | |
| SY4282 | 33673483 | 2 | 16.52542 | 8.26271 | 1.36216E−26 | ******************************************* | | |
| | | | | | | ******* | | |
| SY4244 | 33673647 | 2 | 15.36955 | 7.68478 | 9.5304E−24 | ********************************************* | | |
| SY4264 | 33674572 | 2 | 15.23575 | 7.61788 | 1.64061E−23 | ******************************************** | | |
| SY4249 | 33676079 | 2 | 16.1861 | 8.09305 | 1.09114E−25 | ******************************************** | | |
| | | | | | | **** | | |
| SY4303 | 33676250 | 2 | 15.1569 | 7.57845 | 2.90934E−23 | ******************************************** | | |
| SY4295 | 33676255 | 2 | 15.55092 | 7.77546 | 3.6237E−24 | ********************************************** | | |
| SY4273 | 33676984 | 2 | 15.96304 | 7.98152 | 1.09951E−25 | ******************************************** | | |
| | | | | | | | **** | |
| SY4268 | 33678035 | 2 | 15.74199 | 7.871 | 1.28807E−24 | ********************************************** | | |
| SY4269 | 33679379 | 2 | 16.42154 | 8.21077 | 2.83679E−28 | ****************************************** | | |
| | | | | | | ******** | | |
| SY4254 | 33679893 | 2 | 15.39591 | 7.69795 | 6.74759E−24 | ********************************************* | | |
| SY4256 | 33680025 | 2 | 14.65863 | 7.32931 | 4.3023E−24 | ********************************************** | | |
| SY4272 | 33680071 | 2 | 14.98325 | 7.49163 | 7.13818E−23 | ******************************************* | | |
| SY4281 | 33680257 | 2 | 15.01452 | 7.50726 | 6.0786E−23 | ******************************************* | | |
| SY4416 | 33681630 | 2 | 15.76635 | 7.88317 | 1.12768E−24 | ********************************************** | | |
| SY4360 | 33681946 | 2 | 14.68797 | 7.34398 | 2.17132E−22 | ****************************************** | | |
| SY4210 | 33681961 | 2 | 16.7465 | 8.37325 | 7.07291E−27 | ******************************************** | | |
| | | | | | | ****** | | |
| SY4208 | 33682500 | 2 | t 17.717 | 8.8585 | 1.48266E−29 | ********************************************** | | |
| | | | | | | ********* | | |
| SY4362 | 33712274 | 2 | 15.34542 | 7.67271 | 1.08275E−23 | ******************************************** | | |
| SY4215 | 33728789 | 2 | 14.38978 | 7.19489 | 1.96837E−21 | **************************************** | | |
| SNP608 | 33802005 | 1 | 17.66351 | 17.66351 | 3.74686E−31 | ********************************************* | | |
| | | | | | | ************* | | |
| SY4418 | 33803957 | 2 | 14.92391 | 7.46196 | 7.68774E−23 | ******************************************* | | |
| SY0569AQ | 33853271 | 2 | 14.75802 | 7.37901 | 2.24609E−22 | ****************************************** | | |
| SY4322 | 34838750 | 2 | 12.17477 | 6.08738 | 6.14401E−18 | ********************************* | | |
| SY4433 | 35206878 | 2 | 14.23067 | 7.11533 | 4.3051E−21 | **************************************** | | |
| SY1044BQ | 35208490 | 2 | 15.45473 | 7.72736 | 7.74188E−24 | ********************************************* | | |
| SNP609 | 35218844 | 1 | 17.73376 | 17.73376 | 2.35896E−31 | ******************************************* | | |
| | | | | | | **************** | | |
| SY4437 | 35218844 | 2 | 13.03976 | 6.51988 | 2.97408E−22 | ****************************************** | | |
| SNP610 | 35762786 | 1 | 15.02618 | 15.02618 | 1.83896E−24 | ********************************************** | | |
| SY4440 | 35882270 | 2 | 11.31705 | 5.65852 | 6.35362E−16 | ***************************** | | |
| SY4434 | 35916594 | 2 | 12.12608 | 6.06304 | 6.27991E−17 | ******************************* | | |
| SNP611 | 36257345 | 1 | 13.14691 | 13.14691 | 1.76279E−20 | ************************************** | | |
| SNP612 | 36411870 | 1 | 12.26185 | 12.26185 | 8.92683E−19 | *********************************** | | |
| SNP613 | 36452436 | 1 | 12.01993 | 12.01993 | 2.51239E−18 | ********************************** | | |
| SNP614 | 36484326 | 1 | 11.09312 | 11.09312 | 1.1504E−16 | ****************************** | | |
| SNP615 | 36550306 | 1 | 10.59843 | 10.59843 | 8.176E−16 | ***************************** | | |
| SY0573AQ | 36641894 | 2 | 10.32794 | 5.16397 | 5.14808E−14 | ************************* | | |
| SY0574AQ | 36727283 | 2 | 10.4628 | 5.2314 | 2.68188E−14 | ************************** | | |

TABLE 17
Markers for Hamilton × PI89772

| | | | | | | | Significance level | | |
|---|---|---|---|---|---|---|---|---|---|
| Table 17. | | | | | | | (0.1 = *, 0.05 = **, | | HiSil |
| Marker | Physical | | Sum of | Mean | | | 0.01 = *** | Candidate | interval |
| Name | Position | DF | Squares | Square | F Ratio | Prob > F | and respectively) | Gene region | region |
| IGGY157 | 133098 | 2 | 0.3085745 | 0.154287 | 1.725 | 0.176974 | | | |
| IGGY644 | 133288 | 2 | 0.1061116 | 0.053056 | 0.5645 | 0.563217 | | | |
| IGGY339 | 724481 | 2 | 0.0504603 | 0.02523 | 0.2678 | 0.759802 | | | |
| IGGY117 | 1348464 | 2 | 0.2491671 | 0.124584 | 1.2867 | 0.27305 | | | |
| IGGY332 | 1629988 | 2 | 0.2312231 | 0.115612 | 1.2086 | 0.29448 | | | |
| IGGY1773 | 1630675 | 2 | 0.1759643 | 0.087982 | 0.8895 | 0.405797 | | | |
| IGGY554 | 2746738 | 2 | 0.2478873 | 0.123944 | 1.2785 | 0.275779 | | | |
| IGGY477 | 3534754 | 2 | 0.09811 | 0.049055 | 0.5017 | 0.599938 | | | |
| IGGY1567 | 4008875 | 2 | 0.4077588 | 0.203879 | 2.3314 | 0.097987 | * | | |
| IGGY525 | 6868110 | 2 | 0.2614395 | 0.13072 | 1.4192 | 0.239824 | | | |
| IGGY2746 | 12198849 | 2 | 0.1429733 | 0.071487 | 0.7242 | 0.479739 | | | |
| IGGY2752 | 12975991 | 2 | 0.8242433 | 0.412122 | 5.2996 | 0.006229 | *** | | |
| IGGY2757 | 14550460 | 2 | 0.1124239 | 0.056212 | 0.5808 | 0.553751 | | | |
| IGGY492 | 15662425 | 2 | 0.4060111 | 0.203006 | 1.9949 | 0.135954 | | | |
| IGGY566 | 16552349 | 2 | 0.1319188 | 0.065959 | 0.6493 | 0.517144 | | | |
| IGGY2760 | 17335057 | 2 | 0.6699558 | 0.334978 | 4.4385 | 0.013569 | ** | | |
| IGGY2765 | 18941830 | 2 | 0.7464825 | 0.373241 | 4.067 | 0.018667 | ** | | |
| IGGY2786 | 24608838 | 2 | 0.4515498 | 0.225775 | 2.521 | 0.081525 | * | | |
| IGGY2716 | 25066103 | 2 | 0.6415773 | 0.320789 | 3.8391 | 0.023459 | ** | | |
| IGGY2717 | 26011238 | 2 | 0.3131059 | 0.156553 | 1.8796 | 0.152179 | | | |
| IGGY2718 | 26124486 | 2 | 0.3879167 | 0.193958 | 2.238 | 0.107292 | | | |
| IGGY2721 | 26481028 | 2 | 0.5348341 | 0.267417 | 3.3401 | 0.037668 | ** | | |
| IGGY2722 | 26762918 | 2 | 0.2423423 | 0.121171 | 1.2499 | 0.283176 | | | |
| IGGY2348 | 26779932 | 2 | 0.4729054 | 0.236453 | 2.4891 | 0.084045 | * | | |
| IGGY1569 | 28389568 | 2 | 0.9841681 | 0.492084 | 5.9696 | 0.003356 | **** | | |
| IGGY1570 | 28657055 | 2 | 0.9434522 | 0.471726 | 5.1904 | 0.006584 | *** | | |
| IGGY2363 | 28657775 | 2 | 0.8292107 | 0.414605 | 4.3654 | 0.01435 | ** | | |
| IGGY2364 | 28710930 | 2 | 0.8233316 | 0.411666 | 4.5491 | 0.01186 | ** | | |
| IGGY1572 | 28999468 | 2 | 0.8057474 | 0.402874 | 4.4027 | 0.013638 | ** | | |
| IGGY929 | 29088944 | 2 | 0.9982975 | 0.499149 | 5.4964 | 0.004977 | **** | | |
| IGGY2367 | 29144193 | 2 | 1.3135219 | 0.656761 | 8.0003 | 0.00056 | ***** | | |
| IGGY580 | 29156455 | 2 | 0.8926534 | 0.446327 | 4.9791 | 0.007938 | *** | | |
| IGGY2370 | 30046974 | 2 | 1.579365 | 0.789683 | 9.4542 | 0.000158 | ****** | | |
| IGGY978 | 30151465 | 2 | 1.7116603 | 0.85583 | 10.6525 | 5.84E−05 | ******* | | |
| IGGY2371 | 30153571 | 2 | 1.8795405 | 0.93977 | 12.2797 | 1.64E−05 | ******** | | |
| IGGY2299 | 30331442 | 2 | 1.9166595 | 0.95833 | 13.4211 | 6.63E−06 | ********* | | |
| SY0089B | 31154742 | 2 | 3.4084689 | 1.704234 | 27.5372 | 2.54E−10 | ****************** | | HiSil |
| IGGY741 | 31154850 | 2 | 3.6426032 | 1.821302 | 34.2201 | 2.6E−11 | ******************** | | interval |
| SY3148 | 31192049 | 2 | 3.1322073 | 1.566104 | 25.6986 | 1.16E−09 | **************** | | Region |
| SY3889 | 31256347 | 2 | 3.6420165 | 1.821008 | 30.5667 | 3.98E−11 | ******************** | | |
| IGGY57 | 31860682 | 2 | 4.2726445 | 2.13622 | 39.9664 | 6.72E−13 | *********************** | | |
| SY4354 | 31868259 | 2 | 4.2958423 | 2.147921 | 44.6002 | 2.53E−14 | ************************** | | |
| SY4349 | 31947660 | 2 | 2.6174747 | 1.308737 | 23.0138 | 1.04E−08 | ************** | | |
| SY4343 | 31949260 | 2 | 3.430466 | 1.715233 | 34.4594 | 8.02E−12 | ********************* | | |
| SY4235 | 31952066 | 2 | 4.2484638 | 2.124232 | 43.6753 | 2.58E−14 | ************************** | | |
| SY4358 | 31991011 | 2 | 4.2423151 | 2.121158 | 43.8212 | 3.74E−14 | ************************** | | |
| IGGY2353 | 31996339 | 2 | 3.8734648 | 1.936732 | 33.7048 | 8.18E−12 | ********************* | | |
| SY4316 | 32039454 | 2 | 4.1426309 | 2.071315 | 41.4655 | 1.06E−13 | *********************** | | |
| SY4324 | 32083583 | 2 | 4.231746 | 2.115873 | 43.0226 | 6.76E−14 | ************************* | | |
| IGGY1779 | 32101062 | 2 | 4.5868262 | 2.293413 | 45.7381 | 9.76E−15 | *************************** | | |
| SY4234 | 32145135 | 2 | 3.6929261 | 1.846463 | 33.1526 | 1.2E−11 | ******************** | | |
| SY4225 | 32283031 | 2 | 4.176961 | 2.08848 | 39.9752 | 2E−13 | ************************ | | |
| SY4219 | 32343705 | 2 | 3.7183526 | 1.859176 | 36.3998 | 2.57E−12 | ********************** | | |
| SY3114 | 32474449 | 2 | 4.5250074 | 2.262504 | 45.0267 | 1.07E−14 | ************************** | | |
| SY4231 | 32494752 | 2 | 4.2118733 | 2.105937 | 42.8333 | 5.18E−14 | ************************* | | |
| SY4232 | 32533983 | 2 | 3.8811344 | 1.940567 | 36.8992 | 1.09E−12 | ********************** | | |
| SY4224 | 32843154 | 2 | 4.1041938 | 2.052097 | 36.63 | 1.27E−12 | ********************** | | |
| IGGY2850 | 32848989 | 2 | 5.0099213 | 2.504961 | 68.1297 | 2.35E−17 | ******************************** | | |
| SY0567AQ | 32881385 | 2 | 4.5848802 | 2.29244 | 49.0939 | 1.84E−15 | **************************** | | |
| IGGY1772 | 32881404 | 2 | 4.8616354 | 2.430818 | 53.6366 | 1.23E−15 | **************************** | | |
| IGGY2226 | 32890833 | 2 | 4.711218 | 2.355609 | 50.6433 | 1.89E−15 | **************************** | | |
| SY4335 | 32906255 | 2 | 4.4657107 | 2.232855 | 49.7423 | 1.65E−15 | **************************** | | |
| SY4213 | 32946342 | 2 | 4.8280627 | 2.414031 | 53.2842 | 3.12E−16 | ****************************** | | |
| SY4227 | 33021575 | 2 | 4.657339 | 2.32867 | 49.8694 | 1.56E−15 | **************************** | | |
| SY4426 | 33104446 | 2 | 4.2506796 | 2.12534 | 53.3329 | 5.36E−15 | *************************** | | |
| SY3121 | 33263666 | 2 | 5.0111924 | 2.505596 | 55.66 | 4.13E−17 | ******************************** | | |
| IGGY2357 | 33324609 | 2 | 4.1184615 | 2.059231 | 39.3923 | 1.09E−12 | ********************** | | |
| SY4217 | 33654456 | 2 | 5.0867587 | 2.543379 | 63.534 | 2.97E−18 | *********************************** | | |
| SY4310 | 33655743 | 2 | 4.743259 | 2.37163 | 49.9829 | 1.07E−15 | **************************** | | |
| SY4250 | 33655875 | 2 | 4.9775747 | 2.488787 | 56.4107 | 3.64E−17 | ******************************** | | |
| SY4290 | 33655946 | 2 | 5.0825746 | 2.541287 | 62.8785 | 4.52E−18 | ********************************** | | |
| SY4297 | 33657467 | 2 | 5.0766191 | 2.53831 | 64.202 | 1.69E−18 | ********************************** | | |
| SY4278 | 33658314 | 2 | 5.3053377 | 2.652669 | 63.7125 | 2.08E−18 | ********************************** | | |
| SY4284 | 33660305 | 2 | 5.2867145 | 2.643357 | 61.1883 | 4.74E−18 | ********************************** | | |
| SY4261 | 33661778 | 2 | 5.2009548 | 2.600477 | 57.6881 | 1.8E−17 | ******************************** | | |
| SY4302 | 33662550 | 2 | 5.1267078 | 2.563354 | 61.8146 | 8.17E−18 | ********************************* | | |
| SY4252 | 33667338 | 2 | 5.0867587 | 2.543379 | 63.534 | 2.97E−18 | ********************************** | | |
| SY4307 | 33667499 | 2 | 5.0766191 | 2.53831 | 64.202 | 1.69E−18 | ********************************** | | |
| SY4255 | 33667587 | 2 | 4.8229725 | 2.411486 | 59.0616 | 2.65E−17 | ******************************** | | |
| SY4253 | 33667829 | 2 | 5.2311489 | 2.615574 | 62.9171 | 2.23E−18 | ********************************** | | |
| SY4247 | 33667974 | 2 | 4.4476177 | 2.223809 | 43.7947 | 2.22E−14 | ************************** | | |
| SY4300 | 33668038 | 2 | 5.2978241 | 2.648912 | 63.8267 | 1.72E−18 | ********************************** | | |
| SY4305 | 33668118 | 2 | 4.9573634 | 2.478682 | 54.2705 | 1.56E−16 | ****************************** | | |
| SY4257 | 33668227 | 2 | 5.2785828 | 2.639291 | 62.9034 | 1.51E−18 | ********************************** | | |
| SY4289 | 33668347 | 2 | 5.2717257 | 2.635863 | 64.4284 | 1.16E−18 | ********************************** | | |
| SY4285 | 33668427 | 2 | 5.0766191 | 2.53831 | 64.202 | 1.69E−18 | ********************************** | | |
| SY4276 | 33668501 | 2 | 5.0766191 | 2.53831 | 64.4348 | 1.53E−18 | ********************************** | | |
| SY4279 | 33668652 | 2 | 5.3047481 | 2.652374 | 64.2155 | 1.46E−18 | ********************************** | | |
| SY4246 | 33668680 | 2 | 5.1829674 | 2.591484 | 63.8842 | 2.23E−18 | ********************************** | | |
| SY4306 | 33669577 | 2 | 4.9813009 | 2.49065 | 59.055 | 1.79E−17 | ******************************** | | |
| SY4292 | 33669600 | 2 | 5.0115917 | 2.505796 | 59.1339 | 2.25E−17 | ******************************** | | |
| SY4314 | 33669639 | 2 | 4.9248768 | 2.462438 | 61.1088 | 1.46E−17 | ******************************** | | |
| SY4299 | 33670119 | 2 | 5.0766191 | 2.53831 | 64.202 | 1.69E−18 | ********************************** | | |
| SY4251 | 33670154 | 2 | 5.6130402 | 2.80652 | 94.8063 | 3.41E−20 | ************************************** | | |
| SY4301 | 33670204 | 2 | 4.7926543 | 2.396327 | 50.1686 | 6.55E−16 | ***************************** | | |
| SY4291 | 33670373 | 2 | 5.2971332 | 2.648567 | 65.139 | 7.45E−19 | ************************************ | Hisil [symbol] | |
| SY4207 | 33673022 | 2 | 4.2776157 | 2.138808 | 47.418 | 7.73E−15 | *************************** | | |
| SY4265 | 33673244 | 2 | 4.9212188 | 2.460609 | 59.8477 | 1.65E−17 | ******************************** | | |
| SY4282 | 33673483 | 2 | 5.0801246 | 2.540062 | 63.7063 | 2.4E−18 | ********************************** | | |
| SY4244 | 33673647 | 2 | 5.2144496 | 2.607225 | 63.9812 | 1.23E−18 | ********************************** | | |
| SY4264 | 33674572 | 2 | 5.1110591 | 2.55553 | 58.8881 | 1.93E−17 | ******************************** | | |
| SY4249 | 33676079 | 2 | 5.0867587 | 2.543379 | 63.534 | 2.97E−18 | ********************************** | | |
| SY4303 | 33676250 | 2 | 5.0867587 | 2.543379 | 63.534 | 2.97E−18 | ********************************** | | |
| SY4295 | 33676255 | 2 | 5.0766191 | 2.53831 | 64.202 | 1.69E−18 | ********************************** | | |
| SY4273 | 33676984 | 2 | 5.1071056 | 2.553553 | 63.7484 | 3.14E−18 | ********************************** | | |
| SY4268 | 33678035 | 2 | 5.1116546 | 2.555827 | 61.0581 | 7.46E−18 | ********************************* | | |
| SY4254 | 33679893 | 2 | 4.9562902 | 2.478145 | 60.5918 | 8E−18 | ********************************* | | |
| SY4256 | 33680025 | 2 | 4.7499567 | 2.374978 | 56.7318 | 5.07E−17 | ******************************* | | |
| SY4272 | 33680071 | 2 | 4.9529027 | 2.476451 | 59.8101 | 1.47E−17 | ******************************** | | |
| SY4281 | 33680257 | 2 | 5.2339935 | 2.616997 | 66.6603 | 5.2E−19 | *********************************** | | |
| SY4416 | 33681630 | 2 | 5.4962862 | 2.748143 | 68.7155 | 2.23E−19 | ************************************ | | |
| SY4360 | 33681946 | 2 | 5.1188003 | 2.5594 | 59.4551 | 1.16E−17 | ******************************** | | |
| SY4210 | 33681961 | 2 | 4.9433089 | 2.471654 | 59.9274 | 9.42E−18 | ********************************* | | |
| SY4215 | 33728789 | 2 | 4.9857988 | 2.492899 | 57.7078 | 2.55E−17 | ******************************** | | |
| IGGY515 | 33761413 | 2 | 5.1538024 | 2.576901 | 69.1883 | 4.98E−17 | ******************************** | | |
| IGGY3103 | 33802827 | 2 | 4.7895537 | 2.394777 | 54.0046 | 3.64E−16 | ****************************** | | |
| SY4322 | 34838750 | 2 | 4.7289297 | 2.364465 | 51.4523 | 1.03E−15 | **************************** | | |
| SY4344 | 34838853 | 1 | 3.115028 | 3.115028 | 55.0023 | 2.71E−11 | ******************** | | |
| IGGY2851 | 35127959 | 2 | 5.0307791 | 2.51539 | 54.7949 | 1.23E−16 | ****************************** | | |
| IGGY3104 | 35146338 | 2 | 4.5279721 | 2.263986 | 44.3425 | 6.43E−14 | ************************* | | |
| IGGY476 | 35175117 | 2 | 4.9288214 | 2.464411 | 52.9945 | 4.51E−16 | ****************************** | | |
| SY0571AQ | 35571465 | 2 | 4.8540672 | 2.427034 | 52.8678 | 2.16E−16 | ****************************** | | |
| SY4220 | 35912570 | 2 | 3.666125 | 1.833062 | 34.6852 | 4.99E−12 | ********************** | | |
| IGGY3105 | 36138575 | 2 | 3.9769631 | 1.988482 | 35.6459 | 1.53E−11 | ******************** | | |
| IGGY3106 | 36503493 | 2 | 3.7888135 | 1.894407 | 33.0735 | 1.25E−11 | ******************** | | |
| IGGY282 | 36641894 | 2 | 3.3242547 | 1.662127 | 25.9414 | 6.63E−10 | ***************** | | |
| IGGY260 | 36727283 | 2 | 3.4786383 | 1.739319 | 27.0017 | 4.26E−10 | ****************** | | |
| IGGY683 | 37181573 | 2 | 2.5699486 | 1.284974 | 17.5557 | 3.91E−07 | ************ | | |
| IGGY403 | 37288898 | 2 | 2.5848003 | 1.2924 | 17.3837 | 4.96E−07 | ************ | | |

[symbol]

indicates data missing or illegible when filed

TABLE 18
Favourable Alleles

| Table 18. | PanDa Variant | | Physical | Favorable | Unfavourable |
|---|---|---|---|---|---|
| Marker Name | UId | Marker targeting the DNA polymorphism | Position* | Allele | Allele |
| SY0089B | 12917729 | IGGY1884, IIY26902, IIY26903, IIY526, KY2360A, | 31154742 | A/A | C/C |
| | | SY0089B, SY0089BQ | | | |
| IGGY741 | 12917727 | IGGY741, IIY26809, IIY26810, IIY27145, KY0845A, | 31154850 | T/T | A/A |
| | | SY0089A, SY0089AQ | | | |
| SY3148 | 12980667 | IGGY2378, IIY22259, SY3148 | 31192049 | A/A | G/G |
| SY3889 | 12981395 | IIY31526, SY3889 | 31256347 | A/A | G/G |
| SY4353 | 56017303 | SY4353 | 31848568 | A/A | G/G |
| SY3108 | 12979617 | IGGY57, IIY21912, SY3108 | 31860682 | T/T | A/A |
| SY3110 | 12979670 | IGGY59, IIY21906, SY3110 | 31863327 | A/A | G/G |
| SY4354 | 56017304 | SY4354 | 31868259 | T/T | A/A |
| SY0871AQ | 12981920 | IGGY574, IIY26933, IIY26934, KY2834A, SY0871AQ | 31869001 | T/T | A/A |
| SY4329 | 56017305 | SY4329 | 31898811 | C/C | A/A |
| SY4349 | 56017307 | SY4349 | 31947660 | G/G | A/A |
| SY4343 | 56021709 | SY4343 | 31949260 | T/T | A/A |
| SY4235 | 12981079 | IIY6295, SY4235 | 31952066 | A/A | G/G |
| SY4358 | 56017308 | SY4358 | 31991011 | A/A | C/C |
| SY3005 | 12980686 | IGGY2353, IIY22234, SY3005 | 31996339 | A/A | C/C |
| SY4316 | 56021714 | SY4316 | 32039454 | G/G | A/A |
| SY4324 | 56017310 | SY4324 | 32083583 | G/G | A/A |
| SY3112 | 12979655 | IGGY64, IIY21913, SY3112 | 32084966 | C/C | A/A |
| SY0096C | 12976596 | IIY27048, IIY27049, IIY32229, IIY685, SY0096C, SY0096CQ | 32100624 | T/T | A/A |
| SY0096A | 12976600 | IGGY1779, IIY31492, IIY684, KY0853A, SY0096A, SY0096AQ | 32101062 | G/G | A/A |
| SY4234 | 56017312 | SY4234 | 32145135 | G/G | A/A |
| SY4225 | 56021721 | SY4225 | 32283031 | G/G | C/C |
| SY4219 | 56021724 | SY4219 | 32343705 | G/G | A/A |
| SY3114 | 12979695 | IGGY76, IIY31725, SY3114 | 32474449 | A/A | T/T |
| SY4231 | 56017315 | SY4231 | 32494752 | G/G | A/A |
| SY4326 | 56021730 | SY4326 | 32507776 | A/A | G/G |
| SY4232 | 56021731 | SY4232 | 32533983 | G/G | A/A |
| SY4224 | 56017318 | SY4224 | 32843154 | A/A | C/C |
| IGGY2850 | 12940106 | IGGY2850, IIY31258 | 32848989 | C/C | G/G |
| SY0567AQ | 12933268 | IIY26982, IIY26983, IIY31233, KY2763A, SY0567AQ | 32881385 | T/T | A/A |
| SY0098BQ | 12933267 | IGGY1772, IIY27070, IIY27071, IIY32273, IIY634, SY0098B, | 32881404 | G/G | A/A |
| | | SY0098BQ | | | |
| SY0127AQ | 12948965 | IGGY2226, IIY14700, SY0127A, SY0127AQ | 32890833 | G/G | A/A |
| SY4335 | 56017319 | SY4335 | 32906255 | T/T | A/A |
| SY4213 | 56017321 | SY4213 | 32946342 | T/T | A/A |
| SY4227 | 56017322 | SY4227 | 33021575 | A/A | G/G |
| SY4426 | 353462473 | SY4426, SNP605 | 33104446 | T/T | A/A |
| SY4330 | 56021742 | SY4330 | 33204904 | A/A | G/G |
| SY3121 | 12980630 | IGGY2354, IIY22235, SY3121 | 33263666 | C/C | A/A |
| IGGY2357 | 12980624 | IGGY2357, IIY26917, IIY26918, IIY27306, SY3126 | 33324609 | C/C | G/G |
| SY4336 | 12940400 | SY4336 | 33463159 | C/C | A/A |
| SY0099E | 23543290 | IGGY2310, IIY22189, SY0099E, SY0099EQ | 33474867 | G/G | A/A |
| SY4427 | 412802301 | SY4427, SNP606 | 33527064 | A/A | T/T |
| SY4435 | 12940422 | SY4435 | 33540839 | G/G | A/A |
| SY4325 | 56021749 | SY4325 | 33562531 | A/A | T/T |
| SY4421 | 412802302 | SY4421, SNP607 | 33595090 | A/A | C/C |
| SY4439 | 12940448 | SY4439 | 33611752 | G/G | A/A |
| SY4432 | 12940376 | SY4432 | 33636446 | A/A | G/G |
| SY4217 | 56021750 | SY4217 | 33654456 | A/A | G/G |
| SY4310 | 271724460 | SY4310 | 33655743 | G/G | A/A |
| SY4250 | 271344625 | SY4250 | 33655875 | A/A | G/G |
| SY4290 | 271914417 | SY4290 | 33655946 | A/A | G/G |
| SY4297 | 271534944 | SY4297 | 33657467 | A/A | G/G |
| SY4278 | 270585230 | SY4278 | 33658314 | G/G | A/A |
| SY4284 | 270964571 | SY4284 | 33660305 | A/A | G/G |
| SY4261 | 270964573 | SY4261 | 33661778 | G/G | A/A |
| SY4302 | 270775294 | SY4302 | 33662550 | A/A | G/G |
| SY4252 | 271914434 | SY4252 | 33667338 | G/G | A/A |
| SY4307 | 271344641 | SY4307 | 33667499 | G/G | A/A |
| SY4255 | 270585250 | SY4255 | 33667587 | A/A | T/T |
| SY4253 | 270775313 | SY4253 | 33667829 | C/C | G/G |
| SY4247 | 270775314 | SY4247 | 33667974 | T/T | A/A |
| SY4300 | 270585251 | SY4300 | 33668038 | G/G | A/A |
| SY4305 | 270964584 | SY4305 | 33668118 | G/G | A/A |
| SY4257 | 270775316 | SY4257 | 33668227 | D/D | I/I |
| SY4289 | 271154434 | SY4289 | 33668347 | I/I | D/D |
| SY4285 | 271154435 | SY4285 | 33668427 | G/G | A/A |
| SY4276 | 270964586 | SY4276 | 33668501 | I/I | D/D |
| SY4279 | 271534965 | SY4279 | 33668652 | G/G | A/A |
| SY4246 | 271914435 | SY4246 | 33668680 | A/A | G/G |
| SY4306 | 271154438 | SY4306 | 33669577 | G/G | A/A |
| SY4292 | 271724476 | SY4292 | 33669600 | A/A | G/G |
| SY4314 | 271914437 | SY4314 | 33669639 | C/C | A/A |
| SY4299 | 270964590 | SY4299 | 33670119 | D/D | I/I |
| SY4251 | 271534967 | SY4251 | 33670154 | G/G | A/A |
| SY4301 | 270585256 | SY4301 | 33670204 | A/A | G/G |
| SY4291 | 270964591 | SY4291 | 33670373 | C/C | G/G |
| SY4207 | 266863993 | SY4207 | 33673022 | T/T | A/A |
| SY4265 | 271344646 | SY4265 | 33673244 | A/A | C/C |
| SY4282 | 271154440 | SY4282 | 33673483 | G/G | A/A |
| SY4244 | 270585257 | SY4244 | 33673647 | A/A | C/C |
| SY4264 | 271914440 | SY4264 | 33674572 | D/D | I/I |
| SY4249 | 271534977 | SY4249 | 33676079 | A/A | T/T |
| SY4303 | 270964599 | SY4303 | 33676250 | A/A | G/G |
| SY4295 | 270585267 | SY4295 | 33676255 | A/A | G/G |
| SY4273 | 270964601 | SY4273 | 33676984 | A/A | G/G |
| SY4268 | 271154450 | SY4268 | 33678035 | A/A | G/G |
| SY4269 | 271154453 | SY4269 | 33679379 | A/A | G/G |
| SY4254 | 270585272 | SY4254 | 33679893 | D/D | I/I |
| SY4256 | 270964605 | SY4256 | 33680025 | T/T | A/A |
| SY4272 | 271154454 | SY4272 | 33680071 | C/C | A/A |
| SY4281 | 270775330 | SY4281 | 33680257 | G/G | C/C |
| SY4416 | 272389082 | SY4416 | 33681630 | G/G | A/A |
| SY4360 | 266863987 | SY4206, SY4360 | 33681946 | T/T | A/A |
| SY4210 | 266863990 | SY4210 | 33681961 | A/A | G/G |
| SY4208 | 266863989 | SY4208 | 33682500 | A/A | G/G |
| SY4362 | 999991351 | SY4362 | 33712274 | D/D | I/I |
| SY4215 | 56021751 | SY4215 | 33728789 | A/A | T/T |
| IGGY515 | 12977667 | IGGY515, IIY570, KY0859A, SY0570AQ | 33761413 | G/G | A/A |
| IGGY3103 | 12981397 | IGGY3103, IIY16547, SY3897 | 33802827 | G/G | A/A |
| SY4418 | 12940610 | SY4418 | 33803957 | C/C | A/A |
| SY0569AQ | 12976666 | IGGY343, IIY27050, IIY27051, IIY31493, KY2903A, SY0569AQ | 33853271 | A/A | G/G |
| SY4322 | 56021755 | SY4322 | 34838750 | A/A | G/G |
| SY4344 | 56021756 | SY4344 | 34838853 | A/A | G/G |
| IGGY2851 | 12940655 | IGGY2851, IIY27138 | 35127959 | A/A | G/G |
| IGGY3104 | 12940605 | IGGY3104, IIY27171, SY3898 | 35146338 | C/C | G/G |
| IGGY476 | 12977396 | IGGY476, IIY31353, KY4251A, SY0568AQ | 35175117 | G/G | C/C |
| SY4433 | 12981180 | IIY6383, SY4433 | 35206878 | G/G | C/C |
| SY1044BQ | 12973395 | IGGY768, IIY15423, IIY31340, KY4709A, SY1044AQ, SY1044BQ | 35208490 | A/A | G/G |
| SY4437 | 412802304 | SY4437, SNP609 | 35218844 | A/A | G/G |
| SY0571AQ | 12976368 | IGGY308, IIY32155, IIY97, KY2617A, SY0571AQ | 35571465 | G/G | A/A |
| SY4428 | 412802305 | SY4428, SNP610 | 35762786 | A/A | G/G |
| SY4440 | 12940854 | SY4440 | 35882270 | G/G | C/C |
| SY4220 | 56017329 | SY4220 | 35912570 | G/G | A/A |
| SY4434 | 12981186 | IIY6407, SY4434 | 35916594 | A/A | G/G |
| IGGY3105 | 12981327 | IGGY3105, IIY27172, SY3899 | 36138575 | A/A | G/G |
| IGGY3106 | 12981417 | IGGY3106, IIY27133, SY3900 | 36503493 | G/G | A/A |
| SY0573AQ | 12916916 | IGGY282, IIY249, IIY32182, KY2435A, SY0573AQ | 36641894 | A/A | G/G |
| SY0574AQ | 23543129 | IGGY260, IIY27281, KY2274A, SY0574AQ | 36727283 | G/G | A/A |

TABLE 19
Primers and probes for markers

| | | | | | | |
|---|---|---|---|---|---|---|
| Table 19 | PanDa - Variant | | | Assay component | | |
| Marker Name | UID | Assay id | Type | name | DNA sequence | SEQ ID NO. |

| | | | | | | |
|---|---|---|---|---|---|---|
| IGGY260 | 1065762 | IGGY260 | Illumina Golden Gate | | | |
| IGGY260 | 1065762 | IGGY260 | Illumina Golden Gate | IGGY260F3 | TCAAACGACACCGTCTCAT | 27 |
| IGGY260 | 1065762 | IGGY260 | Iillumina Golden Gate | IGGY260F1 | ACTTCGTCAGTAACGGACGCAAGTTCGAGGGCCAGAGCCTT | 28 |
| IGGY260 | 1065762 | IGGY260 | Illumina Golden Gate | IGGY260F2 | GAGTCGAGGTCATATCGTGCAAGTTCGAGGGCCAGAGCCTC | 29 |
| SY0574AQ | 1078374 | SY0574AQ | Taqman | | | |
| SY0574AQ | 1078374 | SY0574AQ | Taqman | SY0574AF1 | GCGAGGAGGTCGTAGATGAGA | 30 |
| SY0574AQ | 1078374 | SY0574AQ | Taqman | SY0574AR1 | TGAAGGGTAGTTCCGACAAAGAAAC | 31 |
| SY0574AQ | 1078374 | SY0574AQ | Taqman | SY0574AA1FM | TGTCGTTTGACAAGGC | 278 |
| SY0574AQ | 1078374 | SY0574AQ | Taqman | SY0574AA2TT | TCGTTTGACGAGGCT | 279 |
| SY0573AQ | 12916916 | SY0573AQ | Taqman | | | |
| SY0573AQ | 12916916 | SY0573AQ | Taqman | SY0573AF1 | AGTCAACTGCCCAACTTAACCTA | 32 |
| SY0573AQ | 12916916 | SY0573AQ | Taqman | SY0573AR1 | TGCAGTTCTATTCTGGCTATCTTGT | 33 |
| SY0573AQ | 12916916 | SY0573AQ | Taqman | SY0573AA1FM | ACCACTTGTCTGGCC | 280 |
| SY0573AQ | 12916916 | SY0573AQ | Taqman | SY0573AA2TT | CACCACTTGTTTGGC | 281 |
| IGGY741 | 12917727 | IGGY741 | Illumina Golden Gate | | | |
| IGGY741 | 12917727 | IGGY741 | Illumina Golden Gate | IGGY741F3 | AAGGTTCTTTCAAGAAAAGGAA | 34 |
| IGGY741 | 12917727 | IGGY741 | Illumina Golden Gate | IGGY741F1 | ACTTCGTCAGTAACGGACTTTGTGCTTTGATCCTCTGCAGATA | 35 |
| IGGY741 | 12917727 | IGGY741 | Illumina Golden Gate | IGGY741F2 | GAGTCGAGGTCATATCGTTTTGTGCTTTGATCCTCTGCAGATT | 36 |
| SY0089B | 12917729 | SY0089B | Taqman | | | |
| SY0089B | 12917729 | SY0089B | Taqman | SY0089BF1 | TCGAAGCACTTTCCTTTGTATTTCCT | 37 |
| SY0089B | 12917729 | SY0089B | Taqman | SY0089BR1 | CACTTAGGTCACCAACAAGTCGA | 38 |
| SY0089B | 12917729 | SY0089B | Taqman | SY0089BA1FM | CTTCCAATATATAAAAAAAA | 282 |
| SY0089B | 12917729 | SY0089B | Taqman | SY0089BA2VC | TTCCAATATATCAAAAAAA | 283 |
| SY0098BQ | 12933267 | SY0098BQ | Taqman | | | |
| SY0098BQ | 12933267 | SY0098BQ | Taqman | SY0098BF1 | AGTCGATGCAAGAAGAAAGTCTCAAA | 39 |
| SY0098BQ | 12933267 | SY0098BQ | Taqman | SY0098BR1 | CTTTTACTTTCATGTCAGCATGTCTTGT | 40 |
| SY0098BQ | 12933267 | SY0098BQ | Taqman | SY0098BA1FM | CCCTTGCCCTTTAC | 284 |
| SY0098BQ | 12933267 | SY0098BQ | Taqman | SY0098BA2TT | TTACCCTTGCTCTTTAC | 285 |
| SY0567AQ | 12933268 | SY0567AQ | Taqman | | | |
| SY0567AQ | 12933268 | SY0567AQ | Taqman | SY0567AF1 | GTCGATGCAAGAAGAAAGTCTCAA | 41 |
| SY0567AQ | 12933268 | SY0567AQ | Taqman | SY0567AR1 | GTCAGCATGTCTTGTAAAGAAAGGA | 42 |
| SY0567AQ | 12933268 | SY0567AQ | Taqman | SY0567AA1FM | CAAGGGTAACGATTTTCAG | 286 |
| SY0567AQ | 12933268 | SY0567AQ | Taqman | SY0567AA2TT | CAAGGGTAACGATTTTCTG | 287 |
| IGGY2850 | 12940106 | IGGY2850 | Illumina Golden Gate | | | |
| IGGY2850 | 12940106 | IGGY2850 | Illumina Golden Gate | IGGY2850F3 | ATTTGGGTTTTAGAGAACATAAGG | 43 |
| IGGY2850 | 12940106 | IGGY2850 | Illumina Golden Gate | IGGY2850F1 | ACTTCGTCAGTAACGGACGGGGGTTGTTGCATTTGTGCTTAGG | 44 |
| IGGY2850 | 12940106 | IGGY2850 | Illumina Golden Gate | IGGY2850F2 | GAGTCGAGGTCATATCGTGGGGGTTGTTGCATTTGTGCTTAGC | 45 |
| SY4432 | 12940376 | SY4432 | Taqman | | | |
| SY4432 | 12940376 | SY4432 | Taqman | SY4432F1 | TGTGAAGACCCTGACATGTTTC | 46 |
| SY4432 | 12940376 | SY4432 | Taqman | SY4432R1 | GCAACTCTTGCAGATTCAGACAATG | 47 |
| SY4432 | 12940376 | SY4432 | Taqman | SY4432A1FM | AAGACACCGAGCAACATC | 288 |
| SY4432 | 12940376 | SY4432 | Taqman | SY4432A2TT | ACACCGGGCAACATC | 289 |
| SY4336 | 12940400 | SY4336 | Taqman | | | |
| SY4336 | 12940400 | SY4336 | Taqman | SY4336F1 | AGTGGTTCAATTTGAGGTGTCATC | 48 |
| SY4336 | 12940400 | SY4336 | Taqman | SY4336R1 | GGTGAAGAACATCTCTAGAAAACACTTA | 49 |
| SY4336 | 12940400 | SY4336 | Taqman | SY433641FM | CGCCACCATCGTAA | 290 |
| SY4336 | 12940400 | SY4336 | Taqman | SY4336A2TT | ACGCCACCATCTTAA | 291 |
| SY4435 | 12940422 | SY4435 | Taqman | | | |
| SY4435 | 12940422 | SY4435 | Taqman | SY4435F1 | AAGATTCCCGACGAGAGCGT | 50 |
| SY4435 | 12940422 | SY4435 | Taqman | SY4435R1 | CAGTGGTGGCCTCAATGGA | 51 |
| SY4435 | 12940422 | SY4435 | Taqman | SY4435A1FM | ACGCGCCGTAATACG | 292 |
| SY4435 | 12940422 | SY4435 | Taqman | SY4435A2TT | AACGCGCTGTAATACG | 293 |
| SY4439 | 12940448 | SY4439 | Taqman | | | |
| SY4439 | 12940448 | SY4439 | Taqman | SY4439F1 | TGGGTCCACCCGCTTC | 52 |
| SY4439 | 12940448 | SY4439 | Taqman | SY4439R1 | CAAGATCAAGTCAACGGTCAACGA | 53 |
| SY4439 | 12940448 | SY4439 | Taqman | SY4439A1FM | AATCGGCGAAGACAGTGAAC | 294 |
| SY4439 | 12940448 | SY4439 | Taqman | SY4439A2TT | ATCGGCGAAGACGGTGAA | 295 |
| IGGY3104 | 12940605 | IGGY3104 | Illumina Golden Gate | | | |
| IGGY3104 | 12940605 | IGGY3104 | Illumina Golden Gate | IGGY3104F3 | TAGCAGATCCGGTATAATTAACT | 54 |
| IGGY3104 | 12940605 | IGGY3104 | Illumina Golden Gate | IGGY3104F1 | ACTTCGTCAGTAACGGACTCATCATCATCAGAAGTCTCTCTAGTG | 55 |
| IGGY3104 | 12940605 | IGGY3104 | Illumina Golden Gate | IGGY3104F2 | GAGTCGAGGTCATATCGTTCATCATCATCAGAAGTCTCTCTAGTC | 56 |
| SY4418 | 12940610 | SY4418 | Taqman | | | |
| SY4418 | 12940610 | SY4418 | Taqman | SY4418F1 | GGCATTCCCGCTCCATTAGTAG | 57 |
| SY4418 | 12940610 | SY4418 | Taqman | SY4418R1 | CAACAGCTGCAGGAACCAAA | 58 |
| SY4418 | 12940610 | SY4418 | Taqman | SY4418A1FM | CCGGAAGCACTTGTACAG | 296 |
| SY4418 | 12940610 | SY4418 | Taqman | SY4418A2TT | CCGGAAGCACTTTTACAG | 297 |
| IGGY2851 | 12940655 | IGGY2851 | Illumina Golden Gate | | | |
| IGGY2851 | 12940655 | IGGY2851 | Illumina Golden Gate | IGGY2851F3 | TTCAACGGACATGTCATTTT | 59 |
| IGGY2851 | 12940655 | IGGY2851 | Illumina Golden Gate | IGGY2851F1 | ACTTCGTCAGTAACGGACGCAGCTCCTTCGCTTTCTGCTTGT | 60 |
| IGGY2351 | 12940655 | IGGY2851 | Illumina Golden Gate | IGGY2851F2 | GAGTCGAGGTCATATCGTGCAGCTCCTTCGCTTTCTGCTTGC | 61 |
| SY4440 | 12940854 | SY4440 | Taqman | | | |
| SY4440 | 12940854 | SY4440 | Taqman | SY4440F1 | CGTGCATTGAGCAAGAGTATACAGA | 62 |
| SY4440 | 12940854 | SY4440 | Taqman | SY4440R1 | CCATAGTTAAGCTGGCCCAAGAG | 63 |
| SY4440 | 12940854 | SY4440 | Taqman | SY4440A1FM | TCAATTTTAATGATTTCGTGAC | 298 |
| SY4440 | 12940854 | SY4440 | Taqman | SY4440A2TT | TCAATTTTAATGATTTGGTGACA | 299 |
| SY0127AQ | 12948965 | SY0127AQ | Taqman | | | |
| SY0127AQ | 12948965 | SY0127AQ | Taqman | SY0127AF1 | GCAGAATTTCCTTGGAGGTCAAAC | 64 |
| SY0127AQ | 12948965 | SY0127AQ | Taqman | SY0127AR1 | CCCCTCTTTCCAATATTTAATACAAGATTCAGT | 65 |
| SY0127AQ | 12948965 | SY0127AQ | Taqman | SY0127AA1FM | CGGTAAGAGTAATAATACA | 300 |
| SY0127AQ | 12948965 | SY0127AQ | Taqman | SY0127AA2TT | CGGTAAGAGTAACAATACA | 301 |
| SY1044BQ | 12973395 | SY1044BQ | Taqman | | | |
| SY1044BQ | 12973395 | SY1044BQ | Taqman | SY1044BF1 | CAAGAAAACTAAATGAATCACTGT | 66 |
| SY1044BQ | 12973395 | SY1044BQ | Taqman | SY1044BR1 | AGCCACAAGCAAATTCCTC | 67 |
| SY1044BQ | 12973395 | SY1044BQ | Taqman | SY1044BA1FM | CCTTTCTTCACCATG | 302 |
| SY1044BQ | 12973395 | SY1044BQ | Taqman | SY1044BA2TT | AGTTCCTTTCTTCATCA | 303 |
| SY0571AQ | 12976368 | SY0571AQ | Taqman | | | |
| SY0571AQ | 12976368 | SY0571AQ | Taqman | SY0571AF1 | GCTAAGCGGATAGAAGACTTGAC | 68 |
| SY0571AQ | 12976368 | SY0571AQ | Taqman | SY0571AR1 | GCACCAGCCAGAAGACAGTT | 69 |
| SY0571AQ | 12976368 | SY0571AQ | Taqman | SY0571AA1FM | ATTGCTTCATGCCGT | 304 |
| SY0571AQ | 12976368 | SY0571AQ | Taqman | SY0571AA2TT | ATTGCTTCATGCTGTG | 305 |
| SY0096C | 12976596 | SY0096C | Taqman | | | |
| SY0096C | 12976596 | SY0096C | Taqman | SY0096CF1 | AAAACAAACCACATGAGATGTATAGACAGT | 70 |
| SY0096C | 12976596 | SY0096C | Taqman | SY0096CR1 | TTTTGGATTGTGATGCTTTAATAATTGTGGAT | 71 |
| SY0096C | 12976596 | SY0096C | Taqman | SY0096CA1FM | CAGTGGGTAGAGTGAAA | 306 |
| SY0096C | 12976596 | SY0096C | Taqman | SY0096CA2VC | AGTGGGTAGAGAGAAA | 307 |
| SY0096A | 12976600 | SY0096A | Taqman | | | |
| SY0096A | 12976600 | SY0096A | Taqman | SY0096AF1 | AGACAAAACCACCAGCACCAA | 72 |
| SY0096A | 12976600 | SY0096A | Taqman | SY0096AR1 | AGGCACAAGGTAGAAGAGGAGATT | 73 |
| SY0096A | 12976600 | SY0096A | Taqman | SY0096AA1FM | CAGTAGCTGCTGCCGC | 308 |
| SY0096A | 12976600 | SY0096A | Taqman | SY0096AA2VC | CAGTAGCTGCCGCCGC | 309 |
| SY0569AQ | 12976666 | SY0569AQ | Taqman | | | |
| SY0569AQ | 12976666 | SY0569AQ | Taqman | SY0569AF1 | TCGGGAAGATGCTGGACA | 74 |
| SY0569AQ | 12976666 | SY0569AQ | Taqman | SY0569AR1 | TCGAAATACCAAGGCCAAGATG | 75 |
| SY0569AQ | 12976666 | SY0569AQ | Taqman | SY0569AA1FM | AGGACTTTGGAACCATG | 310 |
| SY0569AQ | 12976666 | SY0569AQ | Taqman | SY0569AA2TT | ACTTTGGAACCGTGTC | 311 |
| IGGY476 | 12977396 | IGGY476 | Illumina Golden Gate | | | |
| IGGY476 | 12977396 | IGGY476 | Illumina Golden Gate | IGGY476F3 | TCTTCCATGGTTCACTAGAATG | 76 |
| IGGY476 | 12977396 | IGGY476 | Illumina Golden Gate | IGGY476F1 | ACTTCGTCAGTAACGGACACACTCGCGGAACGTTGAACAG | 77 |
| IGGY476 | 12977396 | IGGY476 | Illumina Golden Gate | IGGY476F2 | GAGTCGAGGTCATATCGTACACTCGCGGAACGTTGAACAC | 78 |
| IGGY515 | 12977667 | IGGY515 | Illumina Golden Gate | | | |
| IGGY515 | 12977667 | IGGY515 | Illumina Golden Gate | IGGY515F3 | AACAGAAGACAAATACCTAGTGTG | 79 |
| IGGY515 | 12977667 | IGGY515 | Illumina Golden Gate | IGGY515F1 | ACTTCGTCAGTAACGGACGGAAAAGGGAACCAAGATAAAGTATATTCCA | 80 |
| IGGY515 | 12977667 | IGGY515 | Illumina Golden Gate | IGGY515F2 | GAGTCGAGGTCATATCGTGGAAAAGGGAACCAAGATAAAGTATATTCCG | 81 |
| SY3108 | 12979617 | SY3108 | Taqman | | | |
| SY3108 | 12979617 | SY3108 | Taqman | SY3108F1 | GTAACCCCAACTGACAAACACA | 82 |
| SY3108 | 12979617 | SY3108 | Taqman | SY3108R1 | TGCTCAAGATGGTGCATGCTT | 83 |
| SY3108 | 12979617 | SY3108 | Taqman | SY3108A1FM | ACTACCATCACAATAAGC | 312 |
| SY3108 | 12979617 | SY3108 | Taqman | SY3108A2TT | ACCATCACAATATGCAT | 313 |
| SY3112 | 12979655 | SY3112 | Taqman | | | |
| SY3112 | 12979655 | SY3112 | Taqman | SY3112F1 | CAAATTTTATGATACGTGCAGTTAAGC | 84 |
| SY3112 | 12979655 | SY3112 | Taqman | SY3112R1 | TCCCTCCCACATATACATCATCACTT | 85 |
| SY3112 | 12979655 | SY3112 | Taqman | SY3112A1FM | TTGGCGCACTTGGT | 314 |
| SY3112 | 12979655 | SY3112 | Taqman | SY3112A2TT | TGGCGCACTTTGTC | 315 |
| SY3110 | 12979670 | SY3110 | Taqman | | | |
| SY3110 | 12979670 | SY3110 | Taqman | SY3110F1 | ACCCCTGATGGTAATCTTTGAAAA | 86 |
| SY3110 | 12979670 | SY3110 | Taqman | SY3110R1 | GAACCGTGAGTATTTGGGACAGA | 87 |
| SY3110 | 12979670 | SY3110 | Taqman | SY3110A1FM | TAAAGATGTGTATTTTAACTT | 316 |
| SY3110 | 12979670 | SY3110 | Taqman | SY3110A2TT | AAAGATGTGTATTTTGACT | 317 |
| SY3114 | 12979695 | SY3114 | Taqman | | | |
| SY3114 | 12979695 | SY3114 | Taqman | SY3114F1 | GGGATCGGGCCTCCAAA | 88 |
| SY3114 | 12979695 | SY3114 | Taqman | SY3114R1 | TGCCAGGCTAAACAAATTGAATAC | 89 |
| SY3114 | 12979695 | SY3114 | Taqman | SY3114A1FM | TTTCCCTCAAGATAGAG | 318 |
| SY3114 | 12979695 | SY3114 | Taqman | SY3114A2TT | TTTCCCTCAAGATTGA | 319 |
| IGGY2357 | 12980624 | IGGY2357 | Illumina Golden Gate | | | |
| IGGY2357 | 12980624 | IGGY2357 | Illumina Golden Gate | IGGY2357F3 | TGAACTTCAACATTTGGTTTTT | 90 |
| IGGY2357 | 12980624 | IGGY2357 | Illumina Golden Gate | IGGY2357F1 | ACTTCGTCAGTAACGGACCGATTGAAAAGTGAAAATTCGTCATG | 91 |
| IGGY2357 | 12980624 | IGGY2357 | Illumina Golden Gate | IGGY2357F2 | GAGTCGAGGTCATATCGTCGATTGAAAAGTGAAAATTCGTCATC | 92 |
| SY3121 | 12980630 | SY3121 | Taqman | | | |
| SY3121 | 12980630 | SY3121 | Taqman | SY3121F1 | TCACCAAAGACAACAGCCTTG | 93 |
| SY3121 | 12980630 | SY3121 | Taqman | SY3121R1 | CGTCTTCGCTAGGACCTGAA | 94 |
| SY3121 | 12980630 | SY3121 | Taqman | SY3121A1FM | TCAACCAACAACCATG | 320 |
| SY3121 | 12980630 | SY3121 | Taqman | SY3121A2TT | TCAACCAACCACCAT | 321 |
| SY3148 | 12980667 | SY3148 | Taqman | | | |
| SY3148 | 12980667 | SY3148 | Taqman | SY3148F1 | GCGTGCGCTACAAGTCAGG | 95 |
| SY3148 | 12980667 | SY3148 | Taqman | SY3148R1 | GGTGAGGCAGCAGAAACAG | 96 |
| SY3148 | 12980667 | SY3148 | Taqman | SY3148A1FM | CAGCGAGTCCAACATT | 322 |
| SY3148 | 12980667 | SY3148 | Taqman | SY3148A2TT | AGCGAGTCCAACGTTT | 323 |
| SY3005 | 12980686 | SY3005 | Taqman | | | |
| SY3005 | 12980686 | SY3005 | Taqman | SY3005F1 | CAGGAAAGGGACCCTTGGAAA | 97 |
| SY3005 | 12980686 | SY3005 | Taqman | SY3005R1 | GTGGCATAAGCCCAAGCATT | 98 |
| SY3005 | 12980686 | SY3005 | Taqman | SY3005A1FM | CTGACCCAGTAAACAAC | 324 |
| SY3005 | 12980686 | SY3005 | Taqman | SY3005A2TT | CTGACCCAGTCAACA | 325 |
| SY4235 | 12981079 | SY4235 | Taqman | | | |
| SY4235 | 12981079 | SY4235 | Taqman | SY4235F1 | AGCCTCTGGTGGTAAGGATAAAG | 93 |
| SY4235 | 12981079 | SY4235 | Taqman | SY4235R1 | TGAAATCCATGCATCCGCAAA | 100 |
| SY4235 | 12981079 | SY4235 | Taqman | SY4235A1FM | CATTGAGATTGCACTTCGT | 326 |
| SY4235 | 12981079 | SY4235 | Taqman | SY4235A2TT | CCATTGAGATTGTACTTCGT | 327 |
| SY4433 | 12981180 | SY4433 | Taqman | | | |
| SY4433 | 12981180 | SY4433 | Taqman | SY4433F1 | TCAGAGCACTCCATATTGCTTCAG | 101 |
| SY4433 | 12981180 | SY4433 | Taqman | SY4433R1 | ATCCCCTGTACGAGGAAGTTTTG | 102 |
| SY4433 | 12981180 | SY4433 | Taqman | SY4433A1FM | TCGTGTGATTTTCATCATC | 328 |
| SY4433 | 12981180 | SY4433 | Taqman | SY4433A2TT | CGTGTGATTTTGATCATCA | 329 |
| SY4434 | 12981186 | SY4434 | Taqman | | | |
| SY4434 | 12981186 | SY4434 | Taqman | SY4434F1 | CGCCGTCTTCATCGTCGTTT | 103 |
| SY4434 | 12981186 | SY4434 | Taqman | SY4434R1 | TTTGCGGAGAACCCAATTTCC | 104 |
| SY4434 | 12981186 | SY4434 | Taqman | SY4434A1FM | ACCCGAAACTCGCACG | 330 |
| SY4434 | 12981186 | SY4434 | Taqman | SY4434A2TT | CGAAACTTGCACGCAC | 331 |
| IGGY3105 | 12981327 | IGGY3105 | Illumina Golden Gate | | | |
| IGGY3105 | 12981327 | IGGY3105 | Illumina Golden Gate | IGGY3105F3 | AAACTAATAGTAATGTAGCTCCTTTG | 105 |
| IGGY3105 | 12981327 | IGGY3105 | Illumina Golden Gate | IGGY3105F1 | ACTTCGTCAGTAACGGACGTCACGATTTCTTCTTCCCAAGTAAAA | 106 |
| IGGY3105 | 12981327 | IGGY3105 | Illumina Golden Gate | IGGY3105F2 | GAGTCGAGGTCATATCGTGTCACGATTTCTTCTTCCCAAGTAAAG | 107 |
| SY3889 | 12981395 | SY3889 | Taqman | | | |
| SY3889 | 12981395 | SY3889 | Taqman | SY3889F1 | GCAAGGCAACAATCTGAATGGT | 108 |
| SY3889 | 12981395 | SY3889 | Taqman | SY3889R1 | TCCACGGCATGCTTGGTATC | 109 |
| SY3889 | 12981395 | SY3889 | Taqman | SY3889A1FM | CCAACATCCATCGAA | 332 |
| SY3889 | 12981395 | SY3889 | Taqman | SY3889A2TT | TACCAACATCCATTGAA | 333 |
| IGGY3103 | 12981397 | IGGY3103 | Illumina Golden Gate | | | |
| IGGY3103 | 12981397 | IGGY3103 | Illumina Golden Gate | IGGY3103F3 | CTTCTCCCCTGAAAGGTATGT | 110 |
| IGGY3103 | 12981397 | IGGY3103 | Illumina Golden Gate | IGGY3103F1 | ACTTCGTCAGTAACGGACGGGTGATGGAGATGATAGCCCATTTT | 111 |
| IGGY3103 | 12981397 | IGGY3103 | Illumina Golden Gate | IGGY3103F2 | GAGTCGAGGTCATATCGTGGGTGATGGAGATGATAGCCCATTTC | 112 |
| IGGY3106 | 12981417 | IGGY3106 | Illumina Golden Gate | | | |
| IGGY3106 | 12931417 | IGGY3106 | Illumina Golden Gate | IGGY3106F3 | CTAACACAAGCTTTACCATTCTT | 113 |
| IGGY3106 | 12981417 | IGGY3106 | Illumina Golden Gate | IGGY3106F1 | ACTTCGTCAGTAACGGACGTGCAGATAGCATACATCATATACAAATGA | 114 |
| IGGY3106 | 12981417 | IGGY3106 | Illumina Golden Gate | IGGY3106F2 | GAGTCGAGGTCATATCGTGTGCAGATAGCATACATCATATACAAATGG | 115 |
| SY0871AQ | 12981920 | SY0871AQ | Taqman | | | |
| SY0871AQ | 12981920 | SY0871AQ | Taqman | SY0871AF1 | GAGGTCCATTGCTTCCTCTGCT | 116 |
| SY0871AQ | 12981920 | SY0871AQ | Taqman | SY0871AR1 | TGGTGGAGATCCACGTTCTAAAG | 117 |
| SY0871AQ | 12981920 | SY0871AQ | Taqman | SY0871AA1FM | CTCGTCAATTTCATCAA | 334 |
| SY0871AQ | 12981920 | SY0871AQ | Taqman | SY0871AA2TT | CTCGTCAATTTCTTCAA | 335 |
| SY0099E | 23543290 | SY0099E | Taqman | | | |
| SY0099E | 23543290 | SY0099E | Taqman | SY0099EF1 | TGTGATGCCGAAGCTAGACATG | 118 |
| SY0099E | 23543290 | SY0099E | Taqman | SY0099ER1 | CTCAACAAGTTCTGTCACCAAAGTT | 119 |
| SY0099E | 23543290 | SY0099E | Taqman | SY0099EA1FM | CAGCAACGAGGTAAG | 336 |
| SY0099E | 23543290 | SY0099E | Taqman | SY0099EA2TT | CAGCAACAAGGTAAG | 337 |
| SY4353 | 56017303 | SY4353 | Taqman | | | |
| SY4353 | 56017303 | SY4353 | Taqman | SY4353F1 | CCTCAGGCCTCATGATTGTCTT | 120 |
| SY4353 | 56017303 | SY4353 | Taqman | SY4353R1 | CACCATTAAATTTTACCCGGCTGT | 121 |
| SY4353 | 56017303 | SY4353 | Taqman | SY4353A1FM | CATGATGTACTAACGCAGTA | 338 |
| SY4353 | 56017303 | SY4353 | Taqman | SY4353A2TT | TCATGATGTACTAATGCAGTA | 339 |
| SY4354 | 56017304 | SY4354 | Taqman | | | |
| SY4354 | 56017304 | SY4354 | Taqman | SY4354F1 | CCACATCCACCCAACATGAAG | 122 |
| SY4354 | 56017304 | SY4354 | Taqman | SY4354R1 | CCTGAAGACTAACTGGTTACGTGAA | 123 |
| SY4354 | 56017304 | SY4354 | Taqman | SY4354A1FM | CCTATAAATAAGGAACCAGGT | 340 |
| SY4354 | 56017304 | SY4354 | Taqman | SY4354A2TT | CCTATAAATAAGGAACCTGG | 341 |
| SY4329 | 56017305 | SY4329 | Taqman | | | |
| SY4329 | 56017305 | SY4329 | Taqman | SY4329F1 | AGTGCTACAACTACACCTACACC | 124 |
| SY4329 | 56017305 | SY4329 | Taqman | SY4329R1 | GGGCTTCTTCTGTCACTGGTT | 125 |
| SY4329 | 56017305 | SY4329 | Taqman | SY4329A1FM | TCATTACACAATAGCATTTTC | 342 |
| SY4329 | 56017305 | SY4329 | Taqman | SY4329A2TT | CATTACACAATAGCCTTTTC | 343 |
| SY4349 | 56017307 | SY4349 | Taqman | | | |
| SY4349 | 56017307 | SY4349 | Taqman | SY4349F1 | CTGTTAATACCCAGTACTATGCTACA | 126 |
| SY4349 | 56017307 | SY4349 | Taqman | SY4349R1 | CTCCCACTATTCCTTGCCATCTC | 127 |
| SY4349 | 56017307 | SY4349 | Taqman | SY4349A1FM | TGAGAATCAACATGTGAGT | 344 |
| SY4349 | 56017307 | SY4349 | Taqman | SY4349A2TT | AGAATCAACATGTGGGTAA | 345 |
| SY4358 | 56017308 | SY4358 | Taqman | | | |
| SY4358 | 56017308 | SY4358 | Taqman | SY4358F1 | CCATAGCTTAATGCCACGATGTTG | 128 |
| SY4358 | 56017308 | SY4358 | Taqman | SY4358R1 | ACCAGACCATCGGCCTTCA | 129 |
| SY4358 | 56017308 | SY4358 | Taqman | SY4358A1FM | AATCCTGTACGACGGTAA | 346 |
| SY4358 | 56017308 | SY4358 | Taqman | SY4358A2TT | TCCTGTACGACTGTAAG | 347 |
| SY4324 | 56017310 | SY4324 | Taqman | | | |
| SY4324 | 56017310 | SY4324 | Taqman | SY4324F1 | GTTGTGGCTCGGCTTTATGATG | 130 |
| SY4324 | 56017310 | SY4324 | Taqman | SY4324R1 | ACAAGGCACAAGTACACATGCTC | 131 |
| SY4324 | 56017310 | SY4324 | Taqman | SY4324A1FM | TTGAACATAAAAGGACAATGG | 348 |
| SY4324 | 56017310 | SY4324 | Taqman | SY4324A2TT | TTGAACATAAAAGGGCAATGG | 349 |
| SY4234 | 56017312 | SY4234 | Taqman | | | |
| SY4234 | 56017312 | SY4234 | Taqman | SY4234F1 | GGTCGTGCTTTCATATTGGTTCCT | 132 |
| SY4234 | 56017312 | SY4234 | Taqman | SY4234R1 | GCGAGTGTGCAAAGGGTTT | 133 |
| SY4234 | 56017312 | SY4234 | Taqman | SY4234A1FM | AGGCATTAGTTGTGCTTCTT | 350 |
| SY4234 | 56017312 | SY4234 | Taqman | SY4234A2TT | AGGCATTAGTTGTGCTTTTT | 351 |
| SY4231 | 56017315 | SY4231 | Taqman | | | |
| SY4231 | 56017315 | SY4231 | Taqman | SY4231F1 | GTAGCAGCCAACAATGCTTTC | 134 |
| SY4231 | 56017315 | SY4231 | Taqman | SY4231R1 | TGGCCCCTGCATGTATACTTTC | 135 |
| SY4231 | 56017315 | SY4231 | Taqman | SY4231A1FM | TCTGCAACAATCAACATTT | 352 |
| SY4231 | 56017315 | SY4231 | Taqman | SY4231A2TT | ATCTGCAACAATCAGCATTT | 353 |
| SY4224 | 56017318 | SY4224 | Taqman | | | |
| SY4224 | 56017318 | SY4224 | Taqman | SY4224F1 | GGCTCCATGAGACGAAATAAAGC | 136 |
| SY4224 | 56017318 | SY4224 | Taqman | SY4224R1 | GAGGGCACAAGATTGGTATTGTTG | 137 |
| SY4224 | 56017318 | SY4224 | Taqman | SY4224A1FM | TGGGAAGTCTACTCTGAT | 354 |
| SY4224 | 56017313 | SY4224 | Taqman | SY4224A2TT | AGTGGGAAGTCTACTCTTAT | 355 |
| SY4335 | 56017319 | SY4335 | Taqman | | | |
| SY4335 | 56017319 | SY4335 | Taqman | SY4335F1 | CAAGCTGGTCTTGTACAGTTGAG | 138 |
| SY4335 | 56017319 | SY4335 | Taqman | SY4335R1 | ACCAACTACTCGTTAAGCAAGGA | 139 |
| SY4335 | 56017319 | SY4335 | Taqman | SY4335A1FM | AAGCTTCTGGCCAAAG | 356 |
| SY4335 | 56017319 | SY4335 | Taqman | SY4335A2TT | TCTGGCCATAGCTA | 357 |
| SY4213 | 56017321 | SY4213 | Taqman | | | |
| SY4213 | 56017321 | SY4213 | Taqman | SY4213F1 | GCTACCAAGTTGCAGAACATTATGA | 140 |
| SY4213 | 56017321 | SY4213 | Taqman | SY4213R1 | TCCGAGAAAGGGACTGAAAATGAG | 141 |
| SY4213 | 56017321 | SY4213 | Taqman | SY4213A1FM | AGACTATAAAAATGACCAAATT | 358 |
| SY4213 | 56017321 | SY4213 | Taqman | SY4213A2TT | AGACTATAAAAATGACCAATTT | 359 |
| SY4227 | 56017322 | SY4227 | Taqman | | | |
| SY4227 | 56017322 | SY4227 | Taqman | SY4227F1 | GCAGAAAACAGATTATCAGGGCTTA | 142 |
| SY4227 | 56017322 | SY4227 | Taqman | SY4227R1 | GAGTTGAATGTCACTTAGGTAGCAA | 143 |
| SY4227 | 56017322 | SY4227 | Taqman | SY4227A1FM | CTTGGATTGGGAGCAAATTAC | 360 |
| SY4227 | 56017322 | SY4227 | Taqman | SY4227A2TT | TGGATTGGGAGCGAATTAC | 361 |
| SY4220 | 56017329 | SY4220 | Taqman | | | |
| SY4220 | 56017329 | SY4220 | Taqman | SY4220F1 | TGAGCCCCATTCAGTTGAGAA | 144 |
| SY4220 | 56017329 | SY4220 | Taqman | SY4220R1 | GCTGTTTTGGGCACATGATGA | 145 |
| SY4220 | 56017329 | SY4220 | Taqman | SY4220A1FM | TTGTAGACTGCGTTTTCTG | 362 |
| SY4220 | 56017329 | SY4220 | Taqman | SY4220A2TT | TACTTGTAGACTGTGTTTTC | 363 |
| SY4343 | 56021709 | SY4343 | Taqman | | | |
| SY4343 | 56021709 | SY4343 | Taqman | SY4343F1 | GGGAAGCTAATCCGAGAACTGA | 146 |
| SY4343 | 56021709 | SY4343 | Taqman | SY4343R1 | TGACACCAGATGAGAAACAGGAG | 147 |
| SY4343 | 56021709 | SY4343 | Taqman | SY4343A1FM | TGCATAACAAAAACCATGATTAAA | 364 |
| SY4343 | 56021709 | SY4343 | Taqman | SY4343A2TT | TGCATAACAAAAACCATGTTTAAA | 365 |
| SY4316 | 56021714 | SY4316 | Taqman | | | |
| SY4316 | 56021714 | SY4316 | Taqman | SY4316F1 | GAAAAGGGAGGAGTGATCTGATAC | 148 |
| SY4316 | 56021714 | SY4316 | Taqman | SY4316R1 | ACCCAGCCTAAGAAATATAATGAAGATAC | 149 |
| SY4316 | 56021714 | SY4316 | Taqman | SY4316A1FM | CCAGTAAAATAATGCTCAA | 366 |
| SY4316 | 56021714 | SY4316 | Taqman | SY4316A2TT | TGCCAGTAAAATAATGTTC | 367 |
| SY4225 | 56021721 | SY4225 | Taqman | | | |
| SY4225 | 56021721 | SY4225 | Taqman | SY4225F1 | TTTGGGTGTTCTGTGATGGA | 150 |
| SY4225 | 56021721 | SY4225 | Taqman | SY4225R1 | GGCATATCATTAGGGAAGTCCGA | 151 |
| SY4225 | 56021721 | SY4225 | Taqman | SY4225A1FM | CACAAACTTGCCAACTA | 368 |
| SY4225 | 56021721 | SY4225 | Taqman | SY4225A2TT | CACAAACTTGCGAACTATT | 369 |
| SY4219 | 56021724 | SY4219 | Taqman | | | |
| SY4219 | 56021724 | SY4219 | Taqman | SY4219F1 | ACGCTTGACTGAAGATGATACAAC | 152 |
| SY4219 | 56021724 | SY4219 | Taqman | SY4219R1 | AAGTTAATGCAGAACCGTGTGTTTT | 153 |
| SY4219 | 56021724 | SY4219 | Taqman | SY4219A1FM | TCATTTAACCGCTCATTTA | 370 |
| SY4219 | 56021724 | SY4219 | Taqman | SY4219A2TT | TGGATCATTTAACCGTTCATTT | 371 |
| SY4326 | 56021730 | SY4326 | Taqman | | | |
| SY4326 | 56021730 | SY4326 | Taqman | SY4326F1 | GCTTGACAGCTTTGGATGTTCTTC | 154 |
| SY4326 | 56021730 | SY4326 | Taqman | SY4326R1 | GTCACTCGCACAACACTATACTAC | 155 |
| SY4326 | 56021730 | SY4326 | Taqman | SY4326A1FM | CTGAGACAGAGATATCAGATT | 372 |
| SY4326 | 56021730 | SY4326 | Taqman | SY4326A2TT | TGAGACAGAGATATCAGGTT | 373 |
| SY4232 | 56021731 | SY4232 | Taqman | | | |
| SY4232 | 56021731 | SY4232 | Taqman | SY4232F1 | CCTTGATATCGAGCATTTCCTTCCT | 156 |
| SY4232 | 56021731 | SY4232 | Taqman | SY4232R1 | TTCGGAGAAGGTTTTGATTTGTTC | 157 |
| SY4232 | 56021731 | SY4232 | Taqman | SY4232A1FM | TCCAGTCCTAACAATTCAA | 374 |
| SY4232 | 56021731 | SY4232 | Taqman | SY4232A2TT | AGTCCAGTCCTAATAATTCAA | 375 |
| SY4330 | 56021742 | SY4330 | Taqman | | | |
| SY4330 | 56021742 | SY4330 | Taqman | SY4330F1 | TGAAGAGAGATCAGATTAAAGAGAGTGA | 158 |
| SY4330 | 56021742 | SY4330 | Taqman | SY4330R1 | TGTAGAGTCTCCTGGCCAAA | 159 |
| SY4330 | 56021742 | SY4330 | Taqman | SY4330A1FM | CACAATGATTTTTCCTG | 376 |
| SY4330 | 56021742 | SY4330 | Taqman | SY4330A2TT | ACAATGATTTTTGTTC | 377 |
| SY4325 | 56021749 | SY4325 | Taqman | | | |
| SY4325 | 56021749 | SY4325 | Taqman | SY4325F1 | CCTCTCCCTCATATTCCATTGCTT | 160 |
| SY4325 | 56021749 | SY4325 | Taqman | SY4325R1 | TGGCACTACCCACATGAAC | 161 |
| SY4325 | 56021749 | SY4325 | Taqman | SY4325A1FM | CATGTGTGCAACGGAAA | 378 |
| SY4325 | 56021749 | SY4325 | Taqman | SY4325A2TT | CATGTGTGCATCGGAAA | 379 |
| SY4217 | 56021750 | SY4217 | Taqman | | | |
| SY4217 | 56021750 | SY4217 | Taqman | SY4217F1 | TGCAAACAATGTAGCCCAATCAC | 162 |
| SY4217 | 56021750 | SY4217 | Taqman | SY4217R1 | TGCATGAATGACTTTTCTATTGGAGA | 163 |
| SY4217 | 56021750 | SY4217 | Taqman | SY4217A1FM | TTGGAGATACTCCTAGG | 380 |
| SY4217 | 56021750 | SY4217 | Taqman | SY4217A2TT | TTGGAGATACTCTTAGGA | 381 |
| SY4215 | 56021751 | SY4215 | Taqman | | | |
| SY4215 | 56021751 | SY4215 | Taqman | SY4215F1 | GTCCAACAGGTAAGTTAAACAACTATGA | 164 |
| SY4215 | 56021751 | SY4215 | Taqman | SY4215R1 | AAACGAACAATCTTGGACAAGCA | 165 |
| SY4215 | 56021751 | SY4215 | Taqman | SY4215A1FM | CCAAATAACACGAGTACT | 382 |
| SY4215 | 56021751 | SY4215 | Taqman | SY4215A2TT | TAACACGAGTTCTCGT | 383 |
| SY4322 | 56021755 | SY4322 | Taqman | | | |
| SY4322 | 56021755 | SY4322 | Taqman | SY4322F1 | ACAAAGAGTACACGTAATATCACACG | 166 |
| SY4322 | 56021755 | SY4322 | Taqman | SY4322R1 | GTCTCGGATATTTTCTGTTAGTCCAA | 167 |
| SY4322 | 56021755 | SY4322 | Taqman | SY4322A1FM | CATGCATCATGACC | 384 |
| SY4322 | 56021755 | SY4322 | Taqman | SY4322A2TT | CATGCATCATGGCC | 385 |
| SY4344 | 56021756 | SY4344 | Taqman | | | |
| SY4344 | 56021756 | SY4344 | Taqman | SY4344F1 | AAGGGTGATGGAGACAGATAGGA | 168 |
| SY4344 | 56021756 | SY4344 | Taqman | SY4344R1 | CGTGTACTCTTTGTCTGATTGGAA | 169 |
| SY4344 | 56021756 | SY4344 | Taqman | SY4344A1FM | CGTATTGCCCCTTTC | 386 |
| SY4344 | 56021756 | SY4344 | Taqman | SY4344A2TT | ATGTCGTATTGCCTCTTTC | 387 |
| SY4360 | 266863987 | SY4360 | Taqman | | | |
| SY4360 | 266863987 | SY4360 | Taqman | SY4360F1 | GGGAGGTTATGTTGCCTTGCT | 170 |
| SY4360 | 266863987 | SY4360 | Taqman | SY4360R1 | GCCAAGGACCAAAGGACTTAC | 171 |
| SY4360 | 266863987 | SY4360 | Taqman | SY4360A1FM | TTTGTTTAATTTCAGCTATATC | 388 |
| SY4360 | 266863987 | SY4360 | Taqman | SY4360A2TT | AATTTGTTTAATTTCTGCTATATC | 389 |
| SY4208 | 266863989 | SY4208 | Taqman | | | |
| SY4208 | 266363989 | SY4208 | Taqman | SY4208F1 | GTGCATAACATGTGCTTCTATAGGTT | 172 |
| SY4208 | 266863989 | SY4208 | Taqman | SY4208R1 | AGCACAAAGGATTCCACAACA | 173 |
| SY4208 | 266863989 | SY4208 | Taqman | SY4208A1FM | CTTTGCATCATTTATCCC | 390 |
| SY4208 | 266863989 | SY4208 | Taqman | SY4208A2TT | CCTTTGCATCATTTGTC | 391 |
| SY4210 | 266863990 | SY4210 | Taqman | | | |
| SY4210 | 266863990 | SY4210 | Taqman | SY4210F1 | GCCAAGGACCAAAGGACTTAC | 174 |
| SY4210 | 266863990 | SY4210 | Taqman | SY4210R1 | GGGAGGTTATGTTGCCTTGCTAT | 175 |
| SY4210 | 266863990 | SY4210 | Taqman | SY4210A1FM | CTATATCAAGTGCCTT | 392 |
| SY4210 | 266863990 | SY4210 | Taqman | SY4210A2TT | CTATATCAAGTGCTTTT | 393 |
| SY4207 | 266363993 | SY4207 | Taqman | | | |
| SY4207 | 266863993 | SY4207 | Taqman | SY4207F1 | CCAAGGAAGGGACAAATGATACAAAG | 176 |
| SY4207 | 266863993 | SY4207 | Taqman | SY4207R1 | TGCAGTCCATGCCATATTCAAAC | 177 |
| SY4207 | 266863993 | SY4207 | Taqman | SY4207A1FM | CCTATTGAAGCACATGT | 394 |
| SY4207 | 266863993 | SY4207 | Taqman | SY4207A2TT | ACCTATTGAAGCACTTGT | 395 |
| SY4278 | 270585230 | SY4278 | Taqman | | | |
| SY4278 | 270585230 | SY4278 | Taqman | SY4278F1 | AACCGGCCTCTCCTAAAGG | 178 |
| SY4278 | 270585230 | SY4278 | Taqman | SY4278R1 | TTGATGAAATATAAGTCGCTTGTTGATAG | 179 |
| SY4278 | 270585230 | SY4278 | Taqman | SY4278A1FM | TATTCAATCACTCATTG | 396 |
| SY4278 | 270585230 | SY4278 | Taqman | SY4278A2TT | TTATTCAATCACTTATTGT | 397 |
| SY4255 | 270585250 | SY4255 | Taqman | | | |
| SY4255 | 270585250 | SY4255 | Taqman | SY4255F1 | ATCTTTGAGATGCAACGTATTTGTA | 180 |
| SY4255 | 270585250 | SY4255 | Taqman | SY4255R1 | CAACGACCTAAATGATGTGCTATATCC | 181 |
| SY4255 | 270585250 | SY4255 | Taqman | SY4255A1FM | TTTTACGTATGCTAGC | 398 |
| SY4255 | 270585250 | SY4255 | Taqman | SY4255A2TT | TTTACGTTTGCTAGC | 399 |
| SY4300 | 270585251 | SY4300 | Taqman | | | |
| SY4300 | 270585251 | SY4300 | Taqman | SY4300F1 | GCAAGTATTCCTTGTACCCTTCATC | 182 |
| SY4300 | 270585251 | SY4300 | Taqman | SY4300R1 | TTGGTCTGAAAGTGTAAATATAGTCACG | 183 |
| SY4300 | 270585251 | SY4300 | Taqman | SY4300A1FM | TTGCTCTTAGCCAATA | 400 |
| SY4300 | 270585251 | SY4300 | Taqman | SY4300A2TT | TGCTCTTAGCCGATA | 401 |
| SY4301 | 270585256 | SY4301 | Taqman | | | |
| SY4301 | 270585256 | SY4301 | Taqman | SY4301F1 | GCTGCAATTCTCTTCCACCATT | 184 |
| SY4301 | 270585256 | SY4301 | Taqman | SY4301R1 | CGTGGTGTCATCTTGCGTAA | 185 |
| SY4301 | 270585256 | SY4301 | Taqman | SY4301A1FM | TCTATGAAAAGCTACGAACTT | 402 |
| SY4301 | 270585256 | SY4301 | Taqman | SY4301A2TT | CTCTATGAAAAGCTATGAACTTT | 403 |
| SY4244 | 270585257 | SY4244 | Taqman | | | |
| SY4244 | 270585257 | SY4244 | Taqman | SY4244F1 | AACAGAAGCCATTTGAAGATTTACCA | 186 |
| SY4244 | 270585257 | SY4244 | Taqman | SY4244R1 | GTGCATGATCTTCCTGCCAA | 187 |
| SY4244 | 270585257 | SY4244 | Taqman | SY4244A1FM | CATGATTAAAAGACGGTCTA | 404 |
| SY4244 | 270585257 | SY4244 | Taqman | SY4244A2TT | TTCATGATTAAAAGACTGTC | 405 |
| SY4295 | 270585267 | SY4295 | Taqman | | | |
| SY4295 | 270585267 | SY4295 | Taqman | SY4295F1 | GAAGGAATTCTCTCATCATGTGTTTAC | 188 |
| SY4295 | 270585267 | SY4295 | Taqman | SY4295R1 | TGAGCCAGTAGCATAACCTGAA | 189 |
| SY4295 | 270585267 | SY4295 | Taqman | SY4295A1FM | TGTTTTAACCAAGTAATACG | 406 |
| SY4295 | 270585267 | SY4295 | Taqman | SY4295A2TT | TTGTTTTAACCAAGTAGTAC | 407 |
| SY4254 | 270585272 | SY4254 | Taqman | | | |
| SY4254 | 270585272 | SY4254 | Taqman | SY4254F1 | GGCGAAAAGTGACCCTCTCT | 190 |
| SY4254 | 270585272 | SY4254 | Taqman | SY4254R1 | ACCAAGTTAAGTTGCCTCTTATGAC | 191 |
| SY4254 | 270585272 | SY4254 | Taqman | SY4254A1FM | TCATGTACACTCTTTGAGTA | 408 |
| SY4254 | 270585272 | SY4254 | Taqman | SY4254A2TT | TTCATGTACACTCTTGAGTAT | 409 |
| SY4302 | 270775294 | SY4302 | Taqman | | | |
| SY4302 | 270775294 | SY4302 | Taqman | SY4302F1 | GCGATGCGAGTGCTAAGTG | 192 |
| SY4302 | 270775294 | SY4302 | Taqman | SY4302R1 | GTGCATGTTGAACAAAGGTCTCTT | 193 |
| SY4302 | 270775294 | SY4302 | Taqman | SY4302A1FM | TAATTATTAACTCTTTCCTTTTG | 410 |
| SY4302 | 270775294 | SY4302 | Taqman | SY4302A2TT | ATTATTAACTCTTTCTTTTTGTCT | 411 |
| SY4253 | 270775313 | SY4253 | Taqman | | | |
| SY4253 | 270775313 | SY4253 | Taqman | SY4253F1 | CCCTAATCATCAAACCCAGCAAA | 194 |
| SY4253 | 270775313 | SY4253 | Taqman | SY4253R1 | AGCACATCATTTAGGTCGTTGAAAG | 195 |
| SY4253 | 270775313 | SY4253 | Taqman | SY4253A1FM | TCATCTATATAAACTTCGACTAA | 412 |
| SY4253 | 270775313 | SY4253 | Taqman | SY4253A2TT | CTCATCTATATAAACTTGGACTA | 413 |
| SY4247 | 270775314 | SY4247 | Taqman | | | |
| SY4247 | 270775314 | SY4247 | Taqman | SY4247F1 | TGCTGGGTTTGATGATTAGGGTAA | 196 |
| SY4247 | 270775314 | SY4247 | Taqman | SY4247R1 | GGAAGAAGATGAAGGGTACAAGGA | 197 |
| SY4247 | 270775314 | SY4247 | Taqman | SY4247A1FM | TGCCCCTAACACAAC | 414 |
| SY4247 | 270775314 | SY4247 | Taqman | SY4247A2TT | TGCCCCTTACACAAC | 415 |
| SY4257 | 270775316 | SY4257 | Taqman | | | |
| SY4257 | 270775316 | SY4257 | Taqman | SY4257F1 | GGTAGGTTCTAGCCCGATAGGA | 198 |
| SY4257 | 270775316 | SY4257 | Taqman | SY4257R1 | CGTGACTATATTTACACTTTCAGACCA | 199 |
| SY4257 | 270775316 | SY4257 | Taqman | SY4257A1FM | ATGGTGTTTTATCTAAGTTT | 416 |
| SY4257 | 270775316 | SY4257 | Taqman | SY4257A2TT | AAATGGTGTTTTATCTTTTAT | 417 |
| SY4281 | 270775330 | SY4281 | Taqman | | | |
| SY4281 | 270775330 | SY4281 | Taqman | SY4281F1 | AAACCACCCATGAAAGCCAGAA | 200 |
| SY4281 | 270775330 | SY4281 | Taqman | SY4281R1 | AAGAATAGCGAGTAAAGTGTGTGC | 201 |
| SY4281 | 270775330 | SY4281 | Taqman | SY4281A1FM | TGATAAGTGTCTCTGTTGTT | 418 |
| SY4281 | 270775330 | SY4281 | Taqman | SY4281A2TT | TGATAAGTGTCTGTGTTGTT | 419 |
| SY4284 | 270964571 | SY4284 | Taqman | | | |
| SY4284 | 270964571 | SY4284 | Taqman | SY4284F1 | AAGAGCCAAACTACCTGCGAAA | 202 |
| SY4284 | 270964571 | SY4284 | Taqman | SY4284R1 | ACGAGAACTGACAGGGTCTGAT | 203 |
| SY4284 | 270964571 | SY4284 | Taqman | SY4284A1FM | TTCTGAGTAGATTTATTATCA | 420 |
| SY4284 | 270964571 | SY4284 | Taqman | SY4284A2TT | TTCTGAGTAGATTTATTGTC | 421 |
| SY4261 | 270964573 | SY4261 | Taqman | | | |
| SY4261 | 270964573 | SY4261 | Taqman | SY4261F1 | AGGTGTTTGCTTCGTTGTGAAA | 204 |
| SY4261 | 270964573 | SY4261 | Taqman | SY4261R1 | CCAAAGTGCACCACCTTCCTT | 205 |
| SY4261 | 270964573 | SY4261 | Taqman | SY4261A1FM | CTTTCGATGAATGCTATGA | 422 |
| SY4261 | 270964573 | SY4261 | Taqman | SY4261A2TT | CTTTCGATGAATGTTATGATA | 423 |
| SY4305 | 270964584 | SY4305 | Taqman | | | |
| SY4305 | 270964584 | SY4305 | Taqman | SY4305F1 | TTGGTCTGAAAGTGTAAATATAGTCACG | 206 |
| SY4305 | 270964584 | SY4305 | Taqman | SY4305R1 | GCAAGTATTCCTTGTACCCTTCATC | 207 |
| SY4305 | 270964584 | SY4305 | Taqman | SY4305A1FM | CTCCTAAACGTAACTGT | 424 |
| SY4305 | 270964584 | SY4305 | Taqman | SY4305A2TT | CTCCTAAACGTAGCTG | 425 |
| SY4276 | 270964586 | SY4276 | Taqman | | | |
| SY4276 | 270964586 | SY4276 | Taqman | SY4276F1 | CTCTTATGTTTAATCGATGTGGTCTCAATC | 208 |
| SY4276 | 270964586 | SY4276 | Taqman | SY4276R1 | AGTGGCCCTTATGCACTATTTTC | 209 |
| SY4276 | 270964586 | SY4276 | Taqman | SY4276A1FM | CCAACTACATCATCATGT | 426 |
| SY4276 | 270964586 | SY4276 | Taqman | SY4276A2TT | TTCCAACTACATCATGTG | 427 |
| SY4299 | 270964590 | SY4299 | Taqman | | | |
| SY4299 | 270964590 | SY4299 | Taqman | SY4299F1 | AGTGGTATGAAGTGGAAGTGTCTTG | 210 |
| SY4299 | 270964590 | SY4299 | Taqman | SY4299R1 | GGCCCGTGGTGTCATCTTG | 211 |
| SY4299 | 270964590 | SY4299 | Taqman | SY4299A1FM | TCAGCTCTGAGGATGC | 428 |
| SY4299 | 270964590 | SY4299 | Taqman | SY4299A2TT | AACAATAGTCTTCAGAGGATGC | 429 |
| SY4291 | 270964591 | SY4291 | Taqman | | | |
| SY4291 | 270964591 | SY4291 | Taqman | SY4291F1 | ACTCAGCAGCATTCTGTTAGAAGGA | 212 |
| SY4291 | 270964591 | SY4291 | Taqman | SY4291R1 | ACTCTTACCTGATAGAGGACTGAAG | 213 |
| SY4291 | 270964591 | SY4291 | Taqman | SY4291A1FM | ATTAGCATATTTCTCTCCATT | 430 |
| SY4291 | 270964591 | SY4291 | Taqman | SY4291A2TT | TATTAGCATATTTGTCTCCAT | 431 |
| SY4303 | 270964599 | SY4303 | Taqman | | | |
| SY4303 | 270964599 | SY4303 | Taqman | SY4303F1 | GGCTCCACTGCCATTACCATT | 214 |
| SY4303 | 270964599 | SY4303 | Taqman | SY4303R1 | TGAGCCAGTAGCATAACCTGAA | 215 |
| SY4303 | 270964599 | SY4303 | Taqman | SY4303A1FM | TCTCTATGGCCGTA | 432 |
| SY4303 | 270964599 | SY4303 | Taqman | SY4303A2TT | CTCTCTATGGTCGTA | 433 |
| SY4273 | 270964601 | SY4273 | Taqman | | | |
| SY4273 | 270964601 | SY4273 | Taqman | SY4273F1 | TTTCTTTTGGGACGAAGGGTTT | 216 |
| SY4273 | 270964601 | SY4273 | Taqman | SY4273R1 | AGAGTAGTGACAGAGTTGAGCAA | 217 |
| SY4273 | 270964601 | SY4273 | Taqman | SY4273A1FM | CTACAATAATACAAAATCTCAATA | 434 |
| SY4273 | 270964601 | SY4273 | Taqman | SY4273A2TT | CTACAATAATACAAAATTTCAATA | 435 |
| SY4256 | 270964605 | SY4256 | Taqman | | | |
| SY4256 | 270964605 | SY4256 | Taqman | SY4256F1 | TGCAACAGAGCTGAAAACTTGTC | 218 |
| SY4256 | 270964605 | SY4256 | Taqman | SY4256R1 | ACACAGTTGCCGCTTATGAC | 219 |
| SY4256 | 270964605 | SY4256 | Taqman | SY4256A1FM | CCCTCTATTTATATATGTGCA | 436 |
| SY4256 | 270964605 | SY4256 | Taqman | SY4256A2TT | CCTCTATTTATATTTGTGCAC | 437 |
| SY4289 | 271154434 | SY4289 | Taqman | | | |
| SY4289 | 271154434 | SY4289 | Taqman | SY4289F1 | AGTGCATAAGGGCCACTAATTTC | 220 |
| SY4289 | 271154434 | SY4289 | Taqman | SY4289R1 | CTATTTTGTTTGTTTGCACCTACCA | 221 |
| SY4289 | 271154434 | SY4289 | Taqman | SY4289A1FM | TGGCACATAGCAATTTTTAA | 438 |
| SY4289 | 271154434 | SY4289 | Taqman | SY4289A2TT | TATGGCACATAGCAATTTAAA | 439 |
| SY4285 | 271154435 | SY4285 | Taqman | | | |
| SY4285 | 271154435 | SY4285 | Taqman | SY4285F1 | AAATCCAAATCTCTTGTTATTCAAACACTA | 222 |
| SY4285 | 271154435 | SY4285 | Taqman | SY4285R1 | CACCTACCAAGTATGGCACATAGC | 223 |
| SY4285 | 271154435 | SY4285 | Taqman | SY4285A1FM | TCAAAGATGTACTCAAGCT | 440 |
| SY4285 | 271154435 | SY4285 | Taqman | SY4285A2TT | CAAAGATGTACTCGAGCT | 441 |
| SY4306 | 271154438 | SY4306 | Taqman | | | |
| SY4306 | 271154438 | SY4306 | Taqman | SY4306F1 | GCCACTCACACATATACTTGCACTT | 224 |
| SY4306 | 271154438 | SY4306 | Taqman | SY4306R1 | TGATGGAAGCAAGACGGAGAGAT | 225 |
| SY4306 | 271154438 | SY4306 | Taqman | SY4306A1FM | ACCATGTTGCAATTGATA | 442 |
| SY4306 | 271154438 | SY4306 | Taqman | SY4306A2TT | CCATGTTGCAATTGGTA | 443 |
| SY4282 | 271154440 | SY4282 | Taqman | | | |
| SY4282 | 271154440 | SY4282 | Taqman | SY4282F1 | GGGACTTAATGGAGCCCTATTCTC | 226 |
| SY4282 | 271154440 | SY4282 | Taqman | SY4282R1 | TGGCAGGAAGATCATGCACTTA | 227 |
| SY4282 | 271154440 | SY4282 | Taqman | SY4282A1FM | TCATGCTAGTGAAACAGCT | 444 |
| SY4282 | 271154440 | SY4282 | Taqman | SY4282A2TT | CATGCTAGTGGAACAGCT | 445 |
| SY4268 | 271154450 | SY4268 | Taqman | | | |
| SY4268 | 271154450 | SY4268 | Taqman | SY4268F1 | AACAACTACGTTTTCTCTTCACTTCAG | 228 |
| SY4268 | 271154450 | SY4268 | Taqman | SY4268R1 | CCTGGGTGGAAACCTCTCAA | 229 |
| SY4268 | 271154450 | SY4268 | Taqman | SY4268A1FM | AAATGGTCGACTTCAAAA | 446 |
| SY4268 | 271154450 | SY4268 | Taqman | SY4268A2TT | TGGTCGACTTCAGAA | 447 |
| SY4269 | 271154453 | SY4269 | Taqman | | | |
| SY4269 | 271154453 | SY4269 | Taqman | SY4269F1 | TGTAACGTATTCGGTTTTATAGGGTGA | 230 |
| SY4269 | 271154453 | SY4269 | Taqman | SY4269R1 | AACAAACATACGAATTTAACGCGACAT | 231 |
| SY4269 | 271154453 | SY4269 | Taqman | SY4269A1FM | AATCATTTTTGTAATAGT | 448 |
| SY4269 | 271154453 | SY4269 | Taqman | SY4269A2TT | AATCATTTTTGTGATAG | 449 |
| SY4272 | 271154454 | SY4272 | Taqman | | | |
| SY4272 | 271154454 | SY4272 | Taqman | SY4272F1 | AGAGGGAATTGCAACAGAGCTGA | 232 |
| SY4272 | 271154454 | SY4272 | Taqman | SY4272R1 | TGCCGCTTATGACTTATCCTTTC | 233 |
| SY4272 | 271154454 | SY4272 | Taqman | SY4272A1FM | TTGTCAAGACACTAATACTT | 450 |
| SY4272 | 271154454 | SY4272 | Taqman | SY4272A2TT | CAAGACACTACTACTTGGT | 451 |
| SY4250 | 271344625 | SY4250 | Taqman | | | |
| SY4250 | 271344625 | SY4250 | Taqman | SY4250F1 | ATTGGTGTTGGCATGGTTCAC | 234 |
| SY4250 | 271344625 | SY4250 | Taqman | SY4250R1 | TAAACAAAGACGCCTCCTGCTA | 235 |
| SY4250 | 271344625 | SY4250 | Taqman | SY4250A1FM | AACAGTGTTTTCTCTTAACAAT | 452 |
| SY4250 | 271344625 | SY4250 | Taqman | SY4250A2TT | ACAGTGTTTTCTCTTGACAATT | 453 |
| SY4307 | 271344641 | SY4307 | Taqman | | | |
| SY4307 | 271344641 | SY4307 | Taqman | SY4307F1 | CCCATAATACAACCCAGAAATGGAA | 236 |
| SY4307 | 271344641 | SY4307 | Taqman | SY4307R1 | GTTTACGTACTATGCATGACCACA | 237 |
| SY4307 | 271344641 | SY4307 | Taqman | SY4307A1FM | ATATGGGATTTCACCGTTATC | 454 |
| SY4307 | 271344641 | SY4307 | Taqman | SY4307A2TT | AATATGGGATTTCATCGTTATC | 455 |
| SY4265 | 271344646 | SY4265 | Taqman | | | |
| SY4265 | 271344646 | SY4265 | Taqman | SY4265F1 | TTCGAAGTTCAGTTGGCACAA | 238 |
| SY4265 | 271344646 | SY4265 | Taqman | SY4265R1 | AGGGCCAAGCTTAGACAAGGTAA | 239 |
| SY4265 | 271344646 | SY4265 | Taqman | SY4265A1FM | AACACAACCGCTTGTAC | 456 |
| SY4265 | 271344646 | SY4265 | Taqman | SY4265A2TT | AACACAACCTCTTGTACA | 457 |
| SY4297 | 271534944 | SY4297 | Taqman | | | |
| SY4297 | 271534944 | SY4297 | Taqman | SY4297F1 | ATGCAGCAGTATGCAATCCAA | 240 |
| SY4297 | 271534944 | SY4297 | Taqman | SY4297R1 | TTCACTTTACATTTCTACTCCAACAATA | 241 |
| SY4297 | 271534944 | SY4297 | Taqman | SY4297A1FM | TTGTTATACTTGGCGTT | 458 |
| SY4297 | 271534944 | SY4297 | Taqman | SY4297A2TT | TTATACTTGGTGTTGGC | 459 |
| SY4279 | 271534965 | SY4279 | Taqman | | | |
| SY4279 | 271534965 | SY4279 | Taqman | SY4279F1 | GGAGAGAAATGACTCACATAGCATAGG | 242 |
| SY4279 | 271534965 | SY4279 | Taqman | SY4279R1 | TGAGACCACATCGATTAAACATAAGAGA | 243 |
| SY4279 | 271534965 | SY4279 | Taqman | SY4279A1FM | AAGGAAATGGAGTATTGATAAA | 460 |
| SY4279 | 271534965 | SY4279 | Taqman | SY4279A2TT | AGGAAATGGAGTATTGATGAA | 461 |
| SY4251 | 271534967 | SY4251 | Taqman | | | |
| SY4251 | 271534967 | SY4251 | Taqman | SY4251F1 | AAGGCCCGTGGTGTCATCTTG | 244 |
| SY4251 | 271534967 | SY4251 | Taqman | SY4251R1 | TGGTATGAAGTGGAAGTGTCTTGA | 245 |
| SY4251 | 271534967 | SY4251 | Taqman | SY4251A1FM | CACCATTTCCCAAACAA | 462 |
| SY4251 | 271534967 | SY4251 | Taqman | SY4251A2TT | TCCACCATTTTCCAAAC | 463 |
| SY4249 | 271534977 | SY4249 | Taqman | | | |
| SY4249 | 271534977 | SY4249 | Taqman | SY4249F1 | ACTGGCTCAACGTGACTCTTA | 246 |
| SY4249 | 271534977 | SY4249 | Taqman | SY4249R1 | TGAACAAAATGTTAGATGGAATGACA | 247 |
| SY4249 | 271534977 | SY4249 | Taqman | SY4249A1FM | TAGAGATACTACTACTACATA | 464 |
| SY4249 | 271534977 | SY4249 | Taqman | SY4249A2TT | AGAGATACTACTACTACTTAA | 465 |
| SY4310 | 271724460 | SY4310 | Taqman | | | |
| SY4310 | 271724460 | SY4310 | Taqman | SY4310F1 | GGGCATGTGCTCTTAATTTCTGA | 248 |
| SY4310 | 271724460 | SY4310 | Taqman | SY4310R1 | GTGAACCATGCCAACACCAA | 249 |
| SY4310 | 271724460 | SY4310 | Taqman | SY4310A1FM | CAAGCATCTAACTGCAA | 466 |
| SY4310 | 271724460 | SY4310 | Taqman | SY4310A2TT | CACAAGCATCTAACTGTAA | 467 |
| SY4292 | 271724476 | SY4292 | Taqman | | | |
| SY4292 | 271724476 | SY4292 | Taqman | SY4292F1 | GGAAGCAAGACGGAGAGATAAGATTG | 250 |
| SY4292 | 271724476 | SY4292 | Taqman | SY4292R1 | GCCACTCACACATATACTTGCACTT | 251 |
| SY4292 | 271724476 | SY4292 | Taqman | SY4292A1FM | CATGGTATCATGTAGTAGT | 468 |
| SY4292 | 271724476 | SY4292 | Taqman | SY4292A2TT | CATGGTATCATGTGGTA | 469 |
| SY4290 | 271914417 | SY4290 | Taqman | | | |
| SY4290 | 271914417 | SY4290 | Taqman | SY4290F1 | CCAGACGCAACTTGTGCAAT | 252 |
| SY4290 | 271914417 | SY4290 | Taqman | SY4290R1 | GTTGGCATGGTTCACCTAACAG | 253 |
| SY4290 | 271914417 | SY4290 | Taqman | SY4290A1FM | CAGCAAACACACCATAAAC | 470 |
| SY4290 | 271914417 | SY4290 | Taqman | SY4290A2TT | AGCAAACACACCGTAAACA | 471 |
| SY4252 | 271914434 | SY4252 | Taqman | | | |
| SY4252 | 271914434 | SY4252 | Taqman | SY4252F1 | ATGTGGTCATGCATAGTACGTAAAC | 254 |
| SY4252 | 271914434 | SY4252 | Taqman | SY4252R1 | CAATGCAAGGACTGCAAGGT | 255 |
| SY4252 | 271914434 | SY4252 | Taqman | SY4252A1FM | TAAGGTAAGACTACGATGT | 472 |
| SY4252 | 271914434 | SY4252 | Taqman | SY4252A2TT | TTATAAGGTAAGACTATGATG | 473 |
| SY4246 | 271914435 | SY4246 | Taqman | | | |
| SY4246 | 271914435 | SY4246 | Taqman | SY4246F1 | GGAGAGAAATGACTCACATAGCATAGG | 256 |
| SY4246 | 271914435 | SY4246 | Taqman | SY4246R1 | TGAGACCACATCGATTAAACATAAGAGA | 257 |
| SY4246 | 271914435 | SY4246 | Taqman | SY4246A1FM | CCTTAATTACTACACCAA | 474 |
| SY4246 | 271914435 | SY4246 | Taqman | SY4246A2TT | TTCCTTAATTACTATACCA | 475 |
| SY4314 | 271914437 | SY4314 | Taqman | | | |
| SY4314 | 271914437 | SY4314 | Taqman | SY4314F1 | GCCACTCACACATATACTTGCACTT | 258 |
| SY4314 | 271914437 | SY4314 | Taqman | SY4314R1 | TGATGGAAGCAAGACGGAGAGAT | 259 |
| SY4314 | 271914437 | SY4314 | Taqman | SY4314A1FM | ACGTCACTTCAAACCA | 476 |
| SY4314 | 271914437 | SY4314 | Taqman | SY4314A2TT | ACGTCACTTCACACC | 477 |
| SY4264 | 271914440 | SY4264 | Taqman | | | |
| SY4264 | 271914440 | SY4264 | Taqman | SY4264F1 | GAGGAAAGTAAATTCTGCTGCCAAA | 260 |
| SY4264 | 271914440 | SY4264 | Taqman | SY4264R1 | GATTGTGATGGTCCTAGCTAAAAGT | 261 |
| SY4264 | 271914440 | SY4264 | Taqman | SY4264A1FM | TTGTTAGGAATGCTTTTGAAAT | 478 |
| SY4264 | 271914440 | SY4264 | Taqman | SY4264A2TT | TTGTTAGGAATGCTTTGAAATT | 479 |
| SY4416 | 272389082 | SY4416 | Taqman | | | |
| SY4416 | 272389082 | SY4416 | Taqman | SY4416F1 | GCAAAGGCTATTGAGCCAAAGAC | 262 |
| SY4416 | 272389082 | SY4416 | Taqman | SY4416R1 | GCCTAGAAGTGGAGGCTTGAAT | 263 |
| SY4416 | 272389082 | SY4416 | Taqman | SY4416A1FM | CTGTTGGAGTAAGAGCCAA | 480 |
| SY4416 | 272389082 | SY4416 | Taqman | SY4416A2TT | TGTTGGAGTAGGAGCCA | 481 |
| SY4426 | 353462473 | SY4426 | Taqman | | | |
| SY4426 | 353462473 | SY4426 | Taqman | SY4426F1 | ATCTTGGAGGCGGTGCTCT | 264 |
| SY4426 | 353462473 | SY4426 | Taqman | SY4426R1 | TGGAGGAAGCTATGAGAAGTGTTG | 265 |
| SY4426 | 353462473 | SY4426 | Taqman | SY4426A1FM | TGGAGATCATAGGCTGTC | 482 |
| SY4426 | 353462473 | SY4426 | Taqman | SY4426A2TT | ATGGAGATCATTGGCTGTCT | 483 |
| SY4427 | 412802301 | SY4427 | Taqman | | | |
| SY4427 | 412802301 | SY4427 | Taqman | SY4427F1 | GGCAGTTCTAAATGCAGCATCA | 266 |
| SY4427 | 412802301 | SY4427 | Taqman | SY4427R1 | GCAGCATGAGCCAAAGCAATG | 267 |
| SY4427 | 412802301 | SY4427 | Taqman | SY4427A1FM | AATTTCCATTCAAGTTTAAA | 484 |
| SY4427 | 412802301 | SY4427 | Taqman | SY4427A2TT | AATTTCCATTCAAGTTTTAA | 485 |
| SY4421 | 412802302 | SY4421 | Taqman | | | |
| SY4421 | 412802302 | SY4421 | Taqman | SY4421F1 | GCAGCTGGGAGAATGTTATTGTATG | 268 |
| SY4421 | 412802302 | SY4421 | Taqman | SY4421R1 | TTCTGCAATTCCTAGGTGTTCA | 269 |
| SY4421 | 412802302 | SY4421 | Taqman | SY4421A1FM | AAGTCCCATAAGTTAGCA | 486 |
| SY4421 | 412802302 | SY4421 | Taqman | SY4421A2TT | AAAGTCCCATAATTTAGCA | 487 |
| SY4437 | 412802304 | SY4437 | Taqman | | | |
| SY4437 | 412802304 | SY4437 | Taqman | SY4437F1 | ATTCATCAATGGCGGCTGCAAA | 270 |
| SY4437 | 412802304 | SY4437 | Taqman | SY4437R1 | AAGATTCTTCATTCTACGTGGCTCT | 271 |
| SY4437 | 412802304 | SY4437 | Taqman | SY4437A1FM | TCTTTCTGTTACACTATTT | 488 |
| SY4437 | 412802304 | SY4437 | Taqman | SY4437A2TT | TTCTTTCTGTTACATTATTT | 489 |
| SY4428 | 412802305 | SY4428 | Taqman | | | |
| SY4428 | 412802305 | SY4428 | Taqman | SY4428F1 | ATTGCTGCTGCACCGGTTGAT | 272 |
| SY4428 | 412802305 | SY4428 | Taqman | SY4428R1 | GTCATCCTCTGCTGCTAATCCA | 273 |
| SY4428 | 412302305 | SY4428 | Taqman | SY4428A1FM | CATCATCATTAATCCAATTGAATA | 490 |
| SY4428 | 412802305 | SY4428 | Taqman | SY4428A2TT | CATCATCATTAATCCGATTGAAT | 491 |
| SY4362 | 999991351 | SY4362 | Taqman | | | |
| SY4362 | 999991351 | SY4362 | Taqman | SY4362F1 | TCATTCTTTGTTGAAAATACTTGATT | 274 |
| SY4362 | 999991351 | SY4362 | Taqman | SY4362R1 | TTGATATTGATATGATGGGTTGAA | 275 |
| SY4362 | 999991351 | SY4362 | Taqman | SY4362A1FM | CAATCTTATTAAATAAGTGCA | 492 |
| SY4362 | 999991351 | SY4362 | Taqman | SY4362A2TT | GTATGACCTAGATAGGAACCT | 493 |
| SY0574AQ | 23543129 | SY0574AQ | Taqman | | | |
| SY0574AQ | 23543129 | SY0574AQ | Taqman | SY0574AF1 | GCGAGGAGGTCGTAGATGAGA | 276 |
| SY0574AQ | 23543129 | SY0574AQ | Taqman | SY0574AR1 | TGAAGGGTAGTTCCGACAAAGAAAC | 277 |
| SY0574AQ | 23543129 | SY0574AQ | Taqman | SY0574AA1FM | TGTCGTTTGACAAGGC | 494 |
| SY0574AQ | 23543129 | SY0574AQ | Taqman | SY0574AA2TT | TCGTTTGACGAGGCT | 495 |

TABLE 20
SNP target sequences

| Table 20. | Assay component | | Allele/Detected | | |
|---|---|---|---|---|---|
| Marker Name | name | DNA sequence | nucleotide | TOP target sequence | SEQ ID NO. |
| IGGY260 | | | | AACCCTCTAACTATACTTATTCTCTGACAACCGTTTATAAAGTTTATCTAAACAGGACTTTACTTAGTTCCA | 496 |
| | | | | CCATTCAAAAAAACATGATTTTGTAGCTTATGCATAAGTTCACTTCAACTTATGGAGGAACTTCTTTCATC | |
| | | | | TCTCTTCTTATTTTCTTCTCATATAAGTACTCAGGGAAAAGTTTATTCAAACAGACCCTAAACCTTGATTTT | |
| | | | | ACTCTCAAACATATTTTTGGAACACTCCCATCGAAAATCCAGACACACCCTTAATTTCCCAGCATTCAAAA | |
| | | | | CCCTCTTTTAGGGTTCCATTCACAGAGCAAACACGTTCCAAACAAAAGAAGACCAAAGATTTCGGCACTC | |
| | | | | AGAGSGGAAAAGYTTYGAACTTTGACACTCCCAAGGAGTCACTYAGAAGGGTTTGTTTCGTGGGGAGTT | |
| | | | | TTGGCGACGATGGAGAGGGCGTGGAGGCCGCTCTGGAGCTCGTCGGCGAGGAGGTCGTAGATGAGAC | |
| | | | | GGTGTCGTTTGAC[A/G]AGGCTCTGGCCCTCGAACTTGGGGGAGACGATGTTGAGGTTGAAGTGGGTT | |
| | | | | TCTTTGTCGGAACTACCCTTCACGGCGGCGTGGCCCGCGTGCTGGTACGACACGTCGTCCACCTCCAAAA | |
| | | | | CGGTGGCTTCCAGCGCCGTTTGCAGCTTCGACCGAATCCTGCTAGGCACGAGTAAATACATGAATATGT | |
| | | | | CTCTGAACTTTTTGAGCATTTTTAATRGTAATTAAGTCCTTAATCTTCAACAAATTTTTTAAACAAATTTCT | |
| | | | | CTAAGTTAGTTTACTACAGCTTGAAACTGCCATAAAAATAACAATATGTGGCWGTTTTACCAACTCCAAG | |
| | | | | ACCCAATTACAAAAATTGTAAGAGATCTAAGAACCCAATTACAATTTATTTTTAATTTTTTTAGAAACTTC | |
| | | | | ATTAAAAATTCCCAAATAATTCAATCACCTATTGATGTATTAACCCTCTAACTTATATTATTCTCTTACAAC | |
| | | | | CGTTTATAAAGTTTATCTAAACAGGACTTTACTTAGTTCCACCATTCAAAAAAACATGATTTTGTAGCTTA | |
| | | | | TGCATAAGTTCACTTCAACTTAT | |
| IGGY260 | IGGY260F3 | TCAAACG | | | 639 |
| | | ACACCGT | | | |
| | | CTCAT | | | |
| IGGY260 | IGGY260F1 | ACTTCGT | A | | 640 |
| | | CAGTAAC | | | |
| | | GGACGCA | | | |
| | | AGTTCGA | | | |
| | | GGGCCAG | | | |
| | | AGCCTT | | | |
| IGGY260 | IGGY260F2 | GAGTCGA | G | | 641 |
| | | GGTCATA | | | |
| | | TCGTGCA | | | |
| | | AGTTCGA | | | |
| | | GGGCCAG | | | |
| | | AGCCTC | | | |
| SY0574AQ | | | | AACCCTCTAACTATACTTATTCTCTGACAACCGTTTATAAAGTTTATCTAAACAGGACTTTACTTAGTTCCA | 497 |
| | | | | CCATTCAAAAAAACATGATTTTGTAGCTTATGCATAAGTTCACTTCAACTTATGGAGGAACTTCTTTCATC | |
| | | | | TCTCTTCTTATTTTCTTCTCATATAAGTACTCAGGGAAAAGTTTATTCAAACAGACCCTAAACCTTGATTTT | |
| | | | | ACTCTCAAACATATTTTTGGAACACTCCCATCGAAAATCCAGACACACCCTTAATTTCCCAGCATTCAAAA | |
| | | | | CCCTCTTTTAGGGTTCCATTCACAGAGCAAACACGTTCCAAACAAAAGAAGACCAAAGATTTCGGCACTC | |
| | | | | AGAGNGGAAAAGNTTNGAACTTTGACACTCCCAAGGAGTCACTNAGAAGGGTTTGTTTCGTGGGGAGT | |
| | | | | TTTGGCGACGATGGAGAGGGCGTGGAGGCCGCTCTGGAGCTCGTCGGCGAGGAGGTCGTAGATGAGA | |
| | | | | CGGTGTCGTTTGAC[A/G]AGGCTCTGGCCCTCGAACTTGGGGGAGACGATGTTGAGGTTGAAGTGGGT | |
| | | | | TTCTTTGTCGGAACTACCCTTCACGGCGGCGTGGCCCGCGTGCTGGTACGACACGTCGTCCACCTCCAAA | |
| | | | | ACGGTGGCTTCCAGCGCCGTTTGCAGCTTCGACCGAATCCTGCTAGGCACGAGTAAATACATGAATATG | |
| | | | | TCTCTGAACTTTTTGAGCATTTTTAATNGTAATTAAGTCCTTAATCTTCAACAAATTTTTTAAACAAATTTC | |
| | | | | TCTAAGTTAGTTTACTACAGCTTGAAACTGCCATAAAAATAACAATATGTGGCNGTTTTACCAACTCCAA | |
| | | | | GACCCAATTACAAAAATTGTAAGAGATCTAAGAACCCAATTACAATTTATTTTTAATTTTTTTAGAAACTT | |
| | | | | CATTAAAAATTCCCAAATAATTCAATCACCTATTGATGTATTAACCCTCTAACTTATATTATTCTCTTACAA | |
| | | | | CCGTTTATAAAGTTTATCTAAACAGGACTTTACTTAGTTCCACCATTCAAAAAAACATGATTTTGTAGCTT | |
| | | | | ATGCATAAGTTCACTTCAACTTAT | |
| SY0574AQ | SY0574AF1 | GCGAGGA | | | 642 |
| | | GGTCGTA | | | |
| | | GATGAGA | | | |
| SY0574AQ | SY0574AR1 | TGAAGGG | | | 643 |
| | | TAGTTCC | | | |
| | | GACAAAG | | | |
| | | AAAC | | | |
| SY0574AQ | SY0574AA1FM | TGTCGTTT | A | | 644 |
| | | GACAAGGC | | | |
| SY0574AQ | SY0574AA2TT | TCGTTTG | G | | 645 |
| | | ACGAGGCT | | | |
| SY0573AQ | | | | TTGCAGTTCTATTCTGGCTATCTTGTATAATTTGGGCCA[A/G]ACAAGTGGTGCCAAGCCGATAGGTTAA | 498 |
| | | | | GTTGGGCAGTTGACTAAAAGTCATAAAAACTGTAACATTTCAAAAATCCACAAAATTACCTCAACTAATT | |
| | | | | CTAGATCAAAAATANTCCACAATCTGTAATATTGCTAACAAGATTTTCAGCGCTCAAGTTCACTAGAATG | |
| | | | | CTATCATTTCCCGCAGAGAAAACAGTCTTTGTTTTTTTGGAGTTACCACCTGTTTTTAGGGGGTTTCACTT | |
| | | | | TATAAATACG | |
| SY0573AQ | SY0573AF1 | AGTCAAC | | | 646 |
| | | TGCCCAA | | | |
| | | CTTAACCTA | | | |
| SY0573AQ | SY0573AR1 | TGCAGTT | | | 647 |
| | | CTATTCTG | | | |
| | | GCTATCTT | | | |
| | | GT | | | |
| SY0573AQ | SY0573AA1FM | ACCACTT | G | | 648 |
| | | GTCTGGCC | | | |
| SY0573AQ | SY0573AA2TT | CACCACT | A | | 649 |
| | | TGTTTGGC | | | |
| IGGY741 | | | | GCAGCCCTTTCTACCATCAATTCATATTGAGAGCAAGTGCTGCTAAGGCTCTTGGATCATTCAGGAGCAA | 499 |
| | | | | CCCCACTATTTGAGTCAATTTCCTCTTCATGGGGGGTTAGTGGAAATGGAAAAAAAAAGATAATTGGAG | |
| | | | | GCAAAAAAAGTTGATATTGCAACAATAATAATAATAATATGGAACTGTTTGTGCTTTGATCCTCTGCAGA | |
| | | | | T[A/T]GAAGGTTCTTTCAAGAAAAGGAAGCCTTTGGTAAATAGTAAAGACCCTTTAGTACTTCGAAGCAC | |
| | | | | TTTCCTTTGTATTTCCTTGTTAGAATTGATGAGCTTTTTTTTKATATATTGGAAGTAAAATCTATTAGATAT | |
| | | | | CTTGTTTTAATATTTTGTGATATGTAATAAGTCGACTTGTTGGTGACCTAAGTGTG | |
| IGGY741 | IGGY741F3 | AAGGTTC | | | 650 |
| | | TTTCAAG | | | |
| | | AAAAGGAA | | | |
| IGGY741 | IGGY741F1 | ACTTCGT | A | | 651 |
| | | CAGTAAC | | | |
| | | GGACTTT | | | |
| | | GTGCTTT | | | |
| | | GATCCTC | | | |
| | | TGCAGATA | | | |
| IGGY741 | IGGY741F2 | GAGTCGA | T | | 652 |
| | | GGTCATA | | | |
| | | TCGTTTTG | | | |
| | | TGCTTTG | | | |
| | | ATCCTCT | | | |
| | | GCAGATT | | | |
| SY0089B | | | | CACACTTAGGTCACCAACAAGTCGACTTATTACATATCACAAAATATTAAAACAAGATATCTAATAGATTT | 500 |
| | | | | TACTTCCAATATAT[A/C]AAAAAAAAGCTCATCAATTCTAACAAGGAAATACAAAGGAAAGTGCTTCGAA | |
| | | | | GTACTAAAGGGTCTTTACTATTTACCAAAGGCTTCCTTTTCTTGAAAGAACCTTCNATCTGCAGAGGATC | |
| | | | | AAAGCACAAACAGTTCCATATTATTATTATTATTGTTGCAATATCAACTTTTTTTGCCTCCAATTATCTTTTT | |
| | | | | TTTTCCATTTCCACTAACCCCCCATGAAGAGGAAATTGACTCAAATAGTGGGGTTGCTCCTGAATGATCC | |
| | | | | AAGAGCCTTAGCAGCACTTGCTCTCAATATGAATTGATGGTAGAAAGGGCTGC | |
| SY0089B | SY0089BF1 | TCGAAGC | | | 653 |
| | | ACTTTCCT | | | |
| | | TTGTATTT | | | |
| | | CCT | | | |
| SY0089B | SY0089BR1 | CACTTAG | | | 654 |
| | | GTCACCA | | | |
| | | ACAAGTC | | | |
| | | GA | | | |
| SY0089B | SY0089BA1FM | CTTCCAAT | A | | 655 |
| | | ATATAAA | | | |
| | | AAAAA | | | |
| SY0089B | SY0089BA2VC | TTCCAAT | C | | 656 |
| | | ATATCAA | | | |
| | | AAAAA | | | |
| SY0098BQ | | | | GGAATTCCCAAATAGTCGATGCAAGAAGAAAGTCTCAAAAGTATGAATGTTGTAAAG[A/G]GCAAGGG | 501 |
| | | | | TAACGATTTTCNGNAATCCTTTCTTTACAAGACATGCTGACATGAAAGTAAAAGATTCAACATATGAATC | |
| | | | | GACTAACTATTTCCAACAAGGAATTAAGCCATTGTTGTATATTTGACATATAGATAGGAAAATGGCTATG | |
| | | | | GTCCTCCAAGTACTGCATTCTCAATGTCTTCTCTGCTTAAAGCATAACTAAACCTCCTCTCCATATTTTTTT | |
| | | | | CAAGATTCCCAGGTCCACTCTTCAAATAC | |
| SY0098BQ | SY0098BF1 | AGTCGAT | | | 657 |
| | | GCAAGAA | | | |
| | | GAAAGTC | | | |
| | | TCAAA | | | |
| SY0098BQ | SY0098BR1 | CTTTTACT | | | 658 |
| | | TTCATGTC | | | |
| | | AGCATGT | | | |
| | | CTTGT | | | |
| SY0098BQ | SY0098BA1FM | CCCTTGC | G | | 659 |
| | | CCTTTAC | | | |
| SY0098BQ | SY0098BA2TT | TTACCCTT | A | | 660 |
| | | GCTCTTTAC | | | |
| SY0567AQ | | | | GGAATTCCCAAATAGTCGATGCAAGAAGAAAGTCTCAAAAGTATGAATGTTGTAAAGNGCAAGGGTAA | 502 |
| | | | | CGATTTTC[A/T]GNAATCCTTTCTTTACAAGACATGCTGACATGAAAGTAAAAGATTCAACATATGAATC | |
| | | | | GACTAACTATTTCCAACAAGGAATTAAGCCATTGTTGTATATTTGACATATAGATAGGAAAATGGCTATG | |
| | | | | GTCCTCCAAGTACTGCATTCTCAATGTCTTCTCTGCTTAAAGCATAACTAAACCTCCTCTCCATATTTTTTT | |
| | | | | CAAGATTCCCAGGTCCACTCTTCAAATAC | |
| SY0567AQ | SY0567AF1 | GTCGATG | | | 661 |
| | | CAAGAAG | | | |
| | | AAAGTCT | | | |
| | | CAA | | | |
| SY0567AQ | SY0567AR1 | GTCAGCA | | | 662 |
| | | TGTCTTGT | | | |
| | | AAAGAAA | | | |
| | | GGA | | | |
| SY0567AQ | SY0567AA1FM | CAAGGGT | A | | 663 |
| | | AACGATT | | | |
| | | TTCAG | | | |
| SY0567AQ | SY0567AA2TT | CAAGGGT | T | | 664 |
| | | AACGATT | | | |
| | | TTCTG | | | |
| IGGY2850 | | | | ATGTAAAGCTGCCTTGANGGNCAATTCTCTGCTGCTAATTGCATAAGACATGGATGAAACACATAAAAT | 503 |
| | | | | AGTGGAAACAAATACTGTAAGAAGTGAAAACAACCACAACGACGATCTTGAAAGTTGTTTCAACTGAAG | |
| | | | | ACTTACACTTAAGCCAACTAAGAACCATCTGAATCCATGAAAAAGTTCTAGCACCCACCCATTAAGTGGA | |
| | | | | AAAAGAGTGTAGCTCTTCCTTATGTTCTCTAAAACCCAAATG[C/G]CTAAGCACAAATGCAACAACCCCA | |
| | | | | GAGAGCCATTGGTAACGGCAGAAATTAGCTGCAAGTTTGAATATCTTTCCACGCAAAACTGACCACGAA | |
| | | | | ATGGTCTGAACAAAGACTTCTGGATCAAAATGAATCCCAACATGACCAGTAGTAGCACATCAACGCAAA | |
| | | | | TGATCAAGAACTGGTTAATGCATGTAGAAGGATCTTTCAAAAACTTTAAATCATGGCAAAAAGTCTTTCC | |
| | | | | CCGGGTCCCAGGACAATC | |
| IGGY2850 | IGGY2850F3 | ATTTGGG | | | 665 |
| | | TTTTAGA | | | |
| | | GAACATA | | | |
| | | AGG | | | |
| IGGY2850 | IGGY2850F1 | ACTTCGT | C | | 666 |
| | | CAGTAAC | | | |
| | | GGACGG | | | |
| | | GGGTTGT | | | |
| | | TGCATTT | | | |
| | | GTGCTTA | | | |
| | | GG | | | |
| IGGY2850 | IGGY2850F2 | GAGTCGA | G | | 667 |
| | | GGTCATA | | | |
| | | TCGTGGG | | | |
| | | GGTTGTT | | | |
| | | GCATTTG | | | |
| | | TGCTTAGC | | | |
| SY4432 | | | | ACAGCAGCATTCAAGATAAGGTCTTCAAATATTCAAATATACATTTCAGTATACTAAAGGTTCTGCAGAG | 504 |
| | | | | AAATGGAAAATCCTTTNNGCTTTTATACCATACAGGTTAAGTCATGTTGCAATANACTAAAACCTCAATT | |
| | | | | CATTTCTGACTGTAACATTGGGAAGAAAGCCCAGCTGTTGGCTGATCTACCTTCCTTCCCAGCAACCTTCC | |
| | | | | TGTTAGTCCACCACTCATAGCCACTGCCAGTAGTAACAGAAACATCACCTTTCCTGTTGTCAAAATTGAAT | |
| | | | | TCTTTGGAAGAATTTACAGAATTTTGCTTGTGAAGGCATTGCTCTCCTTTTCCTACAGGATTGTCAGGCTT | |
| | | | | ATCGTCAGTTTTCTCTGTACANGCNTTANAGCTACTATTGGTGTCTATTTGCACCCTTTCTCTGTCAACAG | |
| | | | | GGTCTTTGGTCAGTATACCTTGTGAAGACCCTGACATGTTTCGAAGCAATACCCCAGTTTTATTTGCAAG | |
| | | | | ACACCG[A/G]GCAACATCTTCCCCGGTTAATTCAAATGAAACTCTGTGATCAACCAATGTTGCATTGCTT | |
| | | | | GGATGTCCATTGTCTGAATCTGCAAGAGTTGCTTCCTTAGAAATCTGGTTTTGCACAGAGATGTTGTTTT | |
| | | | | GATTGGTTGGCCCAGCACTGTCAGGTGTCAAACAGCCAGAACCTAACCTGGATTCCTGCCCCAGACCAT | |
| | | | | CAGGTGTCACACACCCAGAGCCTAATCGCGAAGCAAGCCCAACACCATCAGGAGTCAAGGATCCCGAAC | |
| | | | | CTAGTCTTGAACCTTGCCATGCACTGTCTGGCGTCAATGATCCAGATCCCAGTCTAGAACCCCATCTTCG | |
| | | | | GGTGGAGAAGTGTTCAACACCCAAGATCTTTGGTGTTTCCCCTTTGGGGAATTCAAGNGTAGGGGGTCT | |
| | | | | ATCAGGGAATGGGGTTGAGGTGCCAGAAGTTGAAAATGCTGATCCNGGTGATATGAGCTGGCCACCTG | |
| | | | | GGCTTCCAGGATATTGTTGATAAGG | |
| SY4432 | SY4432F1 | TGTGAAG | | | 668 |
| | | ACCCTGA | | | |
| | | CATGTTTC | | | |
| SY4432 | SY4432R1 | GCAACTC | | | 669 |
| | | TTGCAGA | | | |
| | | TTCAGAC | | | |
| | | AATG | | | |
| SY4432 | SY4432A1FM | AAGACAC | A | | 670 |
| | | CGAGCAA | | | |
| | | CATC | | | |
| SY4432 | SY4432A2TT | ACACCGG | G | | 671 |
| | | GCAACATC | | | |
| SY4336 | | | | AAGAAGGGATCATTGAGAAGCTTTGCTTGCGTAGCCCTTTCAAATTTACATGATTGATTTTTTCTGTTTCT | 505 |
| | | | | GCTCTAATTTTTGAAACCCACTTGGGAGAATGCGAGCTGAACTGAGTTTTGGGAATAGTATTATGACATG | |
| | | | | GTCATTTTGAAGAGATATTCTGTTGGGAGAATGCTGCCTCTTTCATTGTTTGGGCATCTATTATGCCCTAC | |
| | | | | CCATTATAGTATCATTCTTCCGTAGCAGGTCAAGATTTTTAATTAAAGTAAGGGAGAGGTCAATAATCTT | |
| | | | | TCAGTATGATAGGGTTGATTTTGTAAGTAACTGGAAAGTGCAATNGAAAAGGCAATTTGAGATGTTATG | |
| | | | | TGTTGCAAAAGAATGGAAATACAAGCAAAACAAAAAGAAAGTATGGATGGCTTTAGTGGTTCAATTTGA | |
| | | | | GGTGTCATCACAAGTTAAGAATTGGTTCACCAGANAGAGATTTGTAGATACTATATTGAGTNCACTGTA | |
| | | | | TTATGTATTTA[A/C]GATGGTGGCGTAGTTAGGAAAGGATATAAACATTCAAAGTTTAATAGATTGATTA | |
| | | | | AGTGTTTTCTAGAGATGTTCTTCACCTTTTTCGGCTGTTTAATGTGTATTGAATATAAATATTTTCCCGCTT | |
| | | | | AAGATTCTTTTCAGAAGACGGTCATTTCTTTTTATTCTCATATGCTTGATTTATTCTCATAGTCGTTTTTAAT | |
| | | | | AATATTAACAATCAATTTAGCAGCTCCAAGAAAAAATTATGTCAATATCTTTTCGTCTTAATTTATAAAAT | |
| | | | | TGCCTNCATACGTAATATAAAGATAGTTGNNNNNNTTTATTCTTGACCATGTTTTAATATTAANGATGAT | |
| | | | | GAAAGAAAAATACAAGTAAAACACTATTTAACAAATTNGTTTCAAAGTGGCCCAATATGTGTAATGGTG | |
| | | | | TGACACCGGTGGCTAGTATCTTGAACTTTTTAAANGATTTCAATTCTTAATTATTCAAATAAAGTAACATT | |
| | | | | TGTGGGAAGAAAAGTTGTCTCTTAA | |
| SY4336 | SY4336F1 | AGTGGTT | | | 672 |
| | | CAATTTG | | | |
| | | AGGTGTC | | | |
| | | ATC | | | |
| SY4336 | SY4336R1 | GGTGAAG | | | 673 |
| | | AACATCT | | | |
| | | CTAGAAA | | | |
| | | ACACTTA | | | |
| SY4336 | SY4336A1FM | CGCCACC | C | | 674 |
| | | ATCGTAA | | | |
| SY4336 | SY4336A2TT | ACGCCAC | A | | 675 |
| | | CATCTTAA | | | |
| SY4435 | | | | ATTACGCNATTCTGGAAGCCGCATTTGAGGAGAAAACGGAGANNANCAGGTTCTGCGTGGTGGATTTT | 506 |
| | | | | GAGATTGGANANGGNAAGCAGTATTTGCACCTCCTCAACGCNCTCTCGGCGCGTGACCAGAACGCGGT | |
| | | | | GGTGAAGATCGCGGCTGTGGCGGAGAACGGCGGCGAGGAGAGAGTGCGCGCCGTGGGAGACATGCT | |
| | | | | GAGTCTACTCGCNGAGAAGCTGAGGATCAGGTTCGAGTTCAAGATNGTCGCGACNCAGAAAATCACNG | |
| | | | | ANTTGACTCGCGAGTCNCTNGGATGCGAAGNGGACGAGGTTCTCATGGTGAACTTCNCGTTCAACCTG | |
| | | | | AANAAGATTCCCGACGAGAGCGTCTCNACNGAGAATCCTCGNGACGAGCTCCTGCGNCGCGTGAAGCG | |
| | | | | TCTNGCGCCGCGCGTGGTNACAATNGTGGAGCAGGAGATAAACGCNAACACGGCGCCGTTTTTGGCGC | |
| | | | | GCGTGGCNGAGACGCTGTCGTATTAC[A/G]GCGCGTTNTTGGAGTCCATTGAGGCCACCACTGCAGGG | |
| | | | | AGAGAAAATAACAATAACAACCTAGACCGAGTCAGNCTCGAGGAGGGACTGAGTCGAAAATTGCATAA | |
| | | | | CTCGGTGGCGTGCGAAGGAAGAGATCGCGTGGAACGGTGTGAAGTGTTTGGAAAATGGCGCGCGCGT | |
| | | | | ATGAGCATGGCGGGGTTCGNGTTAAAACCACTGAGTCAAAGCATGGCCGAGTCAATAAAATCGCGACT | |
| | | | | CACCACNGCCAACAACCGAGTCAACTCGGGACTGACTGTAAAAGAAGAGAACGGAGGGATTTGCTTTG | |
| | | | | GTTGGATGGGAAGAACACTCACGGTCGCATCTGCTTGGCGTTAACTCGGCTCNCNTTTTTTNCTTTTTTTT | |
| | | | | TNNNAATTTGGTTCGGAATATTATTATATATCACATTGTTACTATATTTTAACGTCATCTAGAGATAATGG | |
| | | | | AAAGGCCATAGATTTGGAAAATGATTATTATTATTANTANTATTATTAT | |
| SY4435 | SY4435F1 | AAGATTC | | | 676 |
| | | CCGACGA | | | |
| | | GAGCGT | | | |
| SY4435 | SY4435R1 | CAGTGGT | | | 677 |
| | | GGCCTCA | | | |
| | | ATGGA | | | |
| SY4435 | SY4435A1FM | ACGCGCC | G | | 678 |
| | | GTAATACG | | | |
| SY4435 | SY4435A2TT | AACGCGC | A | | 679 |
| | | TGTAATA | | | |
| | | CG | | | |
| SY4439 | | | | TTTTTTTTNNNNNNNNNATTGCACTTACCCTAGCAATTTCATGCTTCCAAAAGAAAATCCTTGTCTTGCTC | 507 |
| | | | | CAACCTCTCCATAGCCTCAGGNTCCAANGTTAGTTCCACATCAATGCTCCTCCCTCCACTCTTCCCCGGGT | |
| | | | | ACAAATAAATCATCCCATCAAACTTGTTATTAGTTCCACTCCTCACATTCTCTGGCTTNCCCCACCCAAAAT | |
| | | | | CAATGTCATAAACCTTAAACCTTGGGGAGCTTCCAACAGCCACACAATTCACCCCAGCATCTTTGAATTG | |
| | | | | AAAATTTTGGGTGTACTCTCCCANTCCTTGTTACGTTCATCAATTGCNTTAGCATTGTGNGNTTCAATG | |
| | | | | GCTTTCTGNANNAATGAAGCCCCAAATTGTGGCGGATGGGCGGCTAATANNCCAACCGCNGTAACGGT | |
| | | | | AAAAATAGCTTGAATTAGGTTTCCNAAATAATTNTCCGGCATTGGTGGGTCCACCCGCTTCCGNCAATCG | |
| | | | | GCGAAGAC[A/G]GTGAACACCGTGTAGTCCTCNGGCTTCAAGTTACGTGCATGGCTTACATGGCGCCAA | |
| | | | | ACGTGGGAGGANAGAGCCTGAAATGTTGAGAATGGTTTTGAGCCATCGGATGGNGGGNTNTCGTTGA | |
| | | | | CCGTTGACTTGATCTTGTCGATGGCTGACTCGGAGAATTTGAAGATCTTNTCCCTAAGTGCTGGCGCGG | |
| | | | | GCTTTGCCTCACCGTTGGAAGTTGGTGGGCCGTTGGGCTCAGGAAGCGAGAGGTCCAATTTCACGCGTG | |
| | | | | TGTTCCGGGCCTTNGTTCGGTCCAGGAAGGGTGGTGCTGACGTGGAGGGTGAACCGCTGCAGATCTCG | |
| | | | | GCCCATGAGGTCATGAATTGCCAGGTGGCAGTNCCGTCCAAGACAGCATGGTTGAATGCTAGGCCCATT | |
| | | | | GCAAGCCCATCTTTGAGCTTCGTTAACTGNGAGCAAGAATAGAATGCAAATGAAGTGAGAACATGAAA | |
| | | | | AATAAAAATAAAAAGATATCATATGATTTANAA | |
| SY4439 | SY4439F1 | TGGGTCC | | | 680 |
| | | ACCCGCT | | | |
| | | TC | | | |
| SY4439 | SY4439R1 | CAAGATC | | | 681 |
| | | AAGTCAA | | | |
| | | CGGTCAA | | | |
| | | CGA | | | |
| SY4439 | SY4439A1FM | AATCGGC | A | | 682 |
| | | GAAGACA | | | |
| | | GTGAAC | | | |
| SY4439 | SY4439A2TT | ATCGGCG | G | | 683 |
| | | AAGACGG | | | |
| | | TGAA | | | |
| IGGY3104 | | | | GAAAAATGAACATATTAACAAGAACGTTATTGCTAGTGAGATAATCAACATAGATTATGGACAATTACAT | 508 |
| | | | | TATTACAAGTTTCCTTATCCTTCACCATACTATGCCAGATGTCAGATGATCCTCATAAGTACAAATATATA | |
| | | | | TATATATATCAAAGGAAATGACATATATACCTTTATGATGCAAACTAACTAAAGCACCATTTTGGATTCTG | |
| | | | | CAAAGGTAATTAAGGAACATGAAATTAAACTATGTCTTGCTTCAAAGAAATTGCTACCCTTTCTAGTTAA | |
| | | | | TTATACCGGATCTGCTAT[C/G]ACTAGAGAGACTTCTGATGATGATGATAGCTATTTTATTTCACTCAGTT | |
| | | | | ATGAAGTGGGTTTGGTCCACCTGGAGTAGCTGTATGTGAAACAGTATGAAATCCACTGACCTTGTTGTCT | |
| | | | | TTGATTTCAGCAGAATTATAACTATGTGGGAATGAAGAAAAGGGGATGCTGGTTCTTGTTCTTTGTTCCA | |
| | | | | TGATCTTTTCCTTATCCCCCCAGGAAATGTGCCTGCAGCTTGAAAGATGGAGCAGTATTACTAGTATAAA | |
| | | | | AAGAAGGAACATAATAATTAGAAATCTAGATTTATGAACTC | |
| IGGY3104 | IGGY3104F3 | TAGCAGA | | | 684 |
| | | TCCGGTA | | | |
| | | TAATTAA | | | |
| | | CT | | | |
| IGGY3104 | IGGY3104F1 | ACTTCGT | C | | 685 |
| | | CAGTAAC | | | |
| | | GGACTCA | | | |
| | | TCATCATC | | | |
| | | AGAAGTC | | | |
| | | TCTCTAGTG | | | |
| IGGY3104 | IGGY3104F2 | GAGTCGA | G | | 686 |
| | | GGTCATA | | | |
| | | TCGTTCAT | | | |
| | | CATCATC | | | |
| | | AGAAGTC | | | |
| | | TCTCTAGTC | | | |
| SY4418 | | | | ATCCAGGAGACNTGCCCAACTATGATGATGCTAAGCCANTACACCTTAATACTGAGCAACATGATGAAA | 509 |
| | | | | TAACCAGTTCAAGTGGAAGTGTAAGTTTTGGTTTTCCTGAAACCTATTCTAGTTCGGGTGCTGATAATGA | |
| | | | | GACTGGAATTGTTAGTGTTGTGGTCATTTCTGAGCTAAATAACATGATTTCAGATCCTAAGTTTTTCAATG | |
| | | | | AAGCTGGTCAAGAGAATATTCTGTCAGCTTTAAAGAATGAAAACCTTNACCTGAACAAAATTCCACAGG | |
| | | | | TCTCCGNTGAGGGAAATGAGCCTTCCTTTGAAGAGCGGAGCATTCCNGGAAATGACCTGTTTGAAAAAT | |
| | | | | CATCTATTTCAACANCAGNCAATNCATTGGTAGATGAGCAGGTTAGAAATGATAATTATGAGGTTGATG | |
| | | | | AAGTTAAATCTGAATCTTCAAATTCTGGATCCTTTTTCTCTGTTCCCGGCATTCCCGCTCCATTAGTAGTTT | |
| | | | | CTACAGCTGTA[A/C]AAGTGCTTCCGGGAAAGATTTTGGTTCCTGCAGCTGTTGATCAAGNTCAGGGCC | |
| | | | | AAGCACTAGCTGCATTGCAAGTTTTAAAGGTAGTTATTTGTTCTTATTCTCATGTATCAAGGTGACTATAA | |
| | | | | TGCTATGTGCATTTATAGTTTAATTCTAGTTTTTACGTATATATTTACACTGGTTATTTCTTAAATCTATTTA | |
| | | | | ATTACTCACAATAAAAAAAGATGACCCGGAGTACAAGGTTCCTGGGAAGGGTAGAGCCTATTTCCAGGA | |
| | | | | TTTGAACCCATGACCTCTAGGTCACAAGATAGCAATGTCACTGTTGCACCAAGGCTCCCCCCCTCCCAGT | |
| | | | | GGGCATACTAGCTTAAACTTCAGCCATCTTGATTCACTGTGATTTGTACTAAGCTATACTCTAACCTATTG | |
| | | | | CCAAAGATTTTCTACAAGGAACAATCTATCTTACTGTTGAAGTAACTAAAATCAATAATTTCTGGTAGCCT | |
| | | | | TATGCTTGTTCCCAGGTCAAC | |
| SY4418 | SY4418F1 | GGCATTC | | | 687 |
| | | CCGCTCC | | | |
| | | ATTAGTAG | | | |
| SY4418 | SY4418R1 | CAACAGC | | | 688 |
| | | TGCAGGA | | | |
| | | ACCAAA | | | |
| SY4418 | SY4418A1FM | CCGGAAG | C | | 689 |
| | | CACTTGT | | | |
| | | ACAG | | | |
| SY4418 | SY4418A2TT | CCGGAAG | A | | 690 |
| | | CACTTTTA | | | |
| | | CAG | | | |
| IGGY2851 | | | | ACTGATATGGCTGAAGCTGATCTGAATACCACAGACTTGTTGCCCCTATCACATGACATAGAACACACTG | 510 |
| | | | | GGTTAATTCAGAATACTAGCAGTGAGGTGGTTTCCAGTATAGATAAAAATGAGATCATAGATCTTCTGA | |
| | | | | GCCCTTCCCCACCTAAGAAATCCAATTTATNCTCAAAATGTCAGCAATCAAGTGACCAACATATTGAAGT | |
| | | | | GATTAATTTGAGTGATTCAGAAAATGACATGTCCGTTGAAC[A/G]CAAGCAGAAAGCGAAGGAGCTGA | |
| | | | | GATTGTTCTTAGCTAGTATTAGGAATGAAATTCATTGAACAATGTTTGTACAAGTAATATAAAAGCAGGT | |
| | | | | TTTATTAGTATTGATATCACACATTGACTAGAATTTAGTACTTACAAGATTGACACCCTCCCCAACTTTCA | |
| | | | | ATTCGGTTTTGTAAGATTTAGTTAGATTGAAAGTCCAACTTTTAATAGTGTAATTAAAAAACAAATGAGT | |
| | | | | TACCATTTGTGATCTT | |
| IGGY2851 | IGGY2851F3 | TTCAACG | | | 691 |
| | | GACATGT | | | |
| | | CATTTT | | | |
| IGGY2851 | IGGY2851F1 | ACTTCGT | A | | 692 |
| | | CAGTAAC | | | |
| | | GGACGCA | | | |
| | | GCTCCTTC | | | |
| | | GCTTTCT | | | |
| | | GCTTGT | | | |
| IGGY2851 | IGGY2851F2 | GAGTCGA | G | | 693 |
| | | GGTCATA | | | |
| | | TCGTGCA | | | |
| | | GCTCCTTC | | | |
| | | GCTTTCT | | | |
| | | GCTTGC | | | |
| SY4440 | | | | AATCATGAAAACGGTATCGTTTCGANGGAGTAGCAGGACAACTTGAAAAGATACNATAGAAAAACGAA | 511 |
| | | | | GTCGTAATGGTGTCTGATNATTTTAGGAACTAAAANAGTGATATGCTATGATTGTACTACAAGTGTAGT | |
| | | | | GGCAGAAAGCAAATTTCTATCTCCCGGAAATTAACCAAAAAAGACTACGACTAAAACTAAAGAGACTAA | |
| | | | | GGAAAATAAGATAAACAAATATTACTTGTCAACTTTACCTTGAAGGCCTGGTGTAGAAGGATTNNCCGN | |
| | | | | CATTGGCNATTGAGTTTCTTCANCACTACCCCTGTAAAATTGGAACAATTCAAACCTGAGATTGCAGCTN | |
| | | | | GGATATAAAACTCTTCCCCCTACGCTTCCCAAGCGGAACCATGGAATTTTTCCATTTATATCCTCCAGACA | |
| | | | | NTTCTTACAACCATCACTAGACAAATCCNGCGTGCATTGAGCAAGAGTATACAGAGTTTGCAAATCAGTC | |
| | | | | AATTTTAATGATTT[C/G]GTGACATATCTCTCAGTGGTATCCCCTGCCTCTTGGGCCAGCTTAACTATGGT | |
| | | | | ATCTGATAATGTANAAGTGAAGNAGTCTTGCCCGGGGATGATGCTGGTGGAGGAACTGGTAAGATTCA | |
| | | | | GCATGTCAAAATTTGGACTTTNTTCCACTTCTGAGAAGAAATACAGATTGGAATATCGAATCATGCAGTG | |
| | | | | GCTGTACCAAATGATTCCCTCTTGAACTGAATTACACACTGAGGATATTCGGTGGGTTGCGTTGAGGAC | |
| | | | | GCATTGTTGGCAGAGTTGAGAGGGAAGATNGCCTCGGCACATGAAGAGGCCATACACAGTGTTCTCTA | |
| | | | | CGTTGTCCNTGTAAGATTTTNTCCCATTTGTGGCATTNGAAGACAAGTAAAAGAGAAGGGTCTTGAGAT | |
| | | | | ACATTTGGAAAGTGCTGNCAACANTTACATCGGTTGGGCAACTNTGGTTTAGATAAGTTGGATCTTCTG | |
| | | | | AAAATTGTGAATCCGCATCGGGAAAATTTGTTGC | |
| SY4440 | SY4440F1 | CGTGCAT | | | 694 |
| | | TGAGCAA | | | |
| | | GAGTATA | | | |
| | | CAGA | | | |
| SY4440 | SY4440R1 | CCATAGT | | | 695 |
| | | TAAGCTG | | | |
| | | GCCCAAG | | | |
| | | AG | | | |
| SY4440 | SY4440A1FM | TCAATTTT | C | | 696 |
| | | AATGATT | | | |
| | | TCGTGAC | | | |
| SY4440 | SY4440A2TT | TCAATTTT | G | | 697 |
| | | AATGATT | | | |
| | | TGGTGACA | | | |
| SY0127AQ | | | | TNTGAAAAAANTAATAAAGAAAAATAACATATATTAATATTTGATAGTAGTGTTTAACACACAAGATATT | 512 |
| | | | | ATTAGCAGCACNCTATTTAATATACTCTTTCTAAAACACTTTATTATTGTTGAAATTTATGAAAAATTACAA | |
| | | | | AATCTTGTTAACCCCTCTTTCCAATATTTAATACAAGATTCAGTCATTTTTAATAAATTTCATTTAATAATA | |
| | | | | AAAAGTATATTTGAGGAAGAATCTATAGATATTTGTGTATT [symbol] TTACTCTTACCGGTTTGACCTCCAA | |
| | | | | GGAAATTCTGCCAGAGGTAGTTTGCAAGTTGAGTGGCATCATCAGCTGANCTGAGGGAGTAGCTTCCA | |
| | | | | GCACCACCACCNAGGGAGAGCAACACTTTGATGCCAAGGTCTTGGCAAGTTTTGATGTCACAGTT | |
| SY0127AQ | SY0127AF1 | GCAGAAT | | | 698 |
| | | TTCCTTG | | | |
| | | GAGGTCA | | | |
| | | AAC | | | |
| SY0127AQ | SY0127AR1 | CCCCTCTT | | | 699 |
| | | TCCAATA | | | |
| | | TTTAATAC | | | |
| | | AAGATTC | | | |
| | | AGT | | | |
| SY0127AQ | SY0127AA1FM | CGGTAAG | A | | 700 |
| | | AGTAATA | | | |
| | | ATACA | | | |
| SY0127AQ | SY0127AA2TT | CGGTAAG | G | | 701 |
| | | AGTAACA | | | |
| | | ATACA | | | |
| SY1044BQ | | | | CTTATAAAAGCCACAAGCAAATTCCTCNAAGATGCCGAAAACAAACCAATTTACCAGAGTACAAAAGTG | 513 |
| | | | | TGAACGGATCATAATAANCATG[A/G]TGAAGAAAGGAACTACATACATACAAATGATACAAAAACAGT | |
| | | | | GATTCATTTAGTTTTCTTGAATCCACAAAATGAAACTAGACAGTGGATTTTATTTCATCGATCACTGTCTA | |
| | | | | AGAACCATTAGCTTGAGGAGTTGAAGACTTCTTTCCCTCGATGTCCATCTCTTTTACACTATCACTCAGTG | |
| | | | | AAGACGACTCACATTTC | |
| SY1044BQ | SY1044BF1 | CAAGAAA | | | 702 |
| | | ACTAAAT | | | |
| | | GAATCAC | | | |
| | | TGT | | | |
| SY1044BQ | SY1044BR1 | AGCCACA | | | 703 |
| | | AGCAAAT | | | |
| | | TCCTC | | | |
| SY1044BQ | SY1044BA1FM | CCTTTCTT | G | | 704 |
| | | CACCATG | | | |
| SY1044BQ | SY1044BA2TT | AGTTCCTT | A | | 705 |
| | | TCTTCATCA | | | |
| SY0571AQ | | | | TTTTTCTCTCCCCTCAAGGCAAATAACATGAGACGGAAAAANGGAGGAGAAAAAGTGAAAAACAAGAA | 514 |
| | | | | AGTGAGAAATTAGATAGTANCACTTCTCAATCAGACACACCATTAAGCCACTACCAAAACTAAACAAAAC | |
| | | | | TTTGCACCAGCCAGAAGACAGTTAACATTAACAACAACGCAAATAAGGAAAACATATACAATGCGTTAG | |
| | | | | CTGAGCAAAATTTGCGTCAAGTATGCTGCANTTTAGGCAC[A/G]GCATGAAGCAATCCGATTAACAAGT | |
| | | | | CAAGTCTTCTATCCGCTTAGCAGACAAGAGATGATCTCAAAGATGTAGGTAGTTGAGTGCATGATGACC | |
| | | | | AACGAATGACTGATTCAGTCACCATAAGGTCAAGTTGCTCACACTCACCTAGTCCAATTGTCCTGTTTCTC | |
| | | | | TTGCTGTGGATTCCNAATACCTTATGTCCATTCATTCCNCTTGCCCTTTTGGCCTCTACAGGCTTGCGATC | |
| | | | | NGTTTCTTTAACCTCACGTCCAACNCAAAGAGGAGAATCTTGTAGCATAAGCA | |
| SY0571AQ | SY0571AF1 | GCTAAGC | | | 706 |
| | | GGATAGA | | | |
| | | AGACTTG | | | |
| | | AC | | | |
| SY0571AQ | SY0571AR1 | GCACCAG | | | 707 |
| | | CCAGAAG | | | |
| | | ACAGTT | | | |
| SY0571AQ | SY0571AA1FM | ATTGCTTC | G | | 708 |
| | | ATGCCGT | | | |
| SY0571AQ | SY0571AA2TT | ATTGCTTC | A | | 709 |
| | | ATGCTGTG | | | |
| SY0096C | | | | ACTTCACACCACAGGGCGATGATGTGCCTGGCGAGGTTGATTATAACCTTTAGGAGAAGCACCACAAGC | 515 |
| | | | | ATCTTGAGCACCTTGGGGAACTTGGTGCTGCGGCTGCCGGTGCTTATGCCTTGGTAATTAATTATATATC | |
| | | | | TTCCTTCAATAATATATTTTGTTCACGATCGTTTATTTAATTTGAAATAGATTTATATATTACTTATGTGAG | |
| | | | | ATGATTCACACCCCCTTTTTATATATTTTAGCTTTAAAATGTTACCTTCACCAGAATAAATAAAAGAAGTG | |
| | | | | CAAACTCTTTGNTAATCGAGGGAAATATATATACCTCCCCCACATATACATCATCACTTAGACTTGGACGT | |
| | | | | ATCTAAATCGGTTAATTTTAATATGTTTATATGTATGCGTGTGCATTAATAATTTTCATATNTTTTTTTGTA | |
| | | | | AGCATTTTAAAGCCTTACATATTGAAAAAATTGTCATTAATTTGTGTTTTGGACATGAATTAATCCTATCA | |
| | | | | TCTTGAATCATGTCCACAAATAATTTCAATTTGACATTTTCTTTTTAAGGCGGCCAACATATATACATACTT | |
| | | | | GATCTTTGTACTTTTGGATTGTGATGCTTTAATAATTGTGGATAATAGATATAAAAATATATTATAGCTAT | |
| | | | | ATAGTATTATTTC[A/T]CTCTACCCACTGTGTGTAACTATACTGTCTATACATCTCATGTGGTTTGTTTTTT | |
| | | | | CTTAAATGAAAATTGTTGGGGTCATGGGTGTATAGAGTATAGTAGTACTTTTATGACGCCATCAGAAGAGAA | |
| | | | | ACAATAAAAGTTCATAAAAAATTAGGTGTAGAAAAAGATGGAACTTAAGAAAGAAAAAAGAGAGAGAG | |
| | | | | AAAGTGATTAAGTGATGTAATATATAATGAGAAATGAAGAAAAAGATAGGAAGACAAATAAAGTAAAA | |
| | | | | NAAAGAAAGAAAGAAAGAAAGATATATAACAAAAAATTGAAATGTATATTCTAATATGTATTGAAAACA | |
| | | | | AAATTGATCCCTTTTTGCTGCAATGGTTAATTTTATGACAGCATGAGAAGCATGAGGCCAAGAAAGACCC | |
| | | | | AGAGCATGCTCACAGGCACAAGGTAGAAGAGGAGATTGCGGCNGCAGCTACTGTTGGTGCTGGTGGTT | |
| | | | | TTGTCTTGCATGAACACCATGAGAAAAAGGAAGTTAAGAAAGAGGATGAGGAAGCTCATGGAAAGAAG | |
| | | | | CACCACCATCTTAAGGGTGAACATGATAAATATTCATATATAATTATATC | |
| SY0096C | SY0096CF1 | AAAACAA | | | 710 |
| | | ACCACAT | | | |
| | | GAGATGT | | | |
| | | ATAGACA | | | |
| | | GT | | | |
| SY0096C | SY0096CR1 | TTTTGGA | | | 711 |
| | | TTGTGAT | | | |
| | | GCTTTAA | | | |
| | | TAATTGT | | | |
| | | GGAT | | | |
| SY0096C | SY0096CA1FM | CAGTGGG | A | | 712 |
| | | TAGAGTG | | | |
| | | AAA | | | |
| SY0096C | SY0096CA2VC | AGTGGGT | T | | 713 |
| | | AGAGAGA | | | |
| | | AA | | | |
| SY0096A | | | | ACTTCACACCACAGGGCGATGATGTGCCTGGCGAGGTTGATTATAACCTTTAGGAGAAGCACCACAAGC | 516 |
| | | | | ATCTTGAGCACCTTGGGGAACTTGGTGCTGCGGCTGCCGGTGCTTATGCCTTGGTAATTAATTATATATC | |
| | | | | TTCCTTCAATAATATATTTTGTTCACGATCGTTTATTTAATTTGAAATAGATTTATATATTACTTATGTGAG | |
| | | | | ATGATTCACACCCCCTTTTTATATATTTTAGCTTTAAAATGTTACCTTCACCAGAATAAATAAAAGAAGTG | |
| | | | | CAAACTCTTTGNTAATCGAGGGAAATATATATACCTCCCCCACATATACATCATCACTTAGACTTGGACGT | |
| | | | | ATCTAAATCGGTTAATTTTAATATGTTTATATGTATGCGTGTGCATTAATAATTTTCATATNTTTTTTTGTA | |
| | | | | AGCATTTTAAAGCCTTACATATTGAAAAAATTGTCATTAATTTGTGTTTTGGACATGAATTAATCCTATCA | |
| | | | | TCTTGAATCATGTCCACAAATAATTTCAATTTGACATTTTCTTTTTAAGGCGGCCAACATATATACATACTT | |
| | | | | GATCTTTGTACTTTTGGATTGTGATGCTTTAATAATTGTGGATAATAGATATAAAAATATATTATAGCTAT | |
| | | | | ATAGTATTATTTCNCTCTACCCACTGTGTGTAACTATACTGTCTATACATCTCATGTGGTTTGTTTTTTCTT | |
| | | | | AAATGAAAATTGTTGGGGTCATGGGTGTATAGAGTATAGTACTTTTATGACGCCATCAGAAGAGAAACA | |
| | | | | ATAAAAGTTCATAAAAAATTAGGTGTAGAAAAAGATGGAACTTAAGAAAGAAAAAAGAGAGAGAGAA | |
| | | | | AGTGATTAAGTGATGTAATATATAATGAGAAATGAAGAAAAAGATAGGAAGACAAATAAAGTAAAANA | |
| | | | | AAGAAAGAAAGAAAGAAAGATATATAACAAAAAATTGAAATGTATATTCTAATATGTATTGAAAACAAA | |
| | | | | ATTGATCCCTTTTTGCTGCAATGGTTAATTTTATGACAGCATGAGAAGCATGAGGCCAAGAAAGACCCA | |
| | | | | GAGCATGCTCACAGGCACAAGGTAGAAGAGGAGATTGCGGC[A/G]GCAGCTACTGTTGGTGCTGGTG | |
| | | | | GTTTTGTCTTGCATGAACACCATGAGAAAAAGGAAGTTAAGAAAGAGGATGAGGAAGCTCATGGAAAG | |
| | | | | AAGCACCACCATCTTAAGGGTGAACATGATAAATATTCATATATAATTATATC | |
| SY0096A | SY0096AF1 | AGACAAA | | | 714 |
| | | ACCACCA | | | |
| | | GCACCAA | | | |
| SY0096A | SY0096AR1 | AGGCACA | | | 715 |
| | | AGGTAGA | | | |
| | | AGAGGA | | | |
| | | GATT | | | |
| SY0096A | SY0096AA1FM | CAGTAGC | A | | 716 |
| | | TGCTGCC | | | |
| | | GC | | | |
| SY0096A | SY0096AA2VC | CAGTAGC | G | | 717 |
| | | TGCCGCC | | | |
| | | GC | | | |
| SY0569AQ | | | | CTTTGGGCTGCACAGAAGGGGCAAAAGAAGACATAGAAAATAAAAAATCTACGGATGGTCGCAGTCAA | 517 |
| | | | | GGAGATTTGTTTGAAGAGAATTTTAAAGAACTGAAGAAATGGGTTAATGTGAAGTCAACTAAATATGGG | |
| | | | | ATCCTTTTAGTAACTCGTGAGAGGCGAGCTCAAAGGCTTGGGACTGCGTTGAAGGTATTTTGTTTTACAA | |
| | | | | GTCTTGCACTGGTTGCTGCATGTAACTCATCTATTTATTTATTACTAATTTACTAATAAAATATCATATGAC | |
| | | | | ACTGGAGTCTACTTGAGTATGTGGACTCGATAGTCGATAATATTCTTTGATCCTCAGTGAGATTTGCCTT | |
| | | | | GAAGTATTCACTCAGCTTATAGTAGATAAACNACCAAACTACTTACTTCTGAACCTCTTCTCACTTGATTC | |
| | | | | AGGTACTGTGTGACATAATTCAAGATGACGCAGAGCCTGCCAAGAAGAAATTCTATGACCTTAAGCTCT | |
| | | | | CTTTGCTTGATGAGATTGGATGGACACATTTGGCTGCATATGAGAGACAATGGATGCATGTGCGTTTCC | |
| | | | | CTCCAAGCTTACCTCTTTTCTAGGACCTGCCCATCGGGAAGATGCTGGACAGCAATGTTAGGACTTTGGA | |
| | | | | ACC[A/G]TGTCCTTTTCCTCNATATTTATGTAACACTAGACCCTTTACGTGACCTATCCTTTCTTTTTGTGA | |
| | | | | ACATCTTGGCCTTGGTATTTCGAACATGGCATGGGAACTTTGCCATGCCTTCAGTGTGGCTGCCACATCA | |
| | | | | GTGG | |
| SY0569AQ | SY0569AF1 | TCGGGAA | | | 718 |
| | | GATGCTG | | | |
| | | GACA | | | |
| SY0569AQ | SY0569AR1 | TCGAAAT | | | 719 |
| | | ACCAAGG | | | |
| | | CCAAGATG | | | |
| SY0569AQ | SY0569AA1FM | AGGACTT | A | | 720 |
| | | TGGAACC | | | |
| | | ATG | | | |
| SY0569AQ | SY0569AA2TT | ACTTTGG | G | | 721 |
| | | AACCGTG | | | |
| | | TC | | | |
| IGGY476 | | | | GATCCCACATCAACTAGTGATAYTACCAAAATAATATATATAAGCGAGGAACAACATTCRTCTAGTGAGC | 518 |
| | | | | TAGCTTSTGAGGTTGAGTTAGGTTCAAAKACGAATGTTAGRAATTCTACTATCACTAATATTGGTGATGR | |
| | | | | GGTTGTTGCGTYGGARAGAAACCTTTCRAGGTCGAACTCGACGGGGCATTCCCTTGTGGAGGAGCAAG | |
| | | | | GGAAGGGTGTGGAGAGGTACACGTTGAGGTTGCCCGAAGATGTGAGGAGGTACATTCTAGTGAACCAT | |
| | | | | GGAAGAA[C/G]TGTTCAACGTTCCGCGAGTGTTAARGGGGGGTGTTGGAGTGACAGCGAAGAGAGTTA | |
| | | | | CGTGGGGAAGAGGGTGGAGAAGAGGTGGGTGATCTGCACGCCGCCATTTGTGGCGCAACATGRTTGA | |
| | | | | AGAATTTCGTCGAACAATTGGTCTGCGTTCTGCGTTGCGCCTTCTATAAAGGGTCGTTGTCAATTCTTGC | |
| | | | | AAGAGATTGTGAGAGTTTGGTGTTACAGAGATGAAGCAGAGGACTGAAATGGAAGAAGAG | |
| IGGY476 | IGGY476F3 | TCTTCCAT | | | 722 |
| | | GGTTCAC | | | |
| | | TAGAATG | | | |
| IGGY476 | IGGY476F1 | ACTTCGT | C | | 723 |
| | | CAGTAAC | | | |
| | | GGACACA | | | |
| | | CTCGCGG | | | |
| | | AACGTTG | | | |
| | | AACAG | | | |
| IGGY476 | IGGY476F2 | GAGTCGA | G | | 724 |
| | | GGTCATA | | | |
| | | TCGTACA | | | |
| | | CTCGCGG | | | |
| | | AACGTTG | | | |
| | | AACAC | | | |
| IGGY515 | | | | AAGCTTTCCTTGCACAAAGTAAGCTTTGTCACTTCATGCCTTTGCTGCCCTTTTTGATCAAATGCTTKGCTG | 519 |
| | | | | GGTCTTCAATTAATATTTGCCTAATCAAAACTATTTTCATGCAGGGTGGAAGGAAGCTATCTCATCAGAA | |
| | | | | CATGTGACATTGGTTATTTGCCCCGACTCGGAACCTCCTGGGTGCGAGGAGATACAGGATTTGAAAACA | |
| | | | | GCAGCATGTCAATCTTCTGATATGGATGGATGTGACATTGTGGCAAATGCAGATAAAAGATTGCCTGCA | |
| | | | | ACCTCCAAAGTTGCGAAATCCAAACCCAGGTTGAAGAAGTCTGAAAAGGGAACCAAGATAAAGTATATT | |
| | | | | CC[A/G]AAACAGAAGACAAATACCTAGTGTGAAAGAATAAATTGGAGTTGTTAGTAAGGCAGATATAC | |
| | | | | AGAGTTTGACTTTGTTGCCAATTTATTAAAGAGAGGTCCAAGTTTTACCGGATGGTCTTCTGTCAGAATG | |
| | | | | TGAAAG | |
| IGGY515 | IGGY515F3 | AACAGAA | | | 725 |
| | | GACAAAT | | | |
| | | ACCTAGT | | | |
| | | GTG | | | |
| IGGY515 | IGGY515F1 | ACTTCGT | A | | 726 |
| | | CAGTAAC | | | |
| | | GGACGGA | | | |
| | | AAAGGGA | | | |
| | | ACCAAGA | | | |
| | | TAAAGTA | | | |
| | | TATTCCA | | | |
| IGGY515 | IGGY515F2 | GAGTCGA | G | | 727 |
| | | GGTCATA | | | |
| | | TCGTGGA | | | |
| | | AAAGGGA | | | |
| | | ACCAAGA | | | |
| | | TAAAGTA | | | |
| | | TATTCCG | | | |
| SY3108 | | | | GACACGAATGCCATCCAACATAAAAATGATGCGATCCAGTGATTGTAACCCCAACTGACAAACACAATTT | 520 |
| | | | | TTGTTTTAAATGAAAACTACCATCACAATA[A/T]GCATATAGAAATTGATTAAAAGCTCAAATTCAAGAT | |
| | | | | ACTTCCTTATCTTCCTGAATTCCATAAACCAAAACTAAGCATGCACCATCTTGAGCAGACAGAC | |
| SY3108 | SY3108F1 | GTAACCC | | | 728 |
| | | CAACTGA | | | |
| | | CAAACACA | | | |
| SY3108 | SY3108R1 | TGCTCAA | | | 729 |
| | | GATGGTG | | | |
| | | CATGCTT | | | |
| SY3108 | SY3108A1FM | ACTACCA | A | | 730 |
| | | TCACAAT | | | |
| | | AAGC | | | |
| SY3108 | SY3108A2TT | ACCATCA | T | | 731 |
| | | CAATATG | | | |
| | | CAT | | | |
| SY3112 | | | | CTTTGATAATCGAGGGAAATATATCTCCCTCCCACATATACATCATCACTTAGAATTGGACGTATGTAAAT | 521 |
| | | | | TGGTTAATTCAGCCCAAGACAAAATGGAC[A/C]AAGTGCGCCAACAAAGATAGAGTTAGCTATAGCTTA | |
| | | | | ACTGCACGTATCATAAAATTTGTTTAAAGTAATTACATAGATAGCAAAAACCAGAAGAACTAAA | |
| SY3112 | SY3112F1 | CAAATTTT | | | 732 |
| | | ATGATAC | | | |
| | | GTGCAGT | | | |
| | | TAAGC | | | |
| SY3112 | SY3112R1 | TCCCTCCC | | | 733 |
| | | ACATATA | | | |
| | | CATCATC | | | |
| | | ACTT | | | |
| SY3112 | SY3112A1FM | TTGGCGC | C | | 734 |
| | | ACTTGGT | | | |
| SY3112 | SY3112A2TT | TGGCGCA | A | | 735 |
| | | CTTTGTC | | | |
| SY3110 | | | | CACAAAGAGAATCTTTGTTACCCCTGATGGTAATCTTTGAAAAATATACTTCCAAAAGCTCTCTCCTTAAG | 522 |
| | | | | GGGAAAATTTGGGTAAAGATGTGTATTTT[A/G]ACTTTGATCTATCTCTCTTAATGAACCTATACCCAAAC | |
| | | | | ATTGAATCTGTCCCAAATACTCACGGTTCTAAACAAGACCTGGCACATAATCTTATTTGAAT | |
| SY3110 | SY3110F1 | ACCCCTG | | | 736 |
| | | ATGGTAA | | | |
| | | TCTTTGA | | | |
| | | AAA | | | |
| SY3110 | SY3110R1 | GAACCGT | | | 737 |
| | | GAGTATT | | | |
| | | TGGGACA | | | |
| | | GA | | | |
| SY3110 | SY3110A1FM | TAAAGAT | A | | 738 |
| | | GTGTATT | | | |
| | | TTAACTT | | | |
| SY3110 | SY3110A2TT | AAAGATG | G | | 739 |
| | | TGTATTTT | | | |
| | | GACT | | | |
| SY3114 | | | | GGGCCCTCCAATTTGTTATTAGAACAATCCAACTCAGAAAGTTGAGTTGAGCCAAATAACGAAGATGGG | 523 |
| | | | | ATCGGGCCTCCAAAATTGTTTCCCTCAAGAT[A/T]GAGAGTATTCAATTTGTTTAGCCTGGCAAACACAT | |
| | | | | CTGGGATTTGACCAATAAATTTATTATGTGAAAGATCCAGGTGAATGAGATGTTGAAGATTTGAA | |
| SY3114 | SY3114F1 | GGGATCG | | | 740 |
| | | GGCCTCC | | | |
| | | AAA | | | |
| SY3114 | SY3114R1 | TGCCAGG | | | 741 |
| | | CTAAACA | | | |
| | | AATTGAA | | | |
| | | TAC | | | |
| SY3114 | SY3114A1FM | TTTCCCTC | A | | 742 |
| | | AAGATAG | | | |
| | | AG | | | |
| SY3114 | SY3114A2TT | TTTCCCTC | T | | 743 |
| | | AAGATTGA | | | |
| IGGY2357 | | | | TCTTGATGTGGAAAGGTTCAGAACGAATATTCAAAACTAAAGTGTTACTACTTGTAAAAAGCATTGATCT | 524 |
| | | | | CTCAAGCAATCACTTTTCTGGAGAAATTCCACAGGAAATAGAGAATTTATTTGGATTGGTTTCATTGAAT | |
| | | | | TTATCAAGAAACAATTTGATAGGGAAAATTCCCTCAAAAATTGGAAAGCTAACATCACTTGAATCTCTTG | |
| | | | | ATTTGTCAAGAAACCAGTTGGCTGGTTCAATTCCTCCGAGTCTTACACAAATTTATGGCCTCGGCGTGTT | |
| | | | | AGATTTGTCACATAACCATCTAACTGGAAAAATTCCAGCCAGCACACAGTTACAGAGTTTCAATGCCTCG | |
| | | | | AGTTATGAAGATAATCTTGATCTTTGTGGACAGCCACTTGAGAAATTTTGTATTGATGGGAGACCTACAC | |
| | | | | AAAAACCAAATGTTGAAGTTCAA[C/G]ATGACGAATTTTCACTTTTCAATCGTGAATTTTACATGAGTAT | |
| | | | | GACATTTGGATTTGTTATAAGCTTTTGGATGGTGTTTGGCTCAATCTTATTCAAGCGTTCTTGGAGACATG | |
| | | | | CCTATTTCAAGTTCTTGAACAATCTATCAGACAATATTTATGTCAAGGTAGCAGTATTTGCTAATAAAATG | |
| | | | | TCAAAGGTGTATGGCTGAAGCTTAACTAGGTAATAATATTGCAGCCCTTTCATATATATATATATATATAT | |
| | | | | ATAGTTTCTTTTGCTTTCATATAGTTTATATACATGAAAGATTCCATATATATTATAATTTGGAATTGTGAC | |
| | | | | AGTAAGATTTCATAATTTTTAACTATTTTAGTATAATAAATTTTGAAGAAATATTGAATAAGTTATATTAA | |
| | | | | GATTAATTAATAATATAAAATTATATTGTTACTGTATAATCATTAAAATTATCATTATTGATGTATAATAA | |
| | | | | GCCTGAAACATCGTTGATCTCTATTATTAT | |
| IGGY2357 | IGGY2357F3 | TGAACTT | | | 744 |
| | | CAACATT | | | |
| | | TGGTTTTT | | | |
| IGGY2357 | IGGY2357F1 | ACTTCGT | C | | 745 |
| | | CAGTAAC | | | |
| | | GGACCGA | | | |
| | | TTGAAAA | | | |
| | | GTGAAAA | | | |
| | | TTCGTCATG | | | |
| IGGY2357 | IGGY2357F2 | GAGTCGA | G | | 746 |
| | | GGTCATA | | | |
| | | TCGTCGA | | | |
| | | TTGAAAA | | | |
| | | GTGAAAA | | | |
| | | TTCGTCATC | | | |
| SY3121 | | | | CAATGTACAATTATATTATCTTTCAAGACATCAGGATTTTGGAATTGTTCTAGTTTAGAGGAGAAAAGTC | 525 |
| | | | | ATCTAGTTTATAACTACACTGTTTTTGAATTTTAGCATCTATCAATTTAAGTAATTATAATATTTGATAGAT | |
| | | | | GAATTATATAGTCAGTTATATTAATAGAAAGCAGAGCTTAAAAGGGACAGTAAAACAGAAAGTTGCAAT | |
| | | | | ATATTCACCAAAGACAACAGCCTTGTCCTCTCAACCAAC[A/C]ACCATGAATTCAGGTCCTAGCGAAGAC | |
| | | | | GGACACACCTCATGAAAATAAATAAAAAATTAAAGAAAATAAGTATCTTTAGTTCAGCAGTTAAGCTAAC | |
| | | | | CAACAAAAACAAACCAAAGTATAATCTCACACCAAAATATGTATAACATTGATCCAGAAAATGTCTTAAT | |
| | | | | ATTCCCATTTCTTCAACTCCATGCCATCAGGAGCACTTCCCTCNACCTTCTTTGANCCCACTTCTTTCCAGT | |
| | | | | TTGTAGACAGC | |
| SY3121 | SY3121F1 | TCACCAA | | | 747 |
| | | AGACAAC | | | |
| | | AGCCTTG | | | |
| SY3121 | SY3121R1 | CGTCTTC | | | 748 |
| | | GCTAGGA | | | |
| | | CCTGAA | | | |
| SY3121 | SY3121A1FM | TCAACCA | A | | 749 |
| | | ACAACCA | | | |
| | | TG | | | |
| SY3121 | SY3121A2TT | TCAACCA | C | | 750 |
| | | ACCACCAT | | | |
| SY3148 | | | | CGCTGCCAACACCTCCAAGGCATCATCGGATTCCGAGAATTTCGCTGAGTCGGTGATCAAGGCTCCTAA | 526 |
| | | | | GCAGGCCTCTGGGGAGCACAAGAAGAAAAAGAAGATCAAAGTGACNTTCCCATCAGGTCAAGAGCGG | |
| | | | | AATGCACCATCACAGGCAATTAGGAAATGCTTGCACTGTGAGATAACCAAGACACCACAGTGGAGGGC | |
| | | | | AGGGCCAATGGGGCCGAAAACACTCTGCAATGCTTGTGGCGTGCGCTACAAGTCAGGCCGGCTTTTCCC | |
| | | | | CGAATATCGCCCTGCAGCGAGTCCAAC[G/A]TTTTGTGCGGCCATGCACTCCAACTCCCATAAGAAGGTC | |
| | | | | CTTGAAATGAGGAACAAGACAGGCACCAAATCTGGCTTTGCAACTGTTTCTGCTGCCTCACCAGAACTCA | |
| | | | | TTCCAAACACTAACAGCAGCCTTACCCTTGAATATATGTGAAAGGGGGAAAGGAAGGATTCTAGTTGGA | |
| | | | | GAATTCTCTAATTCTCTTTCAAGTCNTCTCTTGAGTCATGTCTTATACAAGGTTTTGAATTGCATTCTACAA | |
| | | | | ACTGCAATGTTAAAGGTTTTAGAGGTGTCTGCANCTGCGTTGTGGTTGCG | |
| SY3148 | SY3148F1 | GCGTGCG | | | 751 |
| | | CTACAAG | | | |
| | | TCAGG | | | |
| SY3148 | SY3148R1 | GGTGAGG | | | 752 |
| | | CAGCAGA | | | |
| | | AACAG | | | |
| SY3148 | SY3148A1FM | CAGCGAG | A | | 753 |
| | | TCCAACA | | | |
| | | TT | | | |
| SY3148 | SY3148A2TT | AGCGAGT | G | | 754 |
| | | CCAACGT | | | |
| | | TT | | | |
| SY3005 | | | | ATGGATATTTTTTTAAGTGATCATTTATCTATTTGTAATTACTAAAAACATATTTAACTTATTTATTCCGTA | 527 |
| | | | | CGTCTGACCACATGCCAAATCAATTTGTTTCCCACAGACACCCACCACCACAATTCATGTGGATATGATGT | |
| | | | | GAAAGCGTGACATTATTATTTTCAATTATGATTACCTATTTAAATCAACTAGCCAAGTGATCAATAATATT | |
| | | | | GCACTATGGTCCCTCCTCTTGGGTAAGACCTGGTAATCTTACGGAAGTGTCTCACTTCATACATCTATTTA | |
| | | | | TTACACATAGCCTATCCTAAATCATACGTGGCAAGTTCTTACTGGACAGAATAGGTACTTAATGATATTTT | |
| | | | | TTGAGATATTCGATCTATGTTGGTAAGGGAAGGAAAACAAACAACTCATAAGCGAAATAAAATGGACAA | |
| | | | | AATGGATGCCCCAAGCCAATAGAAATTCAGATATAGATCTCGTAAAGATAAGTACCCCTCTCTTCTTTAA | |
| | | | | GCTATATATTGTGCTAAAAAAAAATATATAGGCATCACCAGTAGCCATTCTCTTCAGTTTNAAGTTACATA | |
| | | | | GTTTTTCATTGTTTTACTTAATCTACAATGGCTGCTTCAACAATGGCTCTCTCTTCATCATCATTGGCTGGC | |
| | | | | CAAGCTATCAAGCTTGCCCCCTCCACCCCTGAGCTTGGTGTTGGAAGGGTTAGCATGAGGAAAACAGCC | |
| | | | | TCCAAAACTGTTTCCTCAGGAAGCCCATGGTACGGCCCAGACCGTGTCAAGTACTTGGGCCCATTCTCAG | |
| | | | | GTGAGCCCCCATCCTATCTCACTGGTGAATTCCCAGGTGACTATGGTTGGGACACTGCTGGGCTTTCTGC | |
| | | | | TGACCCAGAGACTTTTGCCAAAAACCGTGAACTGGAAGTCATCCACTCCAGATGGGCCATGTTGGGAGC | |
| | | | | CTTGGGCTGTGTTTTCCCTGAACTCTTGTCCCGCAACGGAGTCAAGTTTGGCGAGGCCGTGTGGTTCAAG | |
| | | | | GCCGGGTCTCAGATATTCAGTGAGGGTGGGCTTGACTACTTGGGCAACCCAAGCCTGATCCATGCACAA | |
| | | | | AGCATTCTTGCCATCTGGGCCACCCAAGTTATCTTGATGGGTGCCGTTGAGGGTTACCGTATTGCTGGTG | |
| | | | | GGCCTCTTGGTGAGGTGACTGACCCAATCTATCCAGGTGGCAGCTTTGACCCATTGGGCCTTGCTGATG | |
| | | | | ACCCAGAGGCTTTTGCTGAGTTGAAGGTGAAAGAGCTCAAGAATGGTAGGTTGGCCATGTTCTCCATGT | |
| | | | | TTGGTTTCTTTGTTCAGGCAATTGTGACAGGAAAGGGACCCTTGGAAAACCTTGCTGATCACCTTGCTGA | |
| | | | | CCCAGT[C/A]AACAACAATGCTTGGGCTTATGCCACAAACTTTGTTCCCGGAAAGTGAAATGACTTGTGA | |
| | | | | ATTTTATGTTATTTAGTTAAATATGTATTGGATCTATCAAGTGAGAATGTGAATTATATTATTATATTTTAT | |
| | | | | ATATCTCTTTTTAGTTCATTTGGATGTATCTCCAAGGTTCTAAGTTTATATATATATGCACTTTATTAACCA | |
| | | | | AACTAATTAAAGCTCAAATGACAAGTCTTAAACATTAGAAGCGAGTTAGGTTCTAAAATTTAAAGCAGTG | |
| | | | | GGATGAAGAAGGAGAAGTAGAAATCACCAAGACATAAATACAAGTGCTTTAAATTTTGCATTTGATTCT | |
| | | | | CCTCATGTGGACCACAAATAATTTTCATCATAAGCTTGAGTAGGTTTACTCTTTTTAGGTTGACTTTCAAG | |
| | | | | ACTCTGGACTTTATTCTTCAAACTATATTAGTCAGTAAAATCAATTAAGCTTTACAAACTGCCAACTGATA | |
| | | | | ACTGTAAATTATTATTGTTCTAGTGAAACCAAAATGAAATATTTTATTTTACTCTGTTTCCTTTTTAATTAC | |
| | | | | ACTCTTTCAGTAAAAATAAAATATGCCATGTTTTAAACCTTGTATCCTTATTTTAATTTTTATTTATAAAAT | |
| | | | | ACGCCAGATATAAAACAGGACGTACATTCTGT | |
| SY3005 | SY3005F1 | CAGGAAA | | | 755 |
| | | GGGACCC | | | |
| | | TTGGAAA | | | |
| SY3005 | SY3005R1 | GTGGCAT | | | 756 |
| | | AAGCCCA | | | |
| | | AGCATT | | | |
| SY3005 | SY3005A1FM | CTGACCC | A | | 757 |
| | | AGTAAAC | | | |
| | | AAC | | | |
| SY3005 | SY3005A2TT | CTGACCC | C | | 758 |
| | | AGTCAACA | | | |
| SY4235 | | | | ACTGTAATCAAATGTGTATTGGGATTTACTGTACAGCTGGTCTGCAGACAGTAGACTTCTTTTAAGTGGC | 528 |
| | | | | AGCAAAGATTCAACACTTAAGGTAAGCATAATTTTCCTTTTCTCTCAAGAAGGGATATGTATCGAAAACA | |
| | | | | TTTTTAGTTTGGTTTATACACCTGAATACATCAATTTTCATTTGCTAAACTTGAACTGAGCTTTCATTATGT | |
| | | | | TTGGTTTTCAAGGTTTGGGATATTCGGACTCGTAAGTTGAAGCAAGATCTTCCAGGCCATTCTGATGAAG | |
| | | | | TATGCCTATTCACATGACAACTTGAGTGTTTTTTTTTTTCCTTTCAAATATTCTTCCGTTCTAGTCCCTGACC | |
| | | | | TAAGTCTATTTTCTTGCTGTAAAGGTTTTTTCGGTCGATTGGAGTCCAGATGGAGAGAAGGTAGCCTCTG | |
| | | | | GTGGTAAGGATAAAGTGTTGAAGTTGTGGATGGGCTAGGCTAATTTTTGGATGAATATTGGGAATCCAA | |
| | | | | CGAAGT[A/G]CAATCTCAATGGAGTTTTGCGGATGCATGGATTTCATGGAAATCAATGGTTGGTATTAT | |
| | | | | GTGGATGCAAGGTCTTTAAATTATATAGACAGCATAGAAGATATGTTTATGACTTATCAGAAATATTACT | |
| | | | | CTCATAGCAATTGAGAATATAGGCACTGGAAGAAGTTGCTCAAAGCGAGTCTCAGAACAGTGGTTGAA | |
| | | | | ATGTTTGGAGGCCTATCACATGCTTGCAAGATGTTTTCTTGTCTGCTTTGAAGTCTTTCTTGGTCTTCTCTT | |
| | | | | CCATTAATAGTTGTAGGTGGTATTGTTTTTCGGTGACAACAATGACTATAATGGGTTGAACTGTCATAGG | |
| | | | | GTTCATTGGTATGAACTCAGAAATTTTTGGGCAGTTTGACACACGATGATTTTTTGTTGGCCTTGCCACTT | |
| | | | | TAGTAGTAGTGTTTGAATGGAGTATTTTAATTTGAGATGTAAGAAAATTAAGCTGTGTCCTTTTCATTTTA | |
| | | | | TAAAATTGTGTTTGGATTG | |
| SY4235 | SY4235F1 | AGCCTCT | | | 759 |
| | | GGTGGTA | | | |
| | | AGGATAA | | | |
| | | AG | | | |
| SY4235 | SY4235R1 | TGAAATC | | | 760 |
| | | CATGCAT | | | |
| | | CCGCAAA | | | |
| SY4235 | SY4235A1FM | CATTGAG | G | | 761 |
| | | ATTGCAC | | | |
| | | TTCGT | | | |
| SY4235 | SY4235A2TT | CCATTGA | A | | 762 |
| | | GATTGTA | | | |
| | | CTTCGT | | | |
| SY4433 | | | | TTCTTGTTTATGAAGCTAAAATAACAAAAACAGAAAACATNAGCAACGTCAACCAAATTCATTTATAGCT | 529 |
| | | | | CTATTTTACTAGGTCTGTCAGAACCCTGATTCCATCATTATCTACACAAGAATCACATAATAAATGTCAAA | |
| | | | | GCAAAAGTAAACAACCTGACTTCACCTTATAATCCTTACAAGTTAAAATATGCAAATTAAGGCAATATTTT | |
| | | | | CAAAGATTTCAATTATTTCGATAAGAAAGCAATAATTAAAACCAAATCTCATAAAGGAGAAGCACAAGC | |
| | | | | ACCCATTAAAGAGTAACAATCATAAAAAAAGCAATAAATGACATGATCTATGAGAAAAACAATCAAATA | |
| | | | | ATAGTACAATACCATCATCATCTTGCTCCATTNCTTCTGGCTCATCCTCAGATGACGTCNAACCACCAGAT | |
| | | | | TGTCCAGTGCCTCTTCTGCTACTGGAGGCTACATCATTCAGAGCACTCCATATTGCTTCAGCATGCATCGT | |
| | | | | GTGATTTT[C/G]ATCATCAATACATGCATCAAAACTTCCTCGTACAGGGGATAGAGAACCTAGAAAAATT | |
| | | | | AGGTAATACATAGTCATTTTCAGAAGAAAAGTTGCTACACATACAATGTCTTTTTAAACTATAACAAAAG | |
| | | | | AGCAAGCCTGAGAATAGAACTTCAAAAAGAGCATCTCCACATACTTATCAACAGAGTTAGCTTAGTATAT | |
| | | | | ATGTTTTAGATTTACAAGTTTACAACCCAAACCATGCAGAGGGTCAACTAAAGAAGAATAAAAGCAATA | |
| | | | | TTATAGTTAAAAGTAGTAGATAAGTCTTAAAATAAAATCAGAAGATTATTTGTATACATAGCCATATACC | |
| | | | | CATACATGCAGACTTAATTAAATAGAAACATAAAGTTTAAGTGATCTTTACATACTGTCATCATCAATCAT | |
| | | | | AGGATGATATGGAGTCTTAGGCTCAGTAATTTTCTGCCTCACAGGTTTGTTTGCCTCAATTTCTCCAATAT | |
| | | | | TAGCCTCATCCCACCTTACAC | |
| SY4433 | SY4433F1 | TCAGAGC | | | 763 |
| | | ACTCCAT | | | |
| | | ATTGCTTC | | | |
| | | AG | | | |
| SY4433 | SY4433R1 | ATCCCCT | | | 764 |
| | | GTACGAG | | | |
| | | GAAGTTT | | | |
| | | TG | | | |
| SY4433 | SY4433A1FM | TCGTGTG | C | | 765 |
| | | ATTTTCAT | | | |
| | | CATC | | | |
| SY4433 | SY4433A2TT | CGTGTGA | G | | 766 |
| | | TTTTGATC | | | |
| | | ATCA | | | |
| SY4434 | | | | ATTGGCTTTAAGACTCTCCAACCCCCCCCCCCCACACACACACACACACAAAATAAAAATAAAAATCATA | 530 |
| | | | | ACTATAAACTACTAGCGCGTTTGGTTCCACATTTGTGATGCCATTTTCACAAGCTAATAGTTATATAGCTT | |
| | | | | CTGCATCCTCAACGAGTTTCCAAACAGGCAAACATACTATACTACACAAAACTATATATATATATAACAG | |
| | | | | ATAGAAAATGGGAAAAATAAAACCTTCGGGTTGGTCATTCTCGGAGAACTCCTTCTCGAGAACGCGATC | |
| | | | | GAACATCTTGGCCAAGGTGTTGTTGGATTCGGGCGACGAGCCATTGGGCATGTTCCCGTAGAACCTCTC | |
| | | | | CCGCGTTTCCTGGTCGGATCTGGCCGCGCCGCAAACCCTAGCGCAGATCNCGAGGCACAAAACGCAGC | |
| | | | | ACCAGAGCCCAAACGCGCGCCGTTTCTTCGTGGCCAGAATCGCCGTCTTCATCGTCGTTTCGATGCGAAG | |
| | | | | TAGCTGTGCGTGC[A/G]AGTTTCGGGTTTGTTTGGGAAATTGGGTTCTCCGCAAAACTGGGAAAAGGCA | |
| | | | | CAGAACACGGATATGGTAAGGAAGGAAGACAATGCAGAGAGGGAAAACGGTTTTTTCTGATTTCAGAA | |
| | | | | GTTTGCTACACTTTTTCTGGTTAGTTGCATTTGCGTTATAGCTGGTGTGAATGTCAATGTCAATGAAACTT | |
| | | | | TATTCATTTGACACTTTTGTCAAATGCTGAGGGGGGGTTTTGTGTAATTTCATTAATATTTTGGTGTCTGT | |
| | | | | GTGTTTTTTTTATAGGAATAATTATGAATGATTTTTGTATACAATAATTGTAGAAGTTAGAAATATTATGA | |
| | | | | TTTTTAATTGATTGATATTATATTATTTTTATAAAAAACTATGGTAATTTTTATAAAATTGAGAGTTTAATT | |
| | | | | TTTATGTAAAAAGTCTATTGTCATCATTTAATTAGAAAAATATTATATATAATATTTAAAGCTAATATAGG | |
| | | | | AAAGTCAATCATCTTTTTTATATA | |
| SY4434 | SY4434F1 | CGCCGTC | | | 767 |
| | | TTCATCGT | | | |
| | | CGTTT | | | |
| SY4434 | SY4434R1 | TTTGCGG | | | 768 |
| | | AGAACCC | | | |
| | | AATTTCC | | | |
| SY4434 | SY4434A1FM | ACCCGAA | G | | 769 |
| | | ACTCGCA | | | |
| | | CG | | | |
| SY4434 | SY4434A2TT | CGAAACT | A | | 770 |
| | | TGCACGC | | | |
| | | AC | | | |
| IGGY3105 | | | | ATGCATTAATGGTAATTTCAATTAGATTAAGAAAGTATAGTANATTCTTTTGCATGTTGGGATCTGATTTT | 531 |
| | | | | GAGATTTCAAAGACAAACTCAAACTCCTATNTATTCAGTGAGAGGAAGCTGATATGCTNTATAATTCTCA | |
| | | | | GGCATTGAGGACTGAATAGTTGAAACCTGAAACATGANNCATAATTNATGAAATGTGTTGGTCATAGG | |
| | | | | GTTTAGTAAGAGCAGACATGATTCCTTGGAGTACAATATTCTTTTCTATTACCATAAATTATGTGTTCACG | |
| | | | | ATTTCTTCTTCCCAAGTAAA[A/G]CAAACTAATAGTAATGTAGCTCCTTTGTGCTGCTTCTAATTAATTGC | |
| | | | | ATGCCAAAGTTTGTGTTTTGAACTGTTGTTGAGGNAAAATAAAAGNCCTGGTATTTCTAGACCAATTTCC | |
| | | | | AAGGAAAATGTGTATTTTTCACCAAGTAGTCTCTTGTTGAAGCTCAAGTGAAAAAATGCCAATTTTATAT | |
| | | | | AAATCACCACCAAAGCATCTNCTGCAGTATGAAACAAANATCAAATTATCATAATATCCTTTTTGCCTGA | |
| | | | | AAACTCCATGGTTAAAAGTAAAGATATTAATAAATGAAAGGAA | |
| IGGY3105 | IGGY3105F3 | AAACTAA | | | 771 |
| | | TAGTAAT | | | |
| | | GTAGCTC | | | |
| | | CTTTG | | | |
| IGGY3105 | IGGY3105F1 | ACTTCGT | A | | 772 |
| | | CAGTAAC | | | |
| | | GGACGTC | | | |
| | | ACGATTT | | | |
| | | CTTCTTCC | | | |
| | | CAAGTAA | | | |
| | | AA | | | |
| IGGY3105 | IGGY3105F2 | GAGTCGA | G | | 773 |
| | | GGTCATA | | | |
| | | TCGTGTC | | | |
| | | ACGATTT | | | |
| | | CTTCTTCC | | | |
| | | CAAGTAA | | | |
| | | AG | | | |
| SY3889 | | | | TAATTAATATCATTAATATATATTTATAAATTTTAAATTAAAACACAAATATTTACCAAACAGTTACTCTGT | 532 |
| | | | | CAAAGCTGACATTTCAGTTAGTTCCTTGGGGATTGAATGAGAAAATTTGTTATTTGAGAGATCCAAGACT | |
| | | | | TCCAGGCTAACCAAATTATCAAAAATTCGGGGAATATAACCACTAATGTGGTTGCCACTAAGATTCAGTG | |
| | | | | ACACCTGCAGAATCCACGGCATGCTTGGTATCACACCACTTAGCTGGTTTTCCCCGAGTTGGCGTTCNAT | |
| | | | | TAGAAGTTTCAAGTTTTC[A/G]ATGGATGTTGGTATGNAACCATTCAGATTGTTGCCTTGCAAGTTCAAC | |
| | | | | AAAGAAAGACTGCTCAAACTTGAAATCTCAGATGGAATTGATCCACCCAGAGAATTGCAGCTCAAATTC | |
| | | | | AGTATTGACAACTTATGAAGTTGGCCAATTTCAATAGGAATTGCACCATTATGCTTGTTCATTTGAAGCTT | |
| | | | | CAAGACTTGAAGATTGGCAAGATTACCTAGCAATGGTGGCAATACACCAGTCAAGTGATTCCAATTTCCT | |
| | | | | GCAAGATTCCAATTCAACCAGTATGGTTCCGGTCAAGTCATT | |
| SY3889 | SY3889F1 | GCAAGGC | | | 774 |
| | | AACAATC | | | |
| | | TGAATGGT | | | |
| SY3889 | SY3889R1 | TCCACGG | | | 775 |
| | | CATGCTT | | | |
| | | GGTATC | | | |
| SY3889 | SY3889A1FM | CCAACAT | G | | 776 |
| | | CCATCGAA | | | |
| SY3889 | SY3889A2TT | TACCAAC | A | | 777 |
| | | ATCCATT | | | |
| | | GAA | | | |
| IGGY3103 | | | | GGGCCATTTTCCAGCTGACAAGATCTTGACGTGATAAAGGACTGCAGAAATTAATATAGGAGAGAAGG | 533 |
| | | | | GAGNTCACAATTGTAGCCNACGCCATACAAAGTTAAAAGCACATCTCAAACAGAGGGTAGTGCAACTGT | |
| | | | | GCACTGAGAAAAATGAGATGAAATATTAAGTCTATTTTTCTATATAAAAAAAGGAGAATTGCAAAAATTT | |
| | | | | TGAACAACAAATGCACAACTGCAGAGGTTAAAAAAATGTAGCCACAAATTAGATATCCAAATTCAGACA | |
| | | | | AAACATACCTTTCAGGGGAGAAGT[G/A]AAATGGGCTATCATCTCCATCACCAAACAACTGTATATCACG | |
| | | | | TCTTGAAAGCCTGCTTTCAATAAGTCCAGCTTCTGCCAAGCCTGAAAATCAATATTTGATATTATTTAGAC | |
| | | | | AGAATATGAGTATAGCAACAAGTTGTATTGTTAAAAATTATTATGATGTCCTGTACTGACCTTGAATGGA | |
| | | | | AGAAAAATCAGGGTCCTCGGGAGTGACATCATCAAATGCAAGCTCAGTAGCATTGTCTATNTACATGGC | |
| | | | | AGGATACACTTTTGAAACTGTGCTCCTAACAAGTTACCAGAAAGGCAC | |
| IGGY3103 | IGGY3103F3 | CTTCTCCC | | | 778 |
| | | CTGAAAG | | | |
| | | GTATGT | | | |
| IGGY3103 | IGGY3103F1 | ACTTCGT | A | | 779 |
| | | CAGTAAC | | | |
| | | GGACGG | | | |
| | | GTGATGG | | | |
| | | AGATGAT | | | |
| | | AGCCCAT | | | |
| | | TIT | | | |
| IGGY3103 | IGGY3103F2 | GAGTCGA | G | | 780 |
| | | GGTCATA | | | |
| | | TCGTGGG | | | |
| | | TGATGGA | | | |
| | | GATGATA | | | |
| | | GCCCATT | | | |
| | | TC | | | |
| IGGY3106 | | | | ACACCTCATACCAAGCATTAGACATGGTTGTTTCCTGTGAAGAGTGCCAATAACAGCATACAATAAAGCA | 534 |
| | | | | TGGAAGTAGTGGGAACAAATTAATCAGCATTCTTCAATATAGAAAGTAACTTCCACGTCAAATTCAGAAA | |
| | | | | CATGTATGGACTTTAAATAGAAAANCTGATTACCTCTTTGTCATCCTCTTGATTCACCCAAGTAAATTGTA | |
| | | | | CTTTATACTGTAAATGGCTTCCTGCACCATAAACACAAGCAATAATATTTGATACTTGTCTTGCAGATAGC | |
| | | | | ATACATCATATACAAATG[G/A]ACTAACACAAGCTTTACCATTCTTAACTAATCCCAGAACAACAGGCAA | |
| | | | | GTAATCCTCAAGAGCCTGCAGAAGGTCAGCCAGTGTTGAACCTCCTGCAGAAGAAATCAAACGATGCCG | |
| | | | | ATGCATCAATNAATTAGAGTAACTTATTCATCATAAAATCATGTTGTTGTTGGTAAAACTAACCATGCTG | |
| | | | | AGTTTTTCTTTTTGTTCTTGTAATTGTAGGACCTTCTTGACCAGCCATTACAACTATACGCGTTCTAAGAGC | |
| | | | | AGACAGGCGTTCCACTATATTCTTGGACAAATAATCACCAA | |
| IGGY3106 | IGGY3106F3 | CTAACAC | | | 781 |
| | | AAGCTTT | | | |
| | | ACCATTCTT | | | |
| IGGY3106 | IGGY3106F1 | ACTTCGT | A | | 782 |
| | | CAGTAAC | | | |
| | | GGACGTG | | | |
| | | CAGATAG | | | |
| | | CATACAT | | | |
| | | CATATAC | | | |
| | | AAATGA | | | |
| IGGY3106 | IGGY3106F2 | GAGTCGA | G | | 783 |
| | | GGTCATA | | | |
| | | TCGTGTG | | | |
| | | CAGATAG | | | |
| | | CATACAT | | | |
| | | CATATAC | | | |
| | | AAATGG | | | |
| SY0871AQ | | | | AGAGGAGGTACCGAGGCCACCCCCTCCGGTCATTTCCCGGGAACCCGAACCAGATCTGGGTGGTGGAG | 535 |
| | | | | ATCCACGTTCTAAAGTTGAAGACGATCTAGATATCGGTGAAGACCTGTTAAAGATATCACAGCGTCGGA | |
| | | | | ATATTGA[A/T]GAAATTGACGAGGACATCCGGAGCAGAGGAAGCAATGGACCTCCCCATAATACTTCTG | |
| | | | | AAGTAGATTCAGTTTTGGGTTCAGATCGCCGGGCCCCAACAATTCGATCTGAAGCAAGGCACTCAAGTG | |
| | | | | AGGGAAGAAGTGAAAGCTGGGAAATCGGGTCTGAGGTCCTTGCCAATTCAACTGTAACTGAAAGCAGA | |
| | | | | AGCTATGTTGTATCAAAGGAGGTGCGCCAAAAACTTGGGGGTTCTTTCTGAGATGTGGGGTTTCTTAGA | |
| | | | | TATTAAAAATGGAAAAGGAAATGGTGTTTGATCAGATTATAGGAATGGGAATTGAATTGATTGAGGCAT | |
| | | | | TAAGTACAAAACTTATTTGATCTTTTTTTGTGGTAAGGTTAGCTCTTTTGG | |
| SY0871AQ | SY0871AF1 | GAGGTCC | | | 784 |
| | | ATTGCTTC | | | |
| | | CTCTGCT | | | |
| SY0871AQ | SY0871AR1 | TGGTGGA | | | 785 |
| | | GATCCAC | | | |
| | | GTTCTAA | | | |
| | | AG | | | |
| SY0871AQ | SY0871AA1FM | CTCGTCA | T | | 786 |
| | | ATTTCATC | | | |
| | | AA | | | |
| SY0871AQ | SY0871AA2TT | CTCGTCA | A | | 787 |
| | | ATTTCTTC | | | |
| | | AA | | | |
| SY0099E | | | | TAAAATTTGTTAGTTTGTGGTGTTGCAAGGATCCCCCCTACATAGGGTGATTGGTTGCTGAGACATCACA | 536 |
| | | | | AGGATTACCCTAAACCAAACAGTTTTTTCTAGTGGGTCTTGGACTCTGACACTGTGGCTTTAGACAATTA | |
| | | | | AAATAAATGTGTTTTAGATTGTTCGTTTTCCATTATAATTGATCTTAGTCTTTATACCTTAAAAAAGATGGC | |
| | | | | CTATGAAAGTCTCCTATTGTTTTAAATAAANAAAAAATAGGCATANTACAATTATTAGTTTNAAAATATA | |
| | | | | CTACAGACTAAGAAAATTTTCCACCAAAAATTGGGGACCATAACACATTTTANCAAGAGAAAAAAAATA | |
| | | | | TATAATTAAATTGACTTAAACAGCGGTTAAGAGACAGGATTCAGAGGAAAGATGAACCAACTGAAAGC | |
| | | | | ATACCTCATGCATGTGCCACAGCGAAGCATGCCAGAGAGCCCTATGAGCCCATCTAGCCCAAAATTCCAT | |
| | | | | ACCCACCTGAAAATCAGACATACCCAGTTGAATACTTGAATCTATCACTCAAAATTCACAAACAACTCACC | |
| | | | | CAATTCTGAACTCAAAAACCATTCAAAACCCGTTTGGAATCATAATTAGTTTAAAGGATAACTCACAGCA | |
| | | | | GCTCCCACTGATAGAGCAAATGTGCCAAACATTTCAGACCAGGCATTTCTCCACCCTACACCAACCAAAC | |
| | | | | CCATTTCATTATAAAATTATCTTCATATGCAAATCATCATCAATCAATCATGCATGACACCAATGTCAGGA | |
| | | | | CATAAAAAATAAGGTTTTAAAAAACGATCCGCAAAATAAAAGTCAAGTCTTTGTGGTGTCTGTCATTCCA | |
| | | | | ATTGTGGCCGCATCACCAGCGTTTGTTTATAATTTTCTATGATATCCAAGATCACAACTGTGGTCGTATCA | |
| | | | | GCAGCATTTGTTTATAACCGCGACAAAACCACGACCAGAGCCGTTTTTTAAAAAACTTGCATAAAAATAA | |
| | | | | GAAGAAAAAAGAAAGAATCAATTTTAAGGCACCACCTACCTCCATTTGCCACGAGAATCTGCAATAAAC | |
| | | | | AGCGAACACTGCCATGGATGTGATGCCGAAGCTAGACATGACAGCAGCAAC[A/G [symbol] AGGTAAGTGAACC | |
| | | | | TCTCAGACTCTTTTCTGGCCANTTTCTCAGCCAACTTTGGTGACAGAACTTGTTGAGCGGGAGGAGGAG | |
| | | | | GAGGAGGAGGAGGTTGTTCTTGTGGCTCAATTTCCATGTGGGTGCCTTGTTTTGGGTCCTGCATGAGGA | |
| | | | | CACAAACGGTGAAAGTTGAAACTTTTTGGGTTCTTGGTGATGCTGTGTGGTGGAAAATTCTCATGAGAG | |
| | | | | GGAAAGGGAGTGTTGTTGGGATTGATTTGGGGATCTTCAATGGAGGTTGGTGGAAACGGAGGAGGGA | |
| | | | | CTTCATGGTTATGGC | |
| SY0099E | SY0099EF1 | TGTGATG | | | 788 |
| | | CCGAAGC | | | |
| | | TAGACATG | | | |
| SY0099E | SY0099ER1 | CTCAACA | | | 789 |
| | | AGTTCTG | | | |
| | | TCACCAA | | | |
| | | AGTT | | | |
| SY0099E | SY0099EA1FM | CAGCAAC | G | | 790 |
| | | GAGGTAAG | | | |
| SY0099E | SY0099EA2TT | CAGCAAC | A | | 791 |
| | | AAGGTAAG | | | |
| SY4353 | | | | AAATTTATAGTGCAGCCAAGGCATCCGAAAGGTCCCCTTAAAAACCGGTTCATAAGGGGGTGGTCTACC | 537 |
| | | | | TAGCTATATAAGCACTTATCATGTTCATGAATTACCCGATGTGGGACTATTCTTAACACGCCCCCTCGAGC | |
| | | | | CAGGGCTCGTCACCACAAAGCGAGGGCTGGCGGCACCCACTGGACAGAAGTAGAAGATGGCTCTGATG | |
| | | | | CCATATTAATGAAACGAAGCAGCGTGAAGAAAAGATACGTAATCAGTGAACGTAAAATGATCAGCCTCA | |
| | | | | GCTTGCTTGTTTATTCATTAATAGTGGAATTTATATACATCTGCAGCATGAAGTTGTTATAACCGACTAAC | |
| | | | | TAATCTAACCAACTCTTTAACTACTAACTGAAAAGTTGTTATAGCTGCAGTTATACTGTTAACAGAAATAC | |
| | | | | TTTACTAAAACTTCCTCAGGCCTCATGATTGTCTTTTAGGAGTGGAAGATACGGGGAAAAAATGACATG | |
| | | | | GCTATTTACTGC[A/G]TTAGTACATCATGAACAGCCGGGTAAAATTTAATGGTGTTTCGTTTCAGGTTTA | |
| | | | | AGAATTAATTTTAGGTCTAAAATTAATTTTAGATGAATTTTTATATATTTGATTTCATTTAAAGAAAAATTT | |
| | | | | AAAATTAATTTTGATCGAATGAGTTGAAATAACTTTTACATTGGATAAAAAAAGTTATACTAAATTTTAAT | |
| | | | | TATAATTATTTGTAAAAAAATATTTAATTGAATAATTATGGTTTTATTATTATGAATGTTAAACAGGCATT | |
| | | | | GATCTGGTCTTGTTAATTTATAATGATTTGAAATTAATTTTGACATATTTAAAAATATTTTAAAAGTTAGA | |
| | | | | CCTGAAAATTGACTCAGATTATATTTTTAGTCATCTTAATCAAATCAAATATCATATTCATTTATAATATAT | |
| | | | | ATTTTTAATAANTATATATTAATAATAAAATTCTAACTTAATACTTTGTGTGATGATATTGCATTTATATTA | |
| | | | | TCATTGNTGTTNTTGTT | |
| SY4353 | SY4353F1 | CCTCAGG | | | 792 |
| | | CCTCATG | | | |
| | | ATTGTCTT | | | |
| SY4353 | SY4353R1 | CACCATT | | | 793 |
| | | AAATTTT | | | |
| | | ACCCGGC | | | |
| | | TGT | | | |
| SY4353 | SY4353A1FM | CATGATG | G | | 794 |
| | | TACTAAC | | | |
| | | GCAGTA | | | |
| SY4353 | SY4353A2TT | TCATGAT | A | | 795 |
| | | GTACTAA | | | |
| | | TGCAGTA | | | |
| SY4354 | | | | TGTTATCGGCTCTGTAGCGGAAGTTCTCATAGGTAGTCTGCAAAACAAGTTATAATCAACCAGGTGATG | 538 |
| | | | | AAGATATTCTTGAAACACAAGGGCTCTTAAGAATCAAAGTTTCAGAATATGGTTTTCTATAAAATTTAGC | |
| | | | | CTTTTCTTGCATACTATGGAAAGCAACTAAAAGGGATGCCTTTCTTCTCGCATCTTGGTACAAGCGAATC | |
| | | | | AGGCTTACACTATTAATGCAGTTTGGATTGAGGGAAGAACACACAGAATTACCGACCTTGTGAGATTTA | |
| | | | | AAATTACTATTTGTTTGGATGAAAGGAAAAGTATGAGGGACAAAAATGAGTTTAAGATTCTAAAGTTAT | |
| | | | | CTTCTCAAAATTTCTTTCCTTTCCATTGCCTGATTTTTGGCTACCTTCAAGTACAACCATTNAGTATTTTTTA | |
| | | | | ATGTCCAGTTTTACTTCATCATTTTGTCCACATCCACCCAACATGAAGTCATAAACTAAATTTCCTATAAAT | |
| | | | | AAGGAACC[A/T [symbol] IGGTGAATTTAGAAAATTAAAATATTTTTTAGAGAGTGGTTCCAATTTAATTTCACCGA | |
| | | | | GGATAATTCTACTAATGCTTTTTATTCACGTAACCAGTTAGTCTTCAGGTACAATGAACAATGAGATAAA | |
| | | | | CCAGATATTATATTCAGAAATGTAAAAGATATCAACTGACCTGATTTGTGCCAATAAGGTACAAATGAAA | |
| | | | | GCCAGTTAGTCCACCGACAAACCACAAAGAGATGAAACAATATGCCATTAATATAACAGATGCAGGGGA | |
| | | | | TTCTTTCATTGCCTTCCAAACTGTCCCCTTATAATGATCCATCAGAACCTTGATGTAAAAAGCTGAGATGG | |
| | | | | AAAACACATAGATACATAGAATAGTTGCCGAAGAAACAAACAGAAAGAAGTAACGGTAGTTCCTCTGCA | |
| | | | | AATTGGAAAAGCCATAAACAAATAAATAAATTACAAGAAACACGTGAGCAAGCCAGGAACATCAATCG | |
| | | | | AACAAAGCAGTTATCATAACATCAA | |
| SY4354 | SY4354F1 | CCACATC | | | 796 |
| | | CACCCAA | | | |
| | | CATGAAG | | | |
| SY4354 | SY4354R1 | CCTGAAG | | | 797 |
| | | ACTAACT | | | |
| | | GGTTACG | | | |
| | | TGAA | | | |
| SY4354 | SY4354A1FM | CCTATAA | A | | 798 |
| | | ATAAGGA | | | |
| | | ACCAGGT | | | |
| SY4354 | SY4354A2TT | CCTATAA | T | | 799 |
| | | ATAAGGA | | | |
| | | ACCTGG | | | |
| SY4329 | | | | ACATGTTTTCCATACATACAATCACCACAATCACAATTCACACATCATTTTATTCCATCCTCATCTCATGTTT | 539 |
| | | | | TACATCTAAAACAACAAATAACGTGTGTATATATCATTATTGCATTATCTCAAACTTGTTTAACCCGATTT | |
| | | | | ATTTTATCTTTTTCCAAACATGCCAACTTGTTGAATAAAACATGCACAATCATTGACATGTATTAAGTATTC | |
| | | | | ATTATTAAGAATTCATCCTTCTTTTCATCCTGCCACCTAGTGCAGGAGAACATCATTCTTGAATCTTATATA | |
| | | | | GCATGCATATAACACTTCATTTTTTTCATTTTGTTTAAACTAAGAAATTTTCATTTTCCAAAATCATATTATC | |
| | | | | ACTAGGAAGATTTCAATAAACTGAACATGGTCATTACTCTTTAGTCACATACTCACATACCAAAATTGGCT | |
| | | | | ATAAAATAGTGCTACAACTACACCTACACCACCATGGTTCATNATTAAGCCATTCATTACACAATAGC[A/ | |
| | | | | C]TTTTCTACACTAGTGAAAACATTTTGTGTGTTTTATCTAAATCATTTTTTACTTCAACCAGTGACAGAAGA | |
| | | | | AGCCCCCACCTCCACAACTACAACTGTAACCGCCGTCACTGAAAACCCACCAGGAGGTGGGGAAAGGA | |
| | | | | GGAGAAAGTACAGAGGAGTGAGGCAGAGGCCATGGGGAAAATGGGCAGCAGAAATCCGTGATCCACA | |
| | | | | CAAAGCAGCAAGAGTTTGGCTAGGCACATTTGACACAGAAGAAGCAGCAGCAAGAGCCTATGATGAAG | |
| | | | | CTGCATTGAGGTTCAGAGGCAACAGAGCAAAGCTTAACTTCCCTGAAAATGTAAGAGCAGTTCCACCCA | |
| | | | | TTCAACCTTTTCAAGCCACCACTAGGCTAACCGTTTCTGATTCCACCACCTCTCAATTCCGGCCACTCTCCG | |
| | | | | CGGTGGCGCCACCCTTCATTCAGCAGCCACAGATTCAGGGCTCCTCTGACTTGATCAGAGACTACTTGCA | |
| | | | | ATACTCTCAGCTTCTA | |
| SY4329 | SY4329F1 | AGTGCTA | | | 800 |
| | | CAACTAC | | | |
| | | ACCTACA | | | |
| | | CC | | | |
| SY4329 | SY4329R1 | GGGCTTC | | | 801 |
| | | TTCTGTCA | | | |
| | | CTGGTT | | | |
| SY4329 | SY4329A1FM | TCATTAC | A | | 802 |
| | | ACAATAG | | | |
| | | CATTTTC | | | |
| SY4329 | SY4329A2TT | CATTACA | C | | 803 |
| | | CAATAGC | | | |
| | | CTTTTC | | | |
| SY4349 | | | | TTGTGTTATTGTCATATTCTCTGGAATTCATGTTGGGATTGCTTGTATTCTGTAGGTCATGCTGAAGCTGT | 540 |
| | | | | ACTAAGTGTTGCCTTCAGTCCTGATGGGCAACAACTGGCAAGTGGTTCTGGTGATACCACTGTTCGATTT | |
| | | | | TGGGACTTGACCACTCAGACACCATTGTACACTTGCACAGGTTTGACATTTAAAGATAATAAGTTACTCT | |
| | | | | GTTATCTGCTAATTAAATCAAGAAAAACTCAATTGATGTTTTGTTATCCTTCTCTTAGTATAAGAATAAAA | |
| | | | | ATGATCAATTTAACTCATGCAAAACAAAGTCAGAATTGTTGGAAGTTGGTAATGCTCATGTTTGTGTTGC | |
| | | | | TAGAAAAAAAAATTCTCAAAGTTAGAATTGTTGGAAGTTGGTAATGCTCATGTTTGCGTTGCTAGAAAA | |
| | | | | AAAATAATTCTCGATAGTTTGTGTAATCTGTTAATACCCAGTACTATGCTACAAGGGAGGGGATGAGAA | |
| | | | | TCAACATGTG[A/G]GTAAGGAGAAAAAATGGGAAATAAAGGGAATGTTCATGCTATTAAATCTTGGGC | |
| | | | | ATAATCAAATGCTGAGATGGCAAGGAATAGTGGGAGGTGGGATAAGAGGGATATATAGGGAAGGTGA | |
| | | | | AGGCAGGAAATTATAGCATAAGAATTTTAGAGTCATATATAGCATTATAGCTGTTTACATTTTATGCAGG | |
| | | | | TCACAAGAACTGGGTCCTTTGTATTGCATGGTCACCAGATGGAAAGTATCTTGTAAGTGGGAGCAAGAC | |
| | | | | TGGAGAACTTATTTGTTGGGACCCNCAAACTGGAAAGTCATTAGGCAATCCACTAATTGTAAGATCTTCA | |
| | | | | ACCTTGAATACCAATTTCTATTAAAAAGCTTGTTTTGTTTTTTCCTCTTAATTTTACATATCATGCCAAACTT | |
| | | | | CCAAGTTCAAACATTCAAAGATTCGAACAAAGATTTATAGAAACTTGAAGCTCTGAACACTGAATAGTCA | |
| | | | | AATGTGGTTATGAAGATTGAAAGCAGT | |
| SY4349 | SY4349F1 | CTGTTAA | | | 804 |
| | | TACCCAG | | | |
| | | TACTATG | | | |
| | | CTACA | | | |
| SY4349 | SY4349R1 | CTCCCACT | | | 805 |
| | | ATTCCTTG | | | |
| | | CCATCTC | | | |
| SY4349 | SY4349A1FM | TGAGAAT | A | | 806 |
| | | CAACATG | | | |
| | | TGAGT | | | |
| SY4349 | SY4349A2TT | AGAATCA | G | | 807 |
| | | ACATGTG | | | |
| | | GGTAA | | | |
| SY4358 | | | | TATTAAAATTTATAAAATTATGTGCCACGGTAAACTGAAGAATATGTAACTTTGTGTGCACAAAAAAAAA | 541 |
| | | | | TTAGAAAAAAAAGGAAGTTGAATTATTTTAAAAACAAACAAAGATCATAATGACTTAATATAAAAAATTA | |
| | | | | TTTAATATAGTTAGTTAAGGTTACTAACTTAAACAAAACAAAAACATTAAATATATTTAAATGTTTTGCAT | |
| | | | | TGGCTGAATCATTAGTTGGTTTTCAATTATGGAAGGGCCAAAGATCCAACATAGCATGTTCATAATCTCC | |
| | | | | CTTATACGCTGGCAGTGCATTTTATTCTCAAAATGCAACGGAAGTATTATCATATATAACCATAGCTTAAT | |
| | | | | GCCACGATGTTGTTAAAAAGTAGTTTAGTGCAATAGGCCATATTTATTTATTTATTAAAAATGGTTATACC | |
| | | | | TGATGGTTAATTGAAATACAATGATAAGGATTGGTATAATTTCATAAGATACTGATAGAGTGTATCTTAT | |
| | | | | AACTTAC[A/C]GTCGTACAGGATTTGCTTTCACAATGAGTGTTTGCTTGCACTTTTGAAGGCCGATGGTC | |
| | | | | TGGTGGCTAGGACATTGAGAGGANTGAACTTCTCTTTATTATGCTCTTTGCTAAGGAAACCATAATAATG | |
| | | | | CACTGTTATGAGGGCCAAATCAGTATAGCATATGTGTATGGTAATTAAATTATAAAACCAAAAAAATTAA | |
| | | | | TCCTTAACAACTGCTCATCATTTCGAACTTTGATGTTAAGGATAGGTCAAATTTGTTCCTTACTTGGGCAA | |
| | | | | ATAATTATCATTTTGGTCGGTTATTCTCAAATATATATATATATAATTATTTTGATCTCTAATGCACATCAA | |
| | | | | TTAATTTAAGTTTTCTTTATATTACACATCAATCATTTTGGTCTGATAGTAGTCTTCAAATTAANTTATCAA | |
| | | | | ATTAATAATGATAGTATTCTAGATTTGATTAATTCTTTAGTCTCCCTAATATATATATATATATATATATTG | |
| | | | | CAGTGAGATGAAGG | |
| SY4358 | SY4358F1 | CCATAGC | | | 808 |
| | | TTAATGC | | | |
| | | CACGATG | | | |
| | | TTG | | | |
| SY4358 | SY4358R1 | ACCAGAC | | | 809 |
| | | CATCGGC | | | |
| | | CTTCA | | | |
| SY4358 | SY4358A1FM | AATCCTG | C | | 810 |
| | | TACGACG | | | |
| | | GTAA | | | |
| SY4358 | SY4358A2TT | TCCTGTA | A | | 811 |
| | | CGACTGT | | | |
| | | AAG | | | |
| SY4324 | | | | TGGATGATGATGATTCTCTAGAAAAATTCAAACTAATCAGTTTTGAGCTAAACTCATTCATCTTCTCAAAA | 542 |
| | | | | AACCCACTTTCAAACCTTAACATTAATCATGACTTATTCAAGCTCATTAGTGATGAGAACTCATCAGTGTT | |
| | | | | GTTGCATCGTTTGAAATCAATGAGAAAGAAGTTGGGAAGGAAAGTTAAGCTAATGACATATTTGAAGA | |
| | | | | AAGCATCAGAAATTTGTGTTGCAGTGTATGACTTAGTGGCTATCACTGCCAATGTTACAGCAGCACATAC | |
| | | | | TTTAAGCACACTAATCATGGGGCCAACAATTCTCAACTTTCCATGCAAGAGTCTTAATAAGAGAGAGCTT | |
| | | | | CCACACTTGAGGTTTTCAAGAAGAAGGTTTCTTAGTAATGTTTGTGATCAGCTTGATATAGCAGCCAAGG | |
| | | | | GAACTTATATATTGAATAAAGACTTTGATACAATGACTAGAGTTGTGGCTCGGCTTTATGATGAAATTGA | |
| | | | | ACATAAAAGG[A/G]CAATGGNGCAATTCTTTTTGGACAAGAAGGATGATAAATTCTCTTTGCAAATGGT | |
| | | | | GAAGGAGCTTAAGAAAAGTGGTGATGGGTTTAGGAAACAAGTGGAAGAACTTAAAGAGCATGTGTACT | |
| | | | | TGTGCCTTGTGACAATTAACCGAGCAAGATGTTTGGTTACTGAGGAAATGACAAAAATGTGTACAGAAG | |
| | | | | GCATTGGGAGTGTAGACATGTAAATTATTATAAACGTATGGATCTTGTATAGTGTGGTTAAGATTTGTTT | |
| | | | | TGTTTTATTTAAATTATTTATTACATTAATTAGTGGAGGTACAGAGAGAAAAAGGTGAAAGTAGCCATGA | |
| | | | | CAATAAAAATTATCATGAGTTTCACTCTGTTCAGACTTAGATGGTAAAAACTCACTTTGAATAAGATTGTT | |
| | | | | GGGATCAATTTTCTTCAACATTAGAAACCGGGGTCTGGTACGTAAGTAACTACCTGCCCCAATATATATA | |
| | | | | TATAGAGACGACTTTCCATTAAAGAGA | |
| SY4324 | SY4324F1 | GTTGTGG | | | 812 |
| | | CTCGGCT | | | |
| | | TTATGATG | | | |
| SY4324 | SY4324R1 | ACAAGGC | | | 813 |
| | | ACAAGTA | | | |
| | | CACATGC | | | |
| | | TC | | | |
| SY4324 | SY4324A1FM | TTGAACA | A | | 814 |
| | | TAAAAGG | | | |
| | | ACAATGG | | | |
| SY4324 | SY4324A2TT | TTGAACA | G | | 815 |
| | | TAAAAGG | | | |
| | | GCAATGG | | | |
| SY4234 | | | | ATATATAGTGTTGAACCTTGCTCCCTGTTTGTTGCGGAATTTTCTGCGAGTGGTACAAGAAACGGCGTTA | 543 |
| | | | | GGGTTTGATCTGGCTTGGTGAGAGGTAATCGTATATAGTGTTTCGGTGGAAACCAATGGATTCACCGAT | |
| | | | | GGGCTTTATCTTTGGGCCGATCAAGTAGCCCAGGACCTTTACTTATTGTAAAAAATAATTTGAATAATAA | |
| | | | | AAAAATTAAAAGATACCCGCACATAATCCTTTTAAATAAATTCATATAATTACTAAATTTTAATCCTGACA | |
| | | | | TTGCCTAAAAAAAAAAACCTTTAATCCTGACAAACATTTAAGTTTTTATAATTCTTGAATACGTTGATTTTT | |
| | | | | TTTTTTCATTATTTACTCATATATGGTCGTGCTTTCATATTGGTTCCTTTAAATATTATATATTTAGCATTTA | |
| | | | | GGTGGGACTTTATATTAAGACTTTATAAAACTTAAAAAATAAGCACAGTTTTATTTTACAGTAAAAAAAA | |
| | | | | AGAAA[A/G]AAGCACAACTAATGCCTTAAAAAAACCCTTTGCACACTCGCAAATTAATGATGACGCTCCC | |
| | | | | AGCATAATAAAGCCAGAAACAATCTGAAGATTATGCTCACCATAGCAACTTCCTCAACTTCCCACATCCA | |
| | | | | AAATTAATGAATTACTCTATNGCATTCATCTATTAATCCCGTTAACAATTTATAAAATAAAAATATAAGAG | |
| | | | | AAATACATGTGAAAAAAATAAATAATCTTTAAGATATTTCAAACAAACATTTTTTCTTTTCTTTTCAAAACC | |
| | | | | TCTTTTTAAAAAAATATTAAAAATACATCTTATGAGAGAAAATATTTTAATAAAAAAATGATTATATTTGA | |
| | | | | ATCGTATAATCATTTATGAAAAATTAAAATTTAATGTCTAAAACTCATATAATTTTAATAAGTGATTACAT | |
| | | | | GATATTAAATTCTAATTATTCATATTTGATTAATTAATGATTACTTTCATATAACATTTTTTTCTCATATGAG | |
| | | | | GTCAATTATCC | |
| SY4234 | SY4234F1 | GGTCGTG | | | 816 |
| | | CTTTCATA | | | |
| | | TTGGTTC | | | |
| | | CT | | | |
| SY4234 | SY4234R1 | GCGAGTG | | | 817 |
| | | TGCAAAG | | | |
| | | GGTTT | | | |
| SY4234 | SY4234A1FM | AGGCATT | G | | 818 |
| | | AGTTGTG | | | |
| | | CTTCTT | | | |
| SY4234 | SY4234A2TT | AGGCATT | A | | 819 |
| | | AGTTGTG | | | |
| | | CTTTTT | | | |
| SY4231 | | | | TGAGATAAATTCATAAAATGTGATAAGACCAGCAACCTAACACTAGCTTGATGCAAATTTTAATGCTCTG | 544 |
| | | | | CCTGCAACTTTTGGCGAAGCAAAAGTGCATGTACAGAAGGGGTCTAACACAAGGATGCAAACATATTG | |
| | | | | GTAGTAACTAGGAACAAATAGTGTATCTTGCATTCTTCTTCTCATGCACAAATTTGATTCACAGTAAGTAT | |
| | | | | AGCTGCACTAGTAGATTATAGTAGAAAGCATTGCTGAAGATGAAATATTAAGTGGTCAAATTTTAGATA | |
| | | | | TCACAGATCCAAGCACAATAATGAAAAACAAACAGCATTACAAATATATTATACAATAAAATACAACAAT | |
| | | | | ATCATACTAAGTTTCTGGAAACAAGATTTTACAGTTCATTACTTACATACAAAACTTTGTAAACTCATATA | |
| | | | | ATAAAGAAATATTTCGCACTTGTAGCAGCCAACAATGCTTTCTTCTCTGATCAGAAGCGTGCAGCACATC | |
| | | | | TGCAACAATCA[A/G]CATTTTTTTTTACAAAAATTAATGTGGTGGATTACAAGATATAGACAAAATTATA | |
| | | | | TTTGTTTATATAAAATTGAAAATGAAAGTATACATGCAGGGGCCATGCCCGCCCAAGTTTTTCACAAAGT | |
| | | | | GTATTTAAAATGCCATGTAAAAAGAATTACGGTCCCCTTAAAATTTATGAAAAAAATATTGAAAATAACA | |
| | | | | CACGTAGTTAACACTTAGGGGGAGAAAAGGAAAAAAAGAGAGAGAAGAGTGTGAGTATAATAGATAA | |
| | | | | TATAATGTGATGGTGTGATAGTAAAAAGTGAGATAAAAAGAAATTAAAGATATTAGTAAAGTATTTGTA | |
| | | | | ATGTTGAAGTATCTATATATAATTATCTAAAATCATTTTATATATGTGGTTATTTTTTAGTTTCTCATACATT | |
| | | | | ACCTCTATTAACATATTTAAAAGTTAAATAACTAATTAATCAAAATCAATAAAAATAATGAAGTAAACTAT | |
| | | | | TTTAATTAAAAATGTCTTAAAATAA | |
| SY4231 | SY4231F1 | GTAGCAG | | | 820 |
| | | CCAACAA | | | |
| | | TGCTTTC | | | |
| SY4231 | SY4231R1 | TGGCCCC | | | 821 |
| | | TGCATGT | | | |
| | | ATACTTTC | | | |
| SY4231 | SY4231A1FM | TCTGCAA | A | | 822 |
| | | CAATCAA | | | |
| | | CATTT | | | |
| SY4231 | SY4231A2TT | ATCTGCA | G | | 823 |
| | | ACAATCA | | | |
| | | GCATTT | | | |
| SY4224 | | | | CGCAACATGGCACGCCCCAAACAGAACAGCTGTCTTTGTCCCATGCTCCAGTTTGATCCCTCTCCAACAA | 545 |
| | | | | CTAGGTAACAATTTCGTACGAATCATTACTAACATGGGAAGAAGTGCTTACATTATATATAGTTAAAATA | |
| | | | | GTGAAAGTTGAAAGTGCACCTGAGGAGTTTAAGCCCTCTTCTTTCTCTTGAACCACTTCTTGCAATTGACA | |
| | | | | CTTGCCAAGAACCTGAGTTTATATTAAGCAAGAATTAGTCCATATTTGCTCTGAATAGCAAAGAAAAGCA | |
| | | | | GAGTGTAGCTCTAGTAGGTGTTACCTCCCATATCTCTTGATCAGAATGTTGAGATAAAGGGTCCAAATTA | |
| | | | | TATCTAACAGTCCCATTGAAAAGAGTAGGATCCTGAGGTATAATACATAAACGTGACCTCAAATCTTGAA | |
| | | | | GGCCAATAGAAGAAATGTTTATGCCATCAACAACGATTTTTCCGCTTGCTGGCTCCATGAGACGAAATAA | |
| | | | | AGCACTGAT[A/C]AGAGTAGACTTCCCACTGCCTGTCCTGCCAACAATACCAATCTTGTGCCCTCCTTCAA | |
| | | | | ATGTGCAAGTGATGCCATGGAGTACAAGTGGCCCTTCAGGCCTATATCTTATCTGTTTTGGTATAGACCA | |
| | | | | CAAAATTATGCATTTCATTTGAACTCCCATGCTTTTTAATATTATATATGTTGCAAATTTTAAAGTATGCAA | |
| | | | | TTAGACAAATACCTGCAGATCATTTATTTCTACTTTGCCTGCATCTGGCCAATTCAAAGGAGGACGATTTC | |
| | | | | CTTCTATTACTTCTTCTGCCTCACTTGGTATATGCATATATTGATTTATCCTTTCTACAGATATTATGTAATT | |
| | | | | TGCTATATTGCATTGACTTTGAATTAAAAATACCAAGGCTGCATTTAGTGAAAAACCATAAGAGAGAGCC | |
| | | | | ATGCCAATGAACCCTGGAAAAAAGGATATCGAAATTAAGAAGCTTGCATCAAGAAAGATATTGATACTT | |
| | | | | TCTGAGGATAAAATCAA | |
| SY4224 | SY4224F1 | GGCTCCA | | | 824 |
| | | TGAGACG | | | |
| | | AAATAAA | | | |
| | | GC | | | |
| SY4224 | SY4224R1 | GAGGGCA | | | 825 |
| | | CAAGATT | | | |
| | | GGTATTG | | | |
| | | TTG | | | |
| SY4224 | SY4224A1FM | TGGGAAG | C | | 826 |
| | | TCTACTCT | | | |
| | | GAT | | | |
| SY4224 | SY4224A2TT | AGTGGGA | A | | 827 |
| | | AGTCTAC | | | |
| | | TCTTAT | | | |
| SY4335 | | | | ATTGAGGTTGAAGCAATTGATGGTGGTTGACTTGTGATCTCCGTAGATATGAGCTAATACCTCTCCTTAC | 546 |
| | | | | CCCTCATTAGGCTTTTAATCAGATGTGGACATTAATTCCTTTTCAGTCTGCTGGGCCTATCATGCGTTGAT | |
| | | | | TTGGTCCTCTGTTTTCTTAATACTGTACTCATTGTTTCCTTACAGTTTGAAATTTGGATACTTAGCCCTTTTT | |
| | | | | ATTATCAGTTTCTATAATAAAATGCAGTAAGTTAATTTCAGTGTCTGGTGGTGNATTGTAAGACATGTAA | |
| | | | | ATAATGATAGAGATATGGCCCAAGTAGTGCTTATGAGGCTTGGATAGTTCTCACCTTACAAGCTGGTCTT | |
| | | | | GTACAGTTGAGTTTTAATCCAAATTCTAAGATACATGTATGATGTTCAAGAATTGGAGACTAGAACAAAA | |
| | | | | TTTGAAATTCAGTCAAAGCATCAATCTCCCATCTTAGGTTCTACATGTCATCAGTAAACCTGATAGGCTAA | |
| | | | | TAGCT[A/T]TGGCCAGAAGCTTTCTCCTTGCTTAACGAGTAGTTGGTAGAAAACTAAAAATAGCTAAAAT | |
| | | | | ATCAGGCATTTCATAAATTGAGTCAATTTGCTATTTGTCTTTATACTATGCTGTGTTGAATATACCTAAAG | |
| | | | | CCTGTAGGGAGCCTCAGNAGTAGTGAAATTATAGTTTATTTATGCTCCAGGTCACTGGTACAGTTCTTGG | |
| | | | | TGTGGAACCTTGGATAACAAAAATAATTTTTTGGTTAAAGTATATCCTTTTATTAATTTAGAATTAGATAC | |
| | | | | AGCTATCCCTGGAATTTGTGCTTAGATTTTGGAGTCAGGAATACATTTGTTTTATGTCGTTCATCAACTTT | |
| | | | | GGTTAGTTGTGACATTTAGATAAATAGATTTAATAGTAAGGCTAATTGTTGTAATTCACTTCACTGTAGA | |
| | | | | ACTGGATTTAAATATTCAGAAATTATGCAGCCTACATGATATGATGAGTTAAGCTGTTCCAAAGAGAAAA | |
| | | | | GATTGTCTTCAAACAA | |
| SY4335 | SY4335F1 | CAAGCTG | | | 828 |
| | | GTCTTGT | | | |
| | | ACAGTTG | | | |
| | | AG | | | |
| SY4335 | SY4335R1 | ACCAACT | | | 829 |
| | | ACTCGTT | | | |
| | | AAGCAAG | | | |
| | | GA | | | |
| SY4335 | SY4335A1FM | AAGCTTC | T | | 830 |
| | | TGGCCAA | | | |
| | | AG | | | |
| SY4335 | SY4335A2TT | TCTGGCC | A | | 831 |
| | | ATAGCTA | | | |
| SY4213 | | | | GCCGTGCCTGACTTCTCACCTACTCATTTTCGCTCTCTAGCTATAATGAGTTTATATTCACAACTTTTTAAA | 547 |
| | | | | TAATTTGAAGCTAACTGTATGCTACGGGGTTTATATAAAAAAAAAAATATTAAAGAATTGTGTTTTTAAT | |
| | | | | CTTTAAACTTTTGGGTAATATGGTCAAATTCAAATCACTGTACTTTTATATTGATGAATTTGGTTATAAACT | |
| | | | | TTGAAAAAAAAATCTGAATTTAGTCTCTCCGCTCAACTTTAAAAAAATAATATAATGATGGGCTCTTACA | |
| | | | | AATGGGAGACTTTTAAGGATCAGATTATTTGATATAAAAGTATGTAGATCTTAATTACTTACCAAAAGTA | |
| | | | | TATATAACAACTAAAAACACATTTTTTCCAAAACTATATGCTACCAAGTTGCAGAACATTATGAAAATATG | |
| | | | | TTAATATTAAATATGCTTTTAATAATCACTGTATAATTAATTTTAAATTTCTAATGTTAGTCCTTATAAAAA | |
| | | | | AAA[A/T]TTGGTCATTTTTATAGTCTCATTTATTTTTCACCATTAATATTAGTTCCTTTAAACAAGGACAAT | |
| | | | | TTTCTTCTTCTTTTTTCTCATTTTCAGTCCCTTTCTCGGACTCATATTGATAATGGAAAATAAACGAGAGGC | |
| | | | | AAAATATGGACAAAAAATTAATAGGAATTAAAATAAAAAAAATCTGAAATTTTACAATGATATATTTAAT | |
| | | | | CCAATAGTTAATTAATCATCATGCACATATATTTACTTGATATATTTTAATTTGAGTCAACTATATGCTACC | |
| | | | | ATAAATTTGACTAAAATGAGTGGCAAGAAATTTTTCATTCTCTTCTAAATACAAGTGAAAATCAAACATTT | |
| | | | | TTTTTAAAATAAAAGACTGAATAAGTTTTAATATTGTATGCATGTACATGATTGTGACTGCAACGTTACTA | |
| | | | | AAAACTATTCCAATAATGTGTCACCTGCCACAATGGCAACTTGCAAGGTAGCAAAAACGAAAATAATTA | |
| | | | | ATGGAGATGA | |
| SY4213 | SY4213F1 | GCTACCA | | | 832 |
| | | AGTTGCA | | | |
| | | GAACATT | | | |
| | | ATGA | | | |
| SY4213 | SY4213R1 | TCCGAGA | | | 833 |
| | | AAGGGAC | | | |
| | | TGAAAAT | | | |
| | | GAG | | | |
| SY4213 | SY4213A1FM | AGACTAT | T | | 834 |
| | | AAAAATG | | | |
| | | ACCAAATT | | | |
| SY4213 | SY4213A2TT | AGACTAT | A | | 835 |
| | | AAAAATG | | | |
| | | ACCAATTT | | | |
| SY4227 | | | | CATTCCGAGAATTTATTTCAATGAATATTTCATACAATAATCTCCATGGTATAATTCCTAATTTTCCAACAA | 548 |
| | | | | AGAATATTCAATATTCCCTAATTCTTGGACCAAATCAATTTGATGGCCCTGTTCCACCATTTCTGCGAGGT | |
| | | | | TCCGTATTTCTTGATTTATCCAAAAATCAATTCTCAGATTCTCTTTCATTTTTATGTGCTAATGGTACAGTT | |
| | | | | GAAACTTTGTACGAATTAGACCTTTCAAATAATCATTTCTCTGGAAAAATTCCGGACTGTTGGAGCCATTT | |
| | | | | CAAGTCATTAACTTATTTGGACTTAAGTCACAATAATTTTTCAGGAAGGATACCCACATCCATGGGATCTC | |
| | | | | TTCTTCATCTTCAAGCATTGCTATTGAGAAACAACAACTTAACAGATGAGATACCTTTCTCCTTGAGGAGT | |
| | | | | TGCACAAATCTAGTAATGTTAGATATTGCAGAAAACAGATTATCAGGGCTTATCCCTGCTTGGATTGGGA | |
| | | | | GC[A/G]AATTACAAGAGTTGCAATTTTTAATTTTGGGAAGAAATAATTTCCATGGAAGTTTACCATTGCA | |
| | | | | AATTTGCTACCTAAGTGACATTCAACTCTTGGATGTCTCACTAAACAACATGTCTGGGCAAATTCCTAAAT | |
| | | | | GCATAAAAAATTTTACTTCAATGACTCAAAAGACATCTTCAAGAGATTATCAAGGTCATTCATATCTTGTC | |
| | | | | TATACCATTGGCATTTCTGGTAATTATACATATGATTTGAATGCACTCTTGATGTGGAAAGGTTCAGAAC | |
| | | | | AAATGTTCAAAAATAATGTGTTACTACTTTTAAAAAGCATTGATCTCTCAAGCAATCACTTTTCTGGAGAA | |
| | | | | ATTCCACTGGAAATAGAGGATTTATTTGGATTGGTTTCATTGAATTTATCAAGAAACCATTTGACCGGAA | |
| | | | | AGATTCCTTCAAATATTGGAAAGTTAACATTACTTGACTTTCTTGATTTGTCAAGAAACCATCTAGTTGGT | |
| | | | | TCAATTCCTTTG | |
| SY4227 | SY4227F1 | GCAGAAA | | | 836 |
| | | ACAGATT | | | |
| | | ATCAGGG | | | |
| | | CTTA | | | |
| SY4227 | SY4227R1 | GAGTTGA | | | 837 |
| | | ATGTCAC | | | |
| | | TTAGGTA | | | |
| | | GCAA | | | |
| SY4227 | SY4227A1FM | CTTGGAT | A | | 838 |
| | | TGGGAGC | | | |
| | | AAATTAC | | | |
| SY4227 | SY4227A2TT | TGGATTG | G | | 839 |
| | | GGAGCGA | | | |
| | | ATTAC | | | |
| SY4220 | | | | TGCCTTGTCCAAGACTACGGAACGATCTAAGAAAGTAAGAAGAATTATTATGATCACATATTTTATACTC | 549 |
| | | | | TTCAAACTGTGAAACCAACAATGTGAAATACAAATCTCCAACTATGCTACAAAACCACACAGTTCGACTG | |
| | | | | ACATTGAATATGATCCCTTAAGTAGTATAAAGCATTTTTTTTTAAAAGATAAATTTAATATCATGAAAAAG | |
| | | | | ATAAAGACAAAAAATGATGTATAATTATATATATATATATATATATTATATATATATATTAAATCTCCTTTT | |
| | | | | TATACACCAGAATGTAAGTAATTGGCTGATTTTATTTTCATTTCATTTATTTACGCAAAAAAAAAAAAAAAA | |
| | | | | GAGTTTGGGGGCCGTGGTCAACAGTAACACAAGAACAAGTATGAGCCCCATTCAGTTGAGAATAGTGA | |
| | | | | TTCAGTACATACTGTTACAGAAAATTCTGGTTATrGAATCTTCAAAATTGTACAAACTGTAAACAAGAGG | |
| | | | | CAGAAAAC[A/G]CAGTCTACAAGTAACAACTAGAATCATAATGAAATGTGCTAAACAAAAATATCATCA | |
| | | | | TGTGCCCAAAACAGCACCTAAATTGTGTCTTCATCCCAGTTAAATAATGCTTAATTTCTTCAACCACCTCA | |
| | | | | ATTGCAAACACCTAAATTCGTCGTTTCTTTGGGAGTCTGCAACCATTATGGGGTTTTCTTGTAGTATCTTC | |
| | | | | AATGTATACAATTGGACTTCTATCCCCTCGAGCTACATTGTTGGGGTACTAGCTAGTGCTATCTCTTTAAT | |
| | | | | TTTCCACACAACCCTTATTTTTTCTGTTTTCTTTCTGCATGTAAAATATGAAGGTCGATTCAAAACTTAATT | |
| | | | | TTATTTTATTCTTTGTGTCCAATATTATTAATTAGATGAAGAAGGCATTGAACGACACATCATGTTCTTAC | |
| | | | | CAAGTGATCATAGCTGCAAGTTGCTCATCGCCCAATTTCACAATGGAGAACATTGCTATTCGCTTTGGGG | |
| | | | | ATGTTCATGGCAACCAA | |
| SY4220 | SY4220F1 | TGAGCCC | | | 840 |
| | | CATTCAG | | | |
| | | TTGAGAA | | | |
| SY4220 | SY4220R1 | GCTGTTTT | | | 841 |
| | | GGGCACA | | | |
| | | TGATGA | | | |
| SY4220 | SY4220A1FM | TTGTAGA | G | | 842 |
| | | CTGCGTT | | | |
| | | TTCTG | | | |
| SY4220 | SY4220A2TT | TACTTGT | A | | 843 |
| | | AGACTGT | | | |
| | | GTTTTC | | | |
| SY4343 | | | | GCCACACACTTGCAATAACATGTGTAAAATGGGGTGGAGATGGCGTGATTTATACTGGGTATTCTCTCCC | 550 |
| | | | | TCTGTCCTTCCAGTTTCTGTGAAGTTCNGCCTGAGTTTTTATTTGTAGTAATTCTTATTGTTATATTGCATA | |
| | | | | TTCCTGCATATTCCTGCATACCCCTGTTGGAAATAGAAACTCATTTAGGTTAAATTCTCATTTTATGATTTT | |
| | | | | AACTCGTGTTTTTATCCAAGAGATCTTATTTTCAGTCCTTTAACTCAACTGATCCTAATGTTGATTGATGAT | |
| | | | | ATGAACAGGGGTTTTCCCCTCTTTTTAATTAAATTAGTGTCTTAATTATCACTCTGCAATTCTTTTTACTGC | |
| | | | | TTGGAGCTTAATTATGTTGTTTTATACAGCTCACAGGATTGTACAATCAAAGTCTGGGAAACCACACAAG | |
| | | | | GGAAGCTAATCCGAGAACTGAAGGTGAGCGTCATCCCTTTGCAGTTTAACTTCTTATCTGCATTTTTTTTA | |
| | | | | A[A/T]CATGGTTTTTGTTATGCATAACTCCTGTTTCTCATCTGGTGTCAACTTTTTCTTCATTTGAGTTATTT | |
| | | | | GATANNNNCTTTAGTGTCACTTTTATGCATACCAGATATAGCTACTAATTGGTATTTTACTATTTATGGTG | |
| | | | | AAGCAAGAATTTTTTGCTGTCACATTTGCTTCCTAGGAAAGTGAAAAATTGCTTTTGAAAGCGTGCAACA | |
| | | | | GGGTTCCTAACGGTTTTCTGAACATACATTTAGTGTTTTATTTTGATTCATTATTCATTGTTAGTGTAAAAT | |
| | | | | TTTTATTCAATGTGTGGATACATCTTTTAAAAGTTTGATTCTCCAGGTATGGTGACCAAACTTCTGTTTATC | |
| | | | | CATTGTTAGAATGTTAGTGTAAAATTTTTATTCAATGTGTGGATACATCTTTTAAAAGTTTGATTCTCCAG | |
| | | | | GTATGGTGACCAAACTTCTGTTTATCCATGTTGCATGAGACTTTTTTCTTTTAATTTTATTTCAATTAATGT | |
| | | | | TTTT | |
| SY4343 | SY4343F1 | GGGAAGC | | | 844 |
| | | TAATCCG | | | |
| | | AGAACTGA | | | |
| SY4343 | SY4343R1 | TGACACC | | | 845 |
| | | AGATGAG | | | |
| | | AAACAGG | | | |
| | | AG | | | |
| SY4343 | SY4343A1FM | TGCATAA | T | | 846 |
| | | CAAAAAC | | | |
| | | CATGATT | | | |
| | | AAA | | | |
| SY4343 | SY4343A2TT | TGCATAA | A | | 847 |
| | | CAAAAAC | | | |
| | | CATGTTT | | | |
| | | AAA | | | |
| SY4316 | | | | TTGGTCTTTGGTGTGAGTTTTTGTTGCGATACCTTAGCTTCGATGAAAGTGAAGAGGATGATATGCGGG | 551 |
| | | | | AGGGGGTGGTTCGTAAATGTTACAGATGAAGTTAATCCATAAAAACGATATGAATCAACTTGATTTGTA | |
| | | | | TGTTGACATTATACTTATACGGATCAAATTGATTTGTGTAAGCCTTCCAGATCAAGTTGATCCATAATATT | |
| | | | | TATACGGATCAACTTGCTTGCATAGCCCAAAATGTCTAGTATCTATAATAGATAATAAATGATTGTATTA | |
| | | | | GTATTTAATAATAAAAAGTTGTCACAAGAAAATAAGAAAAGGGAGGAGTGATCTGATACTAGTAAATTA | |
| | | | | GCATGCGGTTATTAAATATTATCAAGATATTAAAAATTATAGTATTATATATAAATATCTATATTCTCATC | |
| | | | | AAGAAAAAGAAAAATAATTATAAGTAATGAGAGAAAAATAAAAAACAAAATTCAGAAATAGTAAAAAA | |
| | | | | ATAGTTGATTTGA[A/G]CATTATTTTACTGGCAATTTCCAAGCAAGAGTATCTTCATTATATTTCTTAGGC | |
| | | | | TGGGTAAGATATGGAGATGAGAAGCAAGGAAGAGATAGAAATAGGAAGAGACATAACACCAACACTC | |
| | | | | ACTCCTCTCTCTTCATTATATTTCCAAGCAAGAGTATCTTCATTATATTTCCAGTTGTAAAGAATTCATTAA | |
| | | | | CCGCTGCAAAGATATCGTCACCAATGATATTCCAAGTCTTCTTGAAGAATAAAACATTGAAACCATCTGG | |
| | | | | CCCAGGAGCTTTATTGTTATCCATCACAGAAATAACGTTCCAAACCTCTTGCTTAGAAGTAGGACAAAGT | |
| | | | | AAGGTCGCAAAGCAATCGATGGAAACCTTAGGACCCNTGTTGCAGATCGAAATGGAAGGAATTTGGGT | |
| | | | | CAGCTCATGAGCACTAAACAAATTCCAAAGTGATTCACAAAAGCAAGGACAATTTCATCTTGGGAGGA | |
| | | | | AGTGTTATGCCCATCCTCTAGCCTTATGGC | |
| SY4316 | SY4316F1 | GAAAAGG | | | 848 |
| | | GAGGAGT | | | |
| | | GATCTGA | | | |
| | | TAC | | | |
| SY4316 | SY4316R1 | ACCCAGC | | | 849 |
| | | CTAAGAA | | | |
| | | ATATAAT | | | |
| | | GAAGATAC | | | |
| SY4316 | SY4316A1FM | CCAGTAA | G | | 850 |
| | | AATAATG | | | |
| | | CTCAA | | | |
| SY4316 | SY4316A2TT | TGCCAGT | A | | 851 |
| | | AAAATAA | | | |
| | | TGTTC | | | |
| SY4225 | | | | ATGATATGGACCCTAAAACACCTGTCCTAGGCCCAGGATCCAACAAACTACAAATACTTTGACCCAAGG | 552 |
| | | | | GGAAAGAAAAAATTGACTCAAAAAGAGGGTTAACAAGAAAAAAAAAAAACTTGAAATACCTTCAACTC | |
| | | | | GAAGAACACAACCACTGAAAAAAGAGAAAGTGAGAAAAGGGTCGAACAACTGGTGAAGTCATTGATG | |
| | | | | GCTACAGAGGAAGAGAGTGCGGTGAAGGAGCCTTTGGATCTCATTAGGCTCAGCCTCGACGAGCGTAT | |
| | | | | CTATGTCAAACTCCGTTCCGACAGAGAGCTTCGTGGCAAACTTCACGTAATTCTTCAATCTTTTTTTTTTCT | |
| | | | | CCTTTTCATGAATTTGTCTGTTTCTTTAAGCTTTTTTTTTACTCTTTTTGAGACTTCCCTTTAACGCGTAGGG | |
| | | | | TGTTTTGGGTGTTCTGTGATGGAAAATTTAAAATTTTAAAGTTGTTTTCAAGGTAATTGAAATCTGGGTTT | |
| | | | | GTTGCAATAGTT[C/G]GCAAGTTTGTGCCTTTCGGACTTCCCTAATGATATGCCCTATGTAAGAATAATG | |
| | | | | GGGTGTATACTACTTGCGTGTGGGGATGGAATTGAGTCTTTGGTGGTTCCAAATTTTGCGCTTTGGAAG | |
| | | | | AAAGTTGTTTTTTGTTGCTGAATGGAAATTTGAGTGTTTTGAACTATAATTTAGAATAAGCAGGTTTGGG | |
| | | | | ATGAGGAATGATAAGTATGAGTTGTTTATTTTTTTTGCAAAATATAAGTACAACTTGTTAGTTATTTTTCT | |
| | | | | TCACTGCTATTAACTGATGTTAAACTAGATCAATGATTTGGTGCATTTTGCGTGGGTGGGGTGGTGGTTT | |
| | | | | TTATGTATGTGTTTGTTTACTGTTCTTGCATTTTTATAAGATTTTCATGAGCTTGTAAATTGTAATTTAATTT | |
| | | | | ACCGAAGAGTTCCTATCTGAAACTAACCTGAGTTCACAATGATTTTTACAAGTTACACATCACTGTCATTG | |
| | | | | ATCTTGTTCATTTGATAGAAGT | |
| SY4225 | SY4225F1 | TTTGGGT | | | 852 |
| | | GTTCTGT | | | |
| | | GATGGA | | | |
| SY4225 | SY4225R1 | GGCATAT | | | 853 |
| | | CATTAGG | | | |
| | | GAAGTCC | | | |
| | | GA | | | |
| SY4225 | SY4225A1FM | CACAAAC | G | | 854 |
| | | TTGCCAA | | | |
| | | CTA | | | |
| SY4225 | SY4225A2TT | CACAAAC | C | | 855 |
| | | TTGCGAA | | | |
| | | CTATT | | | |
| SY4219 | | | | AAAAGATGAAATGGAGACAAATCTTGTCAATCTTCGAAAGACAAAATACTCAGCAATTATGTCTAGTGTT | 553 |
| | | | | GATTTAGAGGAAGCTGGTCATAAGCTTCTGGAAATTAAGCTAGAGCCTGGCCAAGAGATGGAATTGTG | |
| | | | | CATTATGATTTTGGAATGTTGCAGACAAGAGAAAAACCTATCTCTGATATTAGAGTCTTCTCGAGCAGTG | |
| | | | | TTTGCAGATGTGGCAAAGTTATGATTTTGGAATGTTGCACGATCAACAAAGTACACCAAGAAAATCTCG | |
| | | | | AAAAGTGCTTTTTGCAGCAGTACTCAATGATTAACCGACTTGAAACAAATAAACTGCATAACGTGGCAAA | |
| | | | | GTTTTTCGCTTGTTTATTTGGCACAGATGCTCTACCTTGGCATGTTTTGTCATATATACGCTTGACTGAAG | |
| | | | | ATGATACAACTTCTTCACGTATATTTCTTAAGACTATTTTCCAGGAAATATCAGAACATCTTGGAATCGGG | |
| | | | | CTGNTAAATGA[A/G]CGGTTAAATGATCCAACAATGTAAGAATCTTTTGATGAATCCATATTTCCAAAAG | |
| | | | | ATAATCCAAAAAACACACGGTTCTGCATTAACTTCTTTACATTCATTGGTCTTGGTGGTCTTACTGAGAAC | |
| | | | | CTACGTTAGTATTTGAAGAATATGCCGTGTCTTATCAAGCAACAACAGAAAGATGAGTCAGGTAGTTCT | |
| | | | | GATTCATCAGATTCAGATGCAGAATCAGCAAGTTCGGATCAAAGTGACACTGAGAATGACAGAAGCGG | |
| | | | | AAGAAAGCGGCCGGAGGACAAAGGGTGAAGCAATTTGATGCTATTGTCGAAGGCCCTTGCATCCACAA | |
| | | | | AACTGATTGAGTATCCAAGGTTTCTTAATCTTTAAATATCACTGAACTTACTTGCCTTGTTATGCAAATAT | |
| | | | | GAAAGGGATTTTTTTTTTTATCAGTAATGTGAAAGGGATATGTGGGCTATGCTGGTTGAGATGTTGAGC | |
| | | | | ATGCTCGATGGATTTGTTTAATTTTGTTC | |
| SY4219 | SY4219F1 | ACGCTTG | | | 856 |
| | | ACTGAAG | | | |
| | | ATGATAC | | | |
| | | AAC | | | |
| SY4219 | SY4219R1 | AAGTTAA | | | 857 |
| | | TGCAGAA | | | |
| | | CCGTGTG | | | |
| | | TTTT | | | |
| SY4219 | SY4219A1FM | TCATTTAA | G | | 858 |
| | | CCGCTCA | | | |
| | | TTTA | | | |
| SY4219 | SY4219A2TT | TGGATCA | A | | 859 |
| | | TTTAACC | | | |
| | | GTTCATTT | | | |
| SY4326 | | | | GAATTATGGCCCATTACGCGTAAAGTTGCCAATTCGATCTCTCTTTATAAAAGTATTTAGGAGTAGGAAA | 554 |
| | | | | AGGTGTCCATTTTTCTTCTCGAAATTCAAGAGTAAACAAACTACGGTTGAAAGAAACGTCTACATAATTG | |
| | | | | AAAAAAAATGAAATCTTATTTTATGTGTGTGTGAGTTCTGCCCAACATTGACAACACGGGAGTATATCCC | |
| | | | | TGCTGCTAATAATTTATAATAAAAAAATATTTAATTTTAAATGATTAAAAATTTTAATATAAAAAATTGAA | |
| | | | | ACGTTTATAAATTAATGTGAACTTAGACCTCCAAATGGATTCTTACACTGTAACTGATGTCACNTAGGTA | |
| | | | | GTAGAATTCCAAGATCCAAAGGCACTAGGTGTGTATATTCAATTATACTTAATTTATTGTAATTTTGTATC | |
| | | | | GTTCTGTATGGCTTGACAGCTTTGGATGTTCTTCAAGTCTAAGTATCATATTTATATAAGCTGAGACAGA | |
| | | | | GATATCAG[A/G]TTTTNATTTGTACAGTCTAAGTAGTATAGTGTTGTGCGAGTGACTTGTGACTAAACGA | |
| | | | | TAATATTTTATATGCGTGGAGAGATCGAGAACGAGCTAATATTGTAGTCTTTTCTTAATTTCAACTTTCTT | |
| | | | | TTTTATNTTATTTCCCTCACAATATATATGGGAATGCTATACATGCTTAGATTTTGAGAAACCTAATTAGA | |
| | | | | GGATCAATGCAATGCAAATGCAGAAATAAACTTTCTTCCTTGTTATTTTTTCTTTCTTTCTGAAACAGCCA | |
| | | | | AAAGAATATCATTGAAATAGCACCAGAGGTGCAACAACACAAATTACAGGATTAGAGAAAGACATAAA | |
| | | | | ACAACATTTAACAGAGTTAGCCCTAGCTCCCAAGGCATAACACACTTGCCAAATAATAGATGGCTCGAAC | |
| | | | | CCCCTAGAAGAAACAACATCCGGCAAATACAATAACCTAGGTAACACTTAAACTGAAATAATCGCAGGA | |
| | | | | ATAGAAACAGAACCCAGCGTGA | |
| SY4326 | SY4326F1 | GCTTGAC | | | 860 |
| | | AGCTTTG | | | |
| | | GATGTTC | | | |
| | | TTC | | | |
| SY4326 | SY4326R1 | GTCACTC | | | 861 |
| | | GCACAAC | | | |
| | | ACTATAC | | | |
| | | TAC | | | |
| SY4326 | SY4326A1FM | CTGAGAC | A | | 862 |
| | | AGAGATA | | | |
| | | TCAGATT | | | |
| SY4326 | SY4326A2TT | TGAGACA | G | | 863 |
| | | GAGATAT | | | |
| | | CAGGTT | | | |
| SY4232 | | | | TTATAATTCCATGTTTCACACTTATTTGAGCTTTGATATACAGAAGGATATAATTCCCTAGCCATAGGAGA | 555 |
| | | | | CATTGGACTGTAGTGAAATAATTGTATCTAGTAAAATTGGGTCGTGTTTGATTTATTTAGTTTTTAGCCTA | |
| | | | | AGTGGGTATATATAGCAGCAGCCTTCACAACAATTAATTAATGATCAAGTTTTTAAATATATACCCAGAA | |
| | | | | AAAATTACTGGGTTTCCCCCAATGAATTAGTCAATAGTCAATAATGATTTTGATGGATTTCAGTCTCGTAA | |
| | | | | GCATTCCMGCACCCGGGGGCTATTAGACMMTGTCTATTAATAACTAGAAAATTCAACTAACTAAATT | |
| | | | | AGTGAAATTTGAATTCGGGTATATTGCAGTTCTTATACTAGATAACCTTGATATCGAGCATTTCCTTCCTC | |
| | | | | TTTTTTATTTTTATTTTATACATATTTATTTAAAAACAAAACTGCATTTGCTAAAAGAAAGTCTGGGTATTG | |
| | | | | AATT[A/G]TTAGGACTGGACTATGAAAGTAAAAGCCTTAATTCAATTTTGAAATAGAACAAATCAAAACC | |
| | | | | TTCTCCGAAATCTCCTAATTATGTACTAATTAACTAGTTTAATTTTCTCTGACTTACACATATATTAAATTCA | |
| | | | | TTTTTATAATTTATAAGAATAATTTTTTATATTATTTGAATTAATATATTATTAATGATTTAATTTAAGTTTT | |
| | | | | AATAAATATATATTAATAGATATATCAATAAATATTTAAATATGTTTTTATACACAAACATATTTCAGAAA | |
| | | | | ATTGTTATTATTATAAGTATTGTCCTATATAGAATTTATTTTACTTTAATGATATATTACATATTTTCTTTTG | |
| | | | | ACTTTCTTTTAAAAAATTAATTTTATTCTTTTTTTACACATTTGATTTGAGATAAAAAAAATTGGGTGAAGT | |
| | | | | CATTAATTACAAAAGAATAATTTAATCTAGATTTAAATTATTTTATTGGATGATCAATTAAATTTATGCAC | |
| | | | | GTATA | |
| SY4232 | SY4232F1 | CCTTGAT | | | 864 |
| | | ATCGAGC | | | |
| | | ATTTCCTT | | | |
| | | CCT | | | |
| SY4232 | SY4232R1 | TTCGGAG | | | 865 |
| | | AAGGTTT | | | |
| | | TGATTTG | | | |
| | | TTC | | | |
| SY4232 | SY4232A1FM | TCCAGTC | G | | 866 |
| | | CTAACAA | | | |
| | | TTCAA | | | |
| SY4232 | SY4232A2TT | AGTCCAG | A | | 867 |
| | | TCCTAAT | | | |
| | | AATTCAA | | | |
| SY4330 | | | | ATTTTATTTTTTAGTGGACATTCTTTAACTTGTGCTTCTATTGCTCTCAATAACTTAAAGAATCAATCATTTT | 556 |
| | | | | AAAATTTGGTTACGAAGGACGAGCTTTTTAGTGTGATTTTTAGTATCTTAAATACATTCCATATTGTGGTA | |
| | | | | TAAATTCAAGGATCTTTATATATTTTGAACTTTGTATAGGTTAAAATGTACATATTAAATTATCTTTCAAAA | |
| | | | | AAATAATAGACTTAGAAAGAAAAAACTTGATCAACAGATACTAGAAATAACTTGGAAACTTTTTTAGGTA | |
| | | | | TCATCATGTTCATAAATTTATCATGTTCATAAATTTATAACTGTTGAGTTTTTTAACTCTCTTCTTATATGGA | |
| | | | | ATAAATGCTCAAATGAAAAGGTTTGTATATCTCACTTATTTTAATGAAGAGAGATCAGATTAAAGAGAGT | |
| | | | | GAGATACACTNAGAGGAACTCATTTGAGAGGAAAAAAATAGTTACAAATCATTAAGAGAGAAAGAAGA | |
| | | | | ACA[A/G]GAAAAATCATTGTGATTTTTGCATACCTATCAAAAAGTGTTTTTAAGATTGCAACTGTNGATT | |
| | | | | TGTTCACTGTTGGATCGGNCTGATTTTTGGCCAGGAGACTCTACACACGTGATATTTCAAGTTGTTCGGT | |
| | | | | TGGATCATGAAAAAGATACCTGGAGAGAGAGAGATAAGTGTTTCNTATTATCTGTTCTATATTTTGGAG | |
| | | | | TTTTATCTTCTTGTCTCTATTGTACCAATTGAAAGTTTATTTTGATTTGACTGCTTGTAGCATGTTTTAGTAT | |
| | | | | ACTTGTTGGATGTTTTCTTGTATCTTCATTGATTATAGTGGAGTTATTTTTTTGGTCTAGACGACCTATAGT | |
| | | | | TTTTATCCTTGCATTGAGGGGTTTTCCACGTTACACAAAATGTGTCTGATTTCTTTCATTTTTATTTCGCGC | |
| | | | | TCTATACTTTATTGGTGATCCTCACAAATTCTTGTAAGTTTAACGGAATTTATTTCCACTGTCTATTACTCA | |
| | | | | CTTATAGAA | |
| SY4330 | SY4330F1 | TGAAGAG | | | 868 |
| | | AGATCAG | | | |
| | | ATTAAAG | | | |
| | | AGAGTGA | | | |
| SY4330 | SY4330R1 | TGTAGAG | | | 869 |
| | | TCTCCTG | | | |
| | | GCCAAA | | | |
| SY4330 | SY4330A1FM | CACAATG | G | | 870 |
| | | ATTTTTCC | | | |
| | | TG | | | |
| SY4330 | SY4330A2TT | ACAATGA | A | | 871 |
| | | TTTTTCTT | | | |
| | | GTTC | | | |
| SY4325 | | | | CTTTATTTCTTTTCAAATTTTATTTTCATTTATATGCAAACATGAAATATTGTGTTAGTAAGATGTCTAGAG | 557 |
| | | | | AGATAAATATACCAATTGAGAGAGTGGGGGGGGAATAAGATTGTTGATTACATTGGGCAAGAAAATCT | |
| | | | | ACAACTTAGCCTTAGGCCAAAATTTTAAATGTGAAACCAATGATCTAGCCTTAACTAATAGGGTTGTGAA | |
| | | | | GGTTCATTGGGCTTGTGGACTTTTTTAATGTTAGGCTTTTGACTTTGGGAACAATTATTCCTATTACCAAA | |
| | | | | TTTGCTATTGGCCCTACTAACTTTTTTGAAATTCTTCTTTTGCCCACTCACCAAATCCATACACCCATTCCTT | |
| | | | | TCTCCTCAGATTCAAAATTGTCTCCCCCACCCTAGCATCCCAGATCCATAACCCCCATGTTGCTCTCTCCCT | |
| | | | | CGTGAAACCCCTCCCCTCGTGCCACCCCTCTCCCTCATATTCCATTGCTTTATTTTTTTCANCCCCATGTGT | |
| | | | | GCA[A/T]CGGAAATACAATTATGTTATAGACTTGTTCAACAAAATCGTATTTCCTTTGGAAGGATATTTTT | |
| | | | | GGAAAAAATATTTAAAAGTTCATGTGGGTAGTGCCAATAAAAAAAGTTGATAGTCACAATAGCATGCCC | |
| | | | | CTTTGACTTTATAGGCCTTTTACTTGAACCCATGTGGAATATGAGTGAGCAAGTGGGGTGGAGTGACTC | |
| | | | | ATTTTAATAGCTCTATTCATATCAATAAGGCTAAAACTTTATGCACCTCCAAAAATTTCTTCTAACACCCCA | |
| | | | | TTACATTCTAGAATATGATGGGTGTGCAAGAAGCAACAATCTATCAACAAATGTGTGCAAATTCACACG | |
| | | | | AATTGCAACTTGCAAAGAGTGTCGAATACATTCATACACTGAATCAAATAAATATAAACTAGAGAAAATA | |
| | | | | GGGACCGTAAGATCCAATGATTTGCTCGTNGATGAGTATTGCACGTTATATCTTCACACTTCGTTTTTTTT | |
| | | | | ATCCATATAAAGCTCA | |
| SY4325 | SY4325F1 | CCTCTCCC | | | 872 |
| | | TCATATTC | | | |
| | | CATTGCTT | | | |
| SY4325 | SY4325R1 | TGGCACT | | | 873 |
| | | ACCCACA | | | |
| | | TGAAC | | | |
| SY4325 | SY4325A1FM | CATGTGT | A | | 874 |
| | | GCAACGG | | | |
| | | AAA | | | |
| SY4325 | SY4325A2TT | CATGTGT | T | | 875 |
| | | GCATCGG | | | |
| | | AAA | | | |
| SY4217 | | | | ATTTTGTGTGATCGCTCCACCATCCCAAACATACCAATAATAAACAGCAAATTCATTGTCCTAATGCATAA | 558 |
| | | | | AATGAACACAGTTATATTTAAATTGATTTAGTAAAAAAGCTGCAAGATACACTGAACCTGAGAAGTTAAT | |
| | | | | CTAAAAAACAAGTTCAGTCACCACCTGTGAAGTTTGAAATTTACAGCTGGAAGATCCTATTAGATACTGT | |
| | | | | TCTAATACTTTGGGATGCAATAGCCATCCATGTATGAATATGGAATTGGATAAAAAATCAAAAGCTCTGC | |
| | | | | ATGATATCGGGCCAATTTTAACTAGCCACAAATTAAAATGAAGTGTTGCAAACAATGTAGCCCAATCACA | |
| | | | | AAACCGATAGGAGGTAAAAAAACGGAAATAGAATGAACAGTATTAGAGTAACTTGCTACCTCTTCATCA | |
| | | | | TGTGTATTGTTATAGAAAGATTTTAATAGGATATGTAGAATTCTGTCCAAATGAAAAGAAAAAAAAAAA | |
| | | | | AACAGGTCCTA[A/G]GAGTATCTCCAATCTCCAATAGAAAAGTCATTCATGCATTCTTTAACTTACTTTTA | |
| | | | | AAGAATACTATACAGCCATATAAGATTTAAGAATTTCAACAAATTTTAATTCAATAGTAGAATTCTTAAAA | |
| | | | | GCTTCAAACTAGCTTCACAACGTACCTTACATTTATATGTTTTGTGTTAATTCTGACAATCAAAATAATTGT | |
| | | | | TTATATGTTCTTGGTAGTCACATTATTACAAATCTTAATAGAACTGCTCAGATGTTGTAATTTAATTTTTCA | |
| | | | | TGCAGCAAGTGGACGGAAAAAATTCTTAATTGTTGAAAAGAAAAGACTGATTCAGTAATGAGGGTTACA | |
| | | | | ATAAGAACTCGGTGTCATAATGTTAACTAAGAATGGTTCGACTATATTAACGAATTGGTATATTACAGAA | |
| | | | | ACACAAGTTTAAAAGTTATGGTCTTGAGAACGCAACAAAAGACAGAAAGACTTGATTATTTTCCAAGAA | |
| | | | | GGGATGCCACTATATTAACTT | |
| SY4217 | SY4217F1 | TGCAAAC | | | 876 |
| | | AATGTAG | | | |
| | | CCCAATC | | | |
| | | AC | | | |
| SY4217 | SY4217R1 | TGCATGA | | | 877 |
| | | ATGACTT | | | |
| | | TTCTATTG | | | |
| | | GAGA | | | |
| SY4217 | SY4217A1FM | TTGGAGA | G | | 878 |
| | | TACTCCTA | | | |
| | | GG | | | |
| SY4217 | SY4217A2TT | TTGGAGA | A | | 879 |
| | | TACTCTTA | | | |
| | | GGA | | | |
| SY4215 | | | | TTAAAGTATCTTTGCAAAAACAATTTCACATGAGGCAACCTTAATTTGCAATTGAAAGTTCTTTATTGCAA | 559 |
| | | | | TATCAACTCTTGGCTTTGGATGTGTTTTCACCTTGTACTCCTTCTGGCGTATATTTTATGTCTCTTTAGATCT | |
| | | | | TAATAAAGGTATTTTTGTCAAAATGATCATTTTGTCACCGTACCTTATGTCATTAATATTTTTCTTAAGATG | |
| | | | | CGTATATTACCTTAATTAAGACAAATAGAGCTATGCCATTTTTTATGAAAGTAACATATATACAAAAATAT | |
| | | | | GAAAAACTCTTTTTCACACAAAAAATGATAGTTTTTTTTTGGTTTTTTTATTATTAAAGTAGTTTCCATGCA | |
| | | | | GATTAATGATAGTTTCTGATTAAGGGGATCGTTTCTGTTTGATTTATGCAGGTATAATTAATACGCTGGG | |
| | | | | TAGATTTGTGTTTAAAAATGTCCAACAGGTAAGTTAAACAACTATGATATTTGAAGCCAAATAACACGAG | |
| | | | | T[A/T]CTCGTAAACTATTTAAGTTAGATTATTCATTAAATTAAATGTTTGGTAACTAGAACAAATATGATA | |
| | | | | TTTGAACAGAGATATCAATACAAATGACATGAGTTATTCGTAAACAATTTAAGTTTGATTATTCATTAAAT | |
| | | | | GCATGCTTGTCCAAGATTGTTCGTTTAGTTGGACAAACAAACTTAAACTTATAGTTCAACTCATATCATTG | |
| | | | | ACAAAATAGCAATTAATATTGTTTTGGACTTTTAAAATGACAAATACATGTAATTTTTCTTCTTCTAAAAA | |
| | | | | ACCAATAATGTTAATGCATTTTTTGCTTTATTTATTTAAAAAACGCATAATTTTTTTAAACATCTTTTAATTA | |
| | | | | AAAAAGCACTTTCCTGAAAATAGTTTAAAAGTTTTTGAAATAATTTCCCTTCATATGTTATTGACTTGATCT | |
| | | | | TTCCATATTTGCTTTCTTCCCCATCCTTACTGTAGGGAGCTGCAACAACATGCTATGTAGCATTGCATCCA | |
| | | | | CAAGT | |
| SY4215 | SY4215F1 | GTCCAAC | | | 880 |
| | | AGGTAAG | | | |
| | | TTAAACA | | | |
| | | ACTATGA | | | |
| SY4215 | SY4215R1 | AAACGAA | | | 881 |
| | | CAATCTT | | | |
| | | GGACAAG | | | |
| | | CA | | | |
| SY4215 | SY4215A1FM | CCAAATA | A | | 882 |
| | | ACACGAG | | | |
| | | TACT | | | |
| SY4215 | SY4215A2TT | TAACACG | T | | 883 |
| | | AGTTCTC | | | |
| | | GT | | | |
| SY4322 | | | | TAGAAATTTCTTTATCTTTATTCATAGGGTAGAAATATTGGTAGTAGAACCCAACTGTGATTTCAACCGCA | 560 |
| | | | | ACCTCGTCATAAAATCCAACAAATGTGATCTCCTCTGCCACCATNGCGGCAAAATCCAATGAGCCCCAAC | |
| | | | | TCCTGGCAAACCTCATCATGAAACCCCACATGAAATCCAACTGCGAATCCAGTCGTGTGAAACCTACTGA | |
| | | | | AGTCCACCAAACAGTTTTTCTTTTCTTCATGTTGTGGTTTGATTTTTTGTTGTTAAGATTTTGATGGTTCAT | |
| | | | | ATCTATTCTTTTATCCAGCTTGTTTTTATTTAGTCCACCCACCAGGGGAAAAANTGAGTGGTGAGATATA | |
| | | | | ATGAAGATGAAGGGTGATGGAGACAGATAGGAGAAAGAAGAAAGNGGCAATACGACATCATTTTTATT | |
| | | | | TTTTTAAAAAACTTTTTTCCATGTATAATTTCCAATCAGACAAAGAGTACACGTAATATCACACGTGGCAT | |
| | | | | GCATCATG[A/G]CCNATTANCGATCATGTAGGTAGATAATAATTTTGGACTAACAGAAAATATCCGAGA | |
| | | | | CAAAAAATTTTACGACTTCAAAATTGTAAGGTATTTTTATTACGATTTCAAAGTCATAAGTTTTGTTTTTAT | |
| | | | | TTTCTATTTTTCTTTTTACGATTTTGTAGTCATTTTGGTTTTCTTTTTACTTTTAAATTTTTTTATTTTCTTGTA | |
| | | | | TTATATTTTTTATTTTTTAAATTTTTTTAATAATTTTATTTATTTAATTATTAAACAAATATTTTATTTATTCAT | |
| | | | | TTTAAAAAATTAAAATTCAATAAAATTATTAAAATATTATCTAAATTTTAAATATACAAAATATATTAAATA | |
| | | | | AAAAATATTAAAATGGAAAACTTTGATAGTATTAGTTTTATTTAATTTATTGTATATTTTTTATGGGTTTAT | |
| | | | | TTAATATGTTATATATTTTTTAATGTTTTTTTATTTAAAATATATTATGTTTTCTAATATTGTTTTATTTAATT | |
| | | | | ATT | |
| SY4322 | SY4322F1 | ACAAAGA | | | 884 |
| | | GTACACG | | | |
| | | TAATATC | | | |
| | | ACACG | | | |
| SY4322 | SY4322R1 | GTCTCGG | | | 885 |
| | | ATATTTTC | | | |
| | | TGTTAGT | | | |
| | | CCAA | | | |
| SY4322 | SY4322A1FM | CATGCAT | A | | 886 |
| | | CATGACC | | | |
| SY4322 | SY4322A2TT | CATGCAT | G | | 887 |
| | | CATGGCC | | | |
| SY4344 | | | | CTTAATTTTCTTATTTTTATTTCTTTCCTTAATTCATTTAGCCTTATGCATATTGTTATTTATATTGAATTAGC | 561 |
| | | | | TAATCTTAATTAATTCCAAATATTCAGTGTAGAAATTTCTTTATCTTTATTCATAGGGTAGAAATATTGGTA | |
| | | | | GTAGAACCCAACTGTGATTTCAACCGCAACCTCGTCATAAAATCCAACAAATGTGATCTCCTCTGCCACC | |
| | | | | ATNGCGGCAAAATCCAATGAGCCCCAACTCCTGGCAAACCTCATCATGAAACCCCACATGAAATCCAACT | |
| | | | | GCGAATCCAGTCGTGTGAAACCTACTGAAGTCCACCAAACAGTTTTTCTTTTCTTCATGTTGTGGTTTGAT | |
| | | | | TTTTTGTTGTTAAGATTTTGATGGTTCATATCTATTCTTTTATCCAGCTTGTTTTTATTTAGTCCACCCACCA | |
| | | | | GGGGAAAAANTGAGTGGTGAGATATAATGAAGATGAAGGGTGATGGAGACAGATAGGAGAAAGAAG | |
| | | | | AAAG[A/G]GGCAATACGACATCATTTTTATTTTTTTAAAAAACTTTTTTCCATGTATAATTTCCAATCAGA | |
| | | | | CAAAGAGTACACGTAATATCACACGTGGCATGCATCATGNCCNATTANCGATCATGTAGGTAGATAATA | |
| | | | | ATTTTGGACTAACAGAAAATATCCGAGACAAAAAATTTTACGACTTCAAAATTGTAAGGTATTTTTATTAC | |
| | | | | GATTTCAAAGTCATAAGTTTTGTTTTTATTTTCTATTTTTCTTTTTACGATTTTGTAGTCATTTTGGTTTTCTT | |
| | | | | TTTACTTTTAAATTTTTTTATTTTCTTGTATTATTTTTTATTTTTTAAATTTTTTTAATAATTTTATTTATTT | |
| | | | | AATTATTAAACAAATATTTTATTTATTCATTTTAAAAAATTAAAATTCAATAAAATTATTAAAATATTATCT | |
| | | | | AAATTTTAAATATACAAAATATATTAAATAAAAAATATTAAAATGGAAAACTTTGATAGTATTAGTTTTAT | |
| | | | | TTAAT | |
| SY4344 | SY4344F1 | AAGGGTG | | | 888 |
| | | ATGGAGA | | | |
| | | CAGATAG | | | |
| | | GA | | | |
| SY4344 | SY4344R1 | CGTGTAC | | | 889 |
| | | TCTTTGTC | | | |
| | | TGATTGG | | | |
| | | AA | | | |
| SY4344 | SY4344A1FM | CGTATTG | G | | 890 |
| | | CCCCTTTC | | | |
| SY4344 | SY4344A2TT | ATGTCGT | A | | 891 |
| | | ATTGCCT | | | |
| | | CTTTC | | | |
| SY4360 | | | | ATGGCATTGGCTCTTACTCCAACAGTTGTCTTTGGCTCAATAGCCTTTGCAGTTTTCTGGGTTCTAGCAGT | 562 |
| | | | | TTTCCCATGTGTGCCTTTTCTACCCATTGGGAGAACTGCAGGGTCCCTACTAGGTGCAATGTTTATGGTC | |
| | | | | ATATTCAAAGTTCTTAATCCAGATCAAGCTTTTGCTGCAATTGATCTCCCAATTCTTGGTCTTCTTTTTGGG | |
| | | | | ACAATGGTTGTTACTGTTTTTCTTGAAAGAGCAGACATGTTCAAGTACTTGGGGAAATTGCTCTCTTGGA | |
| | | | | AAAGCCAAGGACCAAAGGACTTACTCTGTAGAATTTGTTTAATTTC[A/T]GCTATATCAAGTGCNTTTTTC | |
| | | | | ACCAATGACACATCTTGTGTTGTATTGACTGAATTTGTGTTGAAAATAGCAAGGCAACATAACCTCCCAC | |
| | | | | CTTACCCTTTCCTTCTTGCACTAGCTTCAAGTGCTAATATTGGATCCTCAGCAACCCCAATTGGGAACCCC | |
| | | | | CAGAATCTAGTTATAGCTATTCAAGGTAAAATATCATTTGGGAGCTTTCTCACTGGTATTCTTCCAGCTAT | |
| | | | | GCTTGTAGGAGTTGTGGTGAATGTTGTAATTCTTATAGCCATGTATTGGAAGGTGTTAACTATTCATAAG | |
| | | | | GATGAAGAGGATCCAATTTCAGAAGTTGCTGAAGAGGAGTTTGTTTCCCATCAGTTTTCTCCAGCCACAA | |
| | | | | TGTCACATTGTGCATCCTTTAATTCTCATGAATGCAATGACAGTCTAGAACCTACTAATGGTCTTCAAAAC | |
| | | | | CCTTCTCAAGTACATCCTATCAGAAACCAAACAACTCCAAGTGTAACTGAAGTTCAGATGGTTCTTAGTA | |
| | | | | GCACAAAGGATTCCACAACAAATGCATCCAAGATGGGGANAAATGATGCAAAGGAGGAAACTAATCCT | |
| | | | | TCAAAAGTTGTTGCAATAGTAGTAGATAAACCTATAGAAGCACATGTTATGCACTCTTCACAAGGAAAG | |
| | | | | GTGGACTATTTGAGAAAAAA | |
| SY4360 | SY4360F1 | GGGAGGT | | | 892 |
| | | TATGTTG | | | |
| | | CCTTGCT | | | |
| SY4360 | SY4360R1 | GCCAAGG | | | 893 |
| | | ACCAAAG | | | |
| | | GACTTAC | | | |
| SY4360 | SY4360A1FM | TTTGTTTA | A | | 894 |
| | | ATTTCAG | | | |
| | | CTATATC | | | |
| SY4360 | SY4360A2TT | AATTTGTT | T | | 895 |
| | | TAATTTCT | | | |
| | | GCTATATC | | | |
| SY4208 | | | | CAGCTGAGGCTGCAACTCGTGCTCCAAGCAATAGAACAGTTGGTACATTTGAAGCCAAATTTGATAGNA | 563 |
| | | | | NAAGTATAACTATAGCCAGTATAGCTATTCCACTAGCATGATCTATTCGAGAATAAGGCTCCATTAAGTC | |
| | | | | CCAAAGAGCACCTGGAATTCCAGTTTTCTTGAATCCATCTACTGTGATAAACATTCCACAAAAGAATATC | |
| | | | | AACAGTGAATAAGAGACCTTGTCTATGCTTGGCCCTGCATCTTTGAAATCAAGAACCACCAAAGTTATTG | |
| | | | | CAGCTGCAATTGCAGCCCATGCCATATTTGCACCAAGAAGCATTGCAATCACCATTATCAACGTGATTGC | |
| | | | | ATAAACACAAGATTTCCACACTATCCTTTTCCATTTTTTTCTCAAATAGTCCACCTTTCCTTGTGAAGAGTG | |
| | | | | CATAACATGTGCTTCTATAGGTTTATCTACTACTATTGCAACAACTTTTGAAGGATTAGTTTCCTCCTTTGC | |
| | | | | ATCATTT[A/G]TCCCCATCTTGGATGCATTTGTTGTGGAATCCTTTGTGCTACTAAGAACCATCTGAACTT | |
| | | | | CAGTTACACTTGGAGTTGTTTGGTTTCTGATAGGATGTACTTGAGAAGGGGAAGACCATTAGTAG | |
| | | | | GTTCTAGACTGTCATTGCATTCATGAGAATTAAAGGATGCACAATGTGACATTGTGGCTGGAGAAAACT | |
| | | | | GATGGGAAACAAACTCCTCTTCAGCAACTTCTGAAATTGGATCCTCTTCATCCTTATGAATAGTTAACACC | |
| | | | | TTCCAATACATGGCTATAAGAATTACAACATTCACCACAACTCCTACAAGCATAGCTGGAAGAATACCAG | |
| | | | | TGAGAAAGCTCCCAAATGATATTTTACCTTGAATAGCTATAACTAGATTCTGGGGGTTCCCAATTGGGGT | |
| | | | | TGCTGAGGATCCAATATTAGCACTTGAAGCTAGTGCAAGAAGGAAAGGGTAAGGTGGGAGGTTATGTT | |
| | | | | GCCTTGCTATTTTCAACACAAAT | |
| SY4208 | SY4208F1 | GTGCATA | | | 896 |
| | | ACATGTG | | | |
| | | CTTCTATA | | | |
| | | GGTT | | | |
| SY4208 | SY4208R1 | AGCACAA | | | 897 |
| | | AGGATTC | | | |
| | | CACAACA | | | |
| SY4208 | SY4208A1FM | CTTTGCAT | A | | 898 |
| | | CATTTATC | | | |
| | | CC | | | |
| SY4208 | SY4208A2TT | CCTTTGC | G | | 899 |
| | | ATCATTT | | | |
| | | GTC | | | |
| SY4210 | | | | TTTTTTCTCAAATAGTCCACCTTTCCTTGTGAAGAGTGCATAACATGTGCTTCTATAGGTTTATCTACTACT | 564 |
| | | | | ATTGCAACAACTTTTGAAGGATTAGTTTCCTCCTTTGCATCATTTNTCCCCATCTTGGATGCATTTGTTGTG | |
| | | | | GAATCCTTTGTGCTACTAAGAACCATCTGAACTTCAGTTACACTTGGAGTTGTTTGGTTTCTGATAGGAT | |
| | | | | GTACTTGAGAAGGGTTTTGAAGACCATTAGTAGGTTCTAGACTGTCATTGCATTCATGAGAATTAAAGG | |
| | | | | ATGCACAATGTGACATTGTGGCTGGAGAAAACTGATGGGAAACAAACTCCTCTTCAGCAACTTCTGAAA | |
| | | | | TTGGATCCTCTTCATCCTTATGAATAGTTAACACCTTCCAATACATGGCTATAAGAATTACAACATTCACC | |
| | | | | ACAACTCCTACAAGCATAGCTGGAAGAATACCAGTGAGAAAGCTCCCAAATGATATTTTACCTTGAATAG | |
| | | | | CTATAACTAGATTCTGGGGGTTCCCAATTGGGGTTGCTGAGGATCCAATATTAGCACTTGAAGCTAGTGC | |
| | | | | AAGAAGGAAAGGGTAAGGTGGGAGGTTATGTTGCCTTGCTATTTTCAACACAAATTCAGTCAATACAAC | |
| | | | | ACAAGATGTGTCATTGGTGAAAAA[A/G]GCACTTGATATAGCNGAAATTAAACAAATTCTACAGAGTAA | |
| | | | | GTCCTTTGGTCCTTGGCTTTTCCAAGAGAGCAATTTCCCCAAGTACTTGAACATGTCTGCTCTTTCAAGAA | |
| | | | | AAACAGTAACAACCATTGTCCCAAAAAGAAGACCAAGAATTGGGAGATCAATTGCAGCAAAAGCTTGAT | |
| | | | | CTGGATTAAGAACTTTGAATATGACCATAAACATTGCACCTAGTAGGGACCCTGCAGTTCTCCCAATGGG | |
| | | | | TAGAAAAGGCACACATGGGAAAACTGCTAGAACCCAGAAAACTGCAAAGGCTATTGAGCCAAAGACAA | |
| | | | | CTGTTGGAGTAAGAGCCAATGCCAT | |
| SY4210 | SY4210F1 | GCCAAGG | | | 900 |
| | | ACCAAAG | | | |
| | | GACTTAC | | | |
| SY4210 | SY4210R1 | GGGAGGT | | | 901 |
| | | TATGTTG | | | |
| | | CCTTGCT | | | |
| | | AT | | | |
| SY4210 | SY4210A1FM | CTATATC | G | | 902 |
| | | AAGTGCC | | | |
| | | TT | | | |
| SY4210 | SY4210A2TT | CTATATC | A | | 903 |
| | | AAGTGCT | | | |
| | | TTT | | | |
| SY4207 | | | | AAAAGTTCCCTGCTATAGTGCTAACCCAAGCTAAGATTAGCCATGCCCTCTCCTCATCTCCTTTGGAAATT | 565 |
| | | | | GCAGCAGCTGAGGCTGCAACTCTTGCTCCAAGCAATAGAACTGTTGGTACGTTTGAAGCCAAATTTGAT | |
| | | | | AGGACAAGTATAACTATAGCAAGTATAGCTGTTNCACTAGCATGATCTATTCGAGAATAGGGCTCCATT | |
| | | | | AAGTCCCACATAGCACTAGGAATTCCAGTGGACTTAAAGCCCTCTACTGTGATAAACATTCCACAGAAGA | |
| | | | | ATACCAAAAGTGAATATGAGACCTTGTCTAAGCTTGGCCCTGCATCTTTGAAATCAAGAACCACCAAAGT | |
| | | | | TATTGCAGCTGCAATTGCAGTCCATGCCATATTCAAACCAATGAGCATTGCAATCAACATTACTAGTGTG | |
| | | | | ATTGCATAAACACAAGATTTCCACAGTACCCTCTTCCATTTTTTGTTCATATAGTCCTTTTCTCCTGAAGAG | |
| | | | | AGTATAACA[A/T]GTGCTTCAATAGGTTTATCCACTACTATTGCAACATTTTTTGAAGGATTAGTTTCCTC | |
| | | | | CTTTGTATCATTTGTCCCTTCCTTGGATGCATTTGAGTTTGTTGTGAAATCCTTTGTGCCACTATGAACCAT | |
| | | | | CTGAACTTCACTTTCACTTGGAGTTGATTGGTCTCTCATAACATGAACTTGGGAAGAGTTTTGAATACTAT | |
| | | | | TAGCAAGTTCTACACTGCCATTGCATTCTTGAGAATTAAAGGATGTAAAATGAGACATTCTGGCTGGAG | |
| | | | | AAAACTGATGAGAAACAACCTCCTCATCCACTACNACTTCTGAAACTGGATCCTCTTCATCCTTAGGACA | |
| | | | | AGATAGTACCTTCCAATACATGGCTATAAGAAATACAACATTCACCACAACTCCTACAAGCATAGCTGGA | |
| | | | | AGAATACCTATCAGAAACCTCCCAAATGATATTTTACCTTGAATAGCTATAACCAAATTCTGGGGGTTCC | |
| | | | | CAATTGGGGTTGCTGAGGAT | |
| SY4207 | SY4207F1 | CCAAGGA | | | 904 |
| | | AGGGACA | | | |
| | | AATGATA | | | |
| | | CAAAG | | | |
| SY4207 | SY4207R1 | TGCAGTC | | | 905 |
| | | CATGCCA | | | |
| | | TATTCAA | | | |
| | | AC | | | |
| SY4207 | SY4207A1FM | CCTATTG | T | | 906 |
| | | AAGCACA | | | |
| | | TGT | | | |
| SY4207 | SY4207A2TT | ACCTATT | A | | 907 |
| | | GAAGCAC | | | |
| | | TTGT | | | |
| SY4278 | | | | GATGTACATGTAGAATTTCACAAAGCATAAATTGTATTTTATGTAACATATTGTTCTTGGTAGACAGGAA | 566 |
| | | | | AGAAATTGCAGAAATGAAAAGAAGACTAGCAGCACAAATATATGGCATACTCCCTAGCTATCATATCAA | |
| | | | | CCAAAAGGATACGTATATACGAGCAAATGCACACAACCTATCATATCAACCAAAAAAAGGATATATGAC | |
| | | | | TCATGTCAGAAGGTGGAGTTTCAATTCCAACATGGTAACGTTGTTCTCACCATGAACTCCCATTTTTGCAA | |
| | | | | AAAAATATGCTCCCAAGTTTGTCTCTCTAAGAACATGGCCAACAGAGCAATCCCTCAAAAGTTGTAGCAA | |
| | | | | GATACGGATGTCTGCTATGATAACCGGCCTCTCCTAAAGGTGAGAAGATGAGTGTGATAGGAAGTGATC | |
| | | | | AACTAGACCAGACACNNNNATAGATGATAGACTTATTAGGTTATTAACTAGCTAGGTTAAACATTAACT | |
| | | | | ACAACAAAACAAT[A/G]AGTGATTGAATAACTATCAACAAGCGACTTATATTTCATCAAATTGTGATTTT | |
| | | | | TTTGGAACCCATAAGAGAGATCTAAAGAAAATTTGTCGAATACTTCTATACTGAATAGATATTCGGTCAA | |
| | | | | AATAGCAGGATAGGTGTTCGGCTCACTACGAGACAAAAAGAGTCCTAAGTAAGAAGCAATGAAGATTA | |
| | | | | AGCCCTTAATCATAGAATCATATATATCACCTAAAAATGGCAATCACCCNNNNGAAAGTTCAAGGGCCA | |
| | | | | AGGTTNAAAAATGTTAGTATAATTAAATAAAAATGAGATGTTTAAGGTAAAGGTAGCTGTTCACCCTCAT | |
| | | | | TTATTAATGATATTTATTACTCTGAACTGGAATCTTACTTGAATTCAGAATGNNNTTTTTTTTTNCACNGA | |
| | | | | TACTCANTTTTTAAAGTATTACTTCATCGATCGAATACTAAAAGAAAACATATTAAAGTAAAAAACACAA | |
| | | | | AAAAAAAGAGAAGGTATCTGATCATCTTTTGGT | |
| SY4278 | SY4278F1 | AACCGGC | | | 908 |
| | | CTCTCCTA | | | |
| | | AAGG | | | |
| SY4278 | SY4278R1 | TTGATGA | | | 909 |
| | | AATATAA | | | |
| | | GTCGCTT | | | |
| | | GTTGATAG | | | |
| SY4278 | SY4278A1FM | TATTCAAT | G | | 910 |
| | | CACTCATTG | | | |
| SY4278 | SY4278A2TT | TTATTCAA | A | | 911 |
| | | TCACTTAT | | | |
| | | TGT | | | |
| SY4255 | | | | TATATAATAGAAAGTCACCTTTCAGCATGTGGTATGAAGTTTTCGTGATAGGTTTTCAGCTTTAAGGATG | 567 |
| | | | | CIGCAGTTCTCTTCTCTTATTTTCCAAATAGATTAAAATATTTCTAAATCTCAATCCCGAAAAAGACTTACA | |
| | | | | AAACGTTTATAGCTTTTTCATGAAGTAAATCCAATGCAAGGACTGCAAGGTGTGGAATTCCAAGTTATAT | |
| | | | | CGAAGCCCATGAANGAATTTTCTTTATAAGGTAAGACTANGATGTAAAAAAGTTTGATAAACGTTTTTGC | |
| | | | | TGTTTTCCTTTTGCAGTTTTATTAAATTAAACGTTATGTATGATTANTTTGATGATTATTTGCACAATATGT | |
| | | | | TTACGTACTATGCATGACCACATAAATTAAAATGAAATAAAGAGAATATGGGATTTCANCGTTATCTTTG | |
| | | | | AGATGCAACGTATTTGTAAATATATTGTTTTAAATATTAATATATATGCTGATTTTACTGAATAANTTTTTT | |
| | | | | ACGT[A/T]TGCTAGCTCTTAATTGTTTTCCATTTCTGGGTTGTATTATGGGTCCTTTTATTTACTTAGAGTA | |
| | | | | GGCTTGTTTATGAAGGAAGTGAATATTCGTGCATTTTGAGATTTTGATCACNTTNTGGGATATAGCACAT | |
| | | | | CATTTAGGTCGTTGAAAGTGTATATNACACTGTCATATTATATAAAAGTNAGNTTTTTTTTTTAAAAAAAA | |
| | | | | TATTACTNAAAGTGTAATCTGCCAATTATTTTAGTCNAAGTTTATATAGATGAGTAGGGTTTAATGATTTT | |
| | | | | TGCTGGGTTTGATGATTAGGGTAATAGTTTACATAGAGGGTTTATTTTTGGGGTGCATATATTTAGGCTA | |
| | | | | GAGGATTCATCATTTTACATAACTATTGAGCTAGTTGTGTNAGGGGCAAGTATTCCTTGTACCCTTCATCT | |
| | | | | TCTTCCTTATATTAATAATGTTTGCTCTTAGCCNATAAACAAAAAATAGTTTAAATTTCANAATATCATTAC | |
| | | | | TTAAATGATAA | |
| SY4255 | SY4255F1 | ATCTTTG | | | 912 |
| | | AGATGCA | | | |
| | | ACGTATT | | | |
| | | TGTA | | | |
| SY4255 | SY4255R1 | CAACGAC | | | 913 |
| | | CTAAATG | | | |
| | | ATGTGCT | | | |
| | | ATATCC | | | |
| SY4255 | SY4255A1FM | TTTTACGT | A | | 914 |
| | | ATGCTAGC | | | |
| SY4255 | SY4255A2TT | TTTACGTT | T | | 915 |
| | | TGCTAGC | | | |
| SY4300 | | | | TTTTAAATATTAATATATATGCTGATTTTACTGAATAANTTTTTACGTNTGCTAGCTCTTAATTGTTTTCC | 568 |
| | | | | ATTTCTGGGTTGTATTATGGGTCCTTTTATTTACTTAGAGTAGGCTTGTTTATGAAGGAAGTGAATATTCG | |
| | | | | TGCATTTTGAGATTTTGATCACNTTNTGGGATATAGCACATCATTTAGGTCGTTGAAAGTGTATATNACA | |
| | | | | CTGTCATATTATATAAAAGTNAGNTTTTTTTTTTAAAAAAAATATTACTNAAAGTGTAATCTGCCAATTAT | |
| | | | | TTTAGTCNAAGTTTATATAGATGAGTAGGGTTTAATGATTTTTGCTGGGTTTGATGATTAGGGTAATAGT | |
| | | | | TTACATAGAGGGTTTATTTTTGGGGTGCATATATTTAGGCTAGAGGATTCATCATTTTACATAACTATTGA | |
| | | | | GCTAGTTCTGTNAGGGGCAAGTATTCCTTGTACCCTTCATCTTCTTCCTTATATTAATAATGTTTGCTCTTA | |
| | | | | GCC[A/G [symbol] ATAAACAAAAAATAGTTTAAATTTCANAATATCATTACTTAAATGATAATATTAATATATTTAA | |
| | | | | GAACAAAGGAAACAGNTACGTTTAGGAGCNNNNTTATTTGACATTTTAGGAACTTTNAAAAAAATGAA | |
| | | | | AATTTGAGAATTTAACGTGACTATATTTACACTTTCAGACCAAAATGGTGTTTTATCNNNNNTTTTATAGT | |
| | | | | CCTATCGGGCTAGAACCTACCCCATATGTAGTTTAATTTCCTCCATGTTAACTATGTAACTACTCTATTTTG | |
| | | | | TTTGTTTGCACCTACCAAGTATGGCACATAGCAANNTTTAAAAAAGAAATTAGTGGCCCTTATGCACTAT | |
| | | | | TTTCTTTTTGAAAGGGGAAAAGAAAAAGGAAACTAATACATAGCTNGAGTACATCTTTGATGTCGTTATA | |
| | | | | GTGTTTGAATAACAAGAGATTTGGATTTAGTAATTATATGGAGGATGCACNNNATGATGTAGTTGGAAA | |
| | | | | ATATCTTATCTTATTAAATAT | |
| SY4300 | SY4300F1 | GCAAGTA | | | 916 |
| | | TTCCTTGT | | | |
| | | ACCCTTC | | | |
| | | ATC | | | |
| SY4300 | SY4300R1 | TTGGTCT | | | 917 |
| | | GAAAGTG | | | |
| | | TAAATAT | | | |
| | | AGTCACG | | | |
| SY4300 | SY4300A1FM | TTGCTCTT | A | | 918 |
| | | AGCCAATA | | | |
| SY4300 | SY4300A2TT | TGCTCTTA | G | | 919 |
| | | GCCGATA | | | |
| SY4301 | | | | CCTAGATTTAATAAAAATATNTGTCAGTATTNAAAAAAAATCTCGTGCTTTTTATTTGATTGCTGAAGAA | 569 |
| | | | | AAAAATTANCAATTATGTAGTATAAGTTANAAAAAAANTCATATTCTCCCTCAACAAGAAATTATAGTTA | |
| | | | | ATAAGAGTTTCAAAAAAGTTACTATAATGATCAACCAGCTTATTTTATGTGATAATTTATTATTAAATTAT | |
| | | | | ATAAAATTATTTTATATTATCAGTGTATAATTTATTTTNNAAAAAAAAATACATAAATAGTTTATTGTGTC | |
| | | | | AGATAAATTCTGATTGGAAGCTATTAAAGTTGCCCTTTAGAACTTTTATATATTTTCCAGTGTGCACATAT | |
| | | | | ANAAAAGAGGGTCACCTTTCACCTAGTGGTATGAAGTGGAAGTGTCTTGAACAATAGTCTTCANNNNN | |
| | | | | GAGGATGCTGCAATTCTCTTCCACCATTTNCCAAACAAATTAAAATGTTTCTGAATCCAATTTGGAAACCA | |
| | | | | AAAAGTTC[A/G]TAGCTTTTCATAGAGCTTTCAAGTAATTAGCCCCGTGAAATATTTTTCTTTTTACGCAA | |
| | | | | GATGACACCACGGGCCTTCTAACTGAAACAAGCAATAATAATAACAGGCCAAGGAAAAACTCAGCAGC | |
| | | | | ATTCTGTTAGAAGGAAAACACTTATCTCTATTAGCATATTTNTCTCCATTAATCTCTTACTTCAGTCCTCTA | |
| | | | | TCAGGTAAGAGTAGGACGTAAAATGTGTGATAGATGTTTGTGTTATTGTTTTCCTTTTCCAGTTCTGTTGA | |
| | | | | ATTAAATGCTGTATTATATGATATCCTTGCTGCTAATATGCTTAACTGAATGAGTTTTTTACGTATGCTCTA | |
| | | | | CGTGTTTTCCAATTCTTGCTTTTTCTAAAAGAATTTCTTGAACCTCCCCTCATATTTCCTTTGTGAACTTACC | |
| | | | | AGATTTCATGCATTTTGAGAGTCATAGAATTTCTTATTATAGTTAGGCCGATTCTGCTAGAATCTAGCTAC | |
| | | | | TGCATGCATGTACT | |
| SY4301 | SY4301F1 | GCTGCAA | | | 920 |
| | | TTCTCTTC | | | |
| | | CACCATT | | | |
| SY4301 | SY4301R1 | CGTGGTG | | | 921 |
| | | TCATCTTG | | | |
| | | CGTAA | | | |
| SY4301 | SY4301A1FM | TCTATGA | G | | 922 |
| | | AAAGCTA | | | |
| | | CGAACTT | | | |
| SY4301 | SY4301A2TT | CTCTATG | A | | 923 |
| | | AAAAGCT | | | |
| | | ATGAACT | | | |
| | | TT | | | |
| SY4244 | | | | TGAGGCTGCAACTCTTGCTCCAAGCAATAGAACTAGAAAACATATGAATAGAAGATTGTGATGCATCAA | 570 |
| | | | | ATGAAAATGGCTCTTGCTAAGTATGGATATAAAAAAAAAAAATACTGGTAGTTTAGACAAGAAATGAAT | |
| | | | | GTGAACCTTCATTATGACTAAATCTAGATTGCATATATGTATTATGACTTAACATTGCATACTTTTTTTTGC | |
| | | | | CCATGACAATTGAAAATTTTAGGGAAAATATAATCCTAACATATTTCTTTTTGTATGCTACTATAGTTTAT | |
| | | | | ATCAAACTTGGTTTAAAANTTTAAAATTGACTAATGTCAAACACATAAATTTAACATTATATCTTATAGCA | |
| | | | | GTCATGAAAGTTCAAATTAATTAAAAAAATGCTNCATGTTACGTATANCTAATTCTNGGAAACAATATAA | |
| | | | | GNCATGCATAACAGAAGCCATTTGAAGATTTACCATTCACTAATTGTCATTTAAGCAAGGTAAAAAAAAT | |
| | | | | TAGTAGAC[A/C]GTCTTTTAATCATGAATCGAACATAAAATAGATGGGTAACATATAAATAAACAAGTTG | |
| | | | | TACCAGAAAAATAATTATTTTGGCAGGAAGATCATGCACTTACCTGTTGGTACGTTTGAAGCCAAATTTG | |
| | | | | ATAGGACAAGTATAACTATAGCAAGTATAGCTGTTNCACTAGCATGATCTATTCGAGAATAGGGCTCCA | |
| | | | | TTAAGTCCCACATAGCACTAGGAATTCCAGTGGACTTAAAGCCCTCTACTGTGATAAACATTCCACAGAA | |
| | | | | GAATACCAAAAGTGAATATGAGACCTGTTCCAAGAAGAAAATCACTTTAAGAGATTGTTCGAAGTTCAG | |
| | | | | TTGGCACAAGAATAACAATAGCTAGCATTTCAAATGGAAAAGAGAGAAAATGAGGTTTTGTACAAGNG | |
| | | | | GTTGTGTTATATAAGCTTTACCTTGTCTAAGCTTGGCCCTGCATCTTTGAAATCAAGAACCACCAAAGTTA | |
| | | | | TTGCAGCTGCAATTGCAGTCCATGC | |
| SY4244 | SY4244F1 | AACAGAA | | | 924 |
| | | GCCATTT | | | |
| | | GAAGATT | | | |
| | | TACCA | | | |
| SY4244 | SY4244R1 | GTGCATG | | | 925 |
| | | ATCTTCCT | | | |
| | | GCCAA | | | |
| SY4244 | SY4244A1FM | CATGATT | C | | 926 |
| | | AAAAGAC | | | |
| | | GGTCTA | | | |
| SY4244 | SY4244A2TT | TTCATGA | A | | 927 |
| | | TTAAAAG | | | |
| | | ACTGTC | | | |
| SY4295 | | | | TAATAAAATAAAATAAATAAAATGACTTATCAAATTTTAAGTTTTCTATAAGTTTTTAGAATATTTTATTTT | 571 |
| | | | | TATTGATTAANTATTTTACTAATTTTATTNCACTTTTTTGAAANTGAGTAAAAAACAAAAATAAAAAATNT | |
| | | | | ATATANNATTTAATTTTAAACTGGATAAATAATTTGATGAATGAGTCTTTTTTTTAGCCAATAAGTGCAT | |
| | | | | CTTTTATTGATTTGATTTACATTATATTTTATTTAATCTGTCACNNCATGTAACTGATGGAGATGGCATCCC | |
| | | | | CATCTTTGGTTCTAGTCATCACTTATGGATTGACCACAACGACTCACTCTTCAATTGCACTGATGGCCTAA | |
| | | | | TTGGTGCTGTTATGGGCTCCACTGCCATTACCATTTCCAACAACTGCTTCATCCCCTAACTAGGTCGATCT | |
| | | | | ACTAATTAATAGTCCTTTTGGTTAAAATATTTGAAGGAATTCTCTCATCATGTGTTTACTTTGTTTTAACCA | |
| | | | | AGTA[A/G]TACGNCCATAGAGAGNTAGTGTTGAGTTTATTGACTTCAAAATTATTCAGGTTATGCTACTG | |
| | | | | GCTCAACGTGACTCTTATGTCCACGATCAGCATATGCAAGGAATCAATGCATACAACCATTTCGGGGAG | |
| | | | | AATCTTAACCAAAGAATGCCCAGGTAATTAACTAACATCTTTTANGTAGTAGTAGTATCTCTAGATATTTT | |
| | | | | ACTTTTTTTTTTNNAATTGTATATGTCATTCCATCTAACATTTTGTTCAATTCTATGATAATAATTTTTATTA | |
| | | | | CTTATTATTTTTAAAAATAGNCTTAGTTACTATTTTGNTCTTNTAATTTAATTTTNAAGTTCAATTTGATTC | |
| | | | | TCTTAATATTAAAAAGTTNAATTTNNTCCTCTNANNNNNNTTTTTTTAAATGACTTAATTAGNTCCTTTTA | |
| | | | | CTTTTAGAAGTTTCAATTAAGTCATTTATTTTTTAAAATAGGTTCAATTTGATCTTATTTTTCTTTCATGTGC | |
| | | | | TNCAANTTT | |
| SY4295 | SY4295F1 | GAAGGAA | | | 928 |
| | | TTCTCTCA | | | |
| | | TCATGTG | | | |
| | | TTTAC | | | |
| SY4295 | SY4295R1 | TGAGCCA | | | 929 |
| | | GTAGCAT | | | |
| | | AACCTGAA | | | |
| SY4295 | SY4295A1FM | TGTTTTAA | A | | 930 |
| | | CCAAGTA | | | |
| | | ATACG | | | |
| SY4295 | SY4295A2TT | TTGTTTTA | G | | 931 |
| | | ACCAAGT | | | |
| | | AGTAC | | | |
| SY4254 | | | | TTGAAATGATTANGTGAATNTAAGCAAAAAATGTCGCGTTAAATTCGTATGTTTGTTATTATTTTTCTTGT | 572 |
| | | | | GGTATAATTTTTATTGTGTTTTTATAATCTTTCGTGTGAAGATTGATTATCCAACACATTTTTAATTATAGGT | |
| | | | | ATGTCCTACATTTGTCTCTATCCAAAATCCTCATGCCTTTAATTTGATANGAATACATGTTTCAACAGCTTC | |
| | | | | TTACAAACNNATATNNAATCTTATAAATATATTCTTTATTATTAATTAAAATTTATAAATTTCACTTCTTAT | |
| | | | | TTAAAAAATTAATATCACTCATAATTTTATAATTTTATTAATAAAAAATATATTAAGAAAAATGTGTTNAA | |
| | | | | AAATATCTCAAATAATAATGATTGGAAGCTATTACTAAAGTTGCCGCATATGACTTTTATAATCCAGAGT | |
| | | | | GTCCGTATAAATAGAGGGTCCACTTTCACCAAGTAGNACCAAGTTAAGTTGCCTCTTATGACTTTTATAC | |
| | | | | TC[*/A]AAGAGTGTACATGAAAAGTGCAAAGAGAGGGTCACTTTTCGCCAAGTGGTATTACACAGTTGC | |
| | | | | CGCTTATGACTTATCCTTTCTCACCAGGTTGCTAGCATGGAAGAAGTGTACTTAATTAGTGTGCACANAT | |
| | | | | ATAAATAGAGGGTCACCTTAATTTCTCATTCTCACACCAAGTANTAGTGTCTTGACAAGTTTTCAGCTCTG | |
| | | | | TTGCAATTCCCTCTTCNACCATTTTGCTAGCAAACTCACATTTTTATACTCAATCCAATTCAGAAAAAAACT | |
| | | | | CTTANAAAAGTTCATATAGCTTTTCATGGAGCCTTCAAGTTATTAAGGTAAGAATAGCGAGTAAAGTGTG | |
| | | | | TGCATGATAAGTGTCTNTGTTGTTTTTCTATTCCAGCTCTTTTGAATTAATTGTTATATGTATATTATATAA | |
| | | | | TATCTTTGCTTCTAATTGGATCAATTTTGTTTTCCAATATAATTCTGGCTTTCATGGGTGGTTTTGATTTAC | |
| | | | | TAATAGCGT | |
| SY4254 | SY4254F1 | GGCGAAA | | | 932 |
| | | AGTGACC | | | |
| | | CTCTCT | | | |
| SY4254 | SY4254R1 | ACCAAGT | | | 933 |
| | | TAAGTTG | | | |
| | | CCTCTTAT | | | |
| | | GAC | | | |
| SY4254 | SY4254A1FM | TCATGTA | I | | 934 |
| | | CACTCTTT | | | |
| | | GAGTA | | | |
| SY4254 | SY4254A2TT | TTCATGT | D | | 935 |
| | | ACACTCTT | | | |
| | | GAGTAT | | | |
| SY4302 | | | | AAAGAAAANTTTCTTATTACATAGATTTACTCTAATGTGAAAGAACCTANAANAGTTTTTTTACTTAAAA | 573 |
| | | | | AATCCTTCACGATACCCGCATTTGAGATGCTTTTAAGTACGTTTGTCAAAAGCTGTAAATATGTTAATGCA | |
| | | | | AATATACAATTAAGTGGTAAGTAACCTAATACGAAATGTTAATTCTTAAAACTATGTAACAAATGATAAA | |
| | | | | GCTTTTATCTGCATGGGGTGCAAAGTGTTGACATTATATATTGACACTCAATTTTGTCCAAACAAAGAAA | |
| | | | | GATGAGAAAGAATAAATTAAAAAATCGGATAGAAAATATTAATTAAAAAAATGGAGAACGTTAATGCA | |
| | | | | ATAAAGTGGTACGTACTTTAATATTTAACTGGTAAAACATCTTACTACAACAACCTATCAAATTATAAAGT | |
| | | | | AAAAGTAACGTGCAGGTGCGATGCGAGTGCTAAGTGTGATGACACTGTGTACTAGCACTCAATTTTGTC | |
| | | | | TAAAGACAAAA[A/G]GAAAGAGTTAATAATTAGAAGAAGAAAAGAGACCTTTGTTCAACATGCACACTT | |
| | | | | GTATTGCATTTTACTTCCACTGTGTTCTATGCTTTAAACTCCCACTTACATGTACATGCACCCTCTCAACAT | |
| | | | | GGACACCTGTATAATTAANTTGCATAGGTGGGATCGATTTGTTTAATTTGCTATATATTTAATTAGGTTAA | |
| | | | | TTTCTTGTTGTCTGGCTGATGAGTGATGACATTATCAGTTGGTGGAAAACACGACAATGGATGATATATG | |
| | | | | GGTCCCTTATTCTATTTTTTAAAAATACTTATCTACATAAGGTGTTTGACAACAANAAAATTGATCGAGCT | |
| | | | | CATTTTTTTCCTTTCTAGTTTTATGAATTTCAGCACTTAATTTTGTGATTTTATTCTAGAGGTTCCATCTCGT | |
| | | | | CTCATTGATTAGCTAGGAATATAATTTTTTTTATTATAAGCCAAANATATATTATTAAAATCAAACCACCC | |
| | | | | TCAGCACAAGTTGTGCAA | |
| SY4302 | SY4302F1 | GCGATGC | | | 936 |
| | | GAGTGCT | | | |
| | | AAGTG | | | |
| SY4302 | SY4302R1 | GTGCATG | | | 937 |
| | | TTGAACA | | | |
| | | AAGGTCT | | | |
| | | CTT | | | |
| SY4302 | SY4302A1FM | TAATTATT | G | | 938 |
| | | AACTCTTT | | | |
| | | CCTTTTG | | | |
| SY4302 | SY4302A2TT | ATTATTA | A | | 939 |
| | | ACTCTTTC | | | |
| | | TTTTTGTCT | | | |
| SY4253 | | | | GTAGGTGCAAACAAACAAAATAGAGTAGTTACATAGTTAACATGGAGGAAATTAAACTACATATGGG | 574 |
| | | | | GTAGGTTCTAGCCCGATAGGACTATAAAANNNNNGATAAAACACCATTTTGGTCTGAAAGTGTAAATAT | |
| | | | | AGTCACGTTAAATTCTCAAATTTTCATTTTTTTNAAAGTTCCTAAAATGTCAAATAANNNNGCTCCTAAAC | |
| | | | | GTANCTGTTTCCTTTGTTCTTAAATATATTAATATTATCATTTAAGTAATGATATTNTGAAATTTAAACTAT | |
| | | | | TTTTTGTTTATNGGCTAAGAGCAAACATTATTAATATAAGGAAGAAGATGAAGGGTACAAGGAATACTT | |
| | | | | GCCCCTNACACAACTAGCTCAATAGTTATGTAAAATGATGAATCCTCTAGCCTAAATATATGCACCCCAA | |
| | | | | AAATAAACCCTCTATGTAAACTATTACCCTAATCATCAAACCCAGCAAAAATCATTAAACCCTACTCATCT | |
| | | | | ATATAAACTT[C/G]GACTAAAATAATTGGCAGATTACACTTTNAGTAATATTTTTTTTAAAAAAAAAANCT | |
| | | | | NACTTTTATATAATATGACAGTGTNATATACACTTTCAACGACCTAAATGATGTGCTATATCCCANAANG | |
| | | | | TGATCAAAATCTCAAAATGCACGAATATTCACTTCCTTCATAAACAAGCCTACTCTAAGTAAATAAAAGG | |
| | | | | ACCCATAATACAACCCAGAAATGGAAAACAATTAAGAGCTAGCANACGTAAAAAANTTATTCAGTAAAA | |
| | | | | TCAGCATATATATTAATATTTAAAACAATATATTTACAAATACGTTGCATCTCAAAGATAACGNTGAAATC | |
| | | | | CCATATTCTCTTTATTTCATTTTAATTTATGTGGTCATGCATAGTACGTAAACATATTGTGCAAATAATCAT | |
| | | | | CAAANTAATCATACATAACGTTTAATTTAATAAAACTGCAAAAGGAAAACAGCAAAAACGTTTATCAAAC | |
| | | | | TTTTTTACATCNTAGTCTTAC | |
| SY4253 | SY4253F1 | CCCTAAT | | | 940 |
| | | CATCAAA | | | |
| | | CCCAGCA | | | |
| | | AA | | | |
| SY4253 | SY4253R1 | AGCACAT | | | 941 |
| | | CATTTAG | | | |
| | | GTCGTTG | | | |
| | | AAAG | | | |
| SY4253 | SY4253A1FM | TCATCTAT | C | | 942 |
| | | ATAAACT | | | |
| | | TCGACTAA | | | |
| SY4253 | SY4253A2TT | CTCATCTA | G | | 943 |
| | | TATAAAC | | | |
| | | TTGGACTA | | | |
| SY4247 | | | | GAAATAAAGAGAATATGGGATTTCANCGTTATCTTTGAGATGCAACGTATTTGTAAATATATTGTTTTAA | 575 |
| | | | | ATATTAATATATATGCTGATTTTACTGAATAANTTTTTTACGTNTGCTAGCTCTTAATTGTTTTCCATTTCT | |
| | | | | GGGTTGTATTATGGGTCCTTTTATTTACTTAGAGTAGGCTTGTTTATGAAGGAAGTGAATATTCGTGCAT | |
| | | | | TTTGAGATTTTGATCACNTTNTGGGATATAGCACATCATTTAGGTCGTTGAAAGTGTATATNACACTGTC | |
| | | | | ATATTATATAAAAGTNAGNTTTTTTTTTTAAAAAAAATATTACTNAAAGTGTAATCTGCCAATTATTTTAG | |
| | | | | TCNAAGTTTATATAGATGAGTAGGGTTTAATGATTTTTGCTGGGTTTGATGATTAGGGTAATAGTTTACA | |
| | | | | TAGAGGGTTTATTTTTGGGGTGCATATATTTAGGCTAGAGGATTCATCATTTTACATAACTATTGAGCTA | |
| | | | | GTTGTGT[A/T]AGGGGCAAGTATTCCTTGTACCCTTCATCTTCTTCCTTATATTAATAATGTTTGCTCTTAG | |
| | | | | CCNATAAACAAAAAATAGTTTAAATTTCANAATATCATTACTTAAATGATAATATTAATATATTTAAGAAC | |
| | | | | AAAGGAAACAGNTACGTTTAGGAGCNNNNTTATTTGACATTTTAGGAACTTTNAAAAAAATGAAAATTT | |
| | | | | GAGAATTTAACGTGACTATATTTACACTTTCAGACCAAAATGGTGTTTTATCNNNNNTTTTATAGTCCTAT | |
| | | | | CGGGCTAGAACCTACCCCATATGTAGTTTAATTTCCTCCATGTTAACTATGTAACTACTCTATTTTGTTTGT | |
| | | | | TTGCACCTACCAAGTATGGCACATAGCAANNTTTAAAAAAGAAATTAGTGGCCCTTATGCACTATTTTCT | |
| | | | | TTTTGAAAGGGGAAAAGAAAAAGGAAACTAATACATAGCTNGAGTACATCTTTGATGTCGTTATAGTGT | |
| | | | | TTGAATAACAAGAGATTTG | |
| SY4247 | SY4247F1 | TGCTGGG | | | 944 |
| | | TTTGATG | | | |
| | | ATTAGGG | | | |
| | | TAA | | | |
| SY4247 | SY4247R1 | GGAAGAA | | | 945 |
| | | GATGAAG | | | |
| | | GGTACAA | | | |
| | | GGA | | | |
| SY4247 | SY4247A1FM | TGCCCCT | T | | 946 |
| | | AACACAAC | | | |
| SY4247 | SY4247A2TT | TGCCCCTT | A | | 947 |
| | | ACACAAC | | | |
| SY4257 | | | | TTCCTTCTTTAGGAGAGAAATGACTCACATAGCATAGGATATAGATTTGGTNTAGTAATTAAGGAAATG | 576 |
| | | | | GAGTATTGATNAAATAAAAAAAACGTGATTCTTTACCNTTGAGATTAAGAGTAATGATAAAAAAANTTA | |
| | | | | TTGAAAATTAAGAGTATCTCTTATGTTTAATCGATGTGGTCTCAATCATTAGACGATATTTAATAAGATAA | |
| | | | | GATATTTTCCAACTACATCATNNNGTGCATCCTCCATATAATTACTAAATCCAAATCTCTTGTTATTCAAA | |
| | | | | CACTATAACGACATCAAAGATGTACTCNAGCTATGTATTAGTTTCCTTTTTCTTTTCCCCTTTCAAAAAGA | |
| | | | | AAATAGTGCATAAGGGCCACTAATTTCTTTTTTAAANNTTGCTATGTGCCATACTTGGTAGGTGCAAACA | |
| | | | | AACAAAATAGAGTAGTTACATAGTTAACATGGAGGAAATTAAACTACATATGGGGTAGGTTCTAGCCCG | |
| | | | | ATAGGACTATAAAA[*/ACTTA]GATAAAACACCATTTTGGTCTGAAAGTGTAAATATAGTCACGTTAAAT | |
| | | | | TCTCAAATTTTCATTTTTTTNAAAGTTCCTAAAATGTCAAATAANNNNGCTCCTAAACGTANCTGTTTCCT | |
| | | | | TTGTTCTTAAATATATTAATATTATCATTTAAGTAATGATATTNTGAAATTTAAACTATTTTTTGTTTATNG | |
| | | | | GCTAAGAGCAAACATTATTAATATAAGGAAGAAGATGAAGGGTACAAGGAATACTTGCCCCTNACACA | |
| | | | | ACTAGCTCAATAGTTATGTAAAATGATGAATCCTCTAGCCTAAATATATGCACCCCAAAAATAAACCCTCT | |
| | | | | ATGTAAACTATTACCCTAATCATCAAACCCAGCAAAAATCATTAAACCCTACTCATCTATATAAACTTNGA | |
| | | | | CTAAAATAATTGGCAGATTACACTTTNAGTAATATTTTTTTTAAAAAAAAAANCTNACTTTTATATAATAT | |
| | | | | GACAGTGTNATATACACTTTCAACGACC | |
| SY4257 | SY4257F1 | GGTAGGT | | | 948 |
| | | TCTAGCC | | | |
| | | CGATAGGA | | | |
| SY4257 | SY4257R1 | CGTGACT | | | 949 |
| | | ATATTTAC | | | |
| | | ACTTTCA | | | |
| | | GACCA | | | |
| SY4257 | SY4257A1FM | ATGGTGT | I | | 950 |
| | | TTTATCTA | | | |
| | | AGTTT | | | |
| SY4257 | SY4257A2TT | AAATGGT | D | | 951 |
| | | GTTTTATC | | | |
| | | TTTTAT | | | |
| SY4281 | | | | TAAAAGGCTATTTATCCATATTCAATATCTCAAATGGGTACCTAGCATGTGTATATGCATCATTTAATGGA | 577 |
| | | | | GTACTGACACAGTAAATATATATAGAATAAAGTACATGCCGTGCATTCCAGCAAAATGGGACTACAATA | |
| | | | | AGGATTTTATTGAACTCTCAAAATGCATGCATGAAATCTATTAAGTACACAAAGATAATATTAGTGGACG | |
| | | | | GTTTCAACCACTTCTTTCAGAAAAACCAAGTTTCTATGTTATTTTTATAGCGTGTTTGGGATGTGATTTTCC | |
| | | | | ACGTTTTAGATATGATTTTCAGAAACTAATACTTATAGCTTTCACATCCCAAACGTTATTTCTTAAACGAG | |
| | | | | TTTCCATAAACACGCTATTAGTAAATCAAAACCACCCATGAAAGCCAGAATTATATTGGAAAACAAAATT | |
| | | | | GATCCAATTAGAAGCAAAGATATTATATAATATACATATAACAATTAATTCAAAAGAGCTGGAATAGAA | |
| | | | | AAACAACA[C/G]AGACACTTATCATGCACACACTTTACTCGCTATTCTTACCTTAATAACTTGAAGGCTCC | |
| | | | | ATGAAAAGCTATATGAACTTTTNTAAGAGTTTTTTTCTGAATTGGATTGAGTATAAAAATGTGAGTTTGC | |
| | | | | TAGCAAAATGGTNGAAGAGGGAATTGCAACAGAGCTGAAAACTTGTCAAGACACTANTACTTGGTGTG | |
| | | | | AGAATGAGAAATTAAGGTGACCCTCTATTTATATNTGTGCACACTAATTAAGTACACTTCTTCCATGCTA | |
| | | | | GCAACCTGGTGAGAAAGGATAAGTCATAAGCGGCAACTGTGTAATACCACTTGGCGAAAAGTGACCCT | |
| | | | | CTCTTTGCACTTTTCATGTACACTCTTNGAGTATAAAAGTCATAAGAGGCAACTTAACTTGGTNCTACTTG | |
| | | | | GTGAAAGTGGACCCTCTATTTATACGGACACTCTGGATTATAAAAGTCATATGCGGCAACTTTAGTAATA | |
| | | | | GCTTCCAATCATTATTATTTGAGA | |
| SY4281 | SY4281F1 | AAACCAC | | | 952 |
| | | CCATGAA | | | |
| | | AGCCAGAA | | | |
| SY4281 | SY4281R1 | AAGAATA | | | 953 |
| | | GCGAGTA | | | |
| | | AAGTGTG | | | |
| | | TGC | | | |
| SY4281 | SY4281A1FM | TGATAAG | G | | 954 |
| | | TGTCTCT | | | |
| | | GTTGTT | | | |
| SY4281 | SY4281A2TT | TGATAAG | C | | 955 |
| | | TGTCTGT | | | |
| | | GTTGTT | | | |
| SY4284 | | | | AAAGTACTAGCCATCGAGACTCTAGTGCGCACCACCAGAGAAAAANGTGATTGCCTCATTCCATCAAAC | 578 |
| | | | | TTTACTACTACAAATTCAAGACATACGATATCCAATATTCCTATAATGTACTAGCCATATAGACTCTAGTG | |
| | | | | TGCACCAGTAGAAAGGTTCTTGTCCTTATTCCATCAAACTACTTCTTGTCACAAGCCAATATATACAACAC | |
| | | | | AATAAGATTTAATTTTGTTTTGTAAGATATTTTAAAATTATAAAGAACTTGTAGTTCAAAATATCTTANTTT | |
| | | | | GATACCTATAATTGTAATCTTTTTACTCTTTTGATCATTGTCATGAAATCTCTACTAATAACACCCTTTAAA | |
| | | | | AGTAGCATGACATAACTCATTTAATCCGTGTTCATCGAGTAAATATTTTGAAGTCTTGACACATTTCTAAA | |
| | | | | AGGAAGAGCCAAACTACCTGCGAAAAAGAAGATTCAATCAATACATTTAGATAAGCTTTCTGAGTAGAT | |
| | | | | TTATT[A/G]TCATNATTTTAAAAATCAGACCCTGTCAGTTCTCGTAAATTTATGTTTTTTTTTAAATACTTG | |
| | | | | GTGCGTAGTCCTCTTCACTTCATTTGATTGCTTATACCTATCACAATCGCTTAGAACATAGTCATAGCTCTC | |
| | | | | TATATGGCTAAACACATGCATTCACAGTACATTTGATGATTTCGAACGTTGCCACCATGTATGTAGACTG | |
| | | | | CAAGTGTCCTNATTACATATTATATTACATATGTGATATTTAGTAATATTTTTTTAGTTTAATACTTAATAA | |
| | | | | AAAATTTGTTAAATATCTTTGATAANATTNAAAAAAATATCTTTAAATTATAAATAAACTTTATAAATATG | |
| | | | | TTTTTATGATATTTTGATANTTTTTAATGTTATAAAAAAANTTTAATAATATTTTTATTAAGTCTCAGACNA | |
| | | | | AAAAAATATTATTAAAGGNCACATATATAATATAANAGTGTGTTACTTTTGTTTCGCTGGGGTTGACATA | |
| | | | | AAATTAATTT | |
| SY4284 | SY4284F1 | AAGAGCC | | | 956 |
| | | AAACTAC | | | |
| | | CTGCGAAA | | | |
| SY4284 | SY4284R1 | ACGAGAA | | | 957 |
| | | CTGACAG | | | |
| | | GGTCTGAT | | | |
| SY4284 | SY4284A1FM | TTCTGAG | A | | 958 |
| | | TAGATTT | | | |
| | | ATTATCA | | | |
| SY4284 | SY4284A2TT | TTCTGAG | G | | 959 |
| | | TAGATTT | | | |
| | | ATTGTCG | | | |
| SY4261 | | | | CCTTATTCTATTTTTTAAAAATACTTATCTACATAAGGTGTTTGACAACAANAAAATTGATCGAGCTCATT | 579 |
| | | | | TTTTTCCTTTCTAGTTTTATGAATTTCAGCACTTAATTTTGTGATTTTATTCTAGAGGTTCCATCTCGTCTCA | |
| | | | | TTGATTAGCTAGGAATATAATTTTTTTTATTATAAGCCAAANATATATTATTAAAATCAAACCACCCTCAG | |
| | | | | CACAAGTTGTGCAAAGATAATGATAGTAAAGTTAGTCCTGAAACCTCATATTAAATAATAATAATAATGG | |
| | | | | CATCCAAACTTTTATTNAGGCACTTGTAAAAAATTTAAAGATGTTTGTTTTGTCAAATTTTTGTTCAGAGA | |
| | | | | TTAAAAAGAATCTTGATCAGTCAAATTTTTATTCAATGATTAAGAANTAAATTTTAAAAAAATCAAGAAC | |
| | | | | AAAAACTTTTATAATCCATATGAAATTGATGATAAACTAGGTGTTTGCTTCGTTGTGAAAATTCTGCTATC | |
| | | | | ATA[A/G]CATTCATCGAAAGAAAAAGGAAGGTGGTGCACTTTGGTGGTTTCATCAAGTGAGGTGCTGTC | |
| | | | | TATTCCAAACAAAACTTGTTTGTGCATCATATGTGTGAGAGACTTACTAAATGCAGGTCAGGCATGGCTT | |
| | | | | GAAAAAAGGGAGACAGGCTAGGTCTGTTTCACACAAAAGAAGCGTGGCCAATTATTAAAAAGAACTTG | |
| | | | | ATTAGATATGAAGGGTGTGTTAATAAATATCTCTCAGCAGTATGATGCTCTGTCTTGTTAATTTTGTTTTT | |
| | | | | CTTTTTTAAAGAAAGAGAAAAGGCTTTAGTCTATACGATAAATAAAAAAGAGAAGAAGGNCTTTCTTTG | |
| | | | | TATCACTTTGAAATCATATAATGACTATCATTTTAATTTTTCTCATCAAGAGAAAACTAAATGCTCAAAAA | |
| | | | | TTTGTTTTTATTTTATTAAAAAGGGTAAATAAATACTATTAATACACATAATGATCCAATCTTACAATTTTG | |
| | | | | ATGAATAATTAACAAAG | |
| SY4261 | SY4261F1 | AGGTGTT | | | 960 |
| | | TGCTTCG | | | |
| | | TTGTGAAA | | | |
| SY4261 | SY4261R1 | CCAAAGT | | | 961 |
| | | GCACCAC | | | |
| | | CTTCCTT | | | |
| SY4261 | SY4261A1FM | CTTTCGAT | G | | 962 |
| | | GAATGCT | | | |
| | | ATGA | | | |
| SY4261 | SY4261A2TT | CTTTCGAT | A | | 963 |
| | | GAATGTT | | | |
| | | ATGATA | | | |
| SY4305 | | | | TTAAGAGTAATGATAAAAAAANTTATTGAAAATTAAGAGTATCTCTTATGTTTAATCGATGTGGTCTCAA | 580 |
| | | | | TCATTAGACGATATTTAATAAGATAAGATATTTTCCAACTACATCATNNNGTGCATCCTCCATATAATTAC | |
| | | | | TAAATCCAAATCTCTTGTTATTCAAACACTATAACGACATCAAAGATGTACTCNAGCTATGTATTAGTTTC | |
| | | | | CTTTTTCTTTTCCCCTTTCAAAAAGAAAATAGTGCATAAGGGCCACTAATTTCTTTTTTAAANNTTGCTATG | |
| | | | | TGCCATACTTGGTAGGTGCAAACAAACAAAATAGAGTAGTTACATAGTTAACATGGAGGAAATTAAACT | |
| | | | | ACATATGGGGTAGGTTCTAGCCCGATAGGACTATAAAANNNNNGATAAAACACCATTTTGGTCTGAAA | |
| | | | | GTGTAAATATAGTCACGTTAAATTCTCAAATTTTCATTTTTTTNAAAGTTCCTAAAATGTCAAATAANNNN | |
| | | | | GCTCCTAAACGTA[A/G]CTGTTTCCTTTGTTCTTAAATATATTAATATTATCATTTAAGTAATGATATTNTG | |
| | | | | AAATTTAAACTATTTTTTGTTTATNGGCTAAGAGCAAACATTATTAATATAAGGAAGAAGATGAAGGGTA | |
| | | | | CAAGGAATACTTGCCCCTNACACAACTAGCTCAATAGTTATGTAAAATGATGAATCCTCTAGCCTAAATA | |
| | | | | TATGCACCCCAAAAATAAACCCTCTATGTAAACTATTACCCTAATCATCAAACCCAGCAAAAATCATTAAA | |
| | | | | CCCTACTCATCTATATAAACTTNGACTAAAATAATTGGCAGATTACACTTTNAGTAATATTTTTTTTAAAA | |
| | | | | AAAAAANCTNACTTTTATATAATATGACAGTGTNATATACACTTTCAACGACCTAAATGATGTGCTATAT | |
| | | | | CCCANAANGTGATCAAAATCTCAAAATGCACGAATATTCACTTCCTTCATAAACAAGCCTACTCTAAGTA | |
| | | | | AATAAAAGGACCCATAATACAAC | |
| SY4305 | SY4305F1 | TTGGTCT | | | 964 |
| | | GAAAGTG | | | |
| | | TAAATAT | | | |
| | | AGTCACG | | | |
| SY4305 | SY4305R1 | GCAAGTA | | | 965 |
| | | TTCCTTGT | | | |
| | | ACCCTTC | | | |
| | | ATC | | | |
| SY4305 | SY4305A1FM | CTCCTAA | A | | 966 |
| | | ACGTAAC | | | |
| | | TGT | | | |
| SY4305 | SY4305A2TT | CTCCTAA | G | | 967 |
| | | ACGTAGC | | | |
| | | TG | | | |
| SY4276 | | | | CAAAACTATACTAATAATTGCAATCCANTTGATAATTATATTNTTACCANTGTTTCTTTTTNGCTAAAAGC | 581 |
| | | | | AAGATACCACCACAATTAAAANGACCCTGAGACTAATTTTTTAAGACGTAGAAATTATTAAAATANTTTT | |
| | | | | TTTTCTTTCTTNNTTAGTAAAGTATTTTCTATGTGTATGAATAAAGAATCAAATTCTCGTCAATATACTCAA | |
| | | | | GAAATCCAATTATCATAGCAGTAAATTGTTATGCTATTTCTACTATATTGTTATTATGTTCCTTCTTTAGGA | |
| | | | | GAGAAATGACTCACATAGCATAGGATATAGATTTGGTNTAGTAATTAAGGAAATGGAGTATTGATNAAA | |
| | | | | TAAAAAAAACGTGATTCTTTACCNTTGAGATTAAGAGTAATGATAAAAAAANTTATTGAAAATTAAGAG | |
| | | | | TATCTCTTATGTTTAATCGATGTGGTCTCAATCATTAGACGATATTTAATAAGATAAGATATTTTCCAACT | |
| | | | | ACATCAT[*/CAT]GTGCATCCTCCATATAATTACTAAATCCAAATCTCTTGTTATTCAAACACTATAACGAC | |
| | | | | ATCAAAGATGTACTCNAGCTATGTATTAGTTTCCTTTTTCTTTTCCCCTTTCAAAAAGAAAATAGTGCATA | |
| | | | | AGGGCCACTAATTTCTTTTTTAAANNTTGCTATGTGCCATACTTGGTAGGTGCAAACAAACAAAATAGAG | |
| | | | | TAGTTACATAGTTAACATGGAGGAAATTAAACTACATATGGGGTAGGTTCTAGCCCGATAGGACTATAA | |
| | | | | AANNNNNGATAAAACACCATTTTGGTCTGAAAGTGTAAATATAGTCACGTTAAATTCTCAAATTTTCATT | |
| | | | | TTTTTNAAAGTTCCTAAAATGTCAAATAANNNNGCTCCTAAACGTANCTGTTTCCTTTGTTCTTAAATATA | |
| | | | | TTAATATTATCATTTAAGTAATGATATTNTGAAATTTAAACTATTTTTTGTTTATNGGCTAAGAGCAAACA | |
| | | | | TTATTAATATAAGGAAGAAGA | |
| SY4276 | SY4276F1 | CTCTTATG | | | 968 |
| | | TTTAATC | | | |
| | | GATGTGG | | | |
| | | TCTCAATC | | | |
| SY4276 | SY4276R1 | AGTGGCC | | | 969 |
| | | CTTATGC | | | |
| | | ACTATTTTC | | | |
| SY4276 | SY4276A1FM | CCAACTA | I | | 970 |
| | | CATCATC | | | |
| | | ATGT | | | |
| SY4276 | SY4276A2TT | TTCCAACT | D | | 971 |
| | | ACATCAT | | | |
| | | GTG | | | |
| SY4299 | | | | TATTAAATATAAAGTGGTNTGAAGTGACGTAAGTGCAAGTATATYGTGTGAGTGGCATTTTCAATTAAA | 582 |
| | | | | AAGACAGTGGTACGTCCTAGATTTAATAAAAATATNTGTCAGTATTNAAAAAAAATCTCGTGCTTTTTAT | |
| | | | | TTGATTGCTGAAGAAAAAAATTANCAATTATGTAGTATAAGTTANAAAAAAANTCATATTCTCCCTCAAC | |
| | | | | AAGAAATTATAGTTAATAAGAGTTTCAAAAAAGTTACTATAATGATCAACCAGCTTATTTTATGTGATAA | |
| | | | | TTTATTATTAAATTATATAAAATTATTTTATATTATCAGTGTATAATTTATTTTNNAAAAAAAAATACATAA | |
| | | | | ATAGTTTATTGTGTCAGATAAATTCTGATTGGAAGCTATTAAAGTTGCCCTTTAGAACTTTTATATATTTTC | |
| | | | | CAGTGTGCACATATANAAAAGAGGGTCACCTTTCACCTAGTGGTATGAAGTGGAAGTGTCTTGAACAAT | |
| | | | | AGTCTTCA[*/GCTCT]GAGGATGCTGCAATTCTCTTCCACCATTTNCCAAACAAATTAAAATGTTTCTGAA | |
| | | | | TCCAATTTGGAAACCAAAAAGTTCNTAGCTTTTCATAGAGCTTTCAAGTAATTAGCCCCGTGAAATATTTT | |
| | | | | TCTTTTTACGCAAGATGACACCACGGGCCTTCTAACTGAAACAAGCAATAATAATAACAGGCCAAGGAA | |
| | | | | AAACTCAGCAGCATTCTGTTAGAAGGAAAACACTTATCTCTATTAGCATATTTNTCTCCATTAATCTCTTA | |
| | | | | CTTCAGTCCTCTATCAGGTAAGAGTAGGACGTAAAATGTGTGATAGATGTTTGTGTTATTGTTTTCCTTTT | |
| | | | | CCAGTTCTGTTGAATTAAATGCTGTATTATATGATATCCTTGCTGCTAATATGCTTAACTGAATGAGTTTT | |
| | | | | TTACGTATGCTCTACGTGTTTTCCAATTCTTGCTTTTTCTAAAAGAATTTCTTGAACCTCCCCTCATATTTCC | |
| | | | | TTTGTGAACTTACCAGAT | |
| SY4299 | SY4299F1 | AGTGGTA | | | 972 |
| | | TGAAGTG | | | |
| | | GAAGTGT | | | |
| | | CTTG | | | |
| SY4299 | SY4299R1 | GGCCCGT | | | 973 |
| | | GGTGTCA | | | |
| | | TCTTG | | | |
| SY4299 | SY4299A1FM | TCAGCTC | I | | 974 |
| | | TGAGGAT | | | |
| | | GC | | | |
| SY4299 | SY4299A2TT | AACAATA | D | | 975 |
| | | GTCTTCA | | | |
| | | GAGGATGC | | | |
| SY4291 | | | | ATCAACCAGCTTATTTTATGTGATAATTTATTATTAAATTATATAAAATTATTTTATATTATCAGTGTATAA | 583 |
| | | | | TTTATTTTNNAAAAAAAAATACATAAATAGTTTATTGTGTCAGATAAATTCTGATTGGAAGCTATTAAAG | |
| | | | | TTGCCCTTTAGAACTTTTATATATTTTCCAGTGTGCACATATANAAAAGAGGGTCACCTTTCACCTAGTGG | |
| | | | | TATGAAGTGGAAGTGTCTTGAACAATAGTCTTCANNNNNGAGGATGCTGCAATTCTCTTCCACCATTTN | |
| | | | | CCAAACAAATRTTAAAATGTTTCTGAATICCAATTTGGAAACCAAAAAGTTCNTAGCTTTTCATAGAGCTTTCA | |
| | | | | AGTAATTAGCCCCGTGAAATATTTTTCTTTTTACGCAAGATGACACCACGGGCCTTCTAACTGAAACAAG | |
| | | | | CAATAATAATAACAGGCCAAGGAAAAACTCAGCAGCATTCTGTTAGAAGGAAAACACTTATCTCTATTA | |
| | | | | GCATATTT[C/G]TCTCCATTAATCTCTTACTTCAGTCCTCTATCAGGTAAGAGTAGGACGTAAAATGTGTG | |
| | | | | ATAGATGTTTGTGTTATTGTTTTCCTTTTCCAGTTCTGTTGAATTAAATGCTGTATTATATGATATCCTTGC | |
| | | | | TGCTAATATGCTTAACTGAATGAGTTTTTTACGTATGCTCTACGTGTTTTCCAATTCTTGCTTTTTCTAAAA | |
| | | | | GAATTTCTTGAACCTCCCCTCATATTTCCTTTGTGAACTTACCAGATTTCATGCATTTTGAGAGTCATAGA | |
| | | | | ATTTCTTATTATAGTTAGGCCGATTCTGCTAGAATCTAGCTACTGCATGCATGTACTTTGTTCAATATGTG | |
| | | | | TCAGTGATCCATAAGTAATGCATAATACACATGTTAGTCCATATGGGATCTGGAATTTGATCAATAATAT | |
| | | | | TTGCAGATGATTTCATAACAGACTGATATACGTGCAACGTAATTGTTAGAATAGATTTCGATACCACCTT | |
| | | | | AATTTTGAATTTAGG | |
| SY4291 | SY4291F1 | ACTCAGC | | | 976 |
| | | AGCATTC | | | |
| | | TGTTAGA | | | |
| | | AGGA | | | |
| SY4291 | SY4291R1 | ACTCTTAC | | | 977 |
| | | CTGATAG | | | |
| | | AGGACTG | | | |
| | | AAG | | | |
| SY4291 | SY4291A1FM | ATTAGCA | C | | 978 |
| | | TATTTCTC | | | |
| | | TCCATT | | | |
| SY4291 | SY4291A2TT | TATTAGC | G | | 979 |
| | | ATATTTGT | | | |
| | | CTCCAT | | | |
| SY4303 | | | | AAATAAAATAAATAAAATGACTTATCAAATTTTAAGTTTTCTATAAGTTTTTAGAATATTTTATTTTTATTG | 584 |
| | | | | ATTAANTATTTTACTAATTTTATTNCACTTTTTTGAAANTGAGTAAAAAACAAAAATAAAAAATNTATATA | |
| | | | | NNATTTAATTTTAAACTGGATAAATAATTTGATGAATGAGTCCTTTTTTTTAGCCAATAAGTGCATCTTTT | |
| | | | | ATTGATTTGATTTACATTATATTTTATTTAATCTGTCACNNCATGTAACTGATGGAGATGGCATCCCCATC | |
| | | | | TTTGGTTCTAGTCATCACTTATGGATTGACCACAACGACTCACTCTTCAATTGCACTGATGGCCTAATTGG | |
| | | | | TGCTGTTATGGGCTCCACTGCCATTACCATTTCCAACAACTGCTTCATCCCCTAACTAGGTCGATCTACTA | |
| | | | | ATTAATAGTCCTTTTGGTTAAAATATTTGAAGGAATTCTCTCATCATGTGTTTACTTTGTTTTAACCAAGTA | |
| | | | | NTACG[A/G]CCATAGAGAGNTAGTGTTGAGTTTATTGACTTCAAAATTATTCAGGTTATGCTACTGGCTC | |
| | | | | AACGTGACTCTTATGTCCACGATCAGCATATGCAAGGAATCAATGCATACAACCATTTCGGGGAGAATCT | |
| | | | | TAACCAAAGAATGCCCAGGTAATTAACTAACATCTTTTANGTAGTAGTAGTATCTCTAGATATTTTACTTT | |
| | | | | TTTTTTTNNAATTGTATATGTCATTCCATCTAACATTTTGTTCAATTCTATGATAATAATTTTTAATTACTTAT | |
| | | | | TATTTTTAAAAATAGNCTTAGTTACTATTTTGNTCTTNTAATTTAATTTTNAAGTTCAATTTGATTCTCTTA | |
| | | | | ATATTAAAAAGTTNAATTTNNTCCTCTNANNNNNNTTTTTTTAAATGACTTAATTAGNTCCTTTTACTTTT | |
| | | | | AGAAGTTTCAATTAAGTCATTTATTTTTTAAAATAGGTTCAATTTGATCTTATTTTTCTTTCATGTGCTNCA | |
| | | | | ANTTTTTTTA | |
| SY4303 | SY4303F1 | GGCTCCA | | | 980 |
| | | CTGCCAT | | | |
| | | TACCATT | | | |
| SY4303 | SY4303R1 | TGAGCCA | | | 981 |
| | | GTAGCAT | | | |
| | | AACCTGAA | | | |
| SY4303 | SY4303A1FM | TCTCTATG | G | | 982 |
| | | GCCGTA | | | |
| SY4303 | SY4303A2TT | CTCTCTAT | A | | 883 |
| | | GGTCGTA | | | |
| SY4273 | | | | CTCCATCAGTTACATGNNGTGACAGATTAAATAAAATATAATGTAAATCAAATCAATAAAAGATGCACTT | 585 |
| | | | | ATTGGCTAAAAAAAAGGACTCATTCATCAAATTATTTATCCAGTTTAAAATTAAATNNTATATANATTTTT | |
| | | | | TATTTTTGTTTTTTACTCANTTTCAAAAAAGTGNAATAAAATTAGTAAAATANTTAATCAATAAAAATAAA | |
| | | | | ATATTCTAAAAACTTATAGAAAACTTAAAATTTGATAAGTCATTTTATTTATTTTATTTTATTATTATGCAA | |
| | | | | ATGGTTGGGATTTTCACTTTCATTTTATTTGCATCTAATATTGTACTTAATAATGCATTTATCAAAATTAAG | |
| | | | | TGAAAAAATAAAATTATTTTAATAAAATTGTNTCCTGATAAATATAAATTCTCTTGAAATATTTATTTTCTT | |
| | | | | TTGGGACGAAGGGTTTTTTTTTTTTGCATTTTAAGATCTAGTTTAATAGAATCTATTATTGTAGTACTATTA | |
| | | | | TTGA[A/G]ATTTTGTATTATTGTAGTCCTATTATGCTATAATCCACCACATGTAGTTTAATTTGCTCAACTC | |
| | | | | TGTCACTACTCTATTTTGTGAGTTTTGCACGTACCAAGTGGGGGTACATAGCAATTTAAAAAGAGATACA | |
| | | | | CAATTTTGGATCACAACTTAGATGTAGATGAATTTCAATCCTTAGATGAAAATCAGGTCATTCATGTTTTT | |
| | | | | CTGATTTCACCAATTACGTCTTCCCCTTACTACATTTCCAAATCTCTGATACTAATAACCCCGGACCCAATA | |
| | | | | TATATAAAGGTGTAAGTCTGCTTTCTCAAACCTCCACCTTTTTCACTCATCAAATAAATCAGAAATTAATA | |
| | | | | TCAAATAAATTGTATCTTCAAAATTTAAATGTTTTCTTAACATGCATATGGTATATTATTTTATGTTTTTAA | |
| | | | | TATAAACTGAATGATTTAACTTTAATTTTTTATTTCTTTTNATTTATCATTTTTAACATTTTTAATTCTAAATT | |
| | | | | GAATTTG | |
| SY4273 | SY4273F1 | TTTCTTTT | | | 984 |
| | | GGGACGA | | | |
| | | AGGGTTT | | | |
| SY4273 | SY4273R1 | AGAGTAG | | | 985 |
| | | TGACAGA | | | |
| | | GTTGAGC | | | |
| | | AA | | | |
| SY4273 | SY4273A1FM | CTACAAT | G | | 986 |
| | | AATACAA | | | |
| | | AATCTCA | | | |
| | | ATA | | | |
| SY4273 | SY4273A2TT | CTACAAT | A | | 987 |
| | | AATACAA | | | |
| | | AATTTCA | | | |
| | | ATA | | | |
| SY4256 | | | | AAACCAAGTTTCTATGTTATTTTTATAGCGTGTTTGGGATGTGATTTTCCACGTTTTAGATATGATTTTCA | 586 |
| | | | | GAAACTAATACTTATAGCTTTCACATCCCAAACGTTATTTCTTAAACGAGTTTCCATAAACACGCTATTAG | |
| | | | | TAAATCAAAACCACCCATGAAAGCCAGAATTATATTGGAAAACAAAATTGATCCAATTAGAAGCAAAGA | |
| | | | | TATTATATAATATACATATAACAATTAATTCAAAAGAGCTGGAATAGAAAAACAACANAGACACTTATCA | |
| | | | | TGCACACACTTTACTCGCTATTCTTACCTTAATAACTTGAAGGCTCCATGAAAAGCTATATGAACTTTTNT | |
| | | | | AAGAGTTTTTTTCTGAATTGGATTGAGTATAAAAATGTGAGTTTGCTAGCAAAATGGTNGAAGAGGGAA | |
| | | | | TTGCAACAGAGCTGAAAACTTGTCAAGACACTANTACTTGGTGTGAGAATGAGAAATTAAGGTGACCCT | |
| | | | | CTATTTATAT[A/T]TGTGCACACTAATTAAGTACACTTCTTCCATGCTAGCAACCTGGTGAGAAAGGATA | |
| | | | | AGTCATAAGCGGCAACTGTGTAATACCACTTGGCGAAAAGTGACCCTCTCTTTGCACTTTTCATGTACAC | |
| | | | | TCTTNGAGTATAAAAGTCATAAGAGGCAACTTAACTTGGTNCTACTTGGTGAAAGTGGACCCTCTATTTA | |
| | | | | TACGGACACTCTGGATTATAAAAGTCATATGCGGCAACTTTAGTAATAGCTTCCAATCATTATTATTTGAG | |
| | | | | ATATTTTTNAACACATTTTTCTTAATATATTTTTTATTAATAAAATTATAAAATTATGAGTGATATTAATTTT | |
| | | | | TTAAATAAGAAGTGAAATTTATAAATTTTAATTAATAATAAAGAATATATTTATAAGATTNNATATNNGT | |
| | | | | TTGTAAGAAGCTGTTGAAACATGTATTCNTATCAAATTAAAGGCATGAGGATTTTGGATAGAGACAAAT | |
| | | | | GTAGGACATACCTATAATTAA | |
| SY4256 | SY4256F1 | TGCAACA | | | 988 |
| | | GAGCTGA | | | |
| | | AAACTTG | | | |
| | | TC | | | |
| SY4256 | SY4256R1 | ACACAGT | | | 989 |
| | | TGCCGCT | | | |
| | | TATGAC | | | |
| SY4256 | SY4256A1FM | CCCTCTAT | A | | 990 |
| | | TTATATAT | | | |
| | | GTGCA | | | |
| SY4256 | SY4256A2TT | CCTCTATT | T | | 991 |
| | | TATATTTG | | | |
| | | TGCAC | | | |
| SY4289 | | | | TAGTAAAGTATTTTCTATGTGTATGAATAAAGAATCAAATTCTCGTCAATATACTCAAGAAATCCAATTAT | 587 |
| | | | | CATAGCAGTAAATTGTTATGCTATTTCTACTATATTGTTATTATGTTCCTTCTTTAGGAGAGAAATGACTC | |
| | | | | ACATAGCATAGGATATAGATTTGGTNTAGTAATTAAGGAAATGGAGTATTGATNAAATAAAAAAAACGT | |
| | | | | GATTCTTTACCNTTGAGATTAAGAGTAATGATAAAAAAANTTATTGAAAATTAAGAGTATCTCTTATGTT | |
| | | | | TAATCGATGTGGTCTCAATCATTAGACGATATTTAATAAGATAAGATATTTTCCAACTACATCATNNNGT | |
| | | | | GCATCCTCCATATAATTACTAAATCCAAATCTCTTGTTATTCAAACACTATAACGACATCAAAGATGTACT | |
| | | | | CNAGCTATGTATTAGTTTCCTTTTTCTTTTCCCCTTTCAAAAAGAAAATAGTGCATAAGGGCCACTAATTT | |
| | | | | CTTTTTTAAA[*/AA]TTGCTATGTGCCATACTTGGTAGGTGCAAACAAACAAAATAGAGTAGTTACATAG | |
| | | | | TTAACATGGAGGAAATTAAACTACATATGGGGTAGGTTCTAGCCCGATAGGACTATAAAANNNNNGAT | |
| | | | | AAAACACCATTTTGGTCTGAAAGTGTAAATATAGTCACGTTAAATTCTCAAATTTTCATTTTTTTNAAAGT | |
| | | | | TCCTAAAATGTCAAATAANNNNGCTCCTAAACGTANCTGTTTCCTTTGTTCTTAAATATATTAATATTATC | |
| | | | | ATTTAAGTAATGATATTNTGAAATTTAAACTATTTTTTGTTTATNGGCTAAGAGCAAACATTATTAATATA | |
| | | | | AGGAAGAAGATGAAGGGTACAAGGAATACTTGCCCCTNACACAACTAGCTCAATAGTTATGTAAAATG | |
| | | | | ATGAATCCTCTAGCCTAAATATATGCACCCCAAAAATAAACCCTCTATGTAAACTATTACCCTAATCATCA | |
| | | | | AACCCAGCAAAAATCATTAAACCCT | |
| SY4289 | SY4289F1 | AGTGCAT | | | 992 |
| | | AAGGGCC | | | |
| | | ACTAATTTC | | | |
| SY4289 | SY4289R1 | CTATTTTG | | | 993 |
| | | TTTGTTTG | | | |
| | | CACCTAC | | | |
| | | CA | | | |
| SY4289 | SY4289A1FM | TGGCACA | I | | 994 |
| | | TAGCAAT | | | |
| | | TTTTAA | | | |
| SY4289 | SY4289A2TT | TATGGCA | D | | 995 |
| | | CATAGCA | | | |
| | | ATTTAAA | | | |
| SY4285 | | | | TACCACCACAATTAAAANGACCCTGAGACTAATTTTTTAAGACGTAGAAATTATTAAAATANTTTTTTTTC | 588 |
| | | | | TTTCTTNNTTAGTAAAGTATTTTTCTATGTGTATGAATAAAGAATCAAATTCTCGTCAATATACTCAAGAAA | |
| | | | | TCCAATTATCATAGCAGTAAATTGTTATGCTATTTCTACTATATTGTTATTATGTTCCTTCTTTAGGAGAGA | |
| | | | | AATGACTCACATAGCATAGGATATAGATTTGGTNTAGTAATTAAGGAAATGGAGTATTGATNAAATAAA | |
| | | | | AAAAACGTGATTCTTTACCNTTGAGATTAAGAGTAATGATAAAAAAANTTATTGAAAATTAAGAGTATCT | |
| | | | | CTTATGTTTAATCGATGTGGTCTCAATCATTAGACGATATTTAATAAGATAAGATATTTTCCAACTACATC | |
| | | | | ATNNNGTGCATCCTCCATATAATTACTAAATCCAAATCTCTTGTTATTCAAACACTATAACGACATCAAAG | |
| | | | | ATGTACTC[A/G]AGCTATGTATTAGTTTCCTTTTTCTTTTCCCCTTTCAAAAAGAAAATAGTGCATAAGGG | |
| | | | | CCACTAATTTCTTTTTTAAANNTTGCTATGTGCCATACTTGGTAGGTGCAAACAAACAAAATAGAGTAGT | |
| | | | | TACATAGTTAACATGGAGGAAATTAAACTACATATGGGGTAGGTTCTAGCCCGATAGGACTATAAAANN | |
| | | | | NNNGATAAAACACCATTTTGGTCTGAAAGTGTAAATATAGTCACGTTAAATTCTCAAATTTTCATTTTTTT | |
| | | | | NAAAGTTCCTAAAATGTCAAATAANNNNGCTCCTAAACGTANCTGTTTCCTTTGTTCTTAAATATATTAA | |
| | | | | TATTATCATTTAAGTAATGATATTNTGAAATTTAAACTATTTTTTGTTTATNGGCTAAGAGCAAACATTAT | |
| | | | | TAATATAAGGAAGAAGATGAAGGGTACAAGGAATACTTGCCCCTNACACAACTAGCTCAATAGTTATGT | |
| | | | | AAAATGATGAATCCTCTAGCCTA | |
| SY4285 | SY4285F1 | AAATCCA | | | 996 |
| | | AATCTCTT | | | |
| | | GTTATTC | | | |
| | | AAACACTA | | | |
| SY4285 | SY4285R1 | CACCTAC | | | 997 |
| | | CAAGTAT | | | |
| | | GGCACAT | | | |
| | | AGC | | | |
| SY4285 | SY4285A1FM | TCAAAGA | A | | 998 |
| | | TGTACTC | | | |
| | | AAGCT | | | |
| SY4285 | SY4285A2TT | CAAAGAT | G | | 999 |
| | | GTACTCG | | | |
| | | AGCT | | | |
| SY4306 | | | | TGAAAGGTGACCCTCTTTTNTATATGTGCACACTGGAAAATATATAAAAGTTCTAAAGGGCAACTTTAAT | 589 |
| | | | | AGCTTCCAATCAGAATTTATCTGACACAATAAACTATTTATGTATTTTTTTTTNNAAAATAAATTATACACT | |
| | | | | GATAATATAAAATAATTTTATATAATTTAATAATAAATTATCACATAAAATAAGCTGGTTGATCATTATAG | |
| | | | | TAACTTTTTTGAAACTCTTATTAACTATAATTTCTTGTTGAGGGAGAATATGANTTTTTTTNTAACTTATAC | |
| | | | | TACATAATTGNTAATTTTTTTCTTCAGCAATCAAATAAAAAGCACGAGATTTTTTTTNAATACTGACANAT | |
| | | | | ATTTTTATTAAATCTAGGACGTACCACTGTCTTTTTAATTGAAAATGCCACTCACACATATACTTGCACTTA | |
| | | | | CGTCACTTCANACCACTTTATATTTAATAAGTAATTTTGAAATAACTACNACATGATACCATGTTGCAATT | |
| | | | | G[A/G]TACTCTCCACAAAACATTAACATAATGATAATTTGGATAGGATGAATTAATATTTTAACATCGAT | |
| | | | | GTGATATTTGTATTTACATTCAATCTTATCTCTCCGTCTTGCTTCCATCAAAAGGTTGAAATATTTTTGAAG | |
| | | | | TATTCACGCAAGGGAAGCCTTTGAGAACCTATTACATTAGTACGTTGTATGGGTATNATTTTTTTTTTATA | |
| | | | | TATAAAAAAAATCCATACAAGAAATGAGTATTTGATATACTAAAATACATAAATTTCGACATAGTACAAA | |
| | | | | ACATATGATTGGAATTTATTTCCACTATTAAATAGTTAATANGATATATTAGACAAGAAAGGAATAT | |
| | | | | TATCCCATTGATAATGATTGTTCTTTTCTTTTCAGTTTGTATAACTAGATCCTGCTATTCAATTAAAAGAAA | |
| | | | | GGATATAGATTCTAAAACATAAATGAAGTAAAATATTTATAAATTACTATGACAATAANCGGAGTATAAA | |
| | | | | ACATGAACAA | |
| SY4306 | SY4306F1 | GCCACTC | | | 1000 |
| | | ACACATA | | | |
| | | TACTTGC | | | |
| | | ACTT | | | |
| SY4306 | SY4306R1 | TGATGGA | | | 1001 |
| | | AGCAAGA | | | |
| | | CGGAGAG | | | |
| | | AT | | | |
| SY4306 | SY4306A1FM | ACCATGT | A | | 1002 |
| | | TGCAATT | | | |
| | | GATA | | | |
| SY4306 | SY4306A2TT | CCATGTT | G | | 1003 |
| | | GCAATTG | | | |
| | | GTA | | | |
| SY4282 | | | | AAAATGTTGCAATAGTAGTGGATAAACCTATTGAAGCACTTGTTATACTCTCTTCAGGAGAAAAGGACTA | 590 |
| | | | | TATGAACAAAAAATGGAAGAGGGTACTGTGGAAATCTTGTGTTTATGCAATCACACTAGTAATGTTGAT | |
| | | | | TGCAATGCTCATTGGTTTGAATATGGCATGGACTGCAATTGCAGCTGCAATAACTTTGGTGGTTCTTGAT | |
| | | | | TTCAAAGATGCAGGGCCAAGCTTAGACAAGGTAAAGCTTATATAACACAACCNCTTGTACAAAACCTCA | |
| | | | | TTTTCTCTCTTTTCCATTTGAAATGCTAGCTATTGTTATTCTTGTGCCAACTGAACTTCGAACAATCTCTTA | |
| | | | | AAGTGATTTTCTTCTTGGAACAGGTCTCATATTCACTTTTGGTATTCTTCTGTGGAATGTTTATCACAGTA | |
| | | | | GAGGGCTTTAAGTCCACTGGAATTCCAGTGCTATGTGGGACTTAATGGAGCCCTATTCTCGAATAGATC | |
| | | | | ATGCTAGTG[A/G]AACAGCTATACTTCCTATAGTTATACTTGTCCTATCAAATTTGGCTTCAAACGTACCA | |
| | | | | ACAGGTAAGTGCATGATCTTCCTGCCAAAATAATTATTTTTCTGGTACAACTTGTTTATTTATATGTTACCC | |
| | | | | ATCTATTTTATGTTCGATTCATGATTAAAAGACNGTCTACTAATTTTTTTTACCTTGCTTAAATGACAATTA | |
| | | | | GTGAATGGTAAATCTTCAAATGGCTTCTGTTATGCATGNCTTATATTGTTTCCNAGAATTAGNTATACGT | |
| | | | | AACATGNAGCATTTTTTTAATTAATTTGAACTTTCATGACTGCTATAAGATATAATGTTAAATTTATGTGT | |
| | | | | TTGACATTAGTCAATTTTAAANTTTTAAACCAAGTTTGATATAAACTATAGTAGCATACAAAAAGAAATA | |
| | | | | TGTTAGGATTATATTTTCCCTAAAATTTTCAATTGTCATGGGCAAAAAAAAGTATGCAATGTTAAGTCATA | |
| | | | | ATACATATATGCAATC | |
| SY4282 | SY4282F1 | GGGACTT | | | 1004 |
| | | AATGGAG | | | |
| | | CCCTATTC | | | |
| | | TC | | | |
| SY4282 | SY4282R1 | TGGCAGG | | | 1005 |
| | | AAGATCA | | | |
| | | TGCACTTA | | | |
| SY4282 | SY4282A1FM | TCATGCT | A | | 1006 |
| | | AGTGAAA | | | |
| | | CAGCT | | | |
| SY4282 | SY4282A2TT | CATGCTA | G | | 1007 |
| | | GTGGAAC | | | |
| | | AGCT | | | |
| SY4268 | | | | GTCTGGGTAGGGTATTGTGCGTAGACATGTACCAGCACCGGCATTTACACGGGTAACAACTTCTGATTCT | 591 |
| | | | | CATTTCTGTATTAGTTTATATCTATACCTGCAAGTCAATAAATCACTAAAAATATTATTGTTAAATTTTTAG | |
| | | | | AACTAAATCGAAAACTCATCCTGAAATCTTCTAAACATAATCTCATGTGATTAATCTAATTAAGTATGTGA | |
| | | | | TTAAGATNTTCATTTCAAACATAAAAAAGTTACATAAATTTCCAACATAGTATAAAACATAATATTTGAAT | |
| | | | | GATCTTTNTTTTTNNGGGTAAAGATTTGAATGATCTATAGTTACTAAGCAAAAGCATATAATTTTTCACCT | |
| | | | | CAAATATAATTATTTATCAATATAATTAATAAACCCTTTAATTTTTTTTTACTGCAAATAAATCCTATNATC | |
| | | | | AATCATGATGAATGTTTCTTTGATAACAACTACGTTTTCTCTTCACTTCAGGATATAAGAAATGGTCGACT | |
| | | | | TCA[A/G]AAACAAAAACGATAAGAAATGGTCAAAATTTTAAAACTTTGTAACTGAAACAGTGTCAGCTTT | |
| | | | | TACATGATATTGATCAACCTTGAGAGGTTTCCACCCAGGCTAATCAAGATTAAATTAAATGCAACCAATA | |
| | | | | TGTGCTGCCAGAATTAATGTGTTCTGAGGTACTTTATTTGATGGGCTATCATAACAGCTTCGCAGGCTTT | |
| | | | | GTTCTCTCATGTGAAGTTTGAAACAGATTACAAGAAACTGCATGCTACATATGGCANAGCTCTAGTCGA | |
| | | | | GGGAGTATATTGGATGAAGGATTTTCCTCACCAAGTTGCCCTCCTGATTCAATCTGATGTAATTTGATTTC | |
| | | | | TGTTTTGGAATAAAATCAGATGAATTACTCTTAAAAAAAATGTAAAACTTCAAGGAAGTAAAATATCTAT | |
| | | | | TTTAATTTGTACATCNNACAAAATAAATTATATTACAACTAAAATTTGTAATAAATAATATTTTTAAACAT | |
| | | | | ATAAATAATATTTTAG | |
| SY4268 | SY4268F1 | AACAACT | | | 1008 |
| | | ACGTTTTC | | | |
| | | TCTTCACT | | | |
| | | TCAG | | | |
| SY4268 | SY4268R1 | CCTGGGT | | | 1009 |
| | | GGAAACC | | | |
| | | TCTCAA | | | |
| SY4268 | SY4268A1FM | AAATGGT | A | | 1010 |
| | | CGACTTC | | | |
| | | AAAA | | | |
| SY4268 | SY4268A2TT | TGGTCGA | G | | 1011 |
| | | CTTCAGAA | | | |
| SY4269 | | | | CTGAATAAATAGATATATGCTCCAATATATATGCATGACGCTCAAAACCGCGCAGGGAGGCAACAAATT | 592 |
| | | | | AACAAACAACAGTAGCTAGTATTTTAACATTATAGAATTTTAAGTCAACAGACATGCACATTANAAATTG | |
| | | | | AATTTTGNAAATTAAATTTTTATTATTAAGATTTAGNTGACTTATATGNAAAGTAAAATATTATATTTTAC | |
| | | | | TANATGAANATTATTTAAATGAATATATTAGAAGTTGTTTTTATTTCAACTTTTAAGAGAGTTTATTTTTGT | |
| | | | | TTCAACTTAATTTATTTATTATAGTTGGTGATAATTTTTACCGTGAAAAAAAATAGTATATGAAGAGAAA | |
| | | | | GTGTGTGAGAAGAAAGATTGTGAAACAACAGTCACTTTGTTGATAGAAAAATGATTTTGTGTAAGAATG | |
| | | | | TTATCATTTTTTGTAACGTATTCGGTTTTATAGGGTGATACACTATTTGGGAAGAGTTACACTCTTNTAAT | |
| | | | | CATTTTTGT[A/G]ATAGTNAAATACTTTTGAAATGATTANGTGAATNTAAGCAAAAAATGTCGCGTTAAA | |
| | | | | TTCGTATGTTTGTTATTATTTTTCTTGTGGTATAATTTTTATTGTGTTTTTATAATCTTTCGTGTGAAGATTG | |
| | | | | ATTATCCAACACATTTTAATTATAGGTATGTCCTACATTTGTCTCTATCCAAAATCCTCATGCCTTTAATTT | |
| | | | | GATANGAATACATGTTTCAACAGCTTCTTACAAACNNATATNNAATCTTATAAATATATTCTTTATTATTA | |
| | | | | ATTAAAATTTATAAATTTCACTTCTTATTTAAAAAATTAATATCACTCATAATTTTATAATTTTATTAATAAA | |
| | | | | AAATATATTAAGAAAAATGTGTTNAAAAATATCTCAAATAATAATGATTGGAAGCTATTACTAAAGTTGC | |
| | | | | CGCATATGACTTTTATAATCCAGAGTGTCCGTATAAATAGAGGGTCCACTTTCACCAAGTAGNACCAAGT | |
| | | | | TAAGTTGCCTCTTA | |
| SY4269 | SY4269F1 | TGTAACG | | | 1012 |
| | | TATTCGG | | | |
| | | TTTTATAG | | | |
| | | GGTGA | | | |
| SY4269 | SY4269R1 | AACAAAC | | | 1013 |
| | | ATACGAA | | | |
| | | TTTAACG | | | |
| | | CGACAT | | | |
| SY4269 | SY4269A1FM | AATCATTT | A | | 1014 |
| | | TTGTAAT | | | |
| | | AGT | | | |
| SY4269 | SY4269A2TT | AATCATTT | G | | 1015 |
| | | TTGTGAT | | | |
| | | AG | | | |
| SY4272 | | | | ACACAAAGATAATATTAGTGGACGGTTTCAACCACTTCTTTCAGAAAAACCAAGTTTCTATGTTATTTTTA | 593 |
| | | | | TAGCGTGTTTGGGATGTGATTTTCCACGTTTTAGATATGATTTTCAGAAACTAATACTTATAGCTTTCACA | |
| | | | | TCCCAAACGTTATTTCTTAAACGAGTTTCCATAAACACGCTATTAGTAAATCAAAACCACCCATGAAAGCC | |
| | | | | AGAATTATATTGGAAAACAAAATTGATCCAATTAGAAGCAAAGATATTATATAATATACATATAACAATT | |
| | | | | AATTCAAAAGAGCTGGAATAGAAAAACAACANAGACACTTATCATGCACACACTTTACTCGCTATTCTTA | |
| | | | | CCTTAATAACTTGAAGGCTCCATGAAAAGCTATATGAACTTTTNTAAGAGTTTTTTTCTGAATTGGATTGA | |
| | | | | GTATAAAAATGTGAGTTTGCTAGCAAAATGGTNGAAGAGGGAATTGCAACAGAGCTGAAAACTTGTCA | |
| | | | | AGACACTA[A/C]TACTTGGTGTGAGAATGAGAAATTAAGGTGACCCTCTATTTATATNTGTGCACACTAA | |
| | | | | TTAAGTACACTTCTTCCATGCTAGCAACCTGGTGAGAAAGGATAAGTCATAAGCGGCAACTGTGTAATA | |
| | | | | CCACTTGGCGAAAAGTGACCCTCTCTTTGCACTTTTCATGTACACTCTTNGAGTATAAAAGTCATAAGAG | |
| | | | | GCAACTTAACTTGGTNCTACTTGGTGAAAGTGGACCCTCTATTTATACGGACACTCTGGATTATAAAAGT | |
| | | | | CATATGCGGCAACTTTAGTAATAGCTTCCAATCATTATTATTTGAGATATTTTTNAACACATTTTTCTTAAT | |
| | | | | ATATTTTTTATTAATAAAATTATAAAATTATGAGTGATATTAATTTTTTAAATAAGAAGTGAAATTTATAA | |
| | | | | ATTTTAATTAATAATAAAGAATATATTTATAAGATTNNATATNNGTTTGTAAGAAGCTGTTGAAACATGT | |
| | | | | ATTCNTATCAAATTAAAGGC | |
| SY4272 | SY4272F1 | AGAGGG | | | 1016 |
| | | AATTGCA | | | |
| | | ACAGAGC | | | |
| | | TGA | | | |
| SY4272 | SY4272R1 | TGCCGCT | | | 1017 |
| | | TATGACT | | | |
| | | TATCCTTTC | | | |
| SY4272 | SY4272A1FM | TTGTCAA | A | | 1018 |
| | | GACACTA | | | |
| | | ATACTT | | | |
| SY4272 | SY4272A2TT | CAAGACA | C | | 1019 |
| | | CTACTACT | | | |
| | | TGGT | | | |
| SY4250 | | | | CAGGTCTGTGTTCAGTATTTGTCTCAAACATCTCTGTTCTATTTTCAATATTCATTGAAGTTAAATCTTTGG | 594 |
| | | | | CTTGTATGACAAATTGAAGGATGTTTACCTTGGTAAACAGAAGTGGAGGTTTAAGGTGAGAGTGGTTTG | |
| | | | | CATTTGTGATATGTGTCAAGTTAGTGATCCCATTATCCACTTGAATTTGTACAGAGATGTTTGCAAAAATA | |
| | | | | TATCACAATGATGCTTTATACAAGGAATTGTGGCATGTCTGTGTCAACCTTTGACACTCTTTTGAGTGAA | |
| | | | | GGAGAGAGGGTTTATTACTTCCCTCAAGGTCATATGGAACACGTACAAGTTGTATGGGCATGTGCTCTT | |
| | | | | AATTTCTGACTTTAATTNCAGTTAGATGCTTGTGCTACTATTATCTTGCTCCATATCATGTTTTGGATACTA | |
| | | | | GCATAATTGTTTTGTTTCCATTTTCCAATTAAGATCTTATTGGTGTTGGCATGGTTCACCTAACAGTGTTTT | |
| | | | | CTCTT[A/G]ACAATTTTATCTATAAAATTTTTATTTTTCTTGATCTGACTTGTATGCTAGCAGGAGGCGTCT | |
| | | | | TTGTTTANGGTGTGTTTGCTGTAAGTTTTAAAATTGCACAAGTTGCGTCTGGTGGCATTTCAGTTTGGAT | |
| | | | | ATGTTAGATATATTGAACTCAGTTACTATTTCTATTTCCCCTTAAGTTTTAAATGTTAAAATAAATAAATAA | |
| | | | | ATTGAGAGATTAAACCAAATTTGGGTGTATTTTAATTATAGATTTCATGCCAGGATTTAGTGATTTTGGA | |
| | | | | TTTCTTCAGTGTCAAGATTTGTTGGTTTATTTAATTTTAATTTATTTATTTTCTTGACTGATTTAATTGCCTT | |
| | | | | TGCTATTTTTGCAGTGTCTATCTCTGAATATCCAAGATCTTCAAAGGCAATGCTATCTCTGAGAAACATTT | |
| | | | | TTCTGAGGAATGTATAAGCTTGATATTTGCTTTCCTTTGATGATTCATATTTGTTCCTTTTTGGGACTTTAC | |
| | | | | TGTTTAAGA | |
| SY4250 | SY4250F1 | ATTGGTG | | | 1020 |
| | | TTGGCAT | | | |
| | | GGTTCAC | | | |
| SY4250 | SY4250R1 | TAAACAA | | | 1021 |
| | | AGACGCC | | | |
| | | TCCTGCTA | | | |
| SY4250 | SY4250A1FM | AACAGTG | A | | 1022 |
| | | TTTTCTCT | | | |
| | | TAACAAT | | | |
| SY4250 | SY4250A2TT | ACAGTGT | G | | 1023 |
| | | TTTCTCTT | | | |
| | | GACAATT | | | |
| SY4307 | | | | AAGGGTACAAGGAATACTTGCCCCTNACACAACTAGCTCAATAGTTATGTAAAATGATGAATCCTCTAGC | 595 |
| | | | | CTAAATATATGCACCCCAAAAATAAACCCTCTATGTAAACTATTACCCTAATCATCAAACCCAGCAAAAAT | |
| | | | | CATTAAACCCTACTCATCTATATAAACTTNGACTAAAATAATTGGCAGATTACACTTTNAGTAATATTTTT | |
| | | | | TTTAAAAAAAAAANCTNACTTTTATATAATATGACAGTGTNATATACACTTTCAACGACCTAAATGATGT | |
| | | | | GCTATATCCCANAANGTGATCAAAATCTCAAAATGCACGAATATTCACTTCCTTCATAAACAAGCCTACT | |
| | | | | CTAAGTAAATAAAAGGACCCATAATACAACCCAGAAATGGAAAACAATTAAGAGCTAGCANACGTAAA | |
| | | | | AAANTTATTCAGTAAAATCAGCATATATATTAATATTTAAAACAATATATTTACAAATACGTTGCATCTCA | |
| | | | | AAGATAACG[A/G]TGAAATCCCATATTCTCTTTATTTCATTTTAATTTATGTGGTCATGCATAGTACGTAA | |
| | | | | ACATATTGTGCAAATAATCATCAAANTAATCATACATAACGTTTAATTTAATAAAACTGCAAAAGGAAAA | |
| | | | | CAGCAAAAACGTTTATCAAACTTTTTTACATCNTAGTCTTACCTTATAAAGAAAATTCNTTCATGGGCTTC | |
| | | | | GATATAACTTGGAATTCCACACCTTGCAGTCCTTGCATTGGATTTACTTCATGAAAAAGCTATAAACGTTT | |
| | | | | TGTAAGTCTTTTTCGGGATTGAGATTTAGAAATATTTTAATCTATTTGGAAAATAAGAGAAGAGAACTGC | |
| | | | | AGCATCCTTAAAGCTGAAAACCTATCACGAAAACTTCATACCACATGCTGAAAGGTGACTTTCTATTATA | |
| | | | | TAAGTGCACCTGGAGGATAAGAAGAAAATGGCAACTTCGGTAATAACTTCCAATTTAGAATTACACGAA | |
| | | | | TGAACAANTTTTTCTTCATTT | |
| SY4307 | SY4307F1 | CCCATAA | | | 1024 |
| | | TACAACC | | | |
| | | CAGAAAT | | | |
| | | GGAA | | | |
| SY4307 | SY4307R1 | GTTTACG | | | 1025 |
| | | TACTATG | | | |
| | | CATGACC | | | |
| | | ACA | | | |
| SY4307 | SY4307A1FM | ATATGGG | G | | 1026 |
| | | ATTTCACC | | | |
| | | GTTATC | | | |
| SY4307 | SY4307A2TT | AATATGG | A | | 1027 |
| | | GATTTCA | | | |
| | | TCGTTATC | | | |
| SY4265 | | | | ATTCTNGGAAACAATATAAGNCATGCATAACAGAAGCCATTTGAAGATTTACCATTCACTAATTGTCATT | 596 |
| | | | | TAAGCAAGGTAAAAAAAATTAGTAGACNGTCTTTTAATCATGAATCGAACATAAAATAGATGGGTAACA | |
| | | | | TATAAATAAACAAGTTGTACCAGAAAAATAATTATTTTGGCAGGAAGATCATGCACTTACCTGTTGGTAC | |
| | | | | GTTTGAAGCCAAATTTGATAGGACAAGTATAACTATAGCAAGTATAGCTGTTNCACTAGCATGATCTATT | |
| | | | | CGAGAATAGGGCTCCATTAAGTCCCACATAGCACTAGGAATTCCAGTGGACTTAAAGCCCTCTACTGTG | |
| | | | | ATAAACATTCCACAGAAGAATACCAAAAGTGAATATGAGACCTGTTCCAAGAAGAAAATCACTTTAAGA | |
| | | | | GATTGTTCGAAGTTCAGTTGGCACAAGAATAACAATAGCTAGCATTTCAAATGGAAAAGAGAGAAAATG | |
| | | | | AGGTTTTGTACAAG[A/C]GGTTGTGTTATATAAGCTTTACCTTGTCTAAGCTTGGCCCTGCATCTTTGAAA | |
| | | | | TCAAGAACCACCAAAGTTATTGCAGCTGCAATTGCAGTCCATGCCATATTCAAACCAATGAGCATTGCAA | |
| | | | | TCAACATTACTAGTGTGATTGCATAAACACAAGATTTCCACAGTACCCTCTTCCATTTTTTGTTCATATAGT | |
| | | | | CCTTTTCTCCTGAAGAGAGTATAACAAGTGCTTCAATAGGTTTATCCACTACTATTGCAACATTTTTTGAA | |
| | | | | GGATTAGTTTCCTCCTTTGTATCATTTGTCCCTTCCTTGGATGCATTTGAGTTTGTTGTGAAATCCTTTGTG | |
| | | | | CCACTATGAACCATCTGAACTTCACTTTCACTTGGAGTTGATTGGTCTCTCATAACATGAACTTGGGAAG | |
| | | | | AGTTTTGAATACTATTAGCAAGTTCTACACTGCCATTGCATTCTTGAGAATTAAAGGATGTAAAATGAGA | |
| | | | | CATTCTGGCTGGAGAAAACTGA | |
| SY4265 | SY4265F1 | TTCGAAG | | | 1028 |
| | | TTCAGTT | | | |
| | | GGCACAA | | | |
| SY4265 | SY4265R1 | AGGGCCA | | | 1029 |
| | | AGCTTAG | | | |
| | | ACAAGGT | | | |
| | | AA | | | |
| SY4265 | SY4265A1FM | AACACAA | C | | 1030 |
| | | CCGCTTG | | | |
| | | TAC | | | |
| SY4265 | SY4265A2TT | AACACAA | A | | 1031 |
| | | CCTCTTGT | | | |
| | | ACA | | | |
| SY4297 | | | | TACGTATCCTTTTGGTTGATATGATAGCTAGGGAGTATGCCATATATTTGTGCTGCTAGTCTTCTTTTCAT | 597 |
| | | | | TTCTGCAATTTCTTTCCTGTCTACCAAGAACAATATGTTACATAAAATACAATTTATGCTTTGTGAAATTCT | |
| | | | | ACATGTACATCGGTACTTTTGCACCAAGGAAATAAGGGGAGGGGGATACTTTAAATTTGACAGTTTTGT | |
| | | | | ACTTTTGCTTGATTATTTGTTCATTTGTAAAAAATAATATATATAATGGTACATATTATTTTTTACACCCTA | |
| | | | | TCATTTATAGGTTGAATTTGAAGTATGGCAAGAGCTAGTATGAGTTGCTTATAATTGAGTTTTGTTCCTTT | |
| | | | | TTTTTACGTGTTTTGTTCCTTCTAAAATGCTGAAAAGTTTTTTTACNGGTAAACATTATTCTACAGTTGGTC | |
| | | | | TATGCAGCAGTATGCAATCCAAATTACACATTTATGCTATCAATACATAGAAAGCCTTTTCTTTCTCGCCA | |
| | | | | AC[A/G]CCAAGTATAACAAATATCTTATATATGAAGTAAAGCTTTTATGTAATAAAGGATATATGCACTA | |
| | | | | TTAATCTAAATATTGTTGGAGTAGAAATGTAAAGTGAAATNTNNNNNNNNNNNNNNNCTCAGATANA | |
| | | | | AGTGGAAAAGTTGAACAACATATAAGTAAGGAGAACAGCTATACACTTTTTAAGGTTTTAGGTTAAAAT | |
| | | | | GAANTGTCAAATCTCCTTTTATGATAAATTATAAAAGAAAGATTCGTTGTTAAAATTAATAAAGTAAAAA | |
| | | | | ATTATAATAAGATTTCTACTATTCAAATAATTGTACAAGAAGTTAAGAAGATATTCAAAAGAAAATAGCT | |
| | | | | AAAGAAGAAAAAGAGTTTATTACTTAATGAATAAATTATTTTATTAGCTTTATTATTTGACTAGGCATCAT | |
| | | | | ATATCTAGAATATAAAATAAGATATAAATTATAAAAGAAAGGTTGGTTGTTAAAATTAATAAAATAGAA | |
| | | | | AATTATAATAAAATTTCTAC | |
| SY4297 | SY4297F1 | ATGCAGC | | | 1032 |
| | | AGTATGC | | | |
| | | AATCCAA | | | |
| SY4297 | SY4297R1 | TTCACTTT | | | 1033 |
| | | ACATTTCT | | | |
| | | ACTCCAA | | | |
| | | CAATA | | | |
| SY4297 | SY4297A1FM | TTGTTATA | G | | 1034 |
| | | CTTGGCG | | | |
| | | TT | | | |
| SY4297 | SY4297A2TT | TTATACTT | A | | 1035 |
| | | GGTGTTG | | | |
| | | GC | | | |
| SY4279 | | | | TATAGATTCTAAAACATAAATGAAGTAAAATATTTATAAATTACTATGACAATAANCGGAGTATAAAACA | 598 |
| | | | | TGAACAAACTCATAGAAATCAAAGTCAAAATATTAAGAAAAAAATGTTAGCTGGCCAACAANNNNNGC | |
| | | | | ATTACCAATAAAAAAGAATCAAAACTATACTAATAATTGCAATCCANTTGATAATTATATTNTTACCANT | |
| | | | | GTTTCTTTTTNGCTAAAAGCAAGATACCACCACAATTAAAANGACCCTGAGACTAATTTTTTAAGACGTA | |
| | | | | GAAATTATTAAAATANTTTTTTTTCTTTCTTTCTTNNTTAGTAAAGTATTTTCTATGTGTATGAATAAAGAATCA | |
| | | | | AATTCTCGTCAATATACTCAAGAAATCCAATTATCATAGCAGTAAATTGTTATGCTATTTCTACTATATTGT | |
| | | | | TATTATGTTCCTTCTTTAGGAGAGAAATGACTCACATAGCATAGGATATAGATTTGGTNTAGTAATTAAG | |
| | | | | GAAATGGAGTATTGAT[A/G]AAATAAAAAAAACGTGATTCTTTACCNTTGAGATTAAGAGTAATGATAA | |
| | | | | AAAAANTTATTGAAAATTAAGAGTATCTCTTATGTTTAATCGATGTGGTCTCAATCATTAGACGATATTTA | |
| | | | | ATAAGATAAGATATTTTCCAACTACATCATNNNGTGCATCCTCCATATAATTACTAAATCCAAATCTCTTG | |
| | | | | TTATTCAAACACTATAACGACATCAAAGATGTACTCNAGCTATGTATTAGTTTCCTTTTTCTTTTCCCCTTT | |
| | | | | CAAAAAGAAAATAGTGCATAAGGGCCACTAATTTCTTTTTTAAANNTTGCTATGTGCCATACTTGGTAGG | |
| | | | | TGCAAACAAACAAAATAGAGTAGTTACATAGTTAACATGGAGGAAATTAAACTACATATGGGGTAGGTT | |
| | | | | CTAGCCCGATAGGACTATAAAANNNNNGATAAAACACCATTTTGGTCTGAAAGTGTAAATATAGTCACG | |
| | | | | TTAAATTCTCAAATTTTCATTTTTTTNAAAGTTC | |
| SY4279 | SY4279F1 | GGAGAG | | | 1036 |
| | | AAATGAC | | | |
| | | TCACATA | | | |
| | | GCATAGG | | | |
| SY4279 | SY4279R1 | TGAGACC | | | 1037 |
| | | ACATCGA | | | |
| | | TTAAACA | | | |
| | | TAAGAGA | | | |
| SY4279 | SY4279A1FM | AAGGAAA | A | | 1038 |
| | | TGGAGTA | | | |
| | | TTGATAAA | | | |
| SY4279 | SY4279A2TT | AGGAAAT | G | | 1039 |
| | | GGAGTAT | | | |
| | | TGATGAA | | | |
| SY4251 | | | | AAGAAATTCTATGACTCTCAAAATGCATGAAATCTGGTAAGTTCACAAAGGAAATATGAGGGGAGGTTC | 599 |
| | | | | AAGAAATTCTTTTAGAAAAAGCAAGAATTGGAAAACACGTAGAGCATACGTAAAAAACTCATTCAGTTA | |
| | | | | AGCATATTAGCAGCAAGGATATCATATAATACAGCATTTAATTCAACAGAACTGGAAAAGGAAAACAAT | |
| | | | | AACACAAACATCTATCACACATTTTACGTCCTACTCTTACCTGATAGAGGACTGAAGTAAGAGATTAATG | |
| | | | | GAGANAAATATGCTAATAGAGATAAGTGTTTTCCTTCTAACAGAATGCTGCTGAGTTTTTCCTTGGCCTG | |
| | | | | TTATTATTATTGCTTGTTTCAGTTAGAAGGCCCGTGGTGTCATCTTGCGTAAAAAGAAAAATATTTCACG | |
| | | | | GGGCTAATTACTTGAAAGCTCTATGAAAAGCTANGAACTTTTTGGTTTCCAAATTGGATTCAGAAACATT | |
| | | | | TTAATTTGTTTGG[A/G]AAATGGTGGAAGAGAATTGCAGCATCCTCNNNNNTGAAGACTATTGTTCAAG | |
| | | | | ACACTTCCACTTCATACCACTAGGTGAAAGGTGACCCTCTTTTNTATATGTGCACACTGGAAAATATATA | |
| | | | | AAAGTTCTAAAGGGCAACTTTAATAGCTTCCAATCAGAATTTATCTGACACAATAAACTATTTATGTATTT | |
| | | | | TTTTTTNNAAAATAAATTATACACTGATAATATAAAATAATTTTATATAATTTAATAATAAATTATCACATA | |
| | | | | AAATAAGCTGGTTGATCATTATAGTAACTTTTTTGAAACTCTTATTAACTATAATTTCTTGTTGAGGGAGA | |
| | | | | ATATGANTTTTTTTNTAACTTATACTACATAATTGNTAATTTTTTTCTTCAGCAATCAAATAAAAAGCACG | |
| | | | | AGATTTTTTTTNAATACTGACANATATTTTTATTAAATCTAGGACGTACCACTGTCTTTTTAATTGAAAAT | |
| | | | | GCCACTCACACATATACTTGCA | |
| SY4251 | SY4251F1 | AAGGCCC | | | 1040 |
| | | GTGGTGT | | | |
| | | CATCTTG | | | |
| SY4251 | SY4251R1 | TGGTATG | | | 1041 |
| | | AAGTGGA | | | |
| | | AGTGTCT | | | |
| | | TGA | | | |
| SY4251 | SY4251A1FM | CACCATTT | G | | 1042 |
| | | CCCAAAC | | | |
| | | AA | | | |
| SY4251 | SY4251A2TT | TCCACCA | A | | 1043 |
| | | TTTTCCAA | | | |
| | | AC | | | |
| SY4249 | | | | GATGAATGAGTCCTTTTTTTTAGCCAATAAGTGCATCTTTTATTGATTTGATTTACATTATATTTTATTTAA | 600 |
| | | | | TCTGTCACNNCATGTAACTGATGGAGATGGCATCCCCATCTTTGGTTCTAGTCATCACTTATGGATTGAC | |
| | | | | CACAACGACTCACTCTTCAATTGCACTGATGGCCTAATTGGTGCTGTTATGGGCTCCACTGCCATTACCAT | |
| | | | | TTCCAACAACTGCTTCATCCCCTAACTAGGTCGATCTACTAATTAATAGTCCTTTTGGTTAAAATATTTGAA | |
| | | | | GGAATTCTCTCATCATGTGTTTACTTTGTTTTAACCAAGTANTACGNCCATAGAGAGNTAGTGTTGAGTT | |
| | | | | TATTGACTTCAAAATTATTCAGGTTATGCTACTGGCTCAACGTGACTCTTATGTCCACGATCAGCATATGC | |
| | | | | AAGGAATCAATGCATACAACCATTTCGGGGAGAATCTTAACCAAAGAATGCCCAGGTAATTAACTAACA | |
| | | | | TCTTTTA[A/T]GTAGTAGTAGTATCTCTAGATATTTTACTTTTTTTTTTNNAATTGTATATGTCATTCCATCT | |
| | | | | AACATTTTGTTCAATTCTATGATAATAATTTTTATTACTTATTATTTTTAAAAATAGNCTTAGTTACTATTTT | |
| | | | | GNTCTTNTAATTTAATTTTNAAGTTCAATTTGATTCTCTTAATATTAAAAAGTTNAATTTNNTCCTCTNAN | |
| | | | | NNNNNTTTTTTTAAATGACTTAATTAGNTCCTTTTACTTTTAGAAGTTTCAATTAAGTCATTTATTTTTTAA | |
| | | | | AATAGGTTCAATTTGATCTTATTTTTCTTTCATGTGCTNCAANTTTTTTTNNNNAAAAATAAGATCANATT | |
| | | | | TGATTTTAAATAGGANAAAGTTGAANTTATTTTAAAAATTAAGGAACCTAATTNAAACTTCTAAANATAA | |
| | | | | AAGNACCTAANNGAAANACTTTTAAAAATTAAAGAACCTAATCAAACATTTTAATAATAANAGGATCAA | |
| | | | | ATTNAACTTCATANT | |
| SY4249 | SY4249F1 | ACTGGCT | | | 1044 |
| | | CAACGTG | | | |
| | | ACTCTTA | | | |
| SY4249 | SY4249R1 | TGAACAA | | | 1045 |
| | | AATGTTA | | | |
| | | GATGGAA | | | |
| | | TGACA | | | |
| SY4249 | SY4249A1FM | TAGAGAT | T | | 1046 |
| | | ACTACTA | | | |
| | | CTACATA | | | |
| SY4249 | SY4249A2TT | AGAGATA | A | | 1047 |
| | | CTACTACT | | | |
| | | ACTTAA | | | |
| SY4310 | | | | CCAGATTTCATTAAGGTGTAAAAAAACATGACAGTGTGTTAGATTTGAAAGTTAGAGCTAAAAAAGTTC | 601 |
| | | | | TTTATGAGATGGTTGTTCATATCTACATCTATATATGAAAGGATTTGTTGTAGTTATGTTCTTCAGGTCTG | |
| | | | | TGTTCAGTATTTGTCTCAAACATCTCTGTTCTATTTTCAATATTCATTGAAGTTAAATCTTTGGCTTGTATG | |
| | | | | ACAAATTGAAGGATGTTTACCTTGGTAAACAGAAGTGGAGGTTTAAGGTGAGAGTGGTTTGCATTTGTG | |
| | | | | ATATGTGTCAAGTTAGTGATCCCATTATCCACTTGAATTTGTACAGAGATGTTTGCAAAAATATATCACAA | |
| | | | | TGATGCTTTATACAAGGAATTGTGGCATGTCTGTGTCAACCTTTGACACTCTTTTGAGTGAAGGAGAGAG | |
| | | | | GGTTTATTACTTCCCTCAAGGTCATATGGAACACGTACAAGTTGTATGGGCATGTGCTCTTAATTTCTGAC | |
| | | | | TTTAATT[A/G]CAGTTAGATGCTTGTGCTACTATTATCTTGCTCCATATCATGTTTTGGATACTAGCATAAT | |
| | | | | TGTTTTGTTTCCATTTTCCAATTAAGATCTTATTGGTGTTGGCATGGTTCACCTAACAGTGTTTTCTCTTNA | |
| | | | | CAATTTTATCTATAAAATTTTTATTTTTCTTGATCTGACTTGTATGCTAGCAGGAGGCGTCTTTGTTTANGG | |
| | | | | TGTGTTTGCTGTAAGTTTTAAAATTGCACAAGTTGCGTCTGGTGGCATTTCAGTTTGGATATGTTAGATAT | |
| | | | | ATTGAACTCAGTTACTATTTCTATTTCCCCTTAAGTTTTAAATGTTAAAATAAATAAATAAATTGAGAGAT | |
| | | | | TAAACCAAATTTGGGTGTATTTTAATTATAGATTTCATGCCAGGATTTAGTGATTTTGGATTTCTTCAGTG | |
| | | | | TCAAGATTTGTTGGTTTATTTAATTTTAATTTATTTATTTTCTTGACTGATTTAATTGCCTTTGCTATTTTTG | |
| | | | | CAGTGTCTA | |
| SY4310 | SY4310F1 | GGGCATG | | | 1048 |
| | | TGCTCTTA | | | |
| | | ATTTCTGA | | | |
| SY4310 | SY4310R1 | GTGAACC | | | 1049 |
| | | ATGCCAA | | | |
| | | CACCAA | | | |
| SY4310 | SY4310A1FM | CAAGCAT | G | | 1050 |
| | | CTAACTG | | | |
| | | CAA | | | |
| SY4310 | SY4310A2TT | CACAAGC | A | | 1051 |
| | | ATCTAAC | | | |
| | | TGTAA | | | |
| SY4292 | | | | ATTGTCATAGTAATTTATAAATATTTTACTTCATTTATGTTTTAGAATCTATATCCTTTCTTTTAATTGAATA | 602 |
| | | | | GCAGGATCTAGTTATACAAACTGAAAAGAAAAGAACAATCATTATCAATGGGATAAAAAATATTCCTTTC | |
| | | | | TTGTCTAATATATCNTATTAACTATTTAATAGTGGAAATAAATTCCAATCATATGTTTTGTACTATGTCGA | |
| | | | | AATTTATGTATTTTAGTATATCAAATACTCATTTCTTGTATGGATTTTTTTTATATATAAAAAAAAAATNAT | |
| | | | | ACCCATACAACGTACTAATGTAATAGGTTCTCAAAGGCTTCCCTTGCGTGAATACTTCAAAAATATTTCAA | |
| | | | | CCTTTTGATGGAAGCAAGACGGAGAGATAAGATTGAATGTAAATACAAATATCACATCGATGTTAAAAT | |
| | | | | ATTAATTCATCCTATCCAAATTATCATTATGTTAATGTTTTGTGGAGAGTANCAATTGCAACATGGTATCA | |
| | | | | TGT[A/G]GTAGTTATTTCAAAATTACTTATTAAATATAAAGTGGTNTGAAGTGACGTAAGTGCAAGTATA | |
| | | | | TGTGTGAGTGGCATTTTCAATTAAAAAGACAGTGGTACGTCCTAGATTTAATAAAAATATNTGTCAGTAT | |
| | | | | TNAAAAAAAATCTCGTGCTTTTTATTTGATTGCTGAAGAAAAAAATTANCAATTATGTAGTATAAGTTAN | |
| | | | | AAAAAAANTCATATTCTCCCTCAACAAGAAATTATAGTTAATAAGAGTTTCAAAAAAGTTACTATAATGA | |
| | | | | TCAACCAGCTTATTTTATGTGATAATTTATTATTAAATTATATAAAATTATTTTATATTATCAGTGTATAATT | |
| | | | | TATTTTNNAAAAAAAAATACATAAATAGTTTATTGTGTCAGATAAATTCTGATTGGAAGCTATTAAAGTT | |
| | | | | GCCCTTTAGAACTTTTATATATTTTCCAGTGTGCACATATANAAAAGAGGGTCACCTTTCACCTAGTGGTA | |
| | | | | TGAAGTGGAAGTG | |
| SY4292 | SY4292F1 | GGAAGCA | | | 1052 |
| | | AGACGGA | | | |
| | | GAGATAA | | | |
| | | GATTG | | | |
| SY4292 | SY4292R1 | GCCACTC | | | 1053 |
| | | ACACATA | | | |
| | | TACTTGC | | | |
| | | ACTT | | | |
| SY4292 | SY4292A1FM | CATGGTA | A | | 1054 |
| | | TCATGTA | | | |
| | | GTAGT | | | |
| SY4292 | SY4292A2TT | CATGGTA | G | | 1055 |
| | | TCATGTG | | | |
| | | GTA | | | |
| SY4290 | | | | AAAAAAGAGGAGAATTTTCAAGGAATAAGTTGCTCTTGTATTTGACCTCTTCACTGCAGAAAGAAAATCT | 603 |
| | | | | CTCTTAAACAGTAAAGTCCCAAAAAGGAACAAATATGAATCATCAAAGGAAAGCAAATATCAAGCTTAT | |
| | | | | ACATTCCTCAGAAAAATGTTTCTCAGAGATAGCATTGCCTTTGAAGATCTTGGATATTCAGAGATAGACA | |
| | | | | CTGCAAAAATAGCAAAGGCAATTAAATCAGTCAAGAAAATAAATAAATTAAAATTAAATAAACCAACAA | |
| | | | | ATCTTGACACTGAAGAAATCCAAAATCACTAAATCCTGGCATGAAATCTATAATTAAAATACACCCAAAT | |
| | | | | TTGGTTTAATCTCTCAATTTATTTATTTATTTTAACATTTAAAACTTAAGGGGAAATAGAAATAGTAACTG | |
| | | | | AGTTCAATATATCTAACATATCCAAACTGAAATGCCACCAGACGCAACTTGTGCAATTTTAAAACTTACA | |
| | | | | GCAAACACACC[A/G]TAAACAAAGACGCCTCCTGCTAGCATACAAGTCAGATCAAGAAAAATAAAAATT | |
| | | | | TTATAGATAAAATTGTNAAGAGAAAACACTGTTAGGTGAACCATGCCAACACCAATAAGATCTTAATTG | |
| | | | | GAAAATGGAAACAAAACAATTATGCTAGTATCCAAAACATGATATGGAGCAAGATAATAGTAGCACAA | |
| | | | | GCATCTAACTGNAATTAAAGTCAGAAATTAAGAGCACATGCCCATACAACTTGTACGTGTTCCATATGAC | |
| | | | | CTTGAGGGAAGTAATAAACCCTCTCTCCTTCACTCAAAAGAGTGTCAAAGGTTGACACAGACATGCCAC | |
| | | | | AATTCCTTGTATAAAGCATCATTGTGATATATTTTTGCAAACATCTCTGTACAAATTCAAGTGGATAATGG | |
| | | | | GATCACTAACTTGACACATATCACAAATGCAAACCACTCTCACCTTAAACCTCCACTTCTGTTTACCAAGG | |
| | | | | TAAACATCCTTCAATTTGTCATACAAGC | |
| SY4290 | SY4290F1 | CCAGACG | | | 1056 |
| | | CAACTTG | | | |
| | | TGCAAT | | | |
| SY4290 | SY4290R1 | GTTGGCA | | | 1057 |
| | | TGGTTCA | | | |
| | | CCTAACAG | | | |
| SY4290 | SY4290A1FM | CAGCAAA | A | | 1058 |
| | | CACACCA | | | |
| | | TAAAC | | | |
| SY4290 | SY4290A2TT | AGCAAAC | G | | 1059 |
| | | ACACCGT | | | |
| | | AAACA | | | |
| SY4252 | | | | TATAAACTTNGACTAAAATAATTGGCAGATTACACTTTNAGTAATATTTTTTTTAAAAAAAAAANCTNAC | 604 |
| | | | | TTTTATATAATATGACAGTGTNATATACACTTTCAACGACCTAAATGATGTGCTATATCCCANAANGTGA | |
| | | | | TCAAAATCTCAAAATGCACGAATATTCACTTCCTTCATAAACAAGCCTACTCTAAGTAAATAAAAGGACC | |
| | | | | CATAATACAACCCAGAAATGGAAAACAATTAAGAGCTAGCANACGTAAAAAANTTATTCAGTAAAATCA | |
| | | | | GCATATATATTAATATTTAAAACAATATATTTACAAATACGTTGCATCTCAAAGATAACGNTGAAATCCCA | |
| | | | | TATTCTCTTTATTTCATTTTAATTTATGTGGTCATGCATAGTACGTAAACATATTGTGCAAATAATCATCAA | |
| | | | | ANTAATCATACATAACGTTTAATTTAATAAAACTGCAAAAGGAAAACAGCAAAAACGTTTATCAAACTTT | |
| | | | | TTTACATC[A/G]TAGTCTTACCTTATAAAGAAAATTCNTTCATGGGCTTCGATATAACTTGGAATTCCACA | |
| | | | | CCTTGCAGTCCTTGCATTGGATTTACTTCATGAAAAAGCTATAAACGTTTTGTAAGTCTTTTTCGGGATTG | |
| | | | | AGATTTAGAAATATTTTAATCTATTTGGAAAATAAGAGAAGAGAACTGCAGCATCCTTAAAGCTGAAAA | |
| | | | | CCTATCACGAAAACTTCATACCACATGCTGAAAGGTGACTTTCTATTATATAAGTGCACCTGGAGGATAA | |
| | | | | GAAGAAAATGGCAACTTCGGTAATAACTTCCAATTTAGAATTACACGAATGAACAANTTTTTCTTCATTTT | |
| | | | | TAAANAAGTAATTAAATTTGAGGCACGTGATAATTTCTCGAGACCAACAACTTTTTAATTAAATCGTGGG | |
| | | | | TATATATATATATACTAGTAGTCCACTACTTATAATTGAAAATGTTAGAGTAAATGATCAATTATATTTTG | |
| | | | | TTTCTGAAAGCGTGTGATG | |
| SY4252 | SY4252F1 | ATGTGGT | | | 1060 |
| | | CATGCAT | | | |
| | | AGTACGT | | | |
| | | AAAC | | | |
| SY4252 | SY4252R1 | CAATGCA | | | 1061 |
| | | AGGACTG | | | |
| | | CAAGGT | | | |
| SY4252 | SY4252A1FM | TAAGGTA | G | | 1062 |
| | | AGACTAC | | | |
| | | GATGT | | | |
| SY4252 | SY4252A2TT | TTATAAG | A | | 1063 |
| | | GTAAGAC | | | |
| | | TATGATG | | | |
| SY4246 | | | | GATCCTGCTATTCAATTAAAAGAAAGGATATAGATTCTAAAACATAAATGAAGTAAAATATTTATAAATT | 605 |
| | | | | ACTATGACAATAANCGGAGTATAAAACATGAACAAACTCATAGAAATCAAAGTCAAAATATTAAGAAAA | |
| | | | | AAATGTTAGCTGGCCAACAANNNNNGCATTACCAATAAAAAAGAATCAAAACTATACTAATAATTGCAA | |
| | | | | TCCANTTGATAATTATATTNTTACCANTGTTTCTTTTTTNGCTAAAAGCAAGATACCACCACAATTAAAAN | |
| | | | | GACCCTGAGACTAATTTTTTAAGACGTAGAAATTATTAAAATANTTTTTTTTCTTTCTTNNTTAGTAAAGT | |
| | | | | ATTTTCTATGTGTATGAATAAAGAATCAAATTCTCGTCAATATACTCAAGAAATCCAATTATCATAGCAGT | |
| | | | | AAATTGTTATGCTATTTCTACTATATTGTTATTATGTTCCTTCTTTAGGAGAGAAATGACTCACATAGCATA | |
| | | | | GGATATAGATTTGGT[A/G]TAGTAATTAAGGAAATGGAGTATTGATNAAATAAAAAAAACGTGATTCTT | |
| | | | | TACCNTTGAGATTAAGAGTAATGATAAAAAAANTTATTGAAAATTAAGAGTATCTCTTATGTTTAATCGA | |
| | | | | TGTGGTCTCAATCATTAGACGATATTTAATAAGATAAGATATTTTCCAACTACATCATNNNGTGCATCCTC | |
| | | | | CATATAATTACTAAATCCAAATCTCTTGTTATTCAAACACTATAACGACATCAAAGATGTACTCNAGCTAT | |
| | | | | GTATTAGTTTCCTTTTTCTTTTCCCCTTTCAAAAAGAAAATAGTGCATAAGGGCCACTAATTTCTTTTTTAA | |
| | | | | ANNTTGCTATGTGCCATACTTGGTAGGTGCAAACAAACAAAATAGAGTAGTTACATAGTTAACATGGAG | |
| | | | | GAAATTAAACTACATATGGGGTAGGTTCTAGCCCGATAGGACTATAAAANNNNNGATAAAACACCATTT | |
| | | | | TGGTCTGAAAGTGTAAATATAGTCACGTTAAAT | |
| SY4246 | SY4246F1 | GGAGAG | | | 1064 |
| | | AAATGAC | | | |
| | | TCACATA | | | |
| | | GCATAGG | | | |
| SY4246 | SY4246R1 | TGAGACC | | | 1065 |
| | | ACATCGA | | | |
| | | TTAAACA | | | |
| | | TAAGAGA | | | |
| SY4246 | SY4246A1FM | CCTTAATT | G | | 1066 |
| | | ACTACAC | | | |
| | | CAA | | | |
| SY4246 | SY4246A2TT | TTCCTTAA | A | | 1067 |
| | | TTACTATA | | | |
| | | CCA | | | |
| SY4314 | | | | AATTGCAGCATCCTCNNNNNTGAAGACTATTGTTCAAGACACTTCCACTTCATACCACTAGGTGAAAGG | 606 |
| | | | | TGACCCTCTTTTNTATATGTGCACACTGGAAAATATATAAAAGTTCTAAAGGGCAACTTTAATAGCTTCCA | |
| | | | | ATCAGAATTTATCTGACACAATAAACTATTTATGTATTTTTTTTTNNAAAATAAATTATACACTGATAATAT | |
| | | | | AAAATAATTTTATATAATTTAATAATAAATTATCACATAAAATAAGCTGGTTGATCATTATAGTAACTTTTT | |
| | | | | TGAAACTCTTATTAACTATAATTTCTTGTTGAGGGAGAATATGANTTTTTTTNTAACTTATACTACATAATT | |
| | | | | GNTAATTTTTTTCTTCAGCAATCAAATAAAAAGCACGAGATTTTTTTTNAATACTGACANATATTTTTATT | |
| | | | | AAATCTAGGACGTACCACTGTCTTTTTAATTGAAAATGCCACTCACACATATACTTGCACTTACGTCACTT | |
| | | | | CA[A/C]ACCACTTTATATTTAATAAGTAATTTTGAAATAACTACNACATGATACCATGTTGCAATTGNTAC | |
| | | | | TCTCCACAAAACATTTAACATAATGATAATTTGGATAGGATGAATTAATATTTTAACATCGATGTGATATTT | |
| | | | | GTATTTACATTCAATCTTATCTCTCCGTCTTGCTTCCATCAAAAGGTTGAAATATTTTTGAAGTATTCACGC | |
| | | | | AAGGGAAGCCTTTGAGAACCTATTACATTAGTACGTTGTATGGGTATNATTTTTTTTTTATATATAAAAAA | |
| | | | | AATCCATACAAGAAATGAGTATTTGATATACTAAAATACATAAATTTCGACATAGTACAAAACATATGAT | |
| | | | | TGGAATTTATTTCCACTATTAAATAGTTAATANGATATATTAGACAAGAAAGGAATATTTTTTATCCCATT | |
| | | | | GATAATGATTGTTCTTTTCTTTTCAGTTTGTATAACTAGATCCTGCTATTCAATTAAAAGAAAGGATATAG | |
| | | | | ATTCTAAAA | |
| SY4314 | SY4314F1 | GCCACTC | | | 1068 |
| | | ACACATA | | | |
| | | TACTTGC | | | |
| | | ACTT | | | |
| SY4314 | SY4314R1 | TGATGGA | | | 1069 |
| | | AGCAAGA | | | |
| | | CGGAGAG | | | |
| | | AT | | | |
| SY4314 | SY4314A1FM | ACGTCAC | A | | 1070 |
| | | TTCAAAC | | | |
| | | CA | | | |
| SY4314 | SY4314A2TT | ACGTCAC | C | | 1071 |
| | | TTCACACC | | | |
| SY4264 | | | | ACTGACTTATATTGATCTATATTTTACTTTTTTATCTAAATTTTTACAGATTCAAAAGAAAAAAATAAAAAT | 607 |
| | | | | TAAAAAAAATATATTATATCAAAATAGGCTCAATTGAATTCACACCAATATTTTTTGAGTCCAATATAACC | |
| | | | | TGATAGTAGACTAGTCCAAATAGTCACAATTCTATTTGGATTAACATTTTATTAGTCTAACTCGACCTGAA | |
| | | | | TCAATAGGCCAACAGTGAACTAGCTGATGCGGTCCATTTTGCGAGCTCTATATGGAAGGATGGTTTTTTT | |
| | | | | GGCACATATATCATGCATATATGTGTCACTTTCATAGTTCAATAGAAAAAAAGTCAAGTAATTGACAAAA | |
| | | | | TTAAAAACCAATTCATTACTTAAAAGTGGGGTCGTAGTTTGCTGAATGCCCCACGCACAGANAGTAATTG | |
| | | | | GAGGAAAGTAAATTCTGCTGCCAAAGATTTCACATAACTCCCAAACTAACATTCATTACTAATTGAAATTT | |
| | | | | CAAA[*/A]GCATTCCTAACAAACTTTTAGCTAGGACCATCACAATCATATACTTATTTGANGATATTAACA | |
| | | | | ACAAAAACAGAGAAAATTCCTTGTTCATCTACCTATATTTTCTAACACAACGTTAAAATACATTACAATTA | |
| | | | | ACTGACTTTGCTTGTGTAAACTTCTACGATAAGATTCTTTGGATTTTTAGTAAACTCTTATCATTTTATGAG | |
| | | | | TGTCAACCCAATAGCAGTGACTATAAGAGTTGAAGGAAGGCCAACTTTTAGATGAGTCCAAAAGGTTAA | |
| | | | | TGTGTATCCAAGGTTTGGAGCTCGACGAGCTTGTTCACACACTATCAAGTTGGCAGCTGATCCTAATAGT | |
| | | | | GAAAAGTTCCCTGCTATAGTGCTAACCCAAGCTAAGATTAGCCATGCCCTCTCCTCATCTCCTTTGGAAAT | |
| | | | | TGCAGCAGCTGAGGCTGCAACTCTTGCTCCAAGCAATAGAACTAGAAAACATATGAATAGAAGATTGTG | |
| | | | | ATGCATCAAATGAAA | |
| SY4264 | SY4264F1 | GAGGAAA | | | 1072 |
| | | GTAAATT | | | |
| | | CTGCTGC | | | |
| | | CAAA | | | |
| SY4264 | SY4264R1 | GATTGTG | | | 1073 |
| | | ATGGTCC | | | |
| | | TAGCTAA | | | |
| | | AAGT | | | |
| SY4264 | SY4264A1FM | TTGTTAG | I | | 1074 |
| | | GAATGCT | | | |
| | | TTTGAAAT | | | |
| SY4264 | SY4264A2TT | TTGTTAG | D | | 1075 |
| | | GAATGCT | | | |
| | | TTGAAAT | | | |
| SY4416 | | | | GAATAGCTATAACTAGATTCTGGGGGTTCCCAATTGGGGTTGCTGAGGATCCAATATTAGCACTTGAAG | 608 |
| | | | | CTAGTGCAAGAAGGAAAGGGTAAGGTGGGAGGTTATGTTGCCTTGCTATTTTCAACACAAATTCAGTCA | |
| | | | | ATACAACACAAGATGTGTCATTGGTGAAAAAGGCACTTGATATAGCNGAAATTAAACAAATTCTACAGA | |
| | | | | GTAAGTCCTTTGGTCCTTGGCTTTTCCAAGAGAGCAATTTCCCCAAGTACTTGAACATGTCTGCTCTTTCA | |
| | | | | AGAAAAACAGTAACAACCATTGTCCCAAAAAGAAGACCAAGAATTGGGAGATCAATTGCAGCAAAAGC | |
| | | | | TTGATCTGGATTAAGAACTTTGAATATGACCATAAACATTGCACCTAGTAGGGACCCTGCAGTTCTCCCA | |
| | | | | ATGGGTAGAAAAGGCACACATGGGAAAACTGCTAGAACCCAGAAAACTGCAAAGGCTATTGAGCCAAA | |
| | | | | GACAACTGTTGGAGTA[A/G]GAGCCAATGCCATTGTTGTGGTTAAACAAGCAACAATTTCCAAACAAAT | |
| | | | | GTTAATTCAAGCCTCCACTTCTAGGCGAACACAATAGAATTTTGTTTGGTACAATTTCCTGAACATGGAA | |
| | | | | AGCCAATTGATTAGTAACTTTGTAAGAGTGCATGAAAAACCAATCTATTATATTCCTCTTCTTATTCAATC | |
| | | | | TACTACTTTGAGACATGAATTAAGTGAATCATTATTATTGAATATTAATGATCAAGCGGGAACGTGGATC | |
| | | | | GGAGTATGAATATATTGTTTATGGTATTTGACAGAGGTCTGCTACATCACATGAATACAGGTATCGTATA | |
| | | | | CTGTGGGTTAATTAAATTTTTGCAAACAGATAAATAAATTAAATATGGACAAGACATTTGAATTTGATGT | |
| | | | | ACTATAAAACATGCCAGCAGTGTATTAAACATAAAATCACATGACTTTCCAAGCATAGCATAAAAATTAA | |
| | | | | AGGAAGCAATTATCTTGAAAAACAACTTAA | |
| SY4416 | SY4416F1 | GCAAAGG | | | 1076 |
| | | CTATTGA | | | |
| | | GCCAAAG | | | |
| | | AC | | | |
| SY4416 | SY4416R1 | GCCTAGA | | | 1077 |
| | | AGTGGAG | | | |
| | | GCTTGAAT | | | |
| SY4416 | SY4416A1FM | CTGTTGG | A | | 1078 |
| | | AGTAAGA | | | |
| | | GCCAA | | | |
| SY4416 | SY4416A2TT | TGTTGGA | G | | 1079 |
| | | GTAGGAG | | | |
| | | CCA | | | |
| SY4426 | | | | ATGTTCTAATGCTCGACCTTCACGGTGGTGAGTTTAATTATATGAGCGGAGAGATCCACAAGTCGTTGAT | 609 |
| | | | | GGAGTTGCAACAATTAAAGTATTTAAACCTCAGTTGGAATTCTTTTCAAGGCAGAGGAATCCCAGAGTTT | |
| | | | | CTTGGTTCTCTCACCAACTTGAGATACCTTGATCTGGAATATTGTCGTTTTGGCGGAAAAATTCCAACTCA | |
| | | | | GTTTGGCTCTCTTTCTCATTTGAAATACTTAAATCTTGCTTTGAATTCTCTGGAGGGTTCAATCCCTCGTCA | |
| | | | | ACTTGGAAATCTCTCCCAGTTGCAGCATCTTGATCTCAGCGCCAATCATTTTGAAGGAAATATACCCTCTC | |
| | | | | AAATTGGAAATCTCTCCCAGTTGCTGCATCTTGATCTCAGCTACAATTCTTTTGAAGGAAGTATACCGTCC | |
| | | | | CAACTTGGGAACCTTTCAAATTTGCANAAGCTTTATCTTGGAGGCGGTGCTCTCAAAATTGANGATGGA | |
| | | | | GATCAT[A/T]GGCTGTCTAATCTCATTTCTTTAACCCATCTTTCCGTGTTACAGATGCCTAATCTCAACACT | |
| | | | | TCTCATAGCTTCCTCCAAATGATTGCCAAGCTACCAAAACTTAGAGAACTGAGTTTAAGTGAATGTAGCC | |
| | | | | TTCCCGATCAGTTTATCCTTCCATTGAGGCCCTCTAAATTCAATTTTTCTAGTTCCCTTTCCGTCCTTGATCT | |
| | | | | TTCCTTCAACAGCCTCACGTCATCAATGATACTCCAGTGGCTGTCCAACGTCACTTCCAACCTTGTTGAGC | |
| | | | | TTGACCTTAGTTATAACCTCTTGGAGGGTTCCACATCAAACCATTTTGGCCGTGTAATGAATTCTCTTGAG | |
| | | | | CACCTCGACCTCTCATATAATATATTCAAGGCTGACGATTTCAAATCCTTCGCGAATATATGCACCTTACA | |
| | | | | TTCTTTATACATGCCAGCAAACCATTTGACTGAAGACCTTCCATCAATCCTTCATAATTTGTCTAGTGGTT | |
| | | | | GTGTTAAACAC | |
| SY4426 | SY4426F1 | ATCTTGG | | | 1080 |
| | | AGGCGGT | | | |
| | | GCTCT | | | |
| SY4426 | SY4426R1 | TGGAGGA | | | 1081 |
| | | AGCTATG | | | |
| | | AGAAGTG | | | |
| | | TTG | | | |
| SY4426 | SY4426A1FM | TGGAGAT | A | | 1082 |
| | | CATAGGC | | | |
| | | TGTC | | | |
| SY4426 | SY4426A2TT | ATGGAGA | T | | 1083 |
| | | TCATTGG | | | |
| | | CTGTCT | | | |
| SY4427 | | | | ACACATGGAGATGANGCAGGCCTAATGCTTCCCCCAAAGATTGCACCAATACAGGTACATTTGGATGCT | 610 |
| | | | | ATTGATGTCTTAACCTTGATGTGTAGACACCATTAGTTAAATTTTGCTGTTGAAAGTTGGAATACTTTGCT | |
| | | | | CCTTGGTCAGGCAAATATGAAAATAAAGGGAATGCTTACTTAAAATGAAAAAGACTCTTCTACTCCGAA | |
| | | | | GTCCAAACTCCTACATGCAGNTACAGGATTATGTTCCATGCCTGTACTTTTATATTGTAATTTAAATAAAT | |
| | | | | TATAANTTTTTCATTTTTGACAAAATCATGGTGTGTGTTGCCTGGTTAGGTGGTAATTGTACCCATTTGGA | |
| | | | | AGAAGGATGATGAAAAAGAGGCAGTTCTAAATGCAGCATCATCTGTAAAAGATGTTCTTCAAAGATCTG | |
| | | | | GGATTAAAGTTAAACTTGACGACTCNGATCAAAGAACTCCTGGATGGAAATTCAATTTCTGGGAAATGA | |
| | | | | AGGTTTGTTTT[A/T]AAACTTGAATGGAAATTCAATTTCTGGGACCAAATAATGCGCATTGCTTTGGCTC | |
| | | | | ATGCTGCAGGATCTTTGAGTGTTTGATTATATTTATATCATTTACTTCATATTTAGGGAGTTCCTCTTAGA | |
| | | | | ATTGAAATTGGTCCTCGTGATGTGGCTAGTGGAAGTGTGGTGATATCCAGGAGAGATATCCCTGGGAA | |
| | | | | GCAAGGGAAAGTGTTTGGAATCTCTATGGAGCCTTTAAATTTGGAGGCTTATGTTAAAGACAAGTTGGA | |
| | | | | TGAAATACAGTCATCTCTTTTGGAAAGGGCAATTGCATTTCGAGACAGGTTCATTCCTTTAATGCTACTTT | |
| | | | | TAGCCTGGAACTCCTTAACATAACCTTGTCTAACATGCGTTGAATTGATTTTTTCAAAATAATCTCATTTAT | |
| | | | | TTATTGATAATAGTCACATTAATCATCTTTCCTGAATTGAAGAATGTTAATGGTAGCAAAATGCATAATCT | |
| | | | | TGGGTTCTGTCAAAACAATGTTG | |
| SY4427 | SY4427F1 | GGCAGTT | | | 1084 |
| | | CTAAATG | | | |
| | | CAGCATCA | | | |
| SY4427 | SY4427R1 | GCAGCAT | | | 1085 |
| | | GAGCCAA | | | |
| | | AGCAATG | | | |
| SY4427 | SY4427A1FM | AATTTCC | T | | 1086 |
| | | ATTCAAG | | | |
| | | TTTAAA | | | |
| SY4427 | SY4427A2TT | AATTTCC | A | | 1087 |
| | | ATTCAAG | | | |
| | | TTTTAAA | | | |
| SY4421 | | | | TTTTTTTTTATCAATCTGCTTAATAAAGATTTGCATTCAACATGTTATCGAGATGAAACTTCAATTCTGATT | 611 |
| | | | | AGAGAGCAGCACCAAAAGACTGCAGTTATGTTTTGTGCTTATCTTTATGGAAAGAGAATGGTGCTCAGG | |
| | | | | AATTGGGTTTTGATTTTGTGTGTTTGTATTTTGCAGCTGGGGTTACCCCTTTTGTGGTGGCAGGGATTGA | |
| | | | | ATTTAGCAAAATAATTGTAAGTCACTTTTTTTGGTTGAGCTGCTAACTCATAAGTTATGAAGCCCGTGTTT | |
| | | | | CATGTCTTCATTGTTGGATATGTTCCAGATAGCTCAAAAAAGATGTGAGGTGTGTGGAGGGTCAGGGCT | |
| | | | | TGTTCTTAGGGAAAAGGAAAAGGACTATCTCCGTTGCCCAGAATGTGGTATGATTGCATTCACTTCTCCT | |
| | | | | TCTCATATCATGTACTCCATTTAACCAATTAAGTATGCAGCTGGGAGAATGTTATTGTATGTCTCTAAAAT | |
| | | | | ATTGCTAA[A/C]TTATGGGACTTTTTGATGTCTCAGTAATTGAACATCATATTCAGTATCTTGAATGTGTG | |
| | | | | TAGTTATAAATTATTTGGATGTACATTGAACACCTAGGAATTGCAGAAATCCCTGCATTCCCAAGCTAATT | |
| | | | | TGAATATCTATTGTCAACATGAGAATATTTTGATGTCGAAGAGGAGATATTTTTCATAGATCCTCTTCTTG | |
| | | | | ATATTAGAAAAGATAGAAGGTATGTTGCTGCCTGCTCCTGTTCTTATTTGCTTTTACATTTCTTTTCCAAG | |
| | | | | AAATTATTAGACACTATGGATTCATCATATGCTTCCTCTTCTTGGTTCTTTTCTGCATTTATGTAATAATAT | |
| | | | | GACTTTTTTACTCTTTCAATTCCAATTAATGTATAGGAGAAATGCTAGTCCAATTTTTACAACTCATTAAGT | |
| | | | | AAATTAATTTTCATGTTACATTATGTGGTGTGGTTGTTCCATGTGACATGTAATCATATTTTTCAATATGA | |
| | | | | AAAAGAGTTAATA | |
| SY4421 | SY4421F1 | GCAGCTG | | | 1088 |
| | | GGAGAAT | | | |
| | | GTTATTG | | | |
| | | TATG | | | |
| SY4421 | SY4421R1 | TTCTGCA | | | 1089 |
| | | ATTCCTA | | | |
| | | GGTGTTCA | | | |
| SY4421 | SY4421A1FM | AAGTCCC | C | | 1090 |
| | | ATAAGTT | | | |
| | | AGCA | | | |
| SY4421 | SY4421A2TT | AAAGTCC | A | | 1091 |
| | | CATAATTT | | | |
| | | AGCA | | | |
| SY4437 | | | | TGGAGGAGATGGTGGTGAGGGTTTTTTGGAATCATTGTTCCGGTTGGATAAAAGATTGAGGAAAGTAC | 612 |
| | | | | GTAAAAGAAAGAGGATGATGAAGGTAACAACTGGGAGGAAGAACCAACTTGAAGAGTTTTCTTGCTGA | |
| | | | | GAGATCCACATTGGGTTTCTCAGTTGATAGATGATGCAATGACTCGGGCTGTGTGTAGTATCTTTTCTTT | |
| | | | | ATAAGGTAGCGTTGGAGGTCCTCACCAGCTACGTGTTTGTGAAAAATATGTATTTTTTTTTATCGGTAGA | |
| | | | | AGTTTATAATATTACGCATCTTATGCAATCGATTGAATTAGAGTAAATAATTAATCTGTCACTTTTTTATCG | |
| | | | | TTGAATATATAGTATGGGATATATAAATTCTTTTCTAGAAGGAAATAAGGATAGAAAAAACAGAAAAAC | |
| | | | | AAGTAATAAATATCACCATCATTCATCAATGGCGGCTGCAAAAATTTGGTAAAGGAATTATCAATTAATT | |
| | | | | AGTCCAAAAAATA[A/G]TGTAACAGAAAGAAAGAGCCACGTAGAATGAAGAATCTTATGCTAGATGAA | |
| | | | | ATTTTCTACACTTATTTATATTATTTTTAGAAGCTTCTACACTTATTTCAGTCGTTATTGATGGCTTTTCGAC | |
| | | | | AAGCTAGGCTCCTATTGGAAGGGCGGACTGGACTGTAAGTCATATATGGATGCTGCAATTAATAATGTA | |
| | | | | AAGCAACTATGAAGTGGATAATATTGTTTGATCCTGACATATATGTTTTAGGGTAATTTCTGCATTAGGT | |
| | | | | AACATGTTTTAATTGATGGTTTAATTATTCATGAATAATATACTTGTAACTATTAATAGTTATATAGAGTA | |
| | | | | TTTGGTAAAGCTAGGTTAACCATGGGGTCTTCCAAGAGAGGTGGGGAGCTAGGTAACAATCTTTC | |
| | | | | GTAGTAGTTTGGAGTAGAATACTAACTAAAAAATTTGGTCGTTTCTTGTTAGATCGAGGATGATACATGG | |
| | | | | ATATTTATGTTTTTTTTTATACATTTA | |
| SY4437 | SY4437F1 | ATTCATC | | | 1092 |
| | | AATGGCG | | | |
| | | GCTGCAAA | | | |
| SY4437 | SY4437R1 | AAGATTC | | | 1093 |
| | | TTCATTCT | | | |
| | | ACGTGGC | | | |
| | | TCT | | | |
| SY4437 | SY4437A1FM | TCTTTCTG | G | | 1094 |
| | | TTACACT | | | |
| | | ATTT | | | |
| SY4437 | SY4437A2TT | TTCTTTCT | A | | 1095 |
| | | GTTACAT | | | |
| | | TATTT | | | |
| SY4428 | | | | TGACAAGAGATATACATTTCAATTGAAGTTGTTAAAATTAGAGTTGAGAACATGTTTTGTCATTATTTTAA | 613 |
| | | | | GATGACATTTGAATATACATTTCTTTTAAATTAATATAATTTTATTAAGAGAAAGCCAATTCATCACAAGG | |
| | | | | AGTACCAAAGACCAGCTTACACAAATATTGTTGGTACAAGAGTATTACAAAATAACAACTGAAACAGAA | |
| | | | | CACCCCTCACATAAATCCATACAAAATAGGATGCATTAGGACCAACTACATTGCCCTCACCATAAAAAAG | |
| | | | | ACCAAAGTAAATGTTCTTGACAGAACCATAAAATCGACAACTTCAAACTACACACACTATAGCCAGAGA | |
| | | | | GTTAAGCATTACATTACAAGATAAAATCACTTCCAAGTGAATCAAAATTGCTTGACCAATTGTCCAATTCC | |
| | | | | ACGCTCTGTGCCACCAAGCTGATCCAATGATATATGAAAGACATTGCTGCTGCACCGGTTGATTCATCAT | |
| | | | | CATTAATCC[A/G]ATTGAATAGGGAAATGATACATATATATGAAATGTGGCTAGACACATTGTTATGATA | |
| | | | | GGCAAAATTATTTAAATGGATTAGCAGCAGAGGATGACTAAAGTGAATCAGGTGCACATTTCCGTTTTCT | |
| | | | | TGTGATGCTGCTTGTGTTCTATATAGAAGGCTACCTCTGATTTTAAAGCTCTGGTCTTGTCCACACACTCT | |
| | | | | TCTATAGTAAATTTTAGTTCTTATCATTATTTTTGTAAATCTTTTATTTGTAGGTTGAAGATGCCTCAAATT | |
| | | | | AGTCTAACACAAATCGACGGTTCATTTTTTTTTTTTTTAATTAGATTAGGGGAAGACATACCCCTTTACAA | |
| | | | | TTGCAAGGTCAAGGAAGACCACCAACTTCACTGAGAATTTAGAAGAATTTACACATCATTTATCAATGAT | |
| | | | | TAATACGAGAGTCGAACTCAAGTCATAGTTTTTATGAGTAGAAGACTCGTTCAGAGGTGTCAACGCTTAT | |
| | | | | TAGTAGAACAGTTCGTTTT | |
| SY4428 | SY4428F1 | ATTGCTG | | | 1096 |
| | | CTGCACC | | | |
| | | GGTTGAT | | | |
| SY4428 | SY4428R1 | GTCATCC | | | 1097 |
| | | TCTGCTG | | | |
| | | CTAATCCA | | | |
| SY4428 | SY4428A1FM | CATCATC | A | | 1098 |
| | | ATTAATC | | | |
| | | CAATTGA | | | |
| | | ATA | | | |
| SY4428 | SY4428A2TT | CATCATC | G | | 1099 |
| | | ATTAATC | | | |
| | | CGATTGA | | | |
| | | AT | | | |
| SY4362 | | | | CTAGGNTGTTATAGTTTTAATTATTTTTCGTTTGTGAGGATAGTTTTTGATATATACTTATTTTTTAAAATC | 614 |
| | | | | AATATACATAATTAAGTAATTAAAAATGTTAAATTAAAATAGATTATGTAATTATTAAAATTTTAAAAATT | |
| | | | | ATCATTCTTTGTTGAAAATACTTGATTTAAATCTTAAGTAGTATAATTTAAAAAGATAAAGACATGCACTT | |
| | | | | ATT[TAAT/AAATTTTCTTTTAAAATTATTGAAGCTAAATTTTAATTTCTCCAATCCCCCCGCAAAAAAAA | |
| | | | | AAGGATCATATTAGCGATTAAGATTTAGCAGGTGGAATGAAATTTCAGAGGTTCCTATCTAGGTCATA | |
| | | | | CAAATTGATAATTCATATCATAATAAAAAATTAATGTGATGAGAAACTTTTGTTTGTTCTATTTCTGTAT | |
| | | | | TTCCCTTCAATATTCCAGTTATTTTGTGAGACACGATATAATGCTTGGGGCAGTGCTGGAGCTTGAAAC | |
| | | | | AAAAAATTGGGAGTCAAAAAT]AAGATTGGAATGAAAAAAATATTCATAGATTTTTCATTTTATAATCTC | |
| | | | | ATCTAAATTTTTTTAATATTTTTTTAAAAAAATCTTAAAATAACTTATCATGCAATAATTTTTTACTAATTAA | |
| | | | | GTTATTCAACCCATCATATCAATATCAAGTAAAGATAATTATATTTTTAAAAAGTTAAGTGC | |
| SY4362 | SY4362F1 | TCATTCTT | | | 1100 |
| | | TGTTGAA | | | |
| | | AATACTT | | | |
| | | GATT | | | |
| SY4362 | SY4362R1 | TTGATATT | | | 1101 |
| | | GATATGA | | | |
| | | TGGGTTG | | | |
| | | AA | | | |
| SY4362 | SY4362A1FM | CAATCTT | D | | 1102 |
| | | ATTAAAT | | | |
| | | AAGTGCA | | | |
| SY4362 | SY4362A2TT | GTATGAC | I | | 1103 |
| | | CTAGATA | | | |
| | | GGAACCT | | | |
| SY0574AQ | | | | AACCCTCTAACTATACTTATTCTCTGACAACCGTTTATAAAGTTTATCTAAACAGGACTTTACTTAGTTCCA | 615 |
| | | | | CCATTCAAAAAAACATGATTTTGTAGCTTATGCATAAGTTCACTTCAACTTATGGAGGAACTTCTTTCATC | |
| | | | | TCTCTTCTTATTTTCTTCTCATATAAGTACTCAGGGAAAAGTTTATTCAAACAGACCCTAAACCTTGATTTT | |
| | | | | ACTCTCAAACATATTTTTGGAACACTCCCATCGAAAATCCAGACACACCCTTAATTTCCCAGCATTCAAAA | |
| | | | | CCCTCTTTTAGGGTTCCATTCACAGAGCAAACACGTTCCAAACAAAAGAAGACCAAAGATTTCGGCACTC | |
| | | | | AGAGNGGAAAAGNTTNGAACTTTGACACTCCCAAGGAGTCACTNAGAAGGGTTTGTTTCGTGGGGAGT | |
| | | | | TTTGGCGACGATGGAGAGGGCGTGGAGGCCGCTCTGGAGCTCGTCGGCGAGGAGGTCGTAGATGAGA | |
| | | | | CGGTGTCGTTTGAC[A/G]AGGCTCTGGCCCTCGAACTTGGGGGAGACGATGTTGAGGTTGAAGTGGGT | |
| | | | | TTCTTTGTCGGAACTACCCTTCACGGCGGCGTGGCCCGCGTGCTGGTACGACACGTCGTCCACCTCCAAA | |
| | | | | ACGGTGGCTTCCAGCGCCGTTTGCAGCTTCGACCGAATCCTGCTAGGCACGAGTAAATACATGAATATG | |
| | | | | TCTCTGAACTTTTTGAGCATTTTTAATNGTAATTAAGTCCTTAATCTTCAACAAATTTTTTAAACAAATTTC | |
| | | | | TCTAAGTTAGTTTACTACAGCTTGAAACTGCCATAAAAATAACAATATGTGGCNGTTTTACCAACTCCAA | |
| | | | | GACCCAATTACAAAAATTGTAAGAGATCTAAGAACCCAATTACAATTTATTTTTAATTTTTTTAGAAACTT | |
| | | | | CATTAAAAATTCCCAAATAATTCAATCACCTATTGATGTATTAACCCTCTAACTTATATTATTCTCTTACAA | |
| | | | | CCGTTTATAAAGTTTATCTAAACAGGACTTTACTTAGTTCCACCATTCAAAAAAACATGATTTTGTAGCTT | |
| | | | | ATGCATAAGTTCACTTCAACTTAT | |
| SY0574AQ | SY0574AF1 | GCGAGGA | | | 1104 |
| | | GGTCGTA | | | |
| | | GATGAGA | | | |
| SY0574AQ | SY0574AR1 | TGAAGGG | | | 1105 |
| | | TAGTTCC | | | |
| | | GACAAAG | | | |
| | | AAAC | | | |
| SY0574AQ | SY0574AA1FM | TGTCGTTT | A | | 1106 |
| | | GACAAGGC | | | |
| SY0574AQ | SY0574AA2TT | TCGTTTG | G | | 1107 |
| | | ACGAGGCT | | | |

### Example 5

### Allele Mining

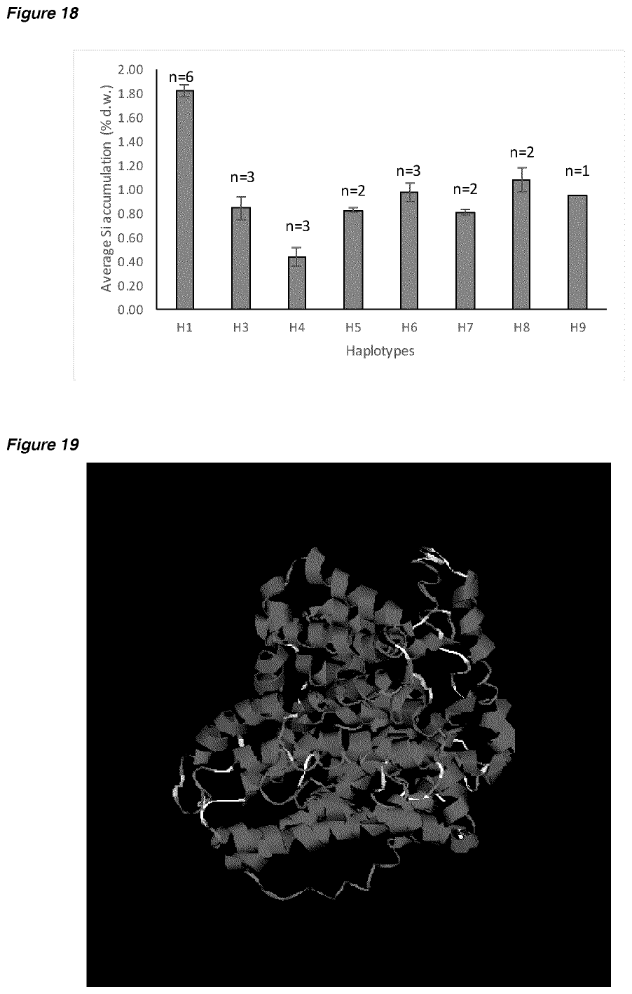

We have performed allele mining in 428 diverse soybean accessions belonging to different maturity groups. As a result, nine haplotype groups were identified based on allelic variations in the coding sequences of Glyma16g30000 and Glyma16g30020 (Table 21). The large majority of genotypes analyzed (94.6%) carry a haplotype similar to Williams 82 (H5). Five accessions were found to carry the haplotype (H1) similar to Hikmok sorip, Plants from the entire set of accessions carrying haplotype H1 were found to accumulate high levels of Si (FIG. 18), thus confirming the association of haplotype H1 with high Si uptake capacity in soybean.

TABLE 21
Details of haplotype groups based on non-synonymous SNPs identified in
coding sequences of Glyma16g30000 and Glyma16g30020 genes evaluated
in 428 soybean accessions belonging to different maturity groups

| | | Glyma16g30000 | | Glyma16g30020 | | | | |
|---|---|---|---|---|---|---|---|---|
| Haplo- | Representative | SEQ ID NO. 16 | | SEQ ID NO. 14 | | | | Total |
| group | PI/cultivar | 33673022 | 33673483 | 33681630 | 33682500 | 33683047 | 33683049 | Lines |
| H5 | Williams 82 | T | A | T | C | C | T | 405 |
| H1 | Hikmok sorip | A | G | C | T | G | C | 6 |
| H2 | PI 567731 | A | G | C | C | C | T | 1 |
| H3 | PI 602991 | A | G | C | C | G | C | 3 |
| H4 | PI 553047 | T | A | C | C | G | C | 3 |
| H6 | PI 548644 | T | G | C | C | C | T | 4 |
| H7 | PI 468916 | T | G | C | C | G | C | 2 |
| H8 | PI 572239 | T | G | T | C | C | T | 3 |
| H9 | PI 407184 | T | G | T | C | G | C | 1 |

Bold - Hikmok sorip type allele,
Italics - Non-synonyrnous SNP

The evaluation of lines belonging to H2 to H9 haplotypes showed low level of Si accumulation compared to the average of Hikmok sorip with other PI lines from Haplotype H1 (FIG. 18),

### Example 6

### Sequence and 3-D Structure of HISil Gene

Si uptake in soybean is facilitated through influx in root by aquaporins GmNIP2-1 and GmNIP2-2 and subsequent efflux toward the aerial part by HiSil. No genetic variation has been observed for GmNIP2-1 and GmNIP2-2 genes. We have shown that the high Si uptake in Hikmok sorip and five other accessions carrying haplotype H1 is directly and uniquely related to the genetic variation at the HiSil locus. The HiSil gene (SEQ ID NO. 14 or 16) codes for a transmembrane protein having specific protein structure comprised with several transmembrane domains (FIG. 19).

### Example 7

### Sequence Homology to Other Monocots and Dicots

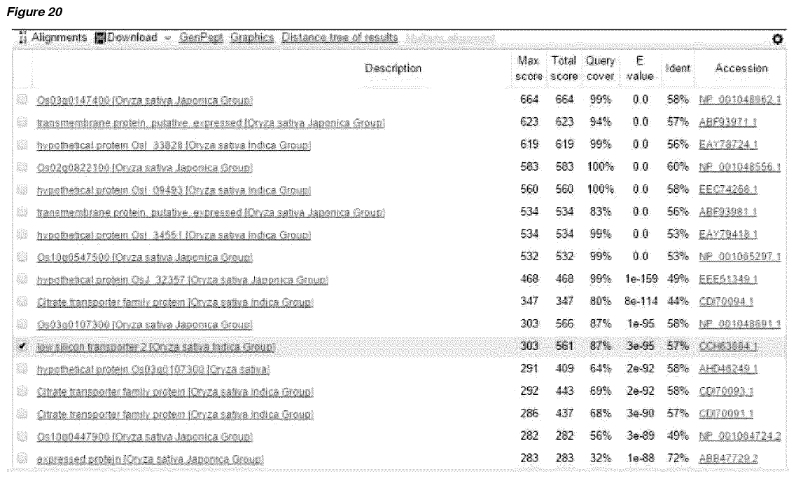

The HiSil protein sequence (SEQ ID NO. 15 or 17) has 57% homology with the low Si transporter 2 (Lsi2, efflux Si transporter) identified in rice (rice being a monocot) (FIG. 20). When looking at HiSil homologs in dicots (like soya), we see around 70% homology. Therefore, the present invention encompasses plants comprising a HiSil protein sequence having greater than 60% homology in monocots and greater than 70% homology in dicots.

### Example 8

### Increased Resistance

### Materials and Method

Overview of Procedure: Watering with AgSiI21 was begun at least one week (7 days) prior to inoculation of soybean with any pest or pathogen. 100× (10,000 ppm) stock solutions of AgSil 21 (CA5684A) was prepared and stored in 1-liter batches. In preparation for application to plants, each 100× stock solution was diluted 100-fold (10 mL 100× stock per liter of onsite water), and pH-adjusted to fall between 6.0 to 7.9 by adding a small portion of concentrated acid (3M HCl: CAS #7647-01-0). 100 ppm (1×) solution was applied to plants within each of the 2 treatments, using a dedicated watering can for each treatment. The 1× diluted solutions was applied each time irrigation was needed. The control was onsite-water and pH of the onsite water was checked to ensure that it fell within the same range as the AgSil21 watering solution.

The experiment was designed as a factorial with split-split plot, where the main plots were the soil amendment (watering regime) and sub-plots were soybean lines, such that soybean lines were randomized within each replicate. Planting was carried out with sterile soilless growing medium (Sun Metro Mix 900) at 8 pats/replications per treatment and 5 seeds per 12-oz. cup were planted around the perimeter and seedlings were covered with W of medium. One susceptible soybean seed (Corsoy 79) was planted in the middle of each pot.

Seeds were started in vermiculite, then just after emergence (3-5 days), they were gently uprooted and the root of each seedling was dipped into Cadaphora gregata spores suspended in solution at rate of approx. 10×10⁶ propagules per ml. In each cup, one plant was left non-inoculated for comparison. Plants were maintained at 70° F. and 14 hours of light.

8A—Evaluation of Soybean (Glycine max) Recombinant Inbred Lines (RIL) with and without Silicon Soil Amendment to Determine Resistance to Brown Stem Rot “BSR” (Cadaphora gregata)

The objective of this study was to evaluate 20 soybean lines, 2 parental lines, plus 7 additional controls (Table 22), with and without a Si soil amendment, to determine resistance to Brown Stem Rot (BSR) under greenhouse conditions. These lines of soybean have an ability to take up higher levels of silicon, and in combination with a silicon soil amendment, have demonstrated resistance to brown stem rot.

TABLE 22
List of soybean lines

| Table 22. | | |
|---|---|---|
| Material Id | Name | Characteristics/trait |
| 14DL880001 | Majesta | Parental line, Low silicon |
| | | accumulator, LoSil allele |
| 14DL880006 | RIL006 | High silicon accumulator, HiSil allele |
| 14DL880016 | RIL016 | Low silicon accumulator, LoSil allele |
| 14DL880017 | RIL017 | High silicon accumulator, HiSil allele |
| 14DL880019 | RIL019 | Low silicon accumulator, LoSil allele |
| 14DL880023 | RIL023 | Low silicon accumulator, LoSil allele |
| 14DL880046 | RIL046 | High silicon accumulator, HiSil allele |
| 14DL880047 | RIL047 | Low silicon accumulator, LoSil allele |
| 14DL880049 | RIL049 | Low silicon accumulator, LoSil allele |
| 14DL880052 | RIL052 | High silicon accumulator, HiSil allele |
| 14DL880057 | RIL057 | Low silicon accumulator, LoSil allele |
| 14DL880062 | RIL062 | High silicon accumulator, HiSil allele |
| 14DL880066 | RIL066 | High silicon accumulator, HiSil allele |
| 14DL880070 | RIL070 | High silicon accumulator, HiSil allele |
| 14DL880074 | RIL074 | Low silicon accumulator, LoSil allele |
| 14DL880080 | RIL080 | Low silicon accumulator, LoSil allele |
| 14DL880096 | RIL096 | Low silicon accumulator, LoSil allele |
| 14DL880109 | RIL109 | Low silicon accumulator, LoSil allele |
| 14DL880110 | RIL110 | High silicon accumulator, HiSil allele |
| 14DL880127 | RIL127 | High silicon accumulator, HiSil allele |

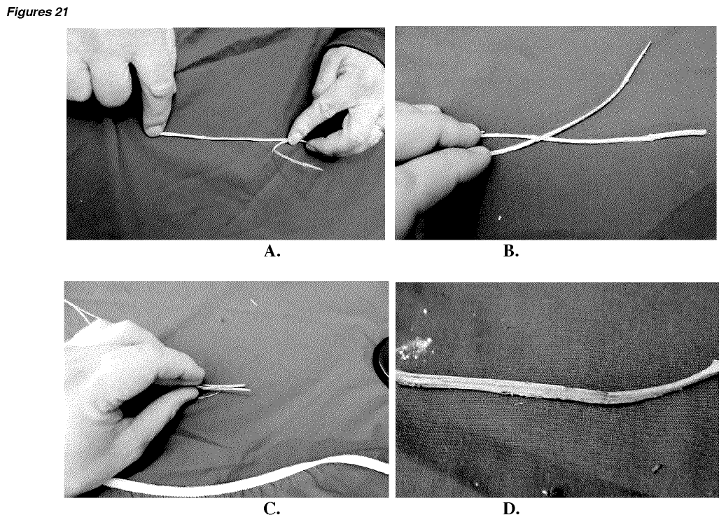

Evaluation was carried out at approx. 35 days post-inoculation where leaf and external stem disease symptoms were evaluated on each plant in each pot, by assessing the percent infected tissue, from 0 to 100%. In addition to foliar symptoms, each plant stem was split and the browning of the vascular tissue due to the fungus was measured and quantified (FIG. 21). Scalpels were used to split each stem and the full height of each stem was recorded (mm) as well and the length of vascular tissue which has turned brown due to the fungus.

Samples of leaves were taken once during each trial. At the end of the trial the first full trifoliate leaf sample was taken. The whole trifoliate leaf was harvested from the first full trifoliate of each plant. Leaf samples were placed into a pollinating bag and labeled. Leaves originating from plants in the same pot were placed into the same pollinating bag. Samples were air-dried until completely dry, crispy.

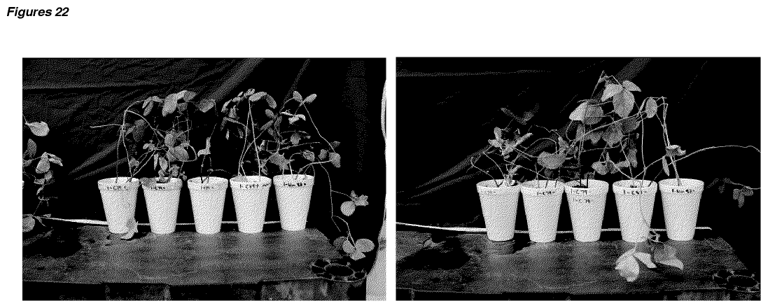

Photographs were taken for each entry per watering regime, if there were any visible differences in plant appearance or growth. Photographs were also taken of: general symptomology, assay layout, and methodology used (FIG. 22).

### Statistical Analysis of the BSR Greenhouse Experiment

The design of the experiment was such that all control replicates were concentrated on the left side of the greenhouse, and all treated replicates were concentrated on the right side of the greenhouse. Therefore, control and treated replicates were not randomized across the greenhouse. The design of the experiment did not allow the joint analysis of data from both treated and control groups. Hence, separate analysis of the data belonging to each group was performed. The analysis also discarded data from the lines named “Corsoy 79Nonlnoc A” and “Corsoy 79Nonlnoc B” because they did not get the same inoculation treatment as all other lines.

### Exploratory Analysis

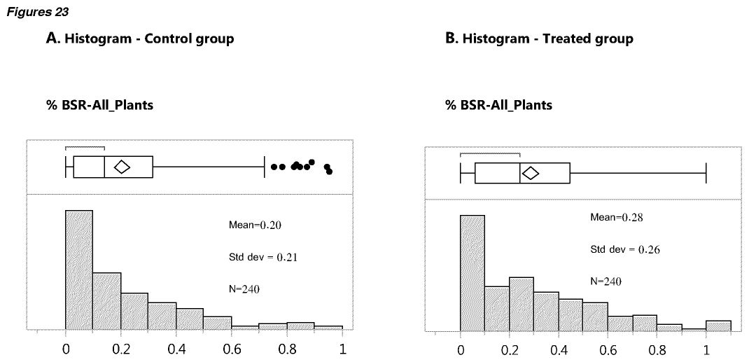

Histograms of the trait % BSR within each group show distributions that are highly skewed to the left and with large numbers of zero. There are 48 observations in the histogram of the control group (FIG. 23A) for which % BSR equals to zero, and there are 26 observations in the histogram of the treated group (FIG. 23B) for which % BSR equals to zero. The mean and the standard deviation of % BSR in the control group were respectively 20.15% and 21.28%. The mean and the standard deviation of % BSR in the treated group were respectively 28.54% and 25.88%, and the total number of observations in both histograms is 240. For the control group, the average of % BSR across all lines with low Si accumulation (“Low”) is 22.33% and the average of % BSR across all lines with high Si accumulation (“High”) is 14.95%. For the treated group, the average of % BSR across all lines with “Low” is 32.90% and the average of % BSR across all lines with “High” is 22.94%.

### Model Fit

We used generalized linear models for our parametric analyses because data of the trait % BSR is not normally distributed (as shown in histograms of FIG. 23), Thus, we assumed exponential distributions for % BSR in each group with reciprocal canonical link functions. We fitted the following model within each group:

% BSR=mean+Plant Height+MATID+REP+error

We included Plant Height as a covariate in the model to factor out a possible linear relationship between % BSR and Plant Height.

Subsequently to model fitting, we used contrasts to test the hypothesis:
Ho: mean of MATID_{low}=mean of MATID_{High}
Ha: mean of MATID_{low}≠mean of MATID_{High}

### Results: Control (Water) Group

The analysis of the data belonging to the control group showed a highly significant effect of MATID (p-value<0.0001) and a 10% significance level for the REP effect (p-value=0.1007). The test for differences in % BSR between lines with “Low” and “High” Si showed a significant difference estimated as 42.97% (low-high) with p-value=0,03, i.e. we rejected the null hypothesis of no differences between % BSR of lines with “Low” and % BSR of lines with “High” at 3% significance level.

### Results: Treated (SI) Group

The analysis of the data belonging to the treated group showed a highly significant effects for both MATID and REP (both p-values<0.0001). The test for differences in % BSR between lines with “Low” and “High” Si accumulation showed a significant difference estimated as 63.21% (“Low”-“High”) with p-value=0.02, i.e. we rejected the null hypothesis of no differences between % BSR of lines with “Low” and % BSR of lines with “High” at 2% significance level.

### Conclusion

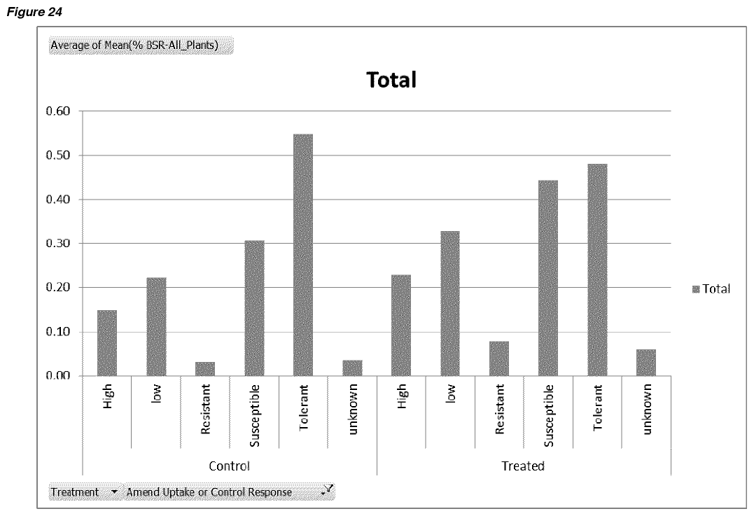

As per FIG. 24, lines with “High” Si accumulation showed significant less BSR damage than lines with “Low” Si accumulation, i.e. lines with “Low” showed around 43% more damage than lines with “High” within the control group, and lines with “Low” showed around 63% more damage than lines with “High” within the treated group.

There is evidence that the treated group had more pressure than the control group, i.e. The overall % BSR mean of the treated group was around 29%, whereas the overall mean of % BSR in the control group was around 20%. Also, the number of lines of zero % BSR damage was lower in the treated group (26) than in the control group (48). This could explain the larger difference in % BSR between “low” and “high” in the treated group than in the control group.

8B—Evaluation of Soybean (Glycine max) Recombinant Inbred Lines (RIL) with and without Silicon Soil Amendment to Determine Resistance to Soybean Cyst Nematode “SCN” (Heterodera glycines—Races 3)

The objective of this study was to evaluate 20 soybean lines (Table 22), with and without Silicon soil amendment, to determine resistance to Soybean Cyst Nematode “SON” under greenhouse conditions.

### Materials & Method

Overview of Procedure: Watering with AgSil21 was begun at least one week (7 days) prior to inoculation of soybean with any pest or pathogen. 100× (10,000 ppm) stock solutions of AgSil 21 (CA5684A) was prepared and stored in 1-liter batches. In preparation for application to plants, each 100× stock solution was diluted 100-fold (10 mL 100× stock per liter of onsite water), and pH-adjusted to fall between 6.0 to 7.9 by adding a small portion of concentrated acid (3M HCl: CAS #7647-01-0). 100 ppm (1×) solution was applied to plants within each of the 2 treatments, using a dedicated watering can for each treatment. The 1× diluted solutions was applied each time irrigation was needed. The control was onsite-water and pH of the onsite water was checked to ensure that it fell within the same range as the AgSil21 watering solution.

The experiment was designed as a factorial with split-split plot, where the main plots were the soil amendment (watering regime) and sub-plots were soybean lines, such that soybean lines were randomized within each replicate. Planting was carried out in 8 pots/replications per treatment. Two seeds were planted per pot, or seeds were pre-germinated and young seedlings were transplanted soon after germination. One seedling per pot was thinned after seeds for all treatments had germinated (approx. 5 days post-planting). Approx. 7 days after planting, SON were inoculated onto each treatment at an approximate rate of 2,000 eggs per pot.

Approximately one month after inoculation of SCN onto plants, the test plants were taken down for evaluation, and cysts removed from roots via washing over sieve screens to collect cysts. The number of cysts was evaluated by visually by counting under microscope.

Samples of leaves were taken once during each trial. The leaf samples were harvested just before the end of the trial. At this time the whole trifoliate was sampled from the first full trifoliate.

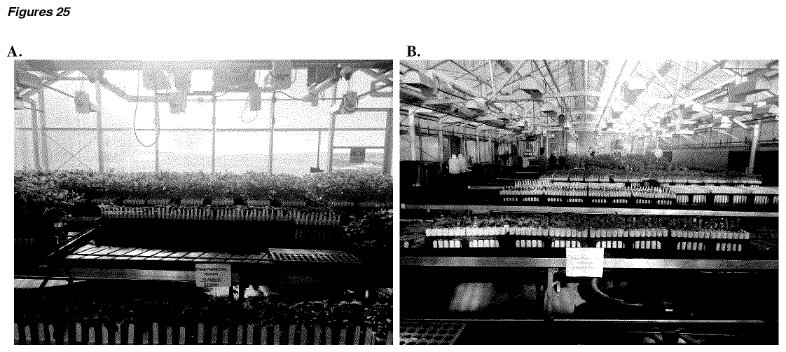

Photographs were taken for each entry per watering regime, if there were any visible differences in plant appearance or growth. Photographs were also taken of: general symptomology, assay layout, and methodology used (FIG. 25).

### Statistical Analysis of the SON Greenhouse Experiment

The design of the experiment was such that all control reps were concentrated on one bench of the greenhouse, and all treated reps were concentrated on a different bench. Therefore, control and treated reps were not randomized across the 2 benches used for the experiment and the design of the experiment does not allow the joint analysis of data from both treated and control groups. Hence, we performed separate analysis of the data belonging to each group.

### Exploratory Analysis

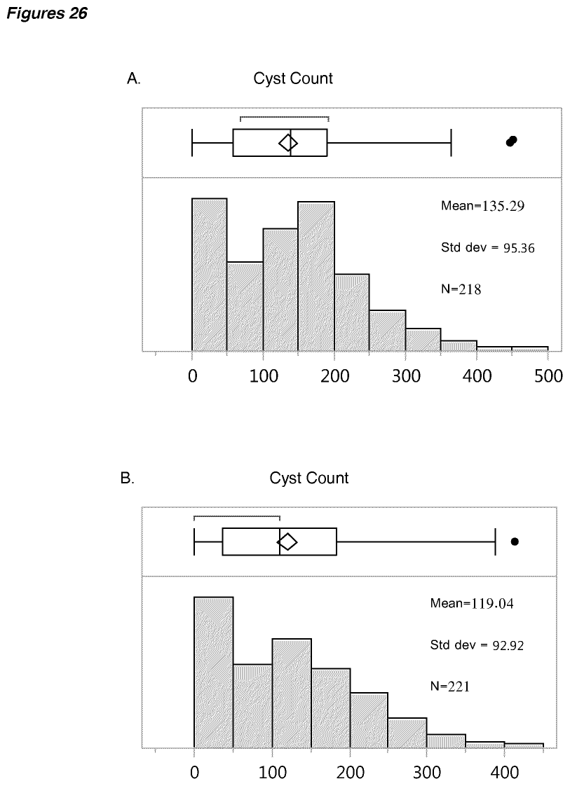

Histograms of the SON cyst counts within each group (control and Si treated; FIGS. 26) show left skewed distributions. There are 17 observations in the histogram of the control group for which Cyst Counts equals to zero, and there are 16 observations in the histogram of the treated group for which Cyst Counts equals to zero. The mean and the standard deviation of Cyst Counts in the control group were respectively 135.3 and 95.4 for 218 observations (FIG. 26A). The mean and the standard deviation of Cyst Counts in the treated group were respectively 119.0 and 93, for 221 observations (FIG. 26B). For the control group, the average of Cyst Counts across all lines with “Low” is 166.8 (sd =83.8) and the average of Cyst Counts across all lines with “High” is 142.2 (sd=83.2). For the treated group the average of Cyst Counts across all lines with “Low” is 158.6 (sd=87.6) and the average of Cyst Counts across all lines with “High” is 124.2 (sd=80) (now shown).

### Model Fit

We used generalized linear models for our parametric analyses because data of the trait Cyst Counts is a discrete variable (as shown in histograms of FIG. 26) that could fit the requirements of a Poisson distribution with overdispersion of the variance. Thus, we assumed for our model fitting Poisson distributions for Cyst Counts in each group with log link functions and overdispersion. We fitted the following model within each group:

Cyst Counts=mean+MATID+Plate+error

We considered Plate as an incomplete block factor. Subsequently to model fitting, we used contrasts to test the hypothesis:
Ho: mean of MATIDlow=mean of MATIDHigh
Ha: mean of MATIDlow≠mean of MATIDHigh
   Results : Control (water) Group

The analysis of the data belonging to the control group showed a highly significant effect of MATID (p-value<0.0001) and Plate effect (p-value=0.0065). The test for differences in Cyst Counts between lines with “Low” and “High” showed a significant difference (low-high) with p-value=0.05, i.e. we rejected the null hypothesis of no differences between Cyst Counts observed in lines with “Low” and Cyst Counts observed in lines with “High” at 5% significance level. However, the difference in Cyst Counts observed in lines with Low and Hi is no longer statistically significant if we do not include parental lines in our contrasts, i.e. “Low” (Majesta) in the low Si accumulator group and “High” (Hikmok) in the high Si accumulator group.

### Results: Treated (Si) Group

The analysis of the data belonging to the treated group showed a highly significant effects for both MATID and Plate effect (both p-values<0.0001). The test for differences in Cyst Counts between lines with “Low” and “High” showed a significant difference (low-high) with p-value=0.01, i.e. we rejected the null hypothesis of no differences between Cyst Counts observed in lines with “Low” and Cyst Counts observed in lines with “High” at 1% significance level. The difference in Cyst Counts between “Low” and “High” is still statistically significant (p-value=0.02) when we did not include the parental lines in our contrasts.

### Conclusions

Lines with “High” showed significantly less Cyst Counts than lines with “Low”. The Si treated group showed stronger (more consistent) results than the control group as the lines with “High” showed consistently less Cyst Counts than lines with “Low” independently of including parental lines in the contrast analysis.

8C—Evaluation of Soybean (Glycine max) Recombinant Inbred Lines (RIL) with and without Silicon Soil Amendment to Determine Resistance to Root-Knot Nematode “RKN”Meloidogyne incognita

The objective of this study was to evaluate 20 soybean lines (see Table 22), with and without Silicon soil amendment, to determine resistance to Root-knot nematode “RKN” under greenhouse conditions.

### Materials & Method

Overview of Procedure: Watering with AgSil21 was begun at least one week (7 days) prior to inoculation of soybean with any pest or pathogen. 100× (10,000 ppm) stock solutions of AgSil 21 (CA5684A) was prepared and stored in 1-liter batches. In preparation for application to plants, each 100X stock solution was diluted 100-fold (10 mL. 100× stock per liter of onsite water), and pH-adjusted to fall between 6.0 to 7.9 by adding a small portion of concentrated acid (3M HCl: CAS #7647-01-0). 100 ppm (1×) solution was applied to plants within each of the 2 treatments, using a dedicated watering can for each treatment. The 1× diluted solutions was applied each time irrigation was needed. The control was onsite-water and pH of the onsite water was checked to ensure that it fell within the same range as the AgSil21 watering solution.

The experiment was designed as a factorial with split-split plot, where the main plots were the soil amendment (watering regime) and sub-plots were soybean lines. Planting was carried out with sterile potting media at 4 pots/replications per treatment and 2 seeds per pot. Alternatively, seeds were pre-germinated and young seedlings were transplanted soon after germination. After seeds for all treatments have germinated (approx. 5 days post-planting) the plants was thinned to one seedling per pot. RKN was inoculated onto each treatment at an approximate rate of 2500 to 3000 eggs per pot. This was done approx. 7 days after planting.

Evaluation was carried out at approximately 45 days after inoculation of RKN onto plants, when the test plants were taken down. The roots were assessed using a rating system to look at the percentage of galled roots (not the number of galls).

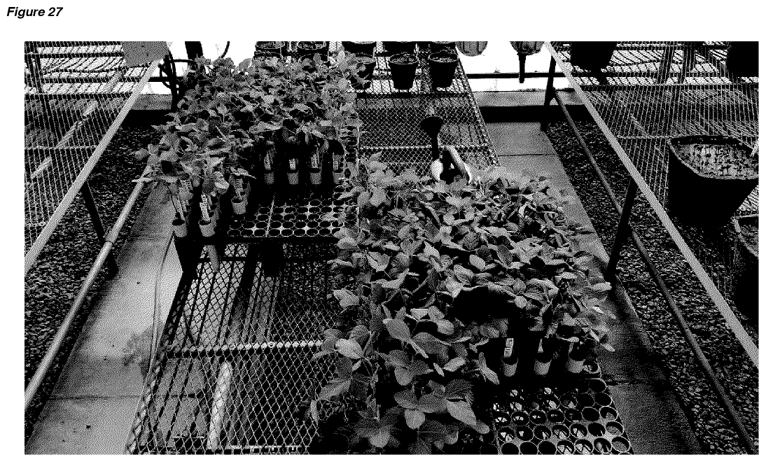

Photographs were taken for each entry per watering regime, if there were any visible differences in plant appearance or growth. Photographs were also taken of: general symptomology, assay layout, and methodology used (FIG. 27).

### Statistical Analysis of the RKN Greenhouse Experiment

There was no actual replication in the RKN experiment because the same arrangement of lines within a replication was repeated throughout all reps. Therefore we cannot make statistical inferences through a test of hypothesis (give p-values etc.). Hence, we performed an exploratory analysis in which we obtained statistical summaries, boxplots and show trends of the data.

### Exploratory Analysis

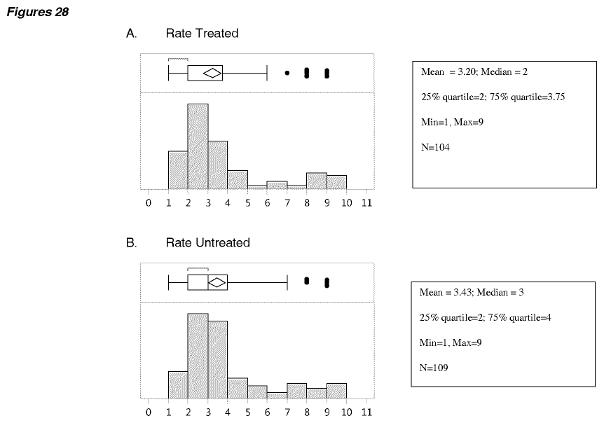

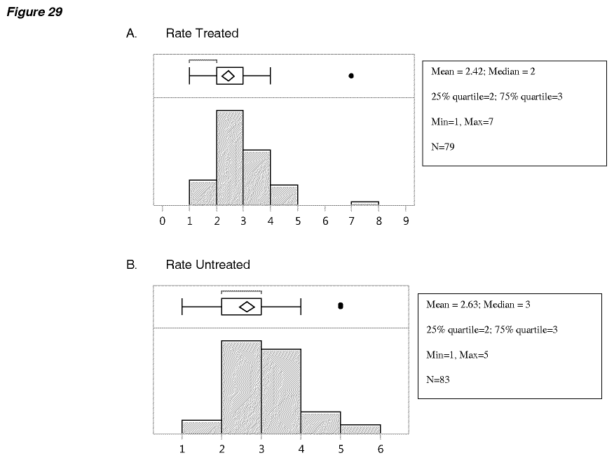

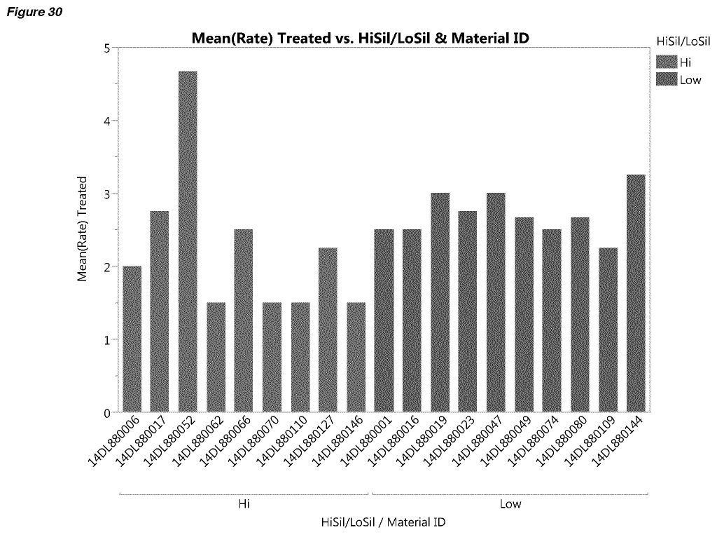

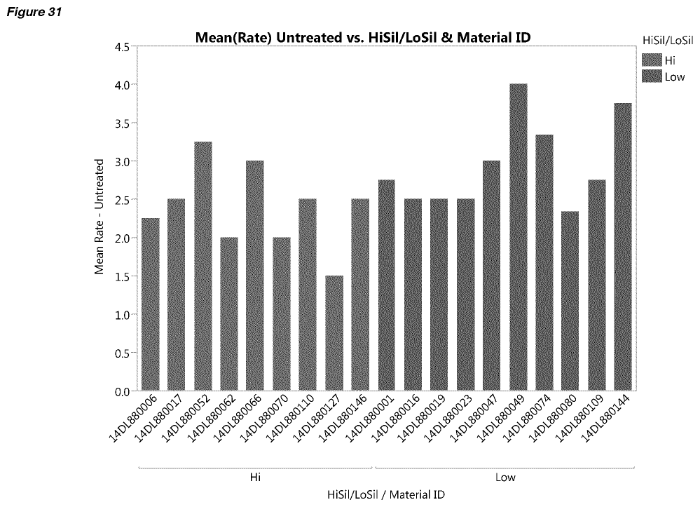

Histograms of RKN damage rates (FIG. 28) show distributions with a long right tail in both treated and untreated groups. The untreated group show slightly larger mean/median (3.43/4) (FIG. 28B) than the treated group (3.2/2) (FIG. 28A). FIG. 29 shows histograms of RKN damage without the checks. We can observe in FIG. 29 that the long tails observed in FIGS. 28 are mostly due to ratings of checks. Without data from the checks, the untreated group still shows slightly larger mean/median (2.63/3) (FIG. 29B) than the treated group (2.42/2) (FIG. 29A). It's important to notice that all checks were placed in neighboring cones at the border of every replicate. We obtained rate means over 4 reps for each line (see excel file with statistical summaries). Barplots of FIGS. 30 and 31 show rates means (over 4 reps) versus MATID, which are arranged according to “High” and “Low” subgroups.

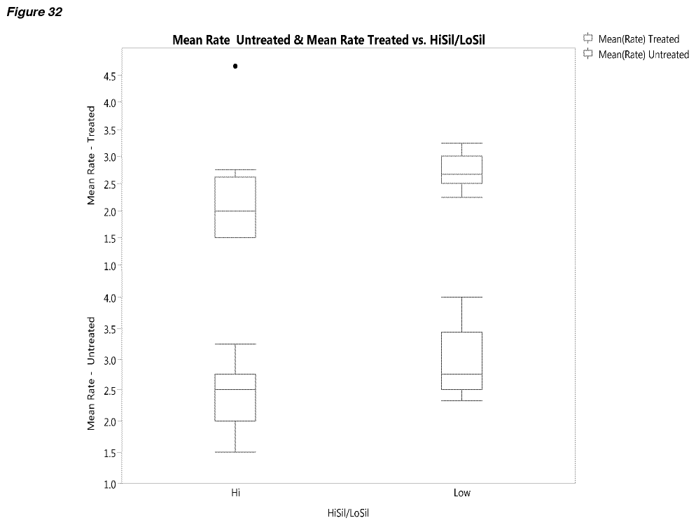

Boxplots of FIG. 32 show a possible difference between rates means of the subgroups “High” and “Low”, i.e. the overall mean of the subgroup Low (2.71 for the treated group and 2.94 for the untreated group) is larger than the overall mean of the subgroup High (2.24 for the treated group and 2.39 for the untreated group).

8D—RILs Carrying HiSil have Better Resistance to Phytophthora sojae

RILs carrying (or not) the HiSil allele from Hikmok sorip were tested for resistance against P. sojae under hydroponic conditions. A set of four RILs each with and without HiSil were grown in a greenhouse along with the parental lines Hikmok sorip and Majesta.

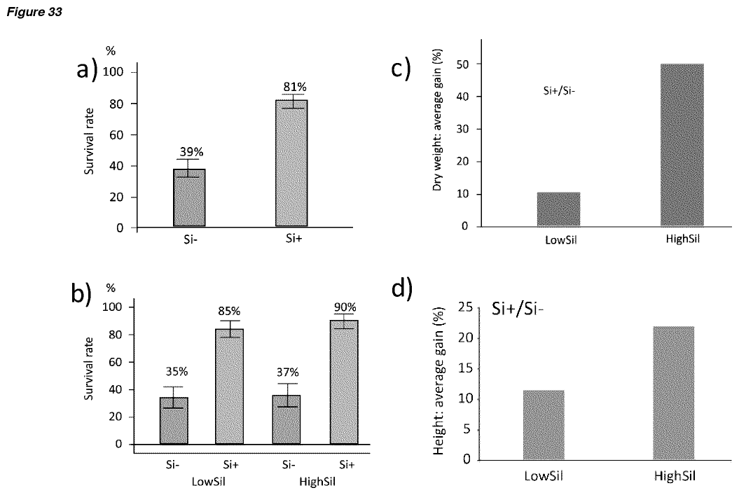

For the evaluation of the effect of Si on Phytophthora root rot (PRR), two independent experiments were performed. First experiments conducted with P. sojae race-25 showed that Si treatment increased survival rate of P. sofa-infected soybean plants by more than twice (FIG. 33a). The increase in survival rate was higher in HiSil RILs compared to LoSil RILs (FIG. 33b). Similarly, plant dry weight and height were higher in Si-treated plants (FIG. 33c, d). These experiments highlighted the prophylactic effect of Si against PRR and supported the hypothesis that the beneficial effects were more prominent in plants carrying the HiSil allele.

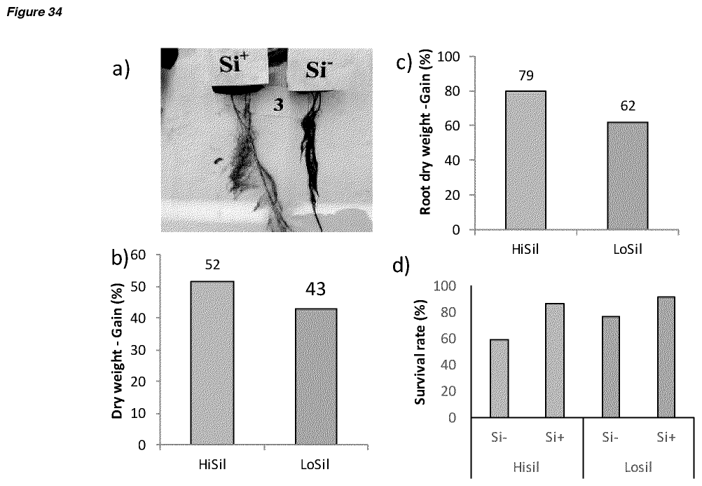

The second experiment was conducted using a cocktail of P. sojae races. For this purpose, the five most virulent races, including 4, 7, 13, 17 and 25, were used to inoculate HiSil and LoSil RILs. Even under this high disease pressure, significantly higher survival rate and root and shoot dry weight were observed following Si treatment (FIG. 34a). For all the measured variables, the gains were significantly higher in HiSil than in LoSil plants (FIG. 34b, c, d)). In conclusion, Si provided horizontal resistance against PRR covering a broad range of P. sojae races and this resistance was more manifest in HiSil plants.

8E—RILs Carrying HiSil have Better Drought Tolerance

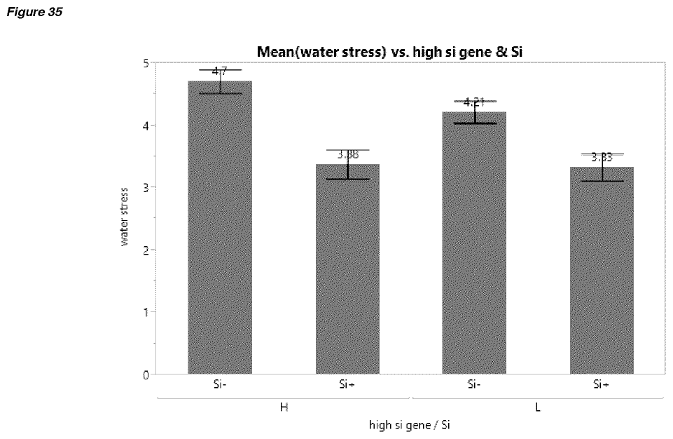

RILs carrying HiSil allele from Hikmok sorip were tested for drought tolerance under Si fertilization. A set of four RILs each with and without HiSil allele were grown in a greenhouse along with parental lines Hikmok sorip and Majesta. Leaf wilting score of soybean plants grown under hydroponic conditions for three weeks and then subjected to water stress by cutting off water supply was recorded. Wilting scale used is—1 for no wilting, 2 very slight wilting, 3 wilting, 4 high wilting, 5 dying, and 6 is for dead. A significantly lower level of wilting was observed as a result of Si fertilization. This difference was more pronounced in RILs carrying HiSil allele than in RILs without it (FIG. 35).

### Methods

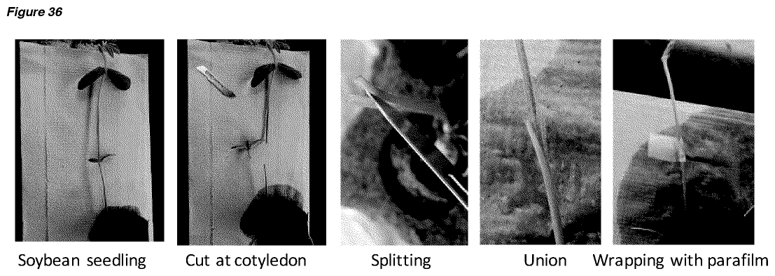

A grafting experiment was conducted to create a situation where the aerial part of the plants had exactly the same genetic background but with differential Si uptake capability from two different rootstocks. This provided a sensible alternative over isogenic lines typically required for the evaluation of allelic effect of a gene. Grafting of soybean plants was performed on one-week-old seedlings grown in Oasis cubes. A cleft grafting approach was used to make the grafts. Shoots were cut at right angle below the cotyledons. The rootstock was then split down at the center at a one-inch depth. The scion was chopped from both sides to form a pointed tip as shown in FIG. 36. Then the scion was inserted into the rootstock split and the union was wrapped with parafilm tape.

The grafted plants were maintained at high humidity under plastic dome for three days before transplanting into a hydroponic system. A total of 20 plants were transplanted into each plastic tunnel. Plants were supplied with a nutrient solution amended with or without Si (1.7 mM). Water stress was imposed three weeks after transplanting by withdrawing water from the tunnels. The leaf wilting symptoms were scored with a wilting scale where: −0—no wilting; 1—very slight wilting; 2—slight wilting; 3—wilting; 4—high; 5—dying, and 6—dead.

### Results

Hikmok plants were the most susceptible to water stress in absence of Si amendment. However, in presence of Si, the wilting symptoms were drastically reduced.

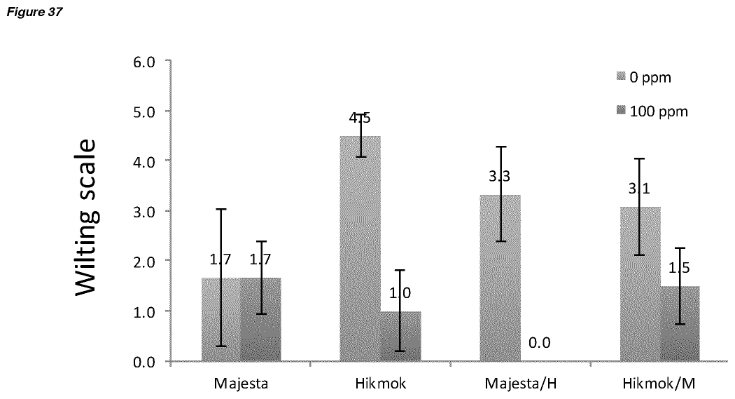

The same phenomenon was observed with Majesta scions grafted on Hikmok roots. By contrast, Majesta plants did not benefit from Si amendments, Finally, a reduction in drought stress was observed with Hikmok scions grafted on Majesta rootstocks (FIG. 37).

### Example 9

### Evaluation of Effect of Glyma16g30000 and Glymal6g30020 in Transgenic Arabidopsis

### Methods

### Plant Material and Growth Conditions

Four different Arabidopsis genotypes [Colombia (Col-0; Ohio State University), TaLsi1 lines (Montpetit et al., 2012), TaLsi1 Hisila and TaLsil Hisilb lines] were used in the present work. For all experiments, seeds were surface-sterilized (5% bleach, 2 min), rinsed five times with water and stored at 4° C. for 3 days to break dormancy. Col-0 seeds were directly sown on Veranda® Container Mix (Fafard et freres) in a growth chamber under long-day conditions (14 h of light at 22° C., 10 h of dark at 19° C., 55-65% humidity and a light intensity of 150 μmol/m2/s) and covered with plastic sheets for one week. TaLsi1 lines and T2 TaLsil HiSil lines were selected on Murashige and Skoog Basal Medium with Gamborg's Vitamins (MS) (Sigma-Aldrich) containing hygromycin (15 mg/L) for TaLsil lines and kanamycin (50 μg/ml) for TaLsi1 HiSil lines. At day 10, seedlings of uniform size were transferred to pots containing Veranda® Container Mix at a density of five plants per pot. Plants were treated with water containing 1.7 mM Si in the form of K₂SiO₃. Only controls (Col-0 and TaLsi1 lines) received a treatment without soluble Si, in which potassium chloride was used to replenish potassium. Plants were maintained in a growth chamber as described above. Arabidopsis plants of different genotypes were used for experiments one month after transplanting.

### Isolation of Promoter Region, Construction of Promoters: GUS Reporters and Plant Transformation

The 2.5 kb region upstream of the initiation codon of N1P5;1 gene (AT4G10380) was amplified from a BAC clone. The 290 bp region upstream of the initiation codon of CASP2 gene (AT3G11550) was amplified by PCR from genomic DNA extracted from Col-0 Arabidopsis plants using high fidelity polymerase (Phusion®, New England BioLabs). Primers were designed to amplify promoters and to introduce Smal and HindlIl or Sbfl restriction sites (see Table 1). PCR products were cloned in pGEM®-T easy using Takara ligation kit (Takara). Promoters were then cloned in TOP 10 E. coli cells and clones were screened for presence of insert with colony PCR. Next, plasmids were recovered from a fresh bacterial culture using the QIAprep Spin Miniprep kit (Qiagen). Finally, 1 μg of pure plasmid DNA was digested with restriction enzymes followed by confirmation of the amplicons by DNA sequencing,

Promoters were inserted into the plasmid pB1121 (Clontech), a binary vector harbouring a GUS reporter gene. Insertion was into the SmaI and HindIII or Sbfl sites in order to replace the CaMV 35s promoter and ligation was assessed using Takara ligation kit (Takara). Cloning in TOP10 E. coli cells for multiplication was made prior to cloning in Agrobacterium tumefaciens strain GV3101 for plant transformation.

Col-0 Arabidopsis plants were transformed by a modified floral dip method (Zhang et al., 2006). Independent transgenic lines (T1) were selected for Kanamycin resistance (50 μg/ml) on MS medium (Sigma-Aldrich) and the presence of the regulatory regions was verified by PCR (see Table 1). T2 transgenic seeds were harvested and sown on MS medium containing Kanamycin (50 μg/ml) for 10 days and transferred into Magenta box for growth. T2 transgenic plants were used for phenotypical analyses.

### Histochemical GUS Staining

The Gus-assays were performed on 3 weeks old transgenic Arabidopsis plants. For histochemical localisation of β-glucuronidase (GUS) activity, β-glucuronidase reporter gene staining kit (Sigma) was used according to the manufacturers instructions. Incubation was in the dark at 37° C. overnight and tissues were washed twice with ethanol 100% until the chlorophyll pigments were completely bleached. Whole plants were observed directly under binocular and light microscopes.

### Construction of Plant Expression Vectors and Plant Transformation

The two HiSil soybean candidate genes, Glyma16g30000 (Hisila) and Glyma16g30020 (Hisilb), genes were amplified from Hikmok sorip and Williams, and verified for sequences correctness. All four alleles (alleles Williams and Hikmok from both genes) were synthesized (Genscript) in pUC57 with Smal and Sac' sites to ensure sequence accuracy. Col-0 and TaLsil line were used to express Hisila and Hisilb. Conventional molecular cloning techniques were applied to construct the plant expression vectors. Binary vector pB1121 containing either NIP5;1 or CASP2 promoter was digested with SmaI and SacI in order to remove the GUS reporter gene. All synthesized alleles were also digested with Smal and Sacl. Ligation of four different alleles in the vector containing one of two promoters for a total of 8 different constructs was made using Takara ligation kit (Takara). Constructs were cloned in TOP 10 E. coli cells and clones were screened for presence of insert with colony PCR. Next, plasmids were recovered from a fresh bacterial culture using the QIAprep Spin Miniprep kit (Qiagen). Pure plasmid DNA was digested with restriction enzymes and minipreps were sent for sequencing for confirmation. One positive clone for each construct was cloned in Agrobacterium strain GV3101 using a modified freeze-thaw method (Jyothishwaran et al., 2007) and after validation with colony PCR, one clone per construct was selected for plant transformation. A. thaliana was transformed according to a modified floral dip method (Zhang et al., 2006). Independent T1 transgenic lines were selected on the MS medium (Sigma-Aldrich) containing Kanamycin (50 μg/ml), and the presence of the HiSil transgene was verified by polymerase chain reaction (PCR) (see table 1). T2 seeds were harvested from independent transgenic lines bearing each construct, respectively, and sown on MS medium containing Kanamycin (50 μg/ml). For all experiments, the phenotype of the T2 transgenic plants was analyzed.

### Determination of Si Concentration in Transgenic Arabidopsis Shoots

Transgenic lines TaLsil, TaLsi1 HiSil and Col-0 plants treated or not with Si were analysed in this study. The Si content in experimental plants was measured by colorimetric analysis following an HCL-HF extraction (Taber et al., 2002). Aerial parts of the plants from each treatment (5 plants per line) were collected and freeze-dried one month after the beginning of Si amendment. Samples were ground to a powder before Si analysis. For each treatment a minimum of five biological replicates were used.

### Statistical Analyses

Statistical significance was assessed with Student's t-test and Dunnett's test using JMP 12 software (SAS institute Inc.). Least square means were used to express the results. Standard error was used as the error bar in figures.

### Results

### Validation of HiSil Activity in a Transgenic Arabidopsis

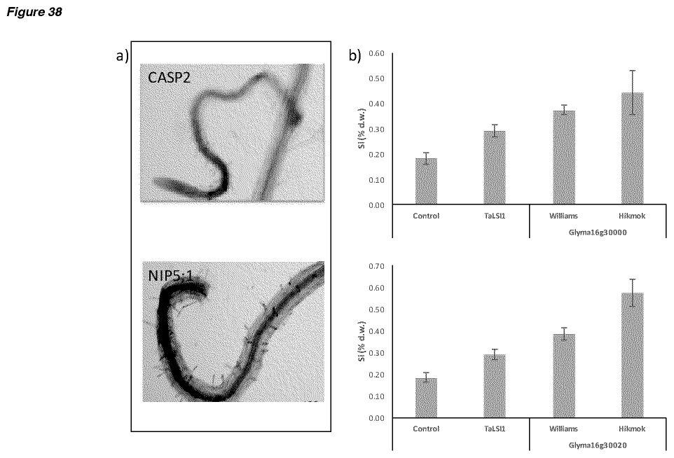

Arabidopsis transformation with alternative alleles for both candidate genes Glyma16g30020 and Glyma16g30000 was performed to validate HiSil activity. To achieve constitutive expression in root tissues, constructs were made with two promoters NIP5;1 and CASP2. Constructs with both promoters showed expression of GUS in the root tissue (FIG. 38a). A total of 8 different constructs representing two promoters, and two alleles representing Williams and Hikmok sequences were prepared. Evaluation of transgenic Arabidopsis lines showed a significantly higher Si accumulation for Hikmok allele compared to Williams allele of Glyma16g30020 (FIG. 38b).

TABLE 23
List of primers used in this study.

| | | SEQ |
|---|---|---|
| Table 23 | | ID |
| Name | Sequence 5′-3′ | NO. |
| ATPRO 11550 fwd | GAC CTG CAG GCA CCT TTA | 616 |
| | CCT ATT TCA TAA TAT AAT TAT C | |
| ATPRO 11550 rev | GAG ACC CGG GGG ATG CTT | 617 |
| | TGG TGG TGA ATG AG | |
| HINDIII-PNIP5 fwd | GAG AAA GCT TGA AAG CAA | 618 |
| | GCA TTC CCT G | |
| SMAI-PNIP5 rev | GAG ACC CGG GCA ACG TTT | 619 |
| | TTT TTT TTG GT | |
| Hyg R JAW fwd | ATG TAG GAG GGC GTG GAT | 620 |
| | ATG T | |
| Hyg R JAW rev | TGC CGT CAA CCA AGC TCT GA | 621 |
| 30000 fwd | TGT GCC TTT TCT ACC CAT TG | 622 |
| 30000 rev | GAT TTC CAC AGT ACC CTC T | 623 |
| HiSil fwd 2 | GGA GTT GTG GTG AAT GTT G | 624 |
| Hisil rev 2 | GGG TTT TCC CAG TCA CGA | 625 |
| ATPRO fwd2 | GTG AGA CCC AAT GAA AGA C | 626 |
| Atpro REV2 | TAA GGT GGG AGG TTA TGT TG | 627 |
| Gamma fwd | TAT ACC CGG GAT GGC ATT | 628 |
| | GGC TCC TAC TCC | |
| Gamma rev | GCG CGA GCT CTC ATT TTA | 629 |
| | TGA GTG TCA ACC | |

### Example 10

### Transgenic Soybean Expressing the HiSil Gene (30020) Under Control by its Native Promoter/Terminator Sequences

### Methods.

Wlliams82 soybean plants were transformed with the HiSil allele (SEQ ID NO. 14) composed of the native promoter (SEQ ID NO. 20) and native terminator regions.

T1-generation seeds from 10 independent events were sown in germination soil and segregation was determined by zygosity using the Taqman® gene expression assay.

Once segregated, homozygous and null siblings were watered with 1.77 mM AgSil (˜pH 7.5) beginning at the V2-stage (no NPK fertilizer was used) and single leaflets from the 1st and/or 2nd trifoliate were sampled at time 0 and at 10, 20 and 30 days post-silicon application. The leaves were then freeze-dried and shipped for analysis.

### Results

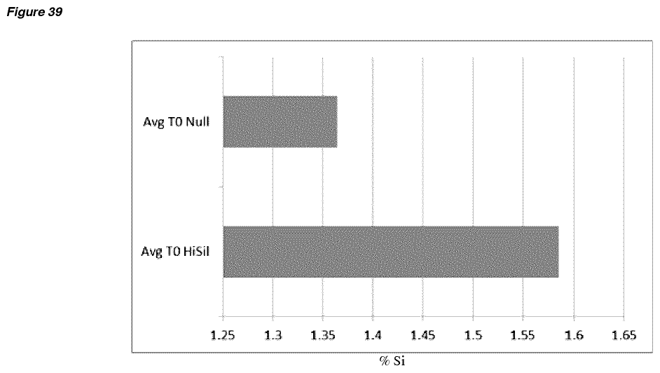

FIG. 39 shows that, on average (averaging all controls & all homozygous pools), plants expressing the HiSil gene (SEQ ID NO. 14) gave an average leaf accumulation of 1.5857 units of Si, whereas “Null” plants averaged 1.364 Si units.

### Conclusion

Plants from the homozygous pool showed an average of 16.22% accumulation of Si over null plants.

### Example 11

### Silicon Efflux Transport Activity of Glyma16g30020 Evaluated in Xenopus oocytes Assay

### Methods

Plasmid Construction for Heterologous Expression in Xenopus oocytes

Complete coding DNA sequence (CDS) for Glyma16g30020 was amplified with primers having extended sequence for Spel and BgIII endonuclease sites. The amplified CDS sequences representing both Hikmok soprip and Majesta alleles were digested with SpeI and BgIII endonucleases, Then, the digested CDS products were cloned into the pre-digested pT7TS vector, a Xenopus laevis oocyte expression vector derived from pGEM4Z, comprises the T7 and SPO promoters, 5′ & 3′ untranslated regions (UTRs) of Xenopus

Beta-globin gene and a poly(A) tract (Addgene plasmid #17091, www.addgene.org). All vectors were transformed into Escherichia coli TOP10 strain and stored at −80° C. The correctness of the constructs was confirmed by sequencing prior to in vitro translation.

Si Transport Assays Using Heterologous Expression in Xenopus oocytes

Plasmids containing the Glyma16g:30020 CDS were recovered from a fresh bacterial culture using a QIAprep Spin Miniprep kit (Qiagen). A total of five μg of each plasmid were linearized using SmaI (Roche, http://www.roche.com). Digested products were column-purified using a PCR purification kit (Qiagen), and 1 μg of DNA was transcribed in vitro using the mMessage mMachine T7 Ultra kit (Ambion, www.invitrogen.com/site/us/en/home/brands/ambion.html). Complementary RNAs (cRNAs) were purified using phenol/chloroform precipitation, and suspended in water treated with 0.1% DEPC (Sigma-Aldrich, www.sigmaaldrich.com/). Defolliculated stage V-VI oocytes were injected with 25 nl of 8.5 nM Si solution (control), or with 25 nl of 500 ng/μl cRNAs solubilized in a 8.5 nM final Si solution. A first pool of ten (10) oocytes for each treatment of injection were recovered (=T0), rinsed in sucrose-HEPES solution and frozen until Si intracellular measurement. Remaining eggs were maintained at 18° C. in modified Barth medium (MBS) (88 mM NaCl, 1 mM KCl, 2.4 mM NaHCO₃, 0.82 mM MgSO₄, 0.33 mM Ca(NO₃)2.4H₂O, 0.41 mM CaCl₂, 15 mM Hepes, pH 7,6) supplemented with 100 μM of penicillin/streptomycin. Seventy-two (72) hours after injection, a second pool of 10 oocytes for each treatment were recovered, rinsed in sucrose-HEPES solution and frozen until Si intracellular measurement.

Dosage of Si in Xenopus oocytes

Concentrated nitric acid (25 μl) was added to each pool of ten (10) oocytes, which were then dried for 2 h at 82° C. Plasma-grade water (100 μl) was added, and samples were incubated for 1 h at room temperature. Samples were vortexed, then centrifuged for 5 min at 13,000 g. The intracellular Si concentration was measured in 10 μl of supernatant by Zeeman atomic absorption using a Zeeman atomic spectrometer AA240Z (Varian; www.varian.com) equipped with a GTA120 Zeeman graphite tube atomizer. The standard curve was obtained using a 1,000 ppm ammonium hexafluorosilicate solution (Fisher Scientific, www.fishersci.com). Data were analyzed with SpectrA software (Varian).

### Results

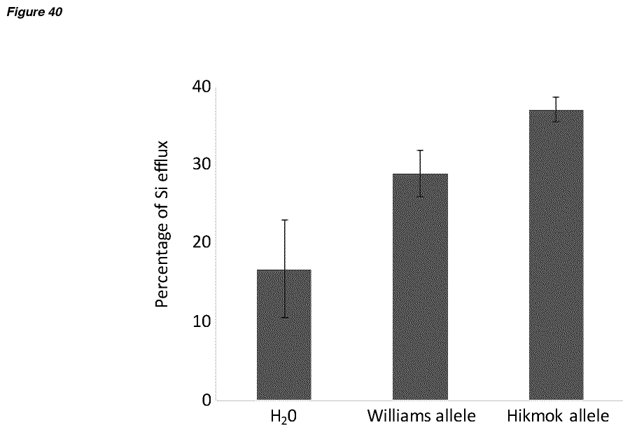

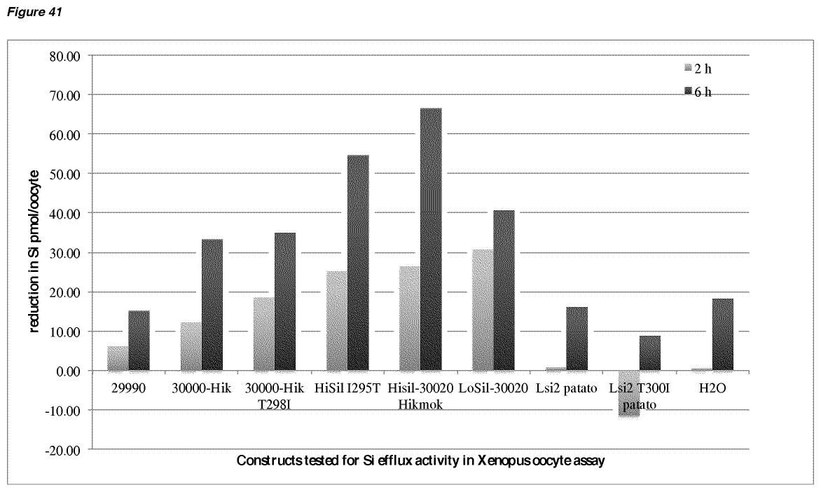

Evaluation of Si transport activity in Xenopus oocytes showed efflux activity for Glyma16g:30020. Significantly higher Si efflux was observed for the Hikmok allele compared to Williams allele (FIG. 40). The Williams allele represents haplotype 5 (H5; see FIG. 18) the most frequent allele type observed in most soybean cultivars including Majesta.

After evaluation of several different constructs, FIG. 41 shows that both genes Glyma16g:30000 and Glyma16g:30020 are functional Si efflux transporters. Interestingly, the position corresponding to position 295 (isoleucine) of Glyma16g30020 (also SEQ ID NO: 15) may be a significant protein structure that enhances or decreases the functionality of the protein. For example, as shown in FIG. 41, HiSil 30020 Hikmok comprising a isoleucine at position 295 demonstrates a increase in Si efflux as opposed to LoSil 30020 not comprising said isoleucine at position 295. Further, when the HiSil 30020 Hikmok isoleucine (I) at position 295 was substituted with a Threonine (T) the protein unexpectedly functioned similar to the LoSil 30020 protein, thus indicating that position 295 may be a important amino acid for protein function (see “HiSil I295T” in FIG. 41). Furthermore, it is noted that there likewise was a enhancement of efflux function when the corresponding position (i.e. position 298) of Glyma16g30000 was changed from a T to I there was an increase in efflux activity (see FIG. 41).

### Example 12

### Elite Soybean Introgression

A donor line having in its genome the HiSil locus is crossed with a with a recipient line such as, for example, an elite soybean line selected from: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, III., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minn., USA); 90M01, 91M30, 92M33, 93M11, 94M30, 95M30, 97B52, P008T22R2; PI6T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRRISTS (Soygenetics, LLC, Lafayette, Ind., USA); S00-K5, S11-L2, 528-Y2, 543-B1, S53-A1, S76-L9, S78-G6, 50009-M2; S007-Y4; 504-D3; 514-A6; 520-T6; 521-M7; 526-P3; 528-N6; 530-V6; 535-C3; 536-Y6; S39-C4; S47-K5; 548-D9; 552-Y2; 558-Z4; 567-R6; S73-S8; and 578-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

The seeds are then collected from the cross of step 1, and a progeny is grown up. The progeny is then selected for having the HiSil Locus using marker assisted breeding to identify markers/QTL associated with the trait, for example, such as markers corresponding to the ones listed in Tables 15-20.

One or more backcrosses are performed with the elite Glycine max. The plants are then selfed and the seeds collected. The plants from the seeds are then evaluated for the presence of HiSil loci (i.e. marker assisted breeding).

Elite Gmax Hisil plants are then grown and produced from the selected plants.

### Example 13

### Generation of Cisgenic Events Containing Genomic Fragment of HiSil Allele from Hikmok sorip Line

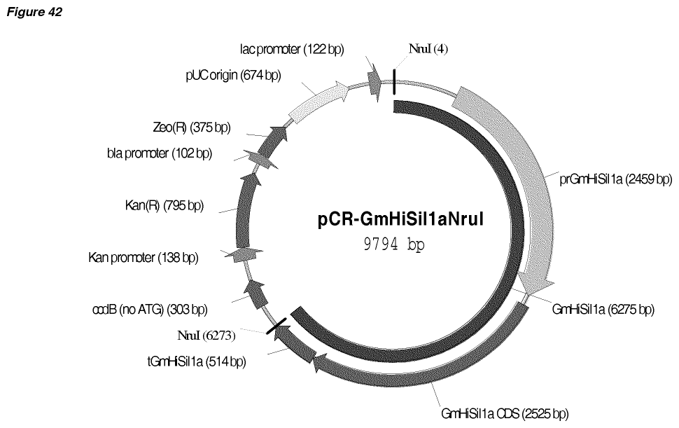

Jack soybean calli are transformed with a Hikmok sorip genomic fragment containing the HiSil allele (SEES ID NO: 630) composed of the native promoter, 5′-untranslated, coding region including introns and 3′-untranslated region. Since both of the 5′-(CGA) and 3′-(TCG) ends of the fragment contain half Nrul cleavage site (5′-TCGCGA-3′), 3 bases are added to both ends so the fragment is flanked by two Nrul sites during synthesis of primers to amplify the fragment for cloning. The GmHiSil genomic DNA sequence is amplified from Hikmok sorip soybean line using high fidelity DNA polymerase and cloned into pCR-TOPO vector, pCR-TOPO clones with PCR product insert are analyzed with DNA sequencing. A GmHiSil clone with no PCR-introduced mutation is named pCR-GmHiSil1aNrul (FIG. 42).

For soybean transformation, the whole Nrul fragment (6275 bps) containing the HiSil gene is released from the plasmid pCR-GmHiSillaNrul and purified using standard method such as preparative gel electrophoresis followed by electroelution. A separate DNA fragment comprising of a selectable marker gene (ALS or PMI) cassette is also prepared for co-delivery into the soybean callus tissues along with the HiSil fragment.

Transformation of soybean calli is done via physical delivery method, preferably biolistic bombardment [McCabe et al. (1988) Transformation of shoot meristems by particle acceleration. Bio/Technol 6:923-926; Finer and McMullen (1991) Transformation of soybean via particle bombardment of embryogenic suspension culture tissue. In Vitro Cell Dev Biol. 27P:175-182; Santarem and Finer (1999) Transformation of soybean [Glycine max(L) Merrill] using proliferative embryogenic tissue maintained on semi-solid medium. In Vitro Cellular & Developmental Biology—Plant 35:451-455.] Callus tissue is induced from immature embryos and used for particle bombardment. Transformed calli are selected on media containing selection agent, such as ALS inhibitor herbicide chlorsulfuron if acetolactate synthase (ALS) gene is used as selectable marker. Alternatively, mannose can be used as selection agent if phosphomannose isomerase (PMI) is used as marker. Selected transgenic calli are placed on regeneration media to form somatic embryos. Somatic embryos are then placed on maturation media and mature somatic embryos are then later desiccated and then germinated to from T0 transgenic plants. TO cisgenic/transgenic plants are assayed for the presence of GmHiSil gene insertion. Optimally, plants with low copy of GmHiSil and ALS or PMI marker gene insertion are selected to be grown to maturity. T0 plants are self-pollinated or backcrossed with other genotypes of soybean to produce progeny seeds. Progeny seeds are planted and individual plants are genotyped to select for lines that only contain a single copy of GmHiSil insertion, but with no ALS or PMI selectable marker transgene. The lines with only GmHiSil insert are “cisgenic” since they do not contain any foreign DNA sequences.

### Example 14

### Generation of Genome Edited Soybean Plants Containing Genotype of HiSil Allele of Hikmok sorip Line

The protein coding sequences of silicone transporter genes (GmLSi) of transformable lines Williams 82 and Jack are only 5 bases different from the Hikmok sorip sequence (GmHiiSil, SEQ ID NO: 630). Only 2 of them lead to change of amino acid sequence in the silicon transporter protein. Genome editing technologies can be used to convert the GmLSi gene in low silicon-accumulating lines such as Jack into high silicon-accumulating GmHiSil allele present in Hikmok sorip. Several types of programmable site-directed nucleases can be used to achieve such a purpose, including but not limited to zinc finger nuclease (ZEN), TAL effector nuclease (TALEN), engineered meganuclease (eMN), CRISPR-Cas9 and DNA-guided Argonaute system (Puchta and Fauser (2014) Synthetic nucleases for genome engineering in plants: prospects for a bright future. Plant Journal 78:727-741; Chen and Gao (2014) Targeted genome modification technologies and their applications in crop improvements. Plant Cell Rep. 33:575-583; Gao et al (2016) DNA-guided genome editing using the Natronobacterium gregoryi Argonaute. Nature Biotech. doi:10.1038/nbt.3547).

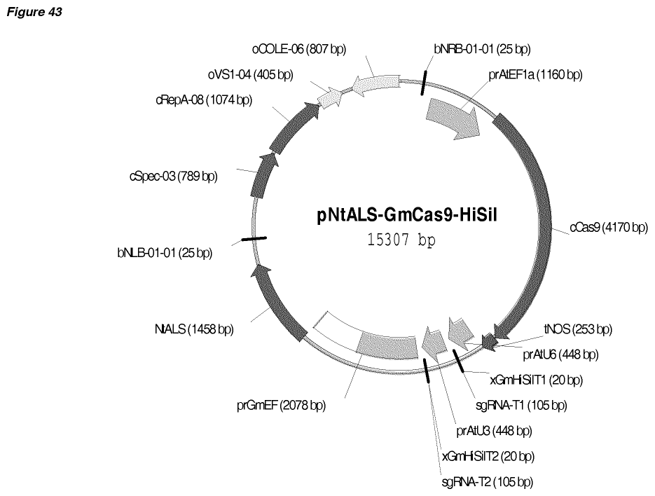

Here, we describe the use of one of the genome editing systems, CRISPR-Cas9 to mediate replacement of nucleotide sequence of GmLSi gene in soybean line Jack with GmHiSil allele from Hikmok sorip. CRISPR-Cas9 -mediated gene modification requires these components: Cas9 nuclease, crRNA (CRISPR RNA) recognizing the mutagenesis target, tracRNA (transactivating RNA) and repair donor DNA template molecule. For easiness of use, crRNA and tracRNA are usually fused and delivered as a single guide RNA molecule (gRNA or sgRNA) [Sander and Joung (2014) CRISPR-Cas systems for editing, regulating and targeting genomes. 32:347-355]. In order to achieve good expression in maize cells, Type II Cas9 gene from Streptococcus pyogenes SF370 is optimized with soybean-preferred codons. Nuclear localization signal is also incorporated into the C-terminus of Cas9 to improve its targeting to nucleus. To express Cas9 in soybean cells, the soybean-optimized Cas9 gene is placed under the control of a strong constitutive Arabidopsis Elongation Factor promoter (prAtEF1a) and followed by a NOS terminator sequences (tNOS) (FIG. 43).

In this example, a transformation vector pNIALS-GmCas9-HiSil (Figure Y-1) contains expression cassettes for selectable marker gene ALS, Cas9 and two sgRNAs (single guide RNAs). The two sgRNAs guide Cas9-medaited cleavage of Jack genomic sequences around the 2 target regions and generate dsDNA breaks. Two repair donor oligonucleotide sequences are co-delivered into the Jack soybean callus tissue to mediate replacement of the GmLSi target sequences with HiSil alleles of Hikmok sorip. Both donor oligonucleotides have one of the nucleotides corresponding to the PAM sequences (5′-NGG) mutated so the replaced allelic sequence will not get cleaved again by Cas9. More specifically, in Jack Target 1 (SEQ ID NO 631: 5′-ATGGC ATTGG CTCTT ACTCC AACAG TTGTC TTTGG-3′), the replaced allele is one nucleotide (underlined) different from Hikmok sorip sequences (SEQ D NO 632: 5′-ATGGC ATTGG CTCCT ACTCC AACAG TTGTC TTTGG-3′), but this difference is a silent mutation resulting in no amino acid sequence change. For this target, a sgRNA-T1 in pNtALS-GmCas9-HiSil (Figure Y-1) containing targeting sequence xGmHiSil-T1 (SEQ ID NO 633: 5′-TTTAA CCACA ACAAT GGCAT-3′) is used to guide Cas9 cleavage. For this target, a donor oligonucleotide of 74 bps (DON-HiSil-T1, SEQ ID NO 634: 5′-GTTTG GAAAT TGTTG CTTGT TTAAC CACAA CAATG GCATT CGCTC CTACT CCAAC AGTTG TCTTT GGCTC AATA-3′) is used to replace the Jack target sequence. For replacement of sequences in Jack Target 2 (SEQ ID NO 635: 5′-AATTT CAGCT ATATC AAGTG CCTTT TTCA -3′) has two bases different than the Hikmok sorip allele (SEQ ID NO 636: 5′-AATTT CTGCT ATATC AAGTG CTTTT TTCA -3′). For this target, a guide RNA sgRNA-T2 in pNtALS-GmCas9-HiSil (FIG. 43) containing targeting sequences xGrnHiSil-T2 (sgRNA-2, SEQ ID NO. 637: 5′-AGATG TGTCA TTGGT GAAAA -3′, targeting the coding strand) is used to guide Cas9 cleavage. For this target, a donor oligonucleotide of 83 bps (DON-HiSil-T2, SEQ ID NO 638: 5′-AAGGA CTTAC TCTGT AGAAT TTGTT TAATT TCTGC TATAT CAAGT GCTTT TTTCA CCAAT GACAC ATCTT GTGTT GTATT GAC -3′) is used to replace the Jack target sequence.

To generate allele replaced soybean lines, transformation vector pNtALS-GmCas9-HiSil (FIG. 43) is co-precipitated with two oligonucleotides (DON-HiSil-T1 and DON-HiSil-T2) onto gold particles and then co-delivered into Jack calli by biolistic bombardment. Bombed calli are selected with ALS herbicide such as chlorasulfuron and selected calli are regenerated into somatic embryos. Somatic embryos are germinated as described above for generating cisgenic plants. After germination, seedlings are sampled for molecular analysis to identify lines containing desirable mutations with Hikmok sorip-type allele. Identification of candidate mutants can be done using restriction digestion if suitable site can be found to distinguish WT than from mutant. Alternatively, highly sensitive SNP-assay or qPCR Taqman assay can be designed to identify desirable edited mutants. Identified potential mutations are typically confirmed by sequencing analysis of PCR products in these candidate mutant lines. It should be noted that other site-directed nucleases can be used to generate sequence-specific breaks to mediate sequence replacement. Also, other DNA, RNA or protein delivery method can be used to deliver components of the editing machinery and donor repair molecules to achieve editing of soybean transporter genes to make them more efficient in transporting silicon.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All patents, patent applications and publications mentioned in this specification are herein incorporated by reference to the same extent as if each independent patent, patent application or publication was specifically and individually indicated to be incorporated by reference.

### REFERENCES

Arsenault-Labrecque, G., Menzies, J. G., & Belanger, R. R. (2012). Effect of silicon absorption on soybean resistance to Phakopsora pachyrhizi in different cultivars. Plant Disease, 96(1), 37-42.
Belanger R R, Benhamou N, Menzies G, 2003. Cytological evidence of an active role of silicon in wheat resistance to powdery mildew (Blumeria gratninis f. sp, tritici). Phytopathology 93(4), 402-412.
Bowen P, Menzies J, Ehret D, et al., 1992. Soluble silicon sprays inhibit powdery mildew development on grape leaves. Journal of the American Society for Horticultural Science 117(6), 906-912.
Bradbury P J, Zhang Z, Kroon D E, et a., 2007. TASSEL: software for association mapping of complex traits in diverse samples. Bioinformatics 23(19), 2633-2635.
Chen W, Yao X, Cai K, Chen J, 2011. Silicon alleviates drought stress of rice plants by improving plant water status, photosynthesis and mineral nutrient absorption. Biological trace element research 142, 67-76.
Cingolani P, Platts A, Coon M, et al., 2012. A program for annotating and predicting the effects of single nucleotide polymorphisms, SnpEff: SNPs in the genome of Drosophila melanogaster strain w1118; iso-2; iso-3. Fly 6, 80-92.
Deshmukh R K, Vivancos J, Guérin V, et al., 2013. Identification and functional characterization of silicon transporters in soybean using comparative genomics of major intrinsic proteins in Arabidopsis and rice. Plant Molecular Biology 83, 303-15.
Elshire R J, Glaubitz J C, Sun Q, et al., 2011. A robust, simple genotyping-by-sequencing (GBS) approach for high diversity species. PLOS ONE 6, e19379.
Epstein E, 1994. The anomaly of silicon in plant biology. Proceedings of the National Academy of Sciences 91(1), 11-17.
Epstein E, 2009. Silicon: its manifold roles in plants. Annals of Applied Biology 155, 155-60.
Fawe A, Menzies J G, Cherif M, et al., 2001. Silicon and disease resistance in dicotyledons. Studies in Plant Science 8, 159-169.
Feng Z, Zhang B, Ding W, et al., 2013. Efficient genorne editing in plants using a CRISPR/Cas system. Cell research 23(10), 1229-1232.
Goodstein D M, Shu S, Howson R, et al., 2012. Phytozome: a comparative platform for green plant genomics. Nucleic Acids Research 40, D1178-D86.
Gu H-H, Qiu H, Tian T, etal., 2011. Mitigation effects of silicon rich amendments on heavy metal accumulation in rice (Oryza sativa L.) planted on multi-metal contaminated acidic soil. Chemosphere 83, 1234-40,
Guérin V, Lebreton A, Cogliati E E, et al., 2014. A zoospore inoculation method with Phytophthora sojae to assess the prophylactic role of silicon on soybean cultivars. Plant Disease, doi: dx.doi.org/10.1094/PDIS-01-14-0102-RE.
Hodson M, White P, Mead A, Broadley M, 2005. Phylogenetic variation in the silicon composition of plants. Annals of Botany 96, 1027-46.
Imsande J & Ralston E J, 1981, Hydroponic Growth and the Nondestructive Assay for Dinitrogen Fixation, Plant Physiol. 68, 1380-1384.
Joung J K & Sander J D, 2013. TALENs: a widely applicable technology for targeted genome editing. Nature Reviews Molecular Cell Biology 14(1), 49-55.
Kang H M, Zaitlen N A, Wade C M, et al., 2008. Efficient control of population structure in model organism association mapping. Genetics 178(3), 1709-1723.
Lipka A E, Tian F, Wang Q, etal., 2012. GAPIT: genome association and prediction integrated tool. Bioinformatics 28(18), 2397-2399.
Loiselle B A, Sork Nason J, etal., 1995. Spatial genetic structure of a tropical understory shrub, Psychotria officinalis (Rubiaceae). American Journal of Botany 1420-1425.
Ma J F, Tamai K, Yamaji N, et al., 2006. A silicon transporter in rice. Nature 440(7084), 688-691.
Ma J F, Yamaji N, Mitani N, et al., 2007. An efflux transporter of silicon in rice. Nature 448, 209-12.
Ma J F, Yamaji N, 2006. Silicon uptake and accumulation in higher plants. Trends in plant science 11(8), 392-397.
Menzies J, Bowen P, Ehret D, et al., 1992. Foliar applications of potassium silicate reduce severity of powdery mildew on cucumber, muskmelon, and zucchini squash. Journal of the American Society for Horticultural Science 117(6), 902-905.
Mitani N, Chiba Y, Yamaji N, Ma J F, 2009. Identification and characterization of maize and barley Lsi2-like silicon efflux transporters reveals a distinct silicon uptake system from that in rice. The Plant Cell Online 21, 2133-42.
Montpetit J, Vivancos J, Mitani-Deno N, et al., 2012. Cloning, functional characterization and heterologous expression of TaLsi1, a wheat silicon transporter gene. Plant Molecular Biology 79, 35-46.
Reidinger S, Ramsey M H, Hartley S E, 2012. Rapid and accurate analyses of silicon and phosphorus in plants using a portable X-ray fluorescence spectrometer. New Phytologist 195(3), 699-706.
Rodrigues F A, Vale F X, Datnoff L E, et al., 2003. Effect of rice growth stages and silicon on sheath blight development. Phytopathology 93(3), 256-261.
Sangster A G, Hodson M J, Tubb H J, 2001. Silicon deposition in higher plants. Studies in Plant Science 8, 85-113.
Scheet P, Stephens M, 2006. A fast and flexible statistical model for large-scale population genotype data: applications to inferring missing genotypes and haplotypic phase. The American Journal of Human Genetics 78, 629-44.
Sonah H; O'Donoughue L, Cober E, et al., 2014. Identification of Loci Governing Eight Agronomic Traits Using a GBS-G WAS Approach and Validation by QTL Mapping in Soybean. Plant Biotechnology Journal, In press.
Sonah H, Bastien M, Iquira E, et al., 2013. An improved genotyping by sequencing (GBS) approach offering increased versatility and efficiency of SNP discovery and genotyping. PLOS ONE 8, e54603.
Tuna A L, Kaya C, Higgs D, Murillo-Amador B, Aydemir S, Girgin A R, 2008. Silicon improves salinity tolerance in wheat plants. Environmental and experimental botany 62, 10-6.
Xiaoyu D, Min T, Weishuang W, 2013. Effects of silicon on physiology and biochemistry of Dendrobiurn moniliforme plantlets under cold stress. Agricultural Biotechnology 2, 18-21.

1-161. (canceled)

162. An elite HiSil Glycine max plant, wherein said elite HiSil Glycine max plant comprises in its genome a chromosomal interval comprising a H1 haplotype.

163. The elite HiSil Glycine max plant of claim 162, wherein said chromosomal interval corresponds to a genomic region or portion thereof from Hikmok sorip chromosome 16 at about 92.6 cM to about 132 cM distance as indicated on a genetic linkage map from Hikmok sorip (PI372415A).

164. The elite HiSil Glycine max plant of claim 162, wherein said chromosomal interval corresponds to a genomic region or portion thereof from Hikmok sorip chromosome 16 corresponding to physical positions 31.15M base-pairs to 36.72 M base-pairs of Williams82 reference genome.

165. The elite HiSil mcix plant of claim 162, wherein the elite Glycine max is a commercially elite Glycine max variety having a commercially significant yield.

166. The plant of claim 162, wherein the chromosomal interval comprises any one of, or a portion of nucleotide base pairs corresponding to positions: 1-2658341 of SEQ NO:1; 567613-569933 of SEQ ID NO:1; 564:321-567612 of SEQ ID NO:1 577172-579696 of SEQ ID NO:1; or 573723-577171 of SEQ NO:1.

167. The plant of claim 162, wherein said plant has increased Si accumulation in any one of the plant leaves, plant stem or plant parts as compared to a LoSil plant.

168. The plant of claim 167, wherein said plant has at least 1.2×, 1.5×, 2×, 3× or higher Si accumulation compared to a LoSil plant.

169. The plant of claim 167, wherein said plant comprises a Si concentration of at least about 1% Si concentration in leaf when said plant is provided with a supply of Si at a concentration of about 0.8 mM, under hydroponic conditions,

170. The plant of claim 162, wherein the chromosomal interval is derived from any one of the plant lines selected from the group consisting of: PI372415A, PI209332, PI404166, PI437655, PI89772, PI372415A or PI90763.

171. The plant of claim 162, wherein said chromosomal interval comprises SEQ ID NOs:14 or 16 or a portion thereof providing increased silicon uptake in a Glycine max plant.

172. The plant of claim 162, wherein said plant comprises a molecular marker associated with increased Si uptake capable of being amplified and identified with the following primer sequences: SEQ ID NOs:2, 3, 4, 5, 6, 7, 8, 9, 1.0, and 11.

173. The plant of claim 162, wherein said plant comprises a marker capable being amplified and identified with the following primer sequences: SEQ NOs:12 and 13.

174. The plant of claim 172, wherein said molecular marker is located within HiSil region genes, as defined by an nucleic acid selected from, the group consisting of: A(33673022), G(33673483), C(33681630), T(33682500), G(33683047), and C(33683049) of genes Glyma30000 or 30020.

175. The plant of claim 162, wherein said chromosomal interval comprises a nucleic acid encoding a polypeptide with an amino acid sequence comprising SEQ ID NO:15 and, further wherein the polypeptide comprises at least one amino acid corresponding to a proline at position 5, a isoleucine at position 295 or a valine at position 439.

176. The plant of claim 162, wherein, said chromosomal interval comprises a nucleic acid encoding a polypeptide with an amino acid sequence comprising SEQ ID NO:17 further, wherein the polypeptide comprises at least one amino acid corresponding to a histidine at position 322 or a glycine at position 431

177. A plant cell, plant seed or plant part derived from the plant of claim 162.

178. An agronomically elite Glycine max plant capable of accumulating Si in leaf tissue at a concentration of at least 1% Si concentration when plants are provided with a supply of Si at a concentration of about 0.8 mM under hydroponic conditions, wherein the Glycine max comprises a genomic region comprising any one of SEQ ID NOs:14 and 16.

179. The plant of claim 178, wherein said plant has a leaf Si concentration of at least around one point two (1.2×), one and a half (1.5×), double (2×), or triple (3×) the concentration of a control plant not comprising said genomic region.

180. A plant of a soybean variety or lineage having high Si uptake, provided that said variety is not Hikmok sorip.

181. The plant of claim 180, wherein the soybean variety or lineage comprises in its genome a chromosomal interval comprising SEQ ID NOs:14 or 16 wherein said chromosomal interval is derived from Hikmok sorip.
